(19) United States
(12) Patent Application Publication
(10) Pub. No.: US 2022/0169739 A1
(43) Pub. Date: Jun. 2, 2022

(54) METHODS FOR TREATING ASTHMA IN PEDIATRIC SUBJECTS BY ADMINISTERING AN IL-4R ANTAGONIST

(71) Applicants: SANOFI BIOTECHNOLOGY, Paris (FR); REGENERON PHARMACEUTICALS, INC., Tarrytown, NY (US)

(72) Inventors: Christine Xu, Bridgewater, NJ (US); Bolanle Akinlade, Tarrytown, NY (US); Nikhil Amin, Tarrytown, NY (US); Marcella Ruddy, Tarrytown, NY (US)

(21) Appl. No.: 17/493,101

(22) Filed: Oct. 4, 2021

Related U.S. Application Data
(60) Provisional application No. 63/157,922, filed on Mar. 8, 2021.
(60) Provisional application No. 63/144,048, filed on Feb. 1, 2021.
(60) Provisional application No. 63/109,719, filed on Nov. 4, 2020.
(60) Provisional application No. 63/087,668, filed on Oct. 5, 2020.

(30) Foreign Application Priority Data
Aug. 31, 2021 (EP) 21315151.7

(51) Int. Cl. C07K 16/28; A61P 11/06

(52) U.S. Cl. CPC C07K 16/2866; A61K 2039/545; A61P 11/06

(57) ABSTRACT

Methods for treating or preventing asthma in a pediatric subject are provided. Methods comprising administering to a pediatric subject in need thereof a therapeutic composition comprising an interleukin-4 receptor (IL-4R) antagonist, such as an anti-IL-4R antibody or antigen-binding fragment thereof, are provided.

### RELATED APPLICATIONS

This application claims priority to U.S. Provisional Patent Application Ser. No. 63/087,668, filed Oct. 5, 2020, 63/109,719, filed Nov. 4, 2020, 63/144,048, filed Feb. 1, 2021, and 63/157,922, filed Mar. 8, 2021; and to European Patent Application No. 21315151.7, filed Aug. 31, 2021. The entire disclosure of each of these applications is hereby incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 4, 2021, is named 721840_SA9-303_SL.txt and is 199,049 bytes in size.

### FIELD OF THE INVENTION

The invention relates to the treatment and/or prevention of asthma in a pediatric subject in need thereof. The invention relates to the administration of an interleukin-4 receptor (IL-4R) antagonist to treat or prevent asthma, in a pediatric subject in need thereof.

### BACKGROUND

Asthma is a chronic inflammatory disease of the airways characterized by airway hyperresponsiveness, acute and chronic bronchoconstriction, airway edema and mucus plugging. The inflammation component of asthma is thought to involve many cell types, including mast cells, eosinophils, T lymphocytes, neutrophils, epithelial cells, and their biological products. Patients with asthma most often present with symptoms of wheezing, shortness of breath, cough, and chest tightness.

A majority of children with asthma have mild or moderate disease and can obtain adequate asthma control through avoidance of triggering factors and/or with the help of medications, such as short-acting inhaled β2-receptor agonists, inhaled corticosteroids (ICS) and, when needed, addition of long-acting β2-receptor agonists (LABA) and leukotriene receptor antagonists (LTRA). However, 2-5% of all asthmatic children have uncontrolled asthma despite maximum treatment with conventional medications. Children with such severe symptoms are heterogeneous with respect to trigger factors, pulmonary function, inflammatory pattern and clinical symptoms. These children have a reduced quality of life, account for a large proportion of the healthcare costs related to asthma, and represent a continuous clinical challenge to the pediatrician.

Long-term adverse effects of systemic and inhaled corticosteroids on bone metabolism, adrenal function, and growth in children has led to attempts to minimize the amount of corticosteroid usage. Further, the consequences of unresponsiveness to therapy or lack of compliance with therapy are evidenced by loss of asthma control (LOAC), which can be severe (i.e., severe asthma exacerbation event) and possibly life-threatening.

Despite standard-of-care therapy such as inhaled corticosteroids, children with uncontrolled moderate-to-severe asthma continue to experience symptoms such as coughing, wheezing, and difficulty breathing, and are at risk of severe asthma attacks. The majority of children with asthma suffer from Type 2 asthma, which often means frequent hospitalizations and emergency room visits. Uncontrolled asthma can cause children to miss school, and can interfere with physical activity and routine tasks including playing sports and walking up stairs.

In addition, the pharmacokinetics of many drugs are different in children compared to adults. The pharmacokinetic processes of absorption, distribution, metabolism and excretion undergo changes due to growth and development. Therefore, finding the correct doses and regimens for children is complicated, and children's doses cannot always be extrapolated directly from adult studies, while maintaining the required efficacy for treatment.

Accordingly, a need exists for novel therapies to treat asthma in children.

### BRIEF SUMMARY OF THE INVENTION

According to one aspect, method for treating asthma in a subject aged 6 years old and older, wherein the subject has moderate-to-severe asthma with type 2 inflammation characterized by an eosinophilic phenotype and/or elevated fraction of exhaled nitric oxide (FeNO), or wherein the subject has oral corticosteroid-dependent asthma, is provided. The method includes administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the antibody or antigen-binding fragment thereof is administered to the subject as an initial dose followed by one or more secondary doses. In certain exemplary embodiments, the initial dose is about 100 mg and each secondary dose is about 100 mg. In certain exemplary embodiments, the subject weighs 15 kg to less than 30 kg.

In certain exemplary embodiments, the initial dose is about 200 mg and each secondary dose is about 200 mg. In certain exemplary embodiments, wherein the subject weighs equal to or greater than 30 kg.

In certain exemplary embodiments, the antibody or antigen-binding fragment thereof is administered to the subject once every other week (q2w).

In certain exemplary embodiments, the initial dose is about 300 mg and each secondary dose is about 300 mg. In certain exemplary embodiments, the subject weighs 15 kg to less than 30 kg. In certain exemplary embodiments, the antibody or antigen-binding fragment thereof is administered to the subject once every four weeks (q4w).

In certain exemplary embodiments, the subject is less than 12 years old.

In certain exemplary embodiments, the FeNO level is ≥20 ppb or the FeNO level is ≥25 ppb.

In certain exemplary embodiments, the subject has asthma with a Type 2 inflammatory phenotype that includes one or both of a baseline blood eosinophil count of greater than or equal to 150 cells/4, and a baseline FeNO of greater than or equal to 20 ppb. In certain exemplary embodiments, the subject has asthma with a Type 2 inflammatory phenotype that includes one or both of a baseline blood eosinophil count of greater than or equal to 150 cells/4, and a baseline FeNO of greater than or equal to 20 ppb.

In certain exemplary embodiments, the antibody or antigen-binding fragment thereof is administered using an autoinjector, a needle and syringe, or a pen. In certain exemplary embodiments, the antibody or antigen-binding fragment thereof is administered subcutaneously.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in an improvement in lung function as measured by forced expiratory volume (FEV1), by forced vital capacity (FVC), by forced expiratory flow at 25-75% of the pulmonary volume (FEF25-75%), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication.

In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine.

In certain exemplary embodiments, the ICS is administered at high dose or at a medium dose.

In certain exemplary embodiments, the subject has a comorbid Type 2 inflammatory condition in addition to asthma. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has a baseline total serum IgE≥30 IU/mL. In certain exemplary embodiments, the subject has a baseline allergen-specific IgE≥0.35 kU/L for at least one aeroallergen. In certain exemplary embodiments, the subject has a baseline total serum IgE≥30 IU/mL, and a baseline allergen-specific IgE≥0.35 kU/L for at least one aeroallergen.

In certain exemplary embodiments, the antibody or antigen-binding fragment thereof is administered to the subject once every other week (q2w).

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the ICS is administered at high dose or at a medium dose.

In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the subject has allergic asthma.

According to another aspect, a method for treating asthma in a subject aged 6 to 11 years old, wherein the subject has severe asthma with type 2 inflammation characterized by raised blood eosinophils and/or raised fraction of exhaled nitric oxide (FeNO), is provided. The method includes administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the antibody or an antigen-binding fragment thereof is administered as add-on maintenance treatment, and wherein the subject is inadequately controlled with medium to high dose inhaled corticosteroid (ICS) plus another medicinal product for the maintenance treatment.

In certain exemplary embodiments, the antibody or antigen-binding fragment thereof is administered to the subject as an initial dose followed by one or more secondary doses. In certain exemplary embodiments, the initial dose is about 100 mg and each secondary dose is about 100 mg. In certain exemplary embodiments, the subject weighs 15 kg to less than 30 kg.

In certain exemplary embodiments, the initial dose is about 200 mg and each secondary dose is about 200 mg. In certain exemplary embodiments, the subject weighs 30 kg to less than 60 kg. In certain exemplary embodiments, the subject weighs 60 kg or more.

In certain exemplary embodiments, the antibody or antigen-binding fragment thereof is administered to the subject once every other week (q2w).

In certain exemplary embodiments, the initial dose is about 300 mg and each secondary dose is about 300 mg. In certain exemplary embodiments, the subject weighs 15 kg to less than 30 kg. In certain exemplary embodiments, the subject weighs 30 kg to less than 60 kg. In certain exemplary embodiments, the antibody or antigen-binding fragment thereof is administered to the subject once every four weeks (q4w).

In certain exemplary embodiments, the FeNO level is ≥20 ppb or the FeNO level is ≥25 ppb.

In certain exemplary embodiments, the blood eosinophil level is greater than or equal to 150 cells/μL or the blood eosinophil level is greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb. In certain exemplary embodiments, the subject has asthma with an eosinophilic phenotype that includes a baseline blood eosinophil count of greater than or equal to 300 cells/μL.

In certain exemplary embodiments, the antibody or antigen-binding fragment thereof is administered using an autoinjector, a needle and syringe, or a pen. In certain exemplary embodiments, the antibody or antigen-binding fragment thereof is administered subcutaneously.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in an improvement in lung function as measured by forced expiratory volume (FEV1), by forced vital capacity (FVC), by forced expiratory flow at 25-75% of the pulmonary volume (FEF25-75%), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the ICS is administered at high dose or at a medium dose.

In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine.

In certain exemplary embodiments, the subject has a comorbid Type 2 inflammatory condition in addition to asthma.

In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has a baseline total serum IgE≥30 IU/mL. In certain exemplary embodiments, the subject has a baseline allergen-specific IgE≥0.35 kU/L for at least one aeroallergen. In certain exemplary embodiments, the subject has a baseline total serum IgE≥30 IU/mL, and a baseline allergen-specific IgE≥0.35 kU/L for at least one aeroallergen.

In certain exemplary embodiments, the antibody or antigen-binding fragment thereof is administered to the subject once every other week (q2w).

In certain exemplary embodiments, the treatment results in an improvement in one or any combination of antigen-specific IgE, antigen-specific IgG4, and antigen-specific IgE/IgG4 ratio. In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV1), by forced vital capacity (FVC), by forced expiratory flow at 25-75% of the pulmonary volume (FEF25-75%), by morning peak expiratory flow (AM PEF), by evening peak expiratory flow (PM PEF) or any combination thereof.

In certain exemplary embodiments, wherein the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication.

In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine.

In certain exemplary embodiments, the ICS is administered at high dose or at a medium dose.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2.

In certain exemplary embodiments, the antibody is dupilumab.

In certain exemplary embodiments, the subject has allergic asthma.

According to another aspect, a method for treating a pediatric subject having asthma is provided. The method includes administering to the pediatric subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

In certain exemplary embodiments, the subject has a body weight of greater than 30 kg and the antibody or antigen-binding fragment thereof is administered to the subject at a dose of about 200 mg. In certain exemplary embodiments, the subject has a body weight of 30 kg or less and the antibody or antigen-binding fragment thereof is administered to the subject at a dose of about 100 mg.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject having asthma comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of greater than 30 kg and the antibody or antigen-binding fragment thereof is administered to the subject at a dose of about 200 mg, is provided.

In certain exemplary embodiments, the antibody or antigen-binding fragment thereof is administered to the subject as an initial dose followed by one or more secondary doses. In certain exemplary embodiments, the initial dose is about 200 mg and each secondary dose is about 200 mg.

In certain exemplary embodiments, the subject is 6 years old to less than 12 years old.

In certain exemplary embodiments, the asthma is uncontrolled persistent asthma or uncontrolled moderate-to-severe asthma.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject having asthma comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of 30 kg or less and the antibody or antigen-binding fragment thereof is administered to the subject at a dose of about 100 mg, is provided.

In certain exemplary embodiments, the antibody or antigen-binding fragment thereof is administered to the subject as an initial dose followed by one or more secondary doses. In certain exemplary embodiments, the initial dose is about 100 mg and each secondary dose is about 100 mg.

In certain exemplary embodiments, the subject is 6 years old to less than 12 years old.

In certain exemplary embodiments, the subject has a body weight of at least 16 kg.

In certain exemplary embodiments, the asthma is uncontrolled persistent asthma or uncontrolled moderate-to-severe asthma.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having uncontrolled moderate-to-severe asthma comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of greater than 30 kg and the antibody or antigen-binding fragment thereof is administered to the subject at a dose of about 200 mg, is provided.

In certain exemplary embodiments, the antibody or antigen-binding fragment thereof is administered to the subject as an initial dose followed by one or more secondary doses. In certain exemplary embodiments, the initial dose is about 200 mg and each secondary dose is about 200 mg.

In certain exemplary embodiments, the subject has asthma with an eosinophilic phenotype that includes a baseline blood eosinophil count of greater than or equal to 300 cells/μL.

In certain exemplary embodiments, the subject has a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having uncontrolled moderate-to-severe asthma comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of 30 kg or less and the antibody or antigen-binding fragment thereof is administered to the subject at a dose of about 100 mg, is provided.

In certain exemplary embodiments, the antibody or antigen-binding fragment thereof is administered to the subject as an initial dose followed by one or more secondary doses.

In certain exemplary embodiments, the initial dose is about 100 mg and each secondary dose is about 100 mg.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for reducing or eliminating a subject's dependence on systemic corticosteroids (SCS), wherein the subject is aged 6 years old to less than 12 years old and has uncontrolled moderate-to-severe asthma, wherein the subject has a body weight of greater than 30 kg, is provided.

In certain exemplary embodiments, the method comprises administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the antibody or antigen-binding fragment thereof is administered to the subject at a dose of about 200 mg or about 300 mg, and wherein the dosage of SCS administered to the subject is gradually reduced or eliminated over the course of a treatment period.

In certain exemplary embodiments, the antibody or antigen-binding fragment thereof is administered to the subject as an initial dose followed by one or more secondary doses.

In certain exemplary embodiments, the initial dose is about 100 mg and each secondary dose is about 100 mg or about 200 mg. In certain exemplary embodiments, the initial dose is about 200 mg and each secondary dose is about 100 mg or about 200 mg.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w) at a dose of about 200 mg.

In certain exemplary embodiments, the antibody or antigen-binding fragment thereof is administered to the subject once every four week (q4w) at a dose of about 300 mg.

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for reducing or eliminating a subject's dependence on systemic corticosteroids (SCS), wherein the subject is aged 6 years old to less than 12 years old and has uncontrolled moderate-to-severe asthma, and wherein the subject has a body weight of 30 kg or less, is provided.

In certain exemplary embodiments, the method comprises administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the antibody or antigen-binding fragment thereof is administered to the subject at a dose of about 100 mg or about 300 mg, and wherein the dosage of SCS administered to the subject is gradually reduced or eliminated over the course of a treatment period.

In certain exemplary embodiments, the antibody or antigen-binding fragment thereof is administered to the subject as an initial dose followed by one or more secondary doses.

In certain exemplary embodiments, the initial dose is about 100 mg and each secondary dose is about 100 mg or about 200 mg. In certain exemplary embodiments, the initial dose is about 200 mg and each secondary dose is about 100 mg or about 200 mg.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w) at a dose of about 100 mg.

In certain exemplary embodiments, the antibody or antigen-binding fragment thereof is administered to the subject once every four week (q4w) at a dose of about 300 mg.

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for decreasing an asthma exacerbation rate in a subject, wherein the subject is aged 6 years old to less than 12 years old and has uncontrolled moderate-to-severe asthma, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of greater than 30 kg and the antibody or antigen-binding fragment thereof is administered to the subject at a dose of about 200 mg or about 300 mg, is provided.

In certain exemplary embodiments, the antibody or antigen-binding fragment thereof is administered to the subject as an initial dose followed by one or more secondary doses.

In certain exemplary embodiments, the initial dose is about 100 mg and each secondary dose is about 100 mg or about 200 mg. In certain exemplary embodiments, the initial dose is about 200 mg and each secondary dose is about 100 mg or about 200 mg.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w) at a dose of about 200 mg.

In certain exemplary embodiments, the antibody or antigen-binding fragment thereof is administered to the subject once every four weeks (q4w) at a dose of about 300 mg.

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for decreasing an asthma exacerbation rate in a subject, wherein the subject is aged 6 years old to less than 12 years old and has uncontrolled moderate-to-severe asthma, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of 30 kg or less and the antibody or antigen-binding fragment thereof is administered to the subject at a dose of about 100 mg or about 300 mg, is provided.

In certain exemplary embodiments, the antibody or antigen-binding fragment thereof is administered to the subject as an initial dose followed by one or more secondary doses.

In certain exemplary embodiments, the initial dose is about 100 mg and each secondary dose is about 100 mg or about 200 mg. In certain exemplary embodiments, the initial dose is about 200 mg and each secondary dose is about 100 mg or about 200 mg.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w) at a dose of about 100 mg.

In certain exemplary embodiments, the antibody or antigen-binding fragment thereof is administered to the subject once every four weeks (q4w) at a dose of about 300 mg.

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having asthma, comprising administering to the subject one or more doses of an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the antibody or antigen-binding fragment thereof is administered to the subject as an initial dose followed by one or more secondary doses, is provided.

In certain exemplary embodiments, each secondary dose is administered 1 to 4 weeks after the immediately preceding dose, and wherein: for a subject having a body weight of ≤30 kg, the initial dose of the antibody or antigen-binding fragment thereof is 100 mg and each secondary dose is 100 mg; or (ii) for a subject having a body weight of >30 kg, the initial dose of the antibody or antigen-binding fragment thereof is 200 mg and each secondary dose is 200 mg.

In certain exemplary embodiments, the subject has uncontrolled moderate-to-severe asthma or uncontrolled persistent asthma.

In certain exemplary embodiments, the subject has asthma with an eosinophilic phenotype that includes a baseline blood eosinophil count of greater than or equal to 300 cells/μL.

In certain exemplary embodiments, the subject has asthma with a Type 2 inflammatory phenotype that includes one or both of a baseline blood eosinophil count of greater than or equal to 150 cells/μL and a baseline FeNO of greater than or equal to 20 ppb.

In certain exemplary embodiments, the antibody or antigen-binding fragment thereof is administered to the subject once every other week (q2w).

In certain exemplary embodiments, a first maintenance dose of antibody or antigen-binding fragment thereof is administered two weeks after an initial dose of antibody or antigen-binding fragment thereof. In certain exemplary embodiments, the maintenance doses of the antibody or antigen-binding fragment thereof are administered for at least 24 weeks.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having asthma, comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, and wherein the subject has a body weight of 30 kg or less, wherein the antibody or antigen-binding fragment thereof is administered to the subject at a dose of about 300 mg every four weeks (q4w), is provided.

In certain exemplary embodiments, the subject aged 6 years old to less than 12 years old has an uncontrolled moderate-to-severe asthma or uncontrolled persistent asthma.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having asthma, comprising administering to the subject an antibody or an antigen-binding fragment thereof that specifically binds interleukin-4 receptor (IL-4R), and wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the antibody or antigen-binding fragment thereof is administered to the subject at a dose of about 300 mg every four weeks (q4w) regardless of body weight, is provided.

In certain exemplary embodiments, the subject aged 6 years old to less than 12 years old has an uncontrolled moderate-to-severe asthma or uncontrolled persistent asthma.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the subject has allergic asthma. In certain exemplary embodiments, the subject has a baseline total serum IgE≥30 IU/mL and/or a baseline allergen-specific IgE≥0.35 kU/L for at least one aeroallergen.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

According to another aspect, a method for treating a subject aged 6 years old to less than 12 years old having asthma, comprising administering to the subject an antibody or an antigen-binding fragment thereof that specifically binds interleukin-4 receptor (IL-4R), and wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively, wherein the antibody or antigen-binding fragment thereof is administered to the subject at an initial loading dose of about 300 mg, and one or more maintenance doses of about 300 mg every four weeks (q4w), wherein a first maintenance dose is administered to the subject two weeks after the initial loading dose, is provided.

In certain exemplary embodiments, the subject aged 6 years old to less than 12 years old has an uncontrolled moderate-to-severe asthma or uncontrolled persistent asthma.

In certain exemplary embodiments, the antibody or antigen-binding fragment thereof is administered using an autoinjector, a needle and syringe, or a pen. In certain exemplary embodiments, the antibody or antigen-binding fragment thereof is administered using a prefilled device. In certain exemplary embodiments, the prefilled device is a prefilled syringe comprising the antibody or antigen-binding fragment thereof at a concentration of 150 mg/mL. In certain exemplary embodiments, the prefilled device is a prefilled syringe comprising the antibody or antigen-binding fragment thereof at a concentration of 175 mg/mL. In certain exemplary embodiments, the antibody or antigen-binding fragment thereof is administered subcutaneously.

In certain exemplary embodiments, the treatment results in an improvement in at least one biomarker level, wherein the at least one biomarker is selected from the group consisting of fractional exhaled nitric oxide (FeNO), thymus and activation regulated chemokine (TARC), urinary leukotriene E4 (LTE4), interleukin 5 (IL-5), and serum total IgE.

In certain exemplary embodiments, the treatment results in an improvement in one or any combination of antigen-specific IgE, antigen-specific IgG4, and antigen-specific IgE/IgG4 ratio.

In certain exemplary embodiments, the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

In certain exemplary embodiments, the treatment results in an improvement of slope of % predicted FEV1.

In certain exemplary embodiments, the treatment results in a reduction in annualized severe asthma exacerbations selected from: (a) a deterioration of asthma requiring use of systemic corticosteroids for at least three days and/or hospitalization or emergency room visit requiring systemic corticosteroids; and (b) loss of asthma control (LOAC) event defined by: (i) ≥6 additional reliever puffs of salbutamol/albuterol or levosalbutamol/levalbuterol in a 24 hour period on two consecutive days; (ii) an increase in ICS dose ≥4 times than a previous dose; (iii) a decrease in AM or PM peak flow of 30% or more on 2 consecutive days of treatment, based on the defined stability limit; or (iv) a severe exacerbation event.

In certain exemplary embodiments, the treatment results in an improvement in lung function as measured by forced expiratory volume (FEV₁), by forced vital capacity (FVC), by forced expiratory flow at 25-75% of the pulmonary volume (FEF_{25-75%}), by morning peak expiratory flow (AM PEF), by evening peak expiratory flow (PM PEF) or any combination thereof.

In certain exemplary embodiments, the subject is administered a background therapy selected from the group consisting of: a TNF inhibitor, an IL-1 inhibitor, an IL-5 inhibitor, an IL-8 inhibitor, an IgE inhibitor, a leukotriene inhibitor, a corticosteroid, a methylxanthine, an NSAID, nedocromil sodium, cromolyn sodium, a long-acting beta2 agonist and an anti-fungal agent or any combinations thereof.

In certain exemplary embodiments, the subject is administered a background therapy comprising inhaled corticosteroid (ICS) optionally in combination with a second controller medication. In certain exemplary embodiments, the second controller medication is selected from the group consisting of a long-acting β2 agonist (LABA), a leukotriene receptor antagonist (LTRA), a long-acting muscarinic antagonist (LAMA), and a methylxanthine. In certain exemplary embodiments, the ICS is administered at high dose or at a medium dose.

In certain exemplary embodiments, the subject has a comorbid Type 2 inflammatory condition in addition to asthma. In certain exemplary embodiments, the comorbid Type 2 inflammatory condition is selected from the group consisting of atopic dermatitis, allergic conjunctivitis, allergic rhinitis, eosinophilic esophagitis, food allergy, hives and any combination thereof.

In certain exemplary embodiments, the antibody or antigen-binding fragment thereof comprises a heavy chain variable region (HCVR) sequence of SEQ ID NO: 1 and a light chain variable region (LCVR) sequence of SEQ ID NO: 2. In certain exemplary embodiments, the antibody is dupilumab.

Other embodiments will become apparent from a review of the ensuing detailed description, drawings, tables and accompanying claims.

### BRIEF DESCRIPTION OF THE FIGURES

The foregoing and other features and advantages of the present invention will be more fully understood from the following detailed description of illustrative embodiments taken in conjunction with the accompanying drawings. The file of this patent contains at least one drawing/photograph executed in color. Copies of this patent with color drawing(s)/photograph(s) will be provided by the Office upon request and payment of the necessary fee.

FIG. 1 graphically depicts the overview of study design. Background medication: medium dose ICS and second controller; or high dose ICS and second controller. D: day; EOT: end of treatment; EOS: end of study; ICS, inhaled corticosteroids; q2w: every 2 weeks; R: randomization; SC, subcutaneous.

FIG. 2 depicts a study flow chart. AE: adverse event; AESI: adverse events of special interest; EQ-5D-Y: EuroQol 5-dimensions questionnaire for children; ETD: early treatment discontinuation visit; FEV1: forced expiratory volume in 1 second; HRQol: health-related quality of life; IgA: Immunoglobulin A; IgE: IgG: Immunoglobulin E; Immunoglobulin G; IgM: Immunoglobulin M; IVRS: Interactive voice response system; IWRS: Interactive web response system, NO: Nitric oxide; ACQ-IA: Asthma Control Questionnaire-Interviewer Administered; PACQLQ: Pediatric Asthma Caregivers Quality of Life Questionnaire; PAQLQ(S)-IA: Pediatric Asthma Quality of Life Questionnaire with Standardised Activities-Interviewer Administered; PD: Pharmacodynamics; PK: Pharmacokinetics; PRQLQ-IA: Pediatric Rhinoconjunctivitis Quality of Life Questionnaire-Interviewer Administered; PEF: peak expiratory flow; SAE: serious adverse event.

FIG. 3 depicts a statistical testing hierarchy indicating that all multiplicity-controlled endpoints were met. The primary endpoint is noted in red.

FIG. 4 graphically depicts a reduction in annualized rate of exacerbation among various groups. EOS=eosinophil; FeNO=fractional exhaled nitrous oxide; ITT=intent-to-treat. High FeNO is defined as 20 ppb.

FIG. 5 graphically depicts an improvement in FEV1 percent predicted (pp) across all Type 2 populations, shown as least squares (LS) mean change from baseline at week 12. EOS=eosinophil; FeNO=fractional exhaled nitrous oxide; ITT=intent-to-treat. High FeNO is defined as 20 ppb.

FIG. 6 graphically depicts an improvement in FEV1pp, shown as LS mean change from baseline at over 52 weeks. A rapid (within two weeks) and sustained (over 52 weeks) improvement in lung function was observed in Type 2 inflammatory asthma phenotype (left panel) and in asthma having a baseline blood eosinophil phenotype of greater than or equal to 300 cells/μL.

FIG. 7 depicts a table showing endpoints in subpopulations of asthma subjects defined by markers of Type 2 inflammation.

FIG. 8 depicts a table showing baseline demographics and disease characteristics.

FIG. 9 depicts a table showing concurrent atopic conditions and base.

FIG. 10 graphically depicts a reduction in annualized rate of exacerbation by weight for subpopulations having a Type 2 inflammatory asthma phenotype (defined as EOS≥0.150 Giga/L or FeNO≥20 ppb) or a baseline blood eosinophil level of ≥0.3 Giga/L. q2w, every two weeks.

FIG. 11 graphically depicts time to first severe exacerbation for subpopulations having a Type 2 inflammatory asthma phenotype (defined as EOS≥0.150 Giga/L or FeNO≥20 ppb) or a baseline blood eosinophil level of ≥0.3 Giga/L.

FIG. 12 depicts systemic corticosteroid (SCS) exposure for subpopulations having a Type 2 inflammatory asthma phenotype (defined as EOS≥0.150 Giga/L or FeNO≥20 ppb) or a baseline blood eosinophil level of ≥0.3 Giga/L.

FIG. 13 depicts SCS exposure breakdowns for subpopulations having a Type 2 inflammatory asthma phenotype (defined as EOS≥0.150 Giga/L or FeNO≥20 ppb) or a baseline blood eosinophil level of ≥0.3 Giga/L. SD, standard deviation.

FIG. 14 graphically depicts FEV1pp results as a mean at baseline and at week 12 in various treatment subpopulations and in the ITT population. Type 2 (EU), EOS≥0.3 Giga/L or FeNO, ≥20 ppb; High FeNO, ≥20 ppb.

FIG. 15 graphically depicts FEV1pp results by weight as a least squares mean change from baseline for subpopulations having a Type 2 inflammatory asthma phenotype or a baseline blood eosinophil level of ≥0.3 Giga/L. q2w, every two weeks.

FIG. 16 graphically depicts Asthma Control Questionnaire 7 (ACQ-7) results as a mean from baseline at week 24 in various treatment subpopulations and in the ITT population. Type 2 (EU), EOS≥0.3 Giga/L or FeNO, ≥20 ppb; High FeNO, ≥20 ppb.

FIG. 17 graphically depicts ACQ-7 results as a mean at baseline and at week 24 in various treatment subpopulations and in the ITT population. Type 2 (EU), EOS≥0.3 Giga/L or FeNO, ≥20 ppb; High FeNO, ≥20 ppb.

FIG. 18 graphically depicts ACQ-7, interviewer administered version (ACQ-7-IA), as a least squares mean from baseline for subpopulations having a Type 2 inflammatory asthma phenotype (defined as EOS≥0.150 Giga/L or FeNO≥20 ppb) or a baseline blood eosinophil level of ≥0.3 Giga/L.

FIG. 19 depicts a forest plot showing relative risk in annualized event rate of severe exacerbations in baseline blood eosinophil subpopulations and in an ITT population.

FIG. 20 depicts a forest plot showing relative risk in annualized event rate of severe exacerbations in fractional exhaled nitric oxide (FeNO) subpopulations.

FIG. 21 depicts a forest plot showing relative risk in annualized event rate of severe exacerbations during the 52-week treatment period by baseline for Type 2 inflammatory asthma subpopulations (defined as EOS≥0.150 Giga/L or FeNO≥20 ppb).

FIG. 22 depicts a forest plot showing Eos/FeNO Quadrants. Exacerbations quadrant analysis indicates efficacy in Type 2 inflammatory asthma subpopulations (defined as EOS≥0.150 Giga/L or FeNO≥20 ppb) and no efficacy in non-Type 2 inflammatory asthma subpopulations.

FIG. 23 depicts a forest plot showing a summary of change in baseline in pre-bronchodilator FEV1 (pre-BD FEV1) at week 12 by baseline for Type 2 inflammatory asthma subpopulations (defined as EOS≥0.150 Giga/L or FeNO≥20 ppb) and the ITT population.

FIG. 24 depicts a forest plot of summary of change from baseline in pre-BD percent predicted FEV1 at week 12 by quadrant defined by baseline blood eosinophil and baseline FeNO in the ITT population.

FIG. 25 depicts FEV1pp (mean) at baseline and at week 12.

FIG. 26 depicts FEV1pp as a least squares change from baseline over 52 weeks.

FIG. 27 depicts pre-BD as a least squares change from baseline over 52 weeks.

FIG. 28 depicts post-bronchodilator (post-BD) as a least squares change from baseline over 52 weeks.

FIG. 29 depicts an FEV1pp slope analysis.

FIG. 30 graphically depicts the mean change from baseline in post-BD percent predicted FEV1 over time in a Type 2 inflammatory asthma phenotype subpopulation (defined as EOS≥0.150 Giga/L or FeNO≥20 ppb).

FIG. 31 graphically depicts the mean change from baseline in post-BD percent predicted FEV1 over time in a baseline blood eosinophils ≥0.3 Giga/L subpopulation.

FIG. 32 graphically depicts forced vital capacity (FVC) as a least squares mean change from baseline over 52 weeks.

FIG. 33 graphically depicts forced expiratory flow at 25%-75% of the pulmonary volume (FEF_{25-75%}) as a least squares mean change from baseline over 52 weeks for subpopulations having a Type 2 inflammatory asthma phenotype (defined as EOS≥0.150 Giga/L or FeNO≥20 ppb) or a baseline blood eosinophil level of ≥0.3 Giga/L.

FIG. 34 graphically depicts the least squares mean change from baseline in percent predicted FEF25-75% over time (mixed effect model repeated measures (MMRM) including measurements up to week 52) in a Type 2 inflammatory asthma phenotype subpopulation (defined as EOS≥0.150 Giga/L or FeNO≥20 ppb).

FIG. 35 graphically depicts the least squares mean change from baseline in percent predicted FEF25-75% over time (MMRM including measurements up to week 52) in a baseline blood eosinophil level of ≥0.3 Giga/L subpopulation.

FIG. 36 graphically depicts the least squares mean change from baseline in FEV1/FVC (%) over time (MMRM including measurements up to week 52) in a Type 2 inflammatory asthma phenotype subpopulation (defined as EOS≥0.150 Giga/L or FeNO≥20 ppb).

FIG. 37 graphically depicts the least squares mean change from baseline in FEV1/FVC (%) over time (MMRM including measurements up to week 52) in a baseline blood eosinophil level of ≥0.3 Giga/L subpopulation.

FIG. 38 graphically depicts the least squares mean change from baseline in morning peak expiratory flow (AM PEF) (L/minute) over time (MMRM including measurements up to week 52) in a Type 2 inflammatory asthma phenotype subpopulation (defined as EOS≥0.150 Giga/L or FeNO≥20 ppb).

FIG. 39 graphically depicts the least squares mean change from baseline in morning peak expiratory flow (AM PEF) (L/minute) over time (MMRM including measurements up to week 52) in a baseline blood eosinophil level of ≥0.3 Giga/L subpopulation.

FIG. 40 graphically depicts the least squares mean change from baseline in evening peak expiratory flow (PM PEF) (L/minute) over time (MMRM including measurements up to week 52) in a Type 2 inflammatory asthma phenotype subpopulation (defined as EOS≥0.150 Giga/L or FeNO≥20 ppb).

FIG. 41 graphically depicts the least squares mean change from baseline in evening peak expiratory flow (PM PEF) (L/minute) over time (MMRM including measurements up to week 52) in a baseline blood eosinophil level of ≥0.3 Giga/L subpopulation.

FIG. 42 graphically depicts Pediatric Asthma Quality of Life Questionnaire (PAQLQ) score as least squares mean change from baseline, showing an improvement in quality of life for both a Type 2 inflammatory asthma phenotype subpopulation (defined as EOS≥0.150 Giga/L or FeNO≥20 ppb) and a baseline blood eosinophils ≥0.3 Giga/L subpopulation.

FIG. 43 graphically depicts Pediatric Asthma Caregiver's Quality of Life Questionnaire (PACQLQ) global score over time in a Type 2 inflammatory asthma phenotype subpopulation (defined as EOS≥0.150 Giga/L or FeNO≥20 ppb) (least squares mean change from baseline, MMRM).

FIG. 44 graphically depicts PACQLQ global score over time in a baseline blood eosinophils ≥0.3 Giga/L subpopulation (least squares mean change from baseline, MMRM).

FIG. 45 graphically depicts Pediatric Rhinoconjunctivitis Quality of Life Questionnaire (PRQLQ) global score over time in a Type 2 inflammatory asthma phenotype subpopulation (defined as EOS≥0.150 Giga/L or FeNO≥20 ppb) (least squares mean change from baseline, MMRM).

FIG. 46 graphically depicts PRQLQ global score over time in a baseline blood eosinophils ≥0.3 Giga/L subpopulation (least squares mean change from baseline, MMRM).

FIG. 47 graphically depicts EuroQol EQ-5D-5L single index score over time in a Type 2 inflammatory asthma phenotype subpopulation (defined as EOS≥0.150 Giga/L or FeNO≥20 ppb) (least squares mean change from baseline, MMRM).

FIG. 48 graphically depicts EQ-5D-5L single index score over time in a baseline blood eosinophils ≥0.3 Giga/L subpopulation (least squares mean change from baseline, MMRM).

FIG. 49 graphically depicts AM symptom score (least squares mean change from baseline).

FIG. 50 graphically depicts PM symptom score (least squares mean change from baseline).

FIG. 51 graphically depicts nocturnal awakenings (least squares mean change from baseline).

FIG. 52 graphically depicts reliever medication use (least squares mean change from baseline).

FIG. 53 graphically depicts a marked decrease in IgE levels over 52 weeks in a safety population.

FIG. 54 graphically depicts a sustained decrease in serum thymus and activation-regulated chemokine) TARC levels over 52 weeks in a safety population.

FIG. 55A-FIG. 55B graphically depict estimated annualized event rate of severe exacerbation during 52-week treatment period. (A) By baseline blood eosinophil (Giga/L) based on a penalized regression spline model in an ITT population. (B) By baseline FeNO (ppb) based on a penalized regression spline model in an ITT population.

FIG. 56A-FIG. 56B graphically depict least squares mean change from baseline in pre-bronchodilator % predicted FEV1 at week 12. (A) By baseline blood eosinophil (Giga/L) based on a penalized regression spline model in an ITT population. (B) By baseline FeNO (ppb) based on a penalized regression spline model in an ITT population.

FIG. 57 depicts baseline measures based on age, sex, and race/ethnicity.

### DETAILED DESCRIPTION

Before the invention is described, it is to be understood that this invention is not limited to particular methods and experimental conditions described, as such methods and conditions may vary. It is also to be understood that the terminology used herein is for the purpose of describing particular embodiments only, and is not intended to be limiting, because the scope of the invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

Although any methods and materials similar or equivalent to those described herein can be used in the practice of the invention, the typical methods and materials are now described. All publications mentioned herein are incorporated herein by reference in their entirety.

### Methods for Reducing the Incidence of Asthma Exacerbations

Methods for reducing the incidence of asthma in a subject in need thereof comprising administering a pharmaceutical composition comprising an IL-4R antagonist to the subject are provided. According to certain embodiments, the IL-4R antagonist is an antibody or antigen-binding fragment thereof that specifically binds IL-4R. Exemplary anti-IL-4R antibodies that can be used in the context of the methods featured here are described elsewhere herein.

As used herein, the expression “asthma exacerbation” means an increase in the severity and/or frequency and/or duration of one or more symptoms or indicia of asthma. An “asthma exacerbation” also includes any deterioration in the respiratory health of a subject that requires and or is treatable by a therapeutic intervention for asthma (such as, e.g., steroid treatment, inhaled corticosteroid treatment, hospitalization, etc.). There are two types of asthma exacerbation events: a loss of asthma control (LOAC) event and a severe exacerbation event.

According to certain embodiments, a loss of asthma control (LOAC) event is defined as one or more of the following: (a) greater than or equal to 6 additional reliever puffs of salbutamol/albuterol or levosalbutamol/levalbuterol in a 24 hour period (compared to baseline) on 2 consecutive days; (b) an increase in ICS greater than or equal to 4 times the dose at visit 2; and (c) use of systemic corticosteroids for greater than or equal to 3 days; or (d) hospitalization or emergency room visit because of asthma, requiring systemic corticosteroids.

In certain instances, an asthma exacerbation may be categorized as a “severe asthma exacerbation event.” A severe asthma exacerbation event means an incident requiring immediate intervention in the form of treatment with either systemic corticosteroids or with inhaled corticosteroids at four or more times the dose taken prior to the incident. According to certain embodiments, a severe asthma exacerbation event is defined as a deterioration of asthma requiring: use of systemic corticosteroids for greater than or equal to 3 days; or hospitalization or emergency room visit because of asthma, requiring systemic corticosteroids. The general expression “asthma exacerbation” therefore includes and encompasses the more specific subcategory of “severe asthma exacerbations.” Accordingly, methods for reducing the incidence of severe asthma exacerbations in a patient in need thereof are included.

A “reduction in the incidence” of an asthma exacerbation means that a subject who has received a pharmaceutical composition comprising an IL-4R antagonist experiences fewer asthma exacerbations (i.e., at least one fewer exacerbation) after treatment than before treatment, or experiences no asthma exacerbations for at least 4 weeks (e.g., 4, 6, 8, 12, 14, or more weeks) following initiation of treatment with the pharmaceutical composition. A “reduction in the incidence” of an asthma exacerbation alternatively means that, following administration of the pharmaceutical composition, the likelihood that a subject experiences an asthma exacerbation is decreased by at least 10% (e.g., 10%, 15%, 20%, 25%, 30%, 35%, 40%, 45%, 50%, or more) as compared to a subject who has not received the pharmaceutical composition.

Methods for reducing the incidence of asthma exacerbations in a subject in need thereof comprising administering a pharmaceutical composition comprising an IL-4R antagonist to the subject are provided. In some embodiments, the methods comprise administering a pharmaceutical composition comprising an IL-4R antagonist to the subject as well as administering to the subject one or more maintenance doses of an inhaled corticosteroid (ICS) and/or one or more maintenance doses of a second controller, e.g., a long-acting beta-agonist (LABA) or a leukotriene receptor antagonist (LTA), are provided. Suitable ICSs include, but are not limited to, fluticasone (e.g., fluticasone propionate, e.g., FLOVENT™), budesonide, mometasone (e.g., mometasone furoate, e.g., ASMANEX™), flunisolide (e.g., AEROBID™), dexamethasone acetate/phenobarbital/theophylline (e.g., AZMACORT™), beclomethasone dipropionate HFA (QVAR™), and the like. Suitable LABAs include, but are not limited to, salmeterol (e.g., SEREVENT™), formoterol (e.g., FORADIL™), and the like. Suitable LTAs include, but are not limited to, montelukast (e.g., SINGULAIRE™), zafirlukast (e.g., ACCOLATE™), and the like.

Methods for reducing the incidence of asthma exacerbations in a subject in need thereof comprising administering a pharmaceutical composition comprising an IL-4R antagonist to the subject as well as administering to the subject one or more reliever medications to eliminate or reduce one or more asthma-associated symptoms, are provided. Suitable reliever medications include, but are not limited to, quick-acting beta2-adrenergic receptor agonists such as, e.g., albuterol (i.e., salbutamol, e.g., PROVENTIL™, VENTOLIN™, and the like), levalbuterol (e.g., XOPENEX™ and the like), pirbuterol (e.g., MAXAIR™), metaproterenol (e.g., ALUPENT™) and the like.

### Methods for Improving Asthma-Associated Parameters

Methods for improving one or more asthma-associated parameters in a subject in need thereof, wherein the methods comprise administering a pharmaceutical composition comprising an IL-4R antagonist to the subject, are also provided. A reduction in the incidence of an asthma exacerbation (as described above) may correlate with an improvement in one or more asthma-associated parameters; however, such a correlation is not necessarily observed in all cases.

Examples of “asthma-associated parameters” include: (1) relative percent change from baseline (e.g., at week 12) in forced expiratory volume in 1 second (FEV₁); (2) a relative percent change from baseline (e.g., at week 12) as measured by forced expiratory flow at 25-75% of the pulmonary volume (FEF_{25-75%}); (3) annualized rate of loss of asthma control events during the treatment period; (4) annualized rate of severe exacerbation events during the treatment period; (5) time to loss of asthma control events during the treatment period; (6) time to severe exacerbation events during the treatment period; (7) time to loss of asthma control events during overall study period; (8) time to severe exacerbation events during overall study period; (9) health care resource utilization; (10) change from baseline (e.g., at week 12) in: i) morning and evening asthma symptom scores, ii) ACQ-5 score, iii) AQLQ score, iv) morning and evening PEF, v) number of inhalations/day of salbutamol/albuterol or levosalbutamol/levalbuterol for symptom relief, vi) nocturnal awakenings; or (11) change from baseline (e.g., at week 12 or week 24) in: i) 22-item Sino Nasal Outcome Test (SNOT-22), ii) Hospital Anxiety and Depression Score (HADS), iii) EuroQual questionnaire (EQ-5D-3L or EQ-5D-5L). An “improvement in an asthma-associated parameter” means an increase from baseline of one or more of FEV₁, AM PEF or PM PEF, and/or a decrease from baseline of one or more of daily albuterol/levalbuterol use, ACQ5 score, average nighttime awakenings or SNOT-22 score. As used herein, the term “baseline,” with regard to an asthma-associated parameter, means the numerical value of the asthma-associated parameter for a patient prior to or at the time of administration of a pharmaceutical composition comprising an IL-4R antagonist.

To determine whether an asthma-associated parameter has “improved,” the parameter is quantified at baseline and at a time point after administration of the pharmaceutical composition described herein. For example, an asthma-associated parameter may be measured at day 1, day 2, day 3, day 4, day 5, day 6, day 7, day 8, day 9, day 10, day 11, day 12, day 13, day 14, or at week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with the pharmaceutical composition. The difference between the value of the parameter at a particular time point following initiation of treatment and the value of the parameter at baseline is used to establish whether there has been an “improvement” in the asthma associated parameter (e.g., an increase or decrease, as the case may be, depending on the specific parameter being measured).

The terms “acquire” or “acquiring” as used herein, refer to obtaining possession of a physical entity, or a value, e.g., a numerical value, by “directly acquiring” or “indirectly acquiring” the physical entity or value, such as an asthma-associated parameter. “Directly acquiring” means performing a process (e.g., performing a synthetic or analytical method) to obtain the physical entity or value. “Indirectly acquiring” refers to receiving the physical entity or value from another party or source (e.g., a third-party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”).

Information that is acquired indirectly can be provided in the form of a report, e.g., supplied in paper or electronic form, such as from an online database or application (an “App”). The report or information can be provided by, for example, a healthcare institution, such as a hospital or clinic; or a healthcare provider, such as a doctor or nurse.

Forced Expiratory Volume in 1 Second (FEV₁). According to certain embodiments, administration of an IL-4R antagonist to a patient results in an increase from baseline of forced expiratory volume in 1 second (FEV₁). Methods for measuring FEV₁ are known in the art. For example, a spirometer that meets the 2005 American Thoracic Society (ATS)/European Respiratory Society (ERS) recommendations can be used to measure FEV₁ in a patient. The ATS/ERS Standardization of Spirometry may be used as a guideline. Spirometry is generally performed between 6 and 10 AM after an albuterol withhold of at least 6 hours. Pulmonary function tests are generally measured in the sitting position, and the highest measure is recorded for FEV₁ (in liters). For pre-bronchodilator measured parameters, including FEV1, peak expiratory flow (PEF), FVC and FEF25-75%, spirometry should be performed after a wash out period of bronchodilators according to their action duration, for example, withholding the last dose of salbutamol/albuterol or levosalbutamol/levalbuterol for at least 6 hours, withholding the last dose of LABA for at least 12 hours, and withholding the last dose of LAMA for at least 24 hours.

In certain exemplary embodiments, FEV1 reversibility is defined as an increase in absolute FEV1 of 10% over the baseline value, demonstrated within 30 minutes of bronchodilator administration. Reversibility can be tested after the administration of 200 to 400 mcg (2 to 4 puffs) of albuterol/salbutamol or 45 to 90 mcg of (2 to 4 puffs) levalbuterol/levosalbutamol reliever medication from a primed MDI (up to 3 opportunities during the same visit are allowed with a maximum of 12 puffs of reliever medication, if tolerated by the patient). Documented reversibility or positive airway hyperresponsiveness to methacholine within 12 months prior to visit 1 is considered acceptable.

All reversibility tests should be administered after pulmonary function testing and after asthma medications have been withheld for the appropriate intervals. Subjects can receive albuterol/salbutamol or levalbuterol/levosalbutamol reliever medication as puff inhalations using the respective MDI. Alternatively, reversibility testing may be performed using inhalation of nebulized albuterol/salbutamol or levalbuterol/levosalbutamol reliever medication. The spirometry for measuring absolute FEV1 may be repeated several times within the 30 minutes after administration of bronchodilator. For post-bronchodilator FEV1, the measure should follow the steps as that at screening test for reversibility validation except a maximum of 4 puffs of reliever medication can be used.

Therapeutic methods that result in an increase of FEV₁ from baseline of at least 0.05 L at week 12 following initiation of treatment with a pharmaceutical composition comprising an anti-IL-4R antagonist are provided. For example, administration of an IL-4R antagonist to a subject in need thereof causes an increase of FEV₁ from baseline of about 0.05 L, 0.10 L, 0.12 L, 0.14 L, 0.16 L, 0.18 L, 0.20 L, 0.22 L, 0.24 L, 0.26 L, 0.28 L, 0.30 L, 0.32 L, 0.34 L, 0.36 L, 0.38 L, 0.40 L, 0.42 L, 0.44 L, 0.46 L, 0.48 L, 0.50 L, or more at week 12.

FEF_{25-75%}. According to certain embodiments, administration of an IL-4R antagonist to a patient results in an increase from baseline of FEF25-75%. Methods for measuring FEV₁ are known in the art. For example, a spirometer that meets the 2005 American Thoracic Society (ATS)/European Respiratory Society (ERS) recommendations can be used to measure FEV₁ in a patient. The FEF_{25-75%} (forced expiratory flow between 25% and 75%) is the speed (in liters per second) at which a person can empty the middle half of his or her air during a maximum expiration (i.e., Forced Vital Capacity or FVC). The parameter relates to the average flow from the point at which 25 percent of the FVC has been exhaled to the point at which 75 percent of the FVC has been exhaled. The FEF25-75% of a subject provides information regarding small airway function, such as the extent of small airway disease and/or inflammation. A change in FEF_{25-75%} is an early indicator of obstructive lung disease. In certain embodiments, an improvement and/or increase in the FEF_{25-75%} parameter is an improvement of at least 10%, 25%, 50% or more as compared to baseline. In certain embodiments, the methods described herein result in normal FEF_{25-75%} values in a subject (e.g., values ranging from 50-60% and up to 130% of the average).

Morning and Evening Peak Expiratory Flow (AM PEF and PM PEF). According to certain embodiments, administration of an IL-4R antagonist to a patient results in an increase from baseline of morning (AM) and/or evening (PM) peak expiratory flow (AM PEF and/or PM PEF). Methods for measuring PEF are known in the art. For example, according to one method for measuring PEF, patients are issued an electronic PEF meter for recording morning (AM) and evening (PM) PEF (as well as daily albuterol use, morning and evening asthma symptom scores, and number of nighttime awakenings due to asthma symptoms that require rescue medications). Patients are instructed on the use of the device, and written instructions on the use of the electronic PEF meter are provided to the patients. In addition, a medical professional may instruct the patients on how to record pertinent variables in the electronic PEF meter. AM PEF is generally performed within 15 minutes after arising (between 6 am and 10 am) prior to taking any albuterol. PM PEF is generally performed in the evening (between 6 pm and 10 pm) prior to taking any albuterol. Subjects should try to withhold albuterol for at least 6 hours prior to measuring their PEF. Three PEF efforts are performed by the patient and all 3 values are recorded by the electronic PEF meter. Usually the highest value is used for evaluation. Baseline AM PEF may be calculated as the mean AM measurement recorded for the 7 days prior to administration of the first dose of pharmaceutical composition comprising the IL-4R antagonist, and baseline PM PEF may be calculated as the mean PM measurement recorded for the 7 days prior to administration of the first dose of pharmaceutical composition comprising the IL-4R antagonist.

Therapeutic methods that result in an increase in AM PEF and/or PM PEF from baseline of at least 1.0 L/min at week 12 following initiation of treatment with a pharmaceutical composition comprising an anti-IL-4R antagonist are provided. For example, according to exemplary embodiments, administration of an IL-4R antagonist to a subject in need thereof causes an increase in PEF from baseline of about 0.5 L/min, 1.0 L/min, 1.5 L/min, 2.0 L/min, 2.5 L/min, 3.0 L/min, 3.5 L/min, 4.0 L/min, 4.5 L/min, 5.0 L/min, 5.5 L/min, 6.0 L/min, 6.5 L/min, 7.0 L/min, 7.5 L/min, 8.0 L/min, 8.5 L/min, 9.0 L/min, 9.5 L/min, 10.0 L/min, 10.5 L/min, 11.0 L/min, 12.0 L/min, 15 L/min, 20 L/min, or more at week 12.

Albuterol/Levalbuterol Use. According to certain embodiments, administration of an IL-4R antagonist to a patient results in a decrease from baseline of daily albuterol or levalbuterol use. The number of albuterol/levalbuterol inhalations can be recorded daily by the patients in a diary, PEF meter, or other recording device. During treatment with the pharmaceutical composition described herein, use of albuterol/levalbuterol typically may be on an as-needed basis for symptoms, not on a regular basis or prophylactically. The baseline number of albuterol/levalbuterol inhalations/day may be calculated based on the mean for the 7 days prior to administration of the first dose of pharmaceutical composition comprising the IL-4R antagonist.

Therapeutic methods are provided that result in a decrease in albuterol/levalbuterol use from baseline of at least 0.25 puffs per day at week 12 following initiation of treatment with a pharmaceutical composition comprising an anti-IL-4R antagonist. For example, administration of an IL-4R antagonist to a subject in need thereof causes a decrease in albuterol/levalbuterol use from baseline of about 0.25 puffs per day, 0.50 puffs per day, 0.75 puffs per day, 1.00 puff per day, 1.25 puffs per day, 1.5 puffs per day, 1.75 puffs per day, 2.00 puffs per day, 2.25 puffs per day, 2.5 puffs per day, 2.75 puffs per day, 3.00 puffs per day, or more at week 12.

OCS Use. According to certain embodiments, administration of an IL-4R antagonist to a patient can be used in conjunction with an OCS such as oral prednisone. The number of OCS administrations can be recorded daily by the patients in a diary, PEF meter, or other recording device. During treatment with the pharmaceutical composition described herein, occasional short-term use of prednisone typically can be used to control acute asthmatic episodes, e.g., episodes in which bronchodilators and other anti-inflammatory agents fail to control symptoms. In other aspects, prednisone is used concurrent with or as a substitution for ICS. Oral prednisone may be administered in dosages of about 5 mg, 10 mg, 15 mg, 20 mg, 25 mg, 30 mg, 35 mg or 40 mg. OCS can optionally be administered once a day or multiple times a day (e.g., twice a day, three times a day, four times a day, etc.)

In certain exemplary embodiments, methods for reducing or eliminating the dependency of the subject on OCS use are provided. The reduction or elimination of steroid dependency is highly advantageous and desirable. In certain embodiments, a reduction of 50% or greater (e.g., 50%, 60%, 70%, 80%, 90% or more) in the OCS dose is achieved after administration of IL-4R antibody therapy at a period of time (e.g., at week 24 In certain embodiments, the OCS is substantially eliminated after 40 weeks, 45 weeks, 50 weeks, 52 weeks, or greater after first dose following administration of the loading dose. In other embodiments, the level of OCS use is reduced to less than 5 mg per day (e.g., less than 5 mg, 4 mg, 3 mg, 2 mg or less per day). In other embodiments, the dependency on OCS use is substantially eliminated after 3 months, 6 months, 9 months or 1 year following treatment with IL4R antibody or fragment thereof.

5-Item Asthma Control Questionnaire (ACQ) Score. According to certain embodiments, administration of an IL-4R antagonist to a patient results in a decrease from baseline of five-item Asthma Control Questionnaire (ACQ5) score. The ACQ5 is a validated questionnaire to evaluate asthma control.

Therapeutic methods are provided that result in a decrease in ACQ5 score from baseline of at least 0.10 points at week 12 following initiation of treatment with a pharmaceutical composition comprising an anti-IL-4R antagonist. For example, administration of an IL-4R antagonist to a subject in need thereof causes a decrease in ACQ score from baseline of about 0.10 points, 0.15 points, 0.20 points, 0.25 points, 0.30 points, 0.35 points, 0.40 points, 0.45 points, 0.50 points, 0.55 points, 0.60 points, 0.65 points, 0.70 points, 0.75 points, 0.80 points, 0.85 points, or more at week 12.

Night-Time Awakenings. According to certain embodiments, administration of an IL-4R antagonist to a patient results in a decrease from baseline of average number of nighttime awakenings.

In certain embodiments, the methods decrease the average number of nighttime awakenings from baseline by at least about 0.10 times per night at week 12 following initiation of treatment. For example, administration of an IL-4R antagonist to a subject in need thereof can cause a decrease in average number of nighttime awakenings from baseline of about 0.10 times per night, 0.15 times per night, 0.20 times per night, 0.25 times per night, 0.30 times per night, 0.35 times per night, 0.40 times per night, 0.45 times per night, 0.50 times per night, 0.55 times per night, 0.60 times per night, 0.65 times per night, 0.70 times per night, 0.75 times per night, 0.80 times per night, 0.85 times per night, 0.90 times per night, 0.95 times per night, 1.0 times per night, 2.0 times per night, or more at week 12.

22-Item Sinonasal Outcome Test (SNOT-22) Score. According to certain embodiments, administration of an IL-4R antagonist to a patient results in a decrease from baseline of 22-item Sinonasal Outcome Test (SNOT-22). The SNOT-22 is a validated questionnaire to assess the impact of chronic rhinosinusitis on quality of life (Hopkins et al 2009, Clin. Otolaryngol. 34: 447-454).

Therapeutic methods are provided that result in a decrease in SNOT-22 score from baseline of at least 1 point at week 12 following initiation of treatment with a pharmaceutical composition comprising an anti-IL-4R antagonist. For example, administration of an IL-4R antagonist to a subject in need thereof can cause a decrease in SNOT-22 score from baseline of about 1, 2, 3, 4, 5, 6, 7, 8, 9, 10, 11, 12, 13 points, or more at week 12.

Biomarkers. In certain embodiments, the subject experiences an improvement in lung function as measured by a biomarker relative to the biomarker measurement at baseline. For example, the biomarker may be fractional exhaled nitric oxide (FeNO), eotaxin-3, total IgE, allergen-specific IgE, allergen-specific IgG4, periostin, eosinophil (EOS) level, or thymus and activation-regulated chemokine (TARC). In certain exemplary embodiments, FeNO level is decreased relative to baseline. In certain exemplary embodiments, TARC level is decreased relative to baseline. In certain exemplary embodiments, total IgE level is decreased relative to baseline. In certain exemplary embodiments, EOS level is decreased relative to baseline. In certain embodiments, an improvement in lung function is indicated by a reduction or an increase (as appropriate) at week 4, week 12, week 24, etc., following treatment relative to baseline lung function.

Patient Reported Outcomes (PROs). In certain embodiments, the subject experiences an improvement in one or more patient reported outcomes (PROs). In certain embodiments, the PROs include but are not limited to, asthma control questionnaire, ACQ-7-IA (Asthma Control Questionnaire-Interviewer Administered, 7-question version), ACQ-5-IA (Asthma Control Questionnaire-Interviewer Administered, 5-question version), PAQLQ (Pediatric Asthma Quality of Life Questionnaire with Standardized Activities), PAQLQ-IA (Pediatric Asthma Quality of Life Questionnaire with Standardized Activities-Interviewer Administered), PACQLQ (Pediatric Asthma Caregiver's Quality of Life Questionnaire), PRQLQ (Pediatric Rhino-conjunctivitis Quality Of Life Questionnaire), PRQLQ-IA (Pediatric Rhino-conjunctivitis Quality Of Life Questionnaire-Interviewer Administered in patients with comorbid allergic rhinitis), EQ-5D-5L (EuroQol 5-level questionnaire), EQ-5D-Y (EuroQol 5 dimension youth questionnaire), Euro QoL (EQ-5D-Y)—for Children, and Health Related Quality of Life (HRQoL), HCRU (healthcare resource utilization), morning (AM) symptom score, evening (PM) symptom score, number of nocturnal awakenings, and reliever medication use frequency. In certain embodiments, the subject experiences an improvement in one or more PROs as measured by one or more standard as described herein.

Infection rates. In certain embodiments, the subject experiences a reduction of infection rate of respiratory and/or overcall infections. In certain embodiments, the respiratory infection is a bacterial, fungal, and/or viral infection. In certain embodiments, the respiratory infection is an upper respiratory tract infection, a lower respiratory tract infection, or a mixture thereof.

### Methods for Treating Asthma

In some embodiments, methods are provided for treating asthma in a subject in need thereof, wherein the methods comprise administering a pharmaceutical composition comprising an IL-4R antagonist to the subject.

As used herein, the term “asthma” can be used interchangeably with “intermittent asthma,” or “bronchial asthma.” “Asthma,” “bronchial asthma” and “intermittent asthma,” and allergic forms of each of these, refer to asthma in which one or any combination of the following are true: symptoms occur 2 or fewer days per week; symptoms do not interfere with normal activities; nighttime symptoms occur fewer than 2 days per month; or one or more lung function tests (e.g., forced expiratory volume in one second (FEV₁) and/or peak expiratory flow (PEF) of greater than 80%) are normal when the subject is not suffering from an asthma attack.

Allergic asthma refers to asthma that is triggered by allergens, e.g., inhaled allergens, such as dust mites, pet dander, pollen, fungi and the like. As used herein, the term “allergic asthma” refers to asthma in combination with one or more allergic markers, e.g., total serum IgE (e.g., a total serum IgE of ≥30 IU/mL), and/or at least one positive allergen-specific IgE value (e.g., an allergen-specific IgE value of ≥0.35 kU/L). In certain embodiments, the allergen is an airborne aeroallergen (e.g., an annual aeroallergen or a perennial aeroallergen).

In certain exemplary embodiments, a subject having allergic asthma has a total serum IgE level of about ≥5 IU/mL, about ≥10 IU/mL, about ≥20 IU/mL, about ≥30 IU/mL, about ≥40 IU/mL, about ≥50 IU/mL, about ≥60 IU/mL, about ≥70 IU/mL, about ≥80 IU/mL, about ≥90 IU/mL, about ≥100 IU/mL, about ≥110 IU/mL, about ≥120 IU/mL, about ≥130 IU/mL, about ≥140 IU/mL, about ≥150 IU/mL, about ≥160 IU/mL, about ≥170 IU/mL, about ≥180 IU/mL, about ≥190 IU/mL, about ≥200 IU/mL, about ≥250 IU/mL, about ≥300 IU/mL, about ≥350 IU/mL, about ≥400 IU/mL, about ≥450 IU/mL, about ≥500 IU/mL, about ≥550 IU/mL, about ≥600 IU/mL, about ≥650 IU/mL, about ≥700 IU/mL, about ≥750 IU/mL, about ≥800 IU/mL, about ≥850 IU/mL, about ≥900 IU/mL, about ≥950 IU/mL, about ≥1000 IU/mL or greater.

In certain exemplary embodiments, a subject having allergic asthma has at least one positive allergen-specific IgE value present in an amount of about ≥0.05 kU/L, about ≥0.10 kU/L, about ≥0.15 kU/L, about ≥0.20 kU/L, about ≥0.21 kU/L, about ≥0.22 kU/L, about ≥0.23 kU/L, about ≥0.24 kU/L, about ≥0.25 kU/L, about ≥0.26 kU/L, about ≥0.27 kU/L, about ≥0.28 kU/L, about ≥0.29 kU/L, about ≥0.30 kU/L, about ≥0.31 kU/L, about ≥0.32 kU/L, about ≥0.33 kU/L, about ≥0.34 kU/L, about ≥0.35 kU/L, about ≥0.36 kU/L, about ≥0.37 kU/L, about ≥0.38 kU/L, about ≥0.39 kU/L, about ≥0.40 kU/L, about ≥0.45 kU/L, about ≥0.50 kU/L, about ≥0.55 kU/L, about ≥0.60 kU/L, about ≥0.65 kU/L, about ≥0.70 kU/L or greater.

As used herein, a “perennial aeroallergen” refers to airborne allergens that can be present in the environment year-round, such as dust mites, fungi, dander and the like. Perennial aeroallergens include, but are not limited to, Alternaria alternata, Aspergillus fumigatus, Aureobasidium pullulans, Candida albicans, Cladosporium herbarum, Dermatofagoides farinae, Dermatofagoides pteronyssinus, Mucor racemosus, Penicillium chrysogenum, Phoma betae, Setomelanomma rostrata, Stemphylium herbarum, cat dander, dog dander, cow dander, chicken feathers, goose feathers, duck feathers, cockroach (e.g., German cockroach, Oriental cockroach), mouse urine, peanut dust, tree nut dust, and the like.

As used herein, a “seasonal aeroallergen” refers to airborne allergens that are present in the environment seasonally, such as pollens and spores. Seasonal aeroallergens include, but are not limited to, tree pollen (e.g., birch, alder, cedar, hazel, hornbeam, horse chestnut, willow, poplar, linden, pine, maple, oak, olive and the like), grass pollen (e.g., ryegrass, cat's tail and the like), weed pollen (e.g., ragweed, plantain, nettles, mugwort, fat hen, sorrel and the like), fungal spores that increase during particular seasons, temperatures, etc. (e.g., molds), and the like.

“IgE” refers to an antibody isotype that contains the c heavy chain, and is a monomer having five domains in the immunoglobulin structure. IgE is typically present in plasma at a concentration of less than 1 μg/mL, and has a half-life of about 2 days in serum (Abbas and Lichtman (2004) Basic Immunology functions and disorders of the immune system. 2nd ed. Philadelphia: Saunders). The units kU/L or IU/mL (which units can be used interchangeably) are often used to express the level of IgE in peripheral blood, with one kU/L is equal to 2.4 ng/mL (Seagroatt and Anderson (1981) E. J. Biol Stand. 9:431).

IgE (e.g., total serum IgE and/or allergen specific IgE) can be determined using a variety of methods known in the art. For example, PRIST (paper radioimmunosorbent test) can be used, in which serum samples react with IgE that has been tagged with radioactive iodine. Bound radioactive iodine is detected, and is proportional to the amount of total IgE in the serum sample. In clinical immunology, levels of individual classes of immunoglobulins can be measured by nephelometry (or turbidimetry) to characterize the antibody profile of a subject. Other methods of measuring IgE levels include, but are not limited to, ELISA, immunofluorescence, Western blot, immunodiffusion, immunoelectrophoresis and the like. Measurement of a serum IgE concentration can be performed using a UniCAP 250® system (Pharmacia, Uppsala, Sweden) (See G. J. Gleich, A. K. Averbach and N. A. Swedlund, Measurement of IgE in normal and allergic serum by radioimmunoassay. J. Lab. Clin. Med. 77 (1971), p. 690.)

Asthma/intermittent asthma, bronchial asthma/intermittent bronchial asthma, and persistent asthma/persistent bronchial asthma, and allergic forms of each of these, can be categorized as “mild,” “moderate,” “severe” or “moderate-to-severe.” “Mild intermittent asthma” or “mild intermittent bronchial asthma” is defined as having symptoms less than once a week, and having forced expiratory volume in one second (FEV₁) or peak expiratory flow (PEF)≥80%. “Mild persistent asthma” or “mild persistent bronchial asthma” differs in that symptoms frequency is greater than once per week but less than once per day, and variability in FEV₁ or PEF is <20%-30%. “Moderate intermittent asthma” or “moderate intermittent bronchial asthma” is defined as having symptoms less than once a week, and having forced expiratory volume in one second (FEV₁) or peak expiratory flow (PEF) of 60-80%. “Moderate persistent asthma” or “moderate persistent bronchial asthma,” or an allergic form thereof, is defined as having daily symptoms, exacerbations that may affect activity and/or sleep, nocturnal symptoms more than once a week, daily use of inhaled short-acting beta-2 agonist and having forced expiratory volume in one second (FEV₁) or peak expiratory flow (PEF) of 60-80%. “Severe intermittent asthma” or “severe intermittent bronchial asthma,” or an allergic form thereof, is defined as having symptoms less than once a week, and having forced expiratory volume in one second (FEV₁) or peak expiratory flow (PEF) of 60%. “Severe persistent asthma” or “severe persistent bronchial asthma” is defined as having daily symptoms, frequent exacerbations that may affect activity and/or sleep, frequent nocturnal symptoms, limitation of physical activities, daily use of inhaled short-acting beta-2 agonist, and having forced expiratory volume in one second (FEV₁) or peak expiratory flow (PEF) of 60%. “Moderate-to-severe intermittent asthma” or “moderate-to-severe intermittent bronchial asthma,” or an allergic form thereof, is defined as having symptoms between those of moderate intermittent asthma/moderate intermittent bronchial asthma and severe intermittent asthma/severe intermittent bronchial asthma. “Moderate-to-severe persistent asthma” or “moderate-to-severe persistent bronchial asthma,” or an allergic form thereof, is defined as having symptoms between those of moderate persistent asthma/moderate persistent bronchial asthma and severe persistent asthma/severe persistent bronchial asthma.

As used herein, the term “inadequately controlled asthma” refers to patients whose asthma is either “not well controlled” or “very poorly controlled” as defined by the “Expert Panel Report 3: Guidelines for the Diagnosis and Management of Asthma,” National Heart, Blood and Lung Institute, NIH, Aug. 28, 2007. “Not well controlled asthma” is defined as having symptoms greater than two days per week, nighttime awakenings one to three times per week, some limitations on normal activity, short-acting beta2-agonist use for symptom control greater than two days per week, FEV₁ of 60-80% of predicted and/or personal best, an ATAQ score of 1-2, an ACQ score of 1.5 or greater, and an ACT score of 16-19. “Very poorly controlled asthma” is defined as having symptoms throughout the day, nighttime awakenings four times or more per week, extreme limitations on normal activity, short-acting beta2-agonist use for symptom control several times per day, FEV₁ of less than 60% of predicted and/or personal best, an ATAQ score of 3-4, an ACQ score of N/A, and an ACT score of less than or equal to 15.

In certain embodiments, a subject is identified as having “uncontrolled persistent” asthma if the subject receives such a diagnosis from a physician, and has symptoms that remain uncontrolled (e.g., manifested by symptoms, exacerbations and/or airflow limitation) despite treatment with medium-to-high dose inhaled corticosteroids and a second controller agent or systemic corticosteroids. (See Wenzel et al. (2016) Lancet 388: 32-44.)

In some embodiments, a subject is identified as having “uncontrolled moderate-to-severe” asthma if the subject receives such a diagnosis from a physician, based on the Global Initiative for Asthma (GINA) 2009 Guidelines, and one or more of the following criteria: i) existing treatment with moderate- or high-dose ICS/LABA (2 fluticasone propionate 250 μg twice daily or equipotent ICS daily dosage) with a stable dose of ICS/LABA for greater than or equal to 1 month prior to administration of the loading dose of IL-4R antagonist; ii) FEV₁ 40 to 80% predicted normal prior to administration of the loading dose of IL-4R antagonist; iii) ACQ-5 score greater than or equal to 1.5 prior to administration of the loading dose of IL-4R antagonist; iv) reversibility of at least 12% and 200 mL in FEV₁ after 200 μg to 400 μg (2 to 4 inhalations) of salbutamol/albuterol prior to administration of the loading dose of IL-4R antagonist; or v) has experienced, within 1 year prior to administration of the loading dose of IL-4R antagonist, any of the following events: (a) treatment with greater than or equal to 1 systemic (oral or parenteral) steroid burst for worsening asthma, (b) hospitalization or an emergency/urgent medical care visit for worsening asthma.

“Severe asthma” refers to asthma in which adequate control cannot be achieved by high-dose treatment with inhaled corticosteroids and additional controllers (e.g., long-acting inhaled beta 2 agonists, montelukast, and/or theophylline) or by oral corticosteroid treatment (e.g., for at least six months per year), or is lost when the treatment is reduced. In certain embodiments, severe asthma includes asthma that is treated with high-dose ICS and at least one additional controller (e.g., LABA, montelukast, or theophylline) or oral corticosteroids >6 months/year, wherein at least one of the following occurs or would occur if treatment is reduced: ACT<20 or ACQ>1.5; at least 2 exacerbations in the last 12 months; at least 1 exacerbation treated in hospital or requiring mechanical ventilation in the last 12 months; or FEV₁<80% (if FEV₁/FVC below the lower limit of normal).

“Steroid-dependent asthma” refers to asthma which requires one or more of the following treatments: frequent, short term oral corticosteroid treatment bursts in the past 12 months; regular use of high dose inhaled corticosteroids in the past 12 months; regular use of injected long acting corticosteroids; daily use of oral corticosteroids; alternate-day oral corticosteroids; or prolonged use of oral corticosteroids in the past year.

“Oral corticosteroid-dependent asthma” refers to a subject having ≥3 30-day oral corticosteroid (OCS) fills over a 12-month period and a primary asthma diagnosis within 12 months of the first OCS fill. Subjects with OCS-dependent asthma may also experience one or any combination of the following: have received physician prescribed LABA and high dose ICS (total daily dose >500 μg fluticasone propionate dry powder formulation equivalent) for at least 3 months (the ICS and LABA can be parts of a combination product, or given by separate inhalers); have received additional maintenance asthma controller medications according to standard practice of care e.g., leukotriene receptor antagonists (LTRAs), theophylline, long-acting muscarinic antagonists (LAMAs), secondary ICS and cromones; received OCS for the treatment of asthma at a dose of between ≥7.5 to ≤30 mg (prednisone or prednisolone equivalent); have received an OCS dose administered every other day (or different doses every other day); morning pre-bronchodilator (BD) FEV₁ of <80% predicted normal; have evidence of asthma as documented by post-BD (albuterol/salbutatomol) reversibility of FEV₁≥12% and ≥200 mL (15-30 min after administration of 4 puffs of albuterol/salbutamol); or have a history of at least one asthma exacerbation event within 12 months.

In one aspect, methods for treating asthma are provided comprising: (a) selecting a subject (e.g., a pediatric subject) that exhibits a blood eosinophil level of at least 300 cells per microliter; and (b) administering to the subject (e.g., the pediatric subject) a pharmaceutical composition comprising an IL-4R antagonist.

In another aspect, methods for treating asthma are provided comprising: (a) selecting a subject (e.g., a pediatric subject) that exhibits a blood eosinophil level of 200-299 cells per microliter; and (b) administering to the subject (e.g., the pediatric subject) a pharmaceutical composition comprising an IL-4R antagonist.

In another aspect, methods for treating asthma are provided comprising: (a) selecting a subject (e.g., a pediatric subject) that exhibits a blood eosinophil level of less than 200 cells per microliter; and (b) administering to the subject (e.g., the pediatric subject) a pharmaceutical composition comprising an IL-4R antagonist.

In one aspect, methods for treating asthma are provided comprising: (a) selecting a subject (e.g., a pediatric subject) that exhibits a blood eosinophil level of at least 150 cells per microliter; and (b) administering to the subject (e.g., the pediatric subject) a pharmaceutical composition comprising an IL-4R antagonist.

In one aspect, methods for treating asthma are provided comprising: (a) selecting a subject (e.g., a pediatric subject) that exhibits a baseline FeNO level of ≥20 ppb; and (b) administering to the subject (e.g., the pediatric subject) a pharmaceutical composition comprising an IL-4R antagonist.

In one aspect, methods for treating asthma are provided comprising: (a) selecting a subject (e.g., a pediatric subject) that exhibits a baseline FeNO level of ≥25 ppb; and (b) administering to the subject (e.g., the pediatric subject) a pharmaceutical composition comprising an IL-4R antagonist.

In one aspect, methods for treating asthma are provided comprising: (a) selecting a subject (e.g., a pediatric subject) that exhibits a baseline FeNO level of ≥50 ppb; and (b) administering to the subject (e.g., the pediatric subject) a pharmaceutical composition comprising an IL-4R antagonist.

In one aspect, methods for treating asthma are provided comprising: (a) selecting a subject (e.g., a pediatric subject) that exhibits (1) a blood eosinophil level of at least 150 cells per microliter, at least 200 cells per microliter, about 200-299 cells per microliter, at least 300 cells per microliter, at least 400 cells per microliter, or at least 500 cells per microliter; and (2) a baseline FeNO level of ≥20 ppb, a baseline FeNO level of ≥25 ppb, or a baseline FeNO level of ≥50 ppb, and (b) administering to the subject (e.g., the pediatric subject) a pharmaceutical composition comprising an IL-4R antagonist.

In a related aspect, methods for treating asthma comprising an add-on therapy to background therapy are provided. In certain embodiments, an IL-4R antagonist is administered as an add-on therapy to a subject (e.g., a pediatric subject) that has asthma who is on background therapy for a certain period of time (e.g., 1 week, 2 weeks, 3 weeks, 1 month, 2 months, 5 months, 12 months, 18 months, 24 months, or longer) (also called the “stable phase”). In some embodiments, the background therapy comprises an inhaled corticosteroid (ICS) and/or a controller medication selected from the group consisting of one or any combination of a long-acting β2 agonist (LABA), a leukotriene receptor antagonist (LTRA), a long-acting muscarinic antagonist (LAMA), and a methylxanthine.

In some embodiments, a method for reducing an asthma patient's dependence on ICS and/or a controller medication selected from the group consisting of one or any combination of a LABA, an LTRA, a LAMA, and a methylxanthine for the treatment of one or more asthma exacerbations comprising: (a) selecting a subject (e.g., a pediatric subject) who has moderate-to-severe asthma that is uncontrolled with a background asthma therapy comprising an ICS, one or any combination of a LABA, an LTRA, a LAMA, and a methylxanthine, or a combination thereof; and administering to the subject (e.g., the pediatric subject) a pharmaceutical composition comprising an IL-4R antagonist, is provided.

In some embodiments, methods to treat or alleviate one or more conditions or complications associated with asthma or comorbid with asthma, such as a Type 2 inflammatory condition, e.g., one or more of chronic rhinosinusitis, allergic rhinitis, allergic fungal rhinosinusitis, chronic sinusitis, allergic bronchopulmonary aspergillosis (ABPA), unified airway disease, eosinophilic granulomatosis with polyangiitis (EGPA, formerly known as Churg-Strauss syndrome), gastroesophageal reflux disease (GERD), allergic conjunctivitis, atopic conjunctivitis, atopic dermatitis, vasculitis, cystic fibrosis (CF), chronic obstructive pulmonary disease (COPD), eosinophilic esophagitis (EoE), chronic rhinosinusitis with nasal polyps (CRSwNP), aspirin hypersensitivity, non-steroidal anti-inflammatory drug (NSAID) hypersensitivity (e.g., NSAIDs exacerbated respiratory disease, or NSAID-ERD), perennial allergic rhinitis (PAR), atopic dermatitis (AD), food allergy, hives or urticaria, chronic eosinophilic pneumonia (CEP) and exercise induced bronchospasm, are provided.

In one aspect, a subject to be treated for asthma is a subject having one or more of the following characteristics: children 6 to <12 years of age, with an investigator diagnosis of persistent asthma for ≥12 months prior to screening, based on clinical history and examination, pulmonary function parameters according to Global Initiative for Asthma (GINA) 2015 Guidelines and the following criteria: existing background therapy of medium-dose ICS with second controller medication (i.e., LABA, LTRA, LAMA, or methylxanthines) or high-dose ICS alone or high-dose ICS with second controller, for at least 3 months with a stable dose ≥1 month prior to screening visit 1; pre-bronchodilator forced expiratory volume in 1 second (FEV₁)≤95% of predicted normal or pre-bronchodilator FEV1/forced vital capacity (FVC) ratio <0.85 at screening and baseline visits; reversibility of at least 10% in FEV1 after the administration of 200 to 400 mcg (2 to 4 puff inhalations with metered-dose inhaler (MDI)) of albuterol/salbutamol or 45 to 90 mcg (2 to 4 puffs with MDI) of levalbuterol/levosalbutamol reliever medication before randomization (Up to 3 opportunities during the same visit are allowed with a maximum of 12 puffs of reliever medication if tolerated by the patient. Note: A maximum of 3 visits to meet the qualifying criterion of reversibility may be made during the screening period and prior to the patient's randomization. Documented reversibility or positive airway hyperresponsiveness to methacholine within 12 months prior to screening V1 is considered acceptable); have experienced, within one year prior to use of reliever medication (i.e., albuterol/salbutamol or levalbuterol/levosalbutamol), other than as a preventive for exercise induced bronchospasm, on 3 or more days per week, on at least one week during the screening period; sleep awakening due to asthma symptoms requiring use of reliever medication at least once during the screening period; and asthma symptoms 3 or more days per week on at least one week during the screening period.

### Interleukin-4 Receptor Antagonists

The methods featured herein comprise administering to a subject in need thereof a therapeutic composition comprising an IL-4R antagonist. As used herein, an “IL-4R antagonist” is any agent that binds to or interacts with IL-4R and inhibits the normal biological signaling function of IL-4R when IL-4R is expressed on a cell in vitro or in vivo. Non-limiting examples of categories of IL-4R antagonists include small molecule IL-4R antagonists, anti-IL-4R aptamers, peptide-based IL-4R antagonists (e.g., “peptibody” molecules), and antibodies or antigen-binding fragments of antibodies that specifically bind human IL-4R. According to certain embodiments, the IL-4R antagonist comprises an anti-IL-4R antibody that can be used in the context of the methods described elsewhere herein. For example, in one embodiment, the IL-4R antagonist is an antibody or antigen-binding fragment thereof that specifically binds to an IL-4R, and comprises the heavy chain and light chain (Complementarity Determining Region) CDR sequences from the Heavy Chain Variable Region (HCVR) and Light Chain Variable Region (LCVR) of SEQ ID NOs:1 and 2, respectively.

The term “human IL4R” (hIL-4R) refers to a human cytokine receptor that specifically binds to interleukin-4 (IL-4), such as IL-4Rα.

The term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2, and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments, the FRs of the anti-IL-4R antibody (or antigen-binding portion thereof) may be identical to the human germline sequences, or may be naturally or artificially modified. An amino acid consensus sequence may be defined based on a side-by-side analysis of two or more CDRs.

The term “antibody” also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds to an antigen to form a complex. Antigen-binding fragments of an antibody may be derived, e.g., from full antibody molecules using any suitable standard techniques, such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment.”

An antigen-binding fragment of an antibody will typically comprise at least one variable domain. The variable domain may be of any size or amino acid composition and will generally comprise at least one CDR that is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids that result in a flexible or semi-flexible linkage between adjacent variable and/or constant domains in a single polypeptide molecule, typically the hinge region may consist of between 2 to 60 amino acids, typically between 5 to 50, or typically between 10 to 40 amino acids. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody described herein using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody” includes antibodies having variable and constant regions derived from human germline immunoglobulin sequences. The human antibodies described herein may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. Antibodies having one or more mutations in the hinge, C_{H}2, or C_{H}3 region, which may be desirable, for example, in production, to improve the yield of the desired antibody form, are provided.

An “isolated antibody” means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody”. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-4R includes antibodies that bind IL-4R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, or less than about 0.5 nM, as measured in a surface plasmon resonance assay. An isolated antibody that specifically binds human IL-4R may, however, have cross-reactivity to other antigens, such as IL-4R molecules from other (non-human) species.

The anti-IL-4R antibodies useful for the methods may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the invention.

Methods involving the use of anti-IL-4R antibodies comprising variants of any of the HCVR, LCVR, and/or CDR amino acid sequences disclosed herein having one or more conservative substitutions. For example, the use of anti-IL-4R antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein, are provided.

The term “surface plasmon resonance” refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}” refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, or at least about 96%, 97%, 98% or 99% of the nucleotide bases, as measured by any well-known algorithm of sequence identity, such as FASTA, BLAST or Gap, as discussed below.

As applied to polypeptides, the term “substantial similarity” or “substantially similar” means that two peptide sequences, when optimally aligned, such as by the programs GAP or BESTFIT using default gap weights, share at least 95% sequence identity, or at least 98% or 99% sequence identity. In exemplary embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. (See, e.g., Pearson (1994) Methods Mol. Biol. 24: 307-331, herein incorporated by reference.) Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Exemplary conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. (See, e.g., GCG Version 6.1.) Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another exemplary algorithm when comparing a sequence of the invention to a database containing a large number of sequences from different organisms is the computer program BLAST, especially BLASTP or TBLASTN, using default parameters. (See, e.g., Altschul et al. (1990) J. Mol. Biol. 215:403-410 and Altschul et al. (1997) Nucleic Acids Res. 25:3389-402, each of which is herein incorporated by reference.)

### Preparation of Human Antibodies

Methods for generating human antibodies in transgenic mice are known in the art. Any such known methods can be used to make human antibodies that specifically bind to human IL-4R.

Using VELOCIMMUNE® technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate a fully human antibody described herein, for example wild-type or modified IgG1 or IgG4. While the constant region selected may vary according to specific use, high affinity antigen-binding and target specificity characteristics reside in the variable region.

In general, the antibodies that can be used in the methods possess high affinities, as described above, when measured by binding to antigen either immobilized on solid phase or in solution phase. The mouse constant regions are replaced with desired human constant regions to generate the fully-human antibodies described herein. While the constant region selected may vary according to specific use, high affinity antigen-binding and target specificity characteristics reside in the variable region.

In one embodiment, human antibody or antigen-binding fragment thereof that specifically binds IL-4R that can be used in the context of the methods described herein comprises the three heavy chain CDRs (HCDR1, HCDR2 and HCDR3) contained within a heavy chain variable region (HCVR) having an amino acid sequence of SEQ ID NO: 1. The antibody or antigen-binding fragment may comprise the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) of SEQ ID NOs: 1 and 2.

In certain embodiments, the antibody or antigen-binding fragment thereof comprises six CDRs (HCDR1/HCDR2/HCDR3/LCDR1/LCDR2/LCDR3) having the amino acid sequences of SEQ ID NOs: 3/4/5/6/7/8.

In certain embodiments, the antibody or antigen-binding fragment thereof comprises HCVR/LCVR amino acid sequence pair of SEQ ID NOs: 1 and 2.

In certain embodiments, the antibody is dupilumab, which comprises the HCVR/LCVR amino acid sequence pair of SEQ ID NOs: 1 and 2.

In certain embodiments, the antibody sequence is dupilumab, which comprises the heavy chain/light chain amino acid sequence pair of SEQ ID NOs: 9 and 10.

Dupilumab HCVR amino acid sequence:
(SEQ ID NO: 1)
EVQLVESGGGLEQPGGSLRLSCAGSGFTFRDYAMTWVRQAPGKGLEWVSSISG
SGGNTYYADSVKGRFTISRDNSKNTLYLQMNSLRAEDTAVYYCAKDRLSITIRPRYYGL
DVWGQGTTVTVS.
Dupilumab LCVR amino acid sequence:
(SEQ ID NO: 2)
DIVMTQSPLSLPVTPGEPASISCRSSQSLLYSIGYNYLDWYLQKSGQSPQLLIYLG
SNRASGVPDRFSGSGSGTDFTLKISRVEAEDVGFYYCMQALQTPYTFGQGTKLEIK.
Dupilumab HCDR1 amino acid sequence:
(SEQ ID NO: 3)
GFTFRDYA.
Dupilumab HCDR2 amino acid sequence:
(SEQ ID NO: 4)
ISGSGGNT.
Dupilumab HCDR3 amino acid sequence:
(SEQ ID NO: 5)
AKDRLSITIRPRYYGL.
Dupilumab LCDR1 amino acid sequence:
(SEQ ID NO: 6)
QSLLYSIGYNY
Dupilumab LCDR2 amino acid sequence:
(SEQ ID NO: 7)
LGS.
Dupilumab LCDR3 amino acid sequence:
(SEQ ID NO: 8)
MQALQTPYT.
Dupilumab HC amino acid sequence:
(SEQ ID NO: 9)
EVQLVESGGGLEQPGGSLRLSCAGSGFTFRDYAMTWVRQAPGKGLEWVSSISG
SGGNTYYADSVKGRFTISRDNSKNTLYLQMNSLRAEDTAVYYCAKDRLSITIRPRYYGL
DVWGQGTTVTVSSASTKGPSVFPLAPCSRSTSESTAALGCLVKDYFPEPVTVSWNSGAL
TSGVHTFPAVLQSSGLYSLSSVVTVPSSSLGTKTYTCNVDHKPSNTKVDKRVESKYGPP
CPPCPAPEFLGGPSVFLFPPKPKDTLMISRTPEVTCVVVDVSQEDPEVQFNWYVDGVEV
HNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWSNGQPENNYKTPPVLDSDG
SFFLYSRLTVDKSRWQEGNVFSCSVMHEALHNHYTQKSLSLSLG
(amino acids 1-124 = HCVR; amino acids 125-451 = HC constant).
Dupilumab LC amino acid sequence:
(SEQ ID NO: 10)
DIVMTQSPLSLPVTPGEPASISCRSSQSLLYSIGYNYLDWYLQKSGQSPQLLIYLG
SNRASGVPDRFSGSGSGTDFTLKISRVEAEDVGFYYCMQALQTPYTFGQGTKLEIKRTV
AAPSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC
(amino acids 1-112 = LCVR; amino acids 112-219 = LC constant).

In certain embodiments, an antibody or antigen-binding fragment thereof of the disclosure comprises light chain variable region (LCVR) and heavy chain variable region (HCVR) sequence pairs (LCVR/HCVR) selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In certain embodiments, an antibody or antigen-binding fragment thereof of the disclosure comprises a LCVR/HCVR sequence pair of SCB-VL-44/SCB-VH-92.

In certain embodiments, an antibody or antigen-binding fragment thereof of the disclosure comprises a LCVR/HCVR sequence pair of SCB-VL-54/SCB-VH-92.

In certain embodiments, an antibody or antigen-binding fragment thereof of the disclosure comprises a LCVR/HCVR sequence pair of SCB-VL-55/SCB-VH-92.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-55-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-55-LCDR3.

In certain embodiments, an antibody or antigen-binding fragment thereof of the disclosure comprises an HCVR comprising an HCDR1 sequence of SCB-92-HCDR1, an HCDR2 sequence of SCB-92-HCDR2, and an HCDR3 sequence of SCB-92-HCDR3, and an LCVR comprising an LCDR1 of SCB-55-LCDR1, and LCDR2 of SCB-54-LCDR2, and an LCDR3 of SCB-44-LCDR3.

The antibodies recited below in Table 1 are described in more detail in U.S. Pat. No. 10,774,141, incorporated herein by reference in its entirety for all purposes.

TABLE 1

| Sequence | | SEQ ID |
|---|---|---|
| ID | Sequence | NO: |
| SCB-VL- | EIVLTQSPGTLSLSPGERATLSCRASQSVSNSYLAWYQQKPGQAPRLLI | 11 |
| 39 | FGASSRATGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSSSYLAWYQQKPGQAPRLLI | 12 |
| 40 | YGASSRATGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSSSYLAWYQQKPGQAPRLLI | 13 |
| 41 | FGASSRAPGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSNSYLAWYQQKPGQAPRLLI | 14 |
| 42 | YGASSRATGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSNSYLAWYQQKPGQAPRLLI | 15 |
| 43 | FGASSRAPGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSSSYLAWYQQKPGQAPRLLI | 16 |
| 44 | YGASSRAPGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSSSYLAWYQQKPGQAPRLLI | 17 |
| 45 | FGASSRATGIPDRFSGSGSGTDFTLTISRLEPEDFAVYYCQQYDHSPPW | |
| | TFGQGTKVEIK | |
| SCB-VL- | EIVLTQSPGTLSLSPGERATLSCRASQSVSSSYLAWYQQKPGQAPRLLI | 18 |
| 46 | FGASSRATGIPDRFSGSGSGTDFTLTISRLEPEDFAVYYCQQYGSSAG | |
| | WTFGQGTKVEIK | |
| SCB-VL- | EIVLTQSPGTLSLSPGERATLSCRASQSVSSSYLAWYQQKPGQAPRLLI | 19 |
| 47 | FGASSRATGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSNSYLAWYQQKPGQAPRLLI | 20 |
| 48 | FGASSRATGIPDRFSGSGSGTDFTLTISRLEPEDFAVYYCQQYDHSPPW | |
| | TFGQGTKVEIK | |
| SCB-VL- | EIVLTQSPGTLSLSPGERATLSCRASQSVSSSYLAWYQQKPGQAPRLLI | 21 |
| 49 | YGASSRATGIPDRFSGSGSGTDFTLTISRLEPEDFAVYYCQQYDHSPP | |
| | WTFGQGTKVEIK | |
| SCB-VL- | EIVLTQSPGTLSLSPGERATLSCRASQSVSSSYLAWYQQKPGQAPRLLI | 22 |
| 50 | FGASSRAPGIPDRFSGSGSGTDFTLTISRLEPEDFAVYYCQQYDHSPPW | |
| | TFGQGTKVEIK | |
| SCB-VL- | EIVLTQSPGTLSLSPGERATLSCRASQSVSSSYLAWYQQKPGQAPRLLI | 23 |
| 51 | YGASSRAPGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSNSYLAWYQQKPGQAPRLLI | 24 |
| 52 | FGASSRAPGIPDRFSGSGSGTDFTLTISRLEPEDFAVYYCQQYDHS AG | |
| | WTFGQGTKVEIK | |
| SCB-VL- | EIVLTQSPGTLSLSPGERATLSCRASQSVSNSYLAWYQQKPGQAPRLLI | 25 |
| 53 | YGASSRATGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSSSYLAWYQQKPGQAPRLLI | 26 |
| 54 | FGASSRAPGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSSSYLAWYQQKPGQAPRLLI | 27 |
| 55 | YGASSRATGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSNSYLAWYQQKPGQAPRLLI | 28 |
| 56 | FGASSRATGIPDRFSGSGSGTDFTLTISRLEPEDFAVYYCQQYDHSAG | |
| | WTFGQGTKVEIK | |
| SCB-VL- | EIVLTQSPGTLSLSPGERATLSCRASQSVSSSYLAWYQQKPGQAPRLLI | 29 |
| 57 | FGASSRATGIPDRFSGSGSGTDFTLTISRLEPEDFAVYYCQQYGSSPPW | |
| | TFGQGTKVEIK | |
| SCB-VL- | EIVLTQSPGTLSLSPGERATLSCRASQSVSNSYLAWYQQKPGQAPRLLI | 30 |
| 58 | YGASSRAPGIPDRFSGSGSGTDFTLTISRLEPEDFAVYYCQQYDHSAG | |
| | WTFGQGTKVEIK | |
| SCB- | EVQLVESGGGLVHPGGSLRLSCAGSGFTFSRNAMFWVRQAPGKGLE | 31 |
| VH-59 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVQPGGSLRLSCAGSGFTFSRNAMFWVRQAPGKGLE | 32 |
| VH-60 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAASGFTFSRNAMFWVRQAPGKGLE | 33 |
| VH-61 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGKGLE | 34 |
| VH-62 | WVSGIGTGGATSYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGKGLE | 35 |
| VH-63 | WVSGIGTGGATNYADSVKGRFTISRDNAKNSLYLQMNSLRAEDTAV | |
| | YYCARGRYYFDYWGQGTLVTVSS | |
| SCB- | EVQLVESGGGLVQPGGSLRLSCAGSGFTFSRNAMFWVRQAPGKGLE | 36 |
| VH-64 | WVSGIGTGGATNYADSVKGRFTISRDNAKNSLYLQMNSLRAEDMAV | |
| | YYCARGRYYFDYWGQGTLVTVSS | |
| SCB- | EVQLVESGGGLVHPGGSLRLSCAASGFTFSRNAMFWVRQAPGKGLE | 37 |
| VH-65 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVQPGGSLRLSCAASGFTFSRNAMFWVRQAPGKGLE | 38 |
| VH-66 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGKGLE | 39 |
| VH-67 | WVSGIGTGGATSYADSVKGRFTISRDNAKNSLYLQMNSLRAEDTAVY | |
| | YCARGRYYFDYWGQGTLVTVSS | |
| SCB- | EVQLVQSGGGLVHPGGSLRLSCAGSGFTFSRNAMFWVRQAPGKGLE | 40 |
| VH-68 | WVSGIGTGGATNYADSVKGRFTISRDNAKNSLYLQMNSLRAEDMAV | |
| | YYCARGRYYFPWWGQGTLVTVSS | |
| SCB- | EVQLVESGGGLVHPGGSLRLSCAGSGFTFSRNAMFWVRQAPGKGLE | 41 |
| VH-69 | WVSGIGTGGATNYADSVKGRFTISRDNAKNSLYLQMNSLRAEDMAV | |
| | YYCARGRYYFPWWGQGTLVTVSS | |
| SCB- | EVQLVQSGGGLVQPGGSLRLSCAGSGFTFSRNAMFWVRQAPGKGLE | 42 |
| VH-70 | WVSGIGTGGATNYADSVKGRFTISRDNAKNSLYLQMNSLRAEDMAV | |
| | YYCARGRYYFPWWGQGTLVTVSS | |
| SCB- | EVQLVQSGGGLVHPGGSLRLSCAASGFTFSRNAMFWVRQAPGKGLE | 43 |
| VH-71 | WVSGIGTGGATNYADSVKGRFTISRDNAKNSLYLQMNSLRAEDMAV | |
| | YYCARGRYYFPWWGQGTLVTVSS | |
| SCB- | EVQLVQSGGGLVHPGGSLRLSCAGSGFTFSRNAMFWVRQAPGKGLE | 44 |
| VH-72 | WVSGIGTGGATSYADSVKGRFTISRDNAKNSLYLQMNSLRAEDMAV | |
| | YYCARGRYYFPWWGQGTLVTVSS | |
| SCB- | EVQLVQSGGGLVHPGGSLRLSCAGSGFTFSRNAMFWVRQAPGKGLE | 45 |
| VH-73 | WVSGIGTGGATNYADSVKGRFTISRDNAKNSLYLQMNSLRAEDTAV | |
| | YYCARGRYYFPWWGQGTLVTVSS | |
| SCB- | EVQLVQSGGGLVHPGRSLRLSCAGSGFTFSRNAMFWVRQAPGKGLE | 46 |
| VH-74 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLTCAGSGFTFSRNAMFWVRQAPGKGLE | 47 |
| VH-75 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMHWVRQAPGKGLE | 48 |
| VH-76 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGEGLE | 49 |
| VH-77 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGKGLE | 50 |
| VH-78 | WVSGIGTGGATNYADSVKGRFTISRDEAKNSLYLQMNSLRAEDMAV | |
| | YYCARGRYYFDYWGQGTLVTVSS | |
| SCB- | EVQLVQSGGGLVHPGGSLRLSCAGSGFTFSRNAMFWVRQAPGKGLE | 51 |
| VH-79 | WVSGIGTGGATNYADSVKGRFTISRDNAKNSLYLQMNSLRAGDMAV | |
| | YYCARGRYYFDYWGQGTLVTVSS | |
| SCB- | EVQLVQSGGGLVHPGGSLRLSCAGSGFTFDDYAMFWVRQAPGKGLE | 52 |
| VH-80 | WVSGIGTGGATNYADSVKGRFTISRDNAKNSLYLQMNSLRAEDMAV | |
| | YYCARGRYYFDYWGQGTLVTVSS | |
| SCB- | EVQLVQSGGGLVQPGGSLRLSCAASGFTFSRNAMFWVRQAPGKGLE | 53 |
| VH-81 | WVSGIGTGGATSYADSVKGRFTISRDNAKNSLYLQMNSLRAEDTAVY | |
| | YCARGRYYFPWWGQGTLVTVSS | |
| SCB- | EVQLVESGGGLVHPGGSLRLSCAASGFTFSRNAMFWVRQAPGKGLE | 54 |
| VH-82 | WVSGIGTGGATSYADSVKGRFTISRDNAKNSLYLQMNSLRAEDTAVY | |
| | YCARGRYYFPWWGQGTLVTVSS | |
| SCB- | EVQLVESGGGLVQPGGSLRLSCAGSGFTFSRNAMFWVRQAPGKGLE | 55 |
| VH-83 | WVSGIGTGGATSYADSVKGRFTISRDNAKNSLYLQMNSLRAEDTAVY | |
| | YCARGRYYFPWWGQGTLVTVSS | |
| SCB- | EVQLVESGGGLVQPGGSLRLSCAASGFTFSRNAMFWVRQAPGKGLE | 56 |
| VH-84 | WVSGIGTGGATNYADSVKGRFTISRDNAKNSLYLQMNSLRAEDTAV | |
| | YYCARGRYYFPWWGQGTLVTVSS | |
| SCB- | EVQLVESGGGLVQPGGSLRLSCAASGFTFSRNAMFWVRQAPGKGLE | 57 |
| VH-85 | WVSGIGTGGATSYADSVKGRFTISRDNAKNSLYLQMNSLRAEDMAV | |
| | YYCARGRYYFPWWGQGTLVTVSS | |
| SCB- | EVQLVQSGGGLVHPGGSLRLSCAASGFTFSRNAMFWVRQAPGKGLE | 58 |
| VH-86 | WVSGIGTGGATSYADSVKGRFTISRDNAKNSLYLQMNSLRAEDTAVY | |
| | YCARGRYYFPWWGQGTLVTVSS | |
| SCB- | EVQLVQSGGGLVQPGGSLRLSCAGSGFTFSRNAMFWVRQAPGKGLE | 59 |
| VH-87 | WVSGIGTGGATSYADSVKGRFTISRDNAKNSLYLQMNSLRAEDTAVY | |
| | YCARGRYYFPWWGQGTLVTVSS | |
| SCB- | EVQLVESGGGLVHPGGSLRLSCAGSGFTFSRNAMFWVRQAPGKGLE | 60 |
| VH-88 | WVSGIGTGGATSYADSVKGRFTISRDNAKNSLYLQMNSLRAEDTAVY | |
| | YCARGRYYFPWWGQGTLVTVSS | |
| SCB- | EVQLVQSGGGLVHPGGSLRLSCAGSGFTFSRNAMFWVRQAPGKGLE | 61 |
| VH-89 | WVSGIGTGGATSYADSVKGRFTISRDNAKNSLYLQMNSLRAEDTAVY | |
| | YCARGRYYFPWWGQGTLVTVSS | |
| SCB- | EVQLVESGGGLVQPGGSLRLSCAASGFTFSRNAMFWVRQAPGKGLE | 62 |
| VH-90 | WVSGIGTGGATNYADSVKGRFTISRDNAKNSLYLQMNSLRAEDMAV | |
| | YYCARGRYYFPWWGQGTLVTVSS | |
| SCB- | EVQLVESGGGLVQPGGSLRLSCAASGFTFSRNAMFWVRQAPGKGLE | 63 |
| VH-91 | WVSGIGTGGATSYADSVKGRFTISRDNAKNSLYLQMNSLRAEDTAVY | |
| | YCARGRYYFDYWGQGTLVTVSS | |
| SCB- | EVQLVQSGGGLVHPGGSLRLSCAGSGFTFSRNAMFWVRQAPGKGLE | 64 |
| VH-92 | WVSGIGTGGATNYADSVKGRFTISRDNAKNSLYLQMNSLRAEDMAV | |
| | YYCARGRYYFDYWGQGTLVTVSS | |
| SCB- | EVQLVESGGGLVQPGGSLRLSCAASGFTFSRNAMFWVRQAPGKGLE | 65 |
| VH-93 | WVSGIGTGGATSYADSVKGRFTISRDNAKNSLYLQMNSLRAEDTAVY | |
| | YCARGRYYFPWWGQGTLVTVSS | |
| SCB-92- | RNAMF | 66 |
| HCDR1 | | |
| SCB-92- | GIGTGGATSYADSVKG | 67 |
| HCDR3 | | |
| SCB-92- | GRYYFDY | 68 |
| HCDR3 | | |
| SCB-55- | RASQSVSSSYLA | 69 |
| LCDR1 | | |
| SCB-55- | GASSRAT | 70 |
| LCDR2 | | |
| SCB-55- | QQYDHSAGWT | 71 |
| LCDR3 | | |
| SCB-54- | GASSRAP | 72 |
| LCDR2 | | |
| SCB-44- | QQYGSSPPWT | 73 |
| LCDR3 | | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of MEDI-1-VL/MEDI-1-VH through MEDI-42-VL/MEDI-42-VH.

In certain embodiments, an antibody or antigen-binding fragment thereof of the disclosure comprises a LCVR/HCVR sequence pair of MEDI-37GL-VL/MEDI-37GL-VH.

In certain embodiments, an antibody or antigen-binding fragment thereof of the disclosure comprises an HCVR comprising an HCDR1 sequence of MEDI-37GL-HCDR1, an HCDR2 sequence of MEDI-37GL-HCDR2, and an HCDR3 sequence of MEDI-37GL-HCDR3, and an LCVR comprising an LCDR1 of MEDI-37GL-LCDR1, and LCDR2 of MEDI-37GL-LCDR2, and an LCDR3 of MEDI-37GL-LCDR3.

The antibodies recited below in Table 2 are described in more detail in U.S. Pat. No. 8,877,189, incorporated herein by reference in its entirety for all purposes.

TABLE 2

| | | SEQ |
|---|---|---|
| Sequence | | ID |
| ID | Sequence | NO: |
| MEDI-1- | QVQLVQSGAEVKKPGASVKVSCKASGYAFTSYYMHWARQAPGQGL | 74 |
| VH | EWMGIINPSGGSTSYAQKFQGRVTMTRDTSTSTVYMELSSLRSEDTA | |
| | VYYCARGKWWLDYWGKGTLVTVSS | |
| MEDI-1- | QSVLTQPPSVSAAPGQKVTISCSGGSSNIGNSYVSWYQQLPGTAPKLLI | 75 |
| VL | YDNNKRPSGIPDRFSGSKSGTSATLAITGLQTGDEADYYCGTWDTSLS | |
| | ANYVFGTGTKLTVL | |
| MEDI-2- | QVQLVQSGAEVKKPGASVKVSCKASGYAFTSYYMHWARQAPGQGL | 76 |
| VH | EWMGIINPSGGSTSYAQKFQGRVTMTRDTSTSTVYMELSSLRSEDTA | |
| | VYYCARGKWWLYNWGKGTLVTVSS | |
| MEDI-2- | QSVLTQPPSVSAAPGQKVTISCSGGSSNIGNSYVSWYQQLPGTAPKLLI | 77 |
| VL | YDNNKRPSGIPDRFSGSKSGTSATLAITGLQTGDEADYYCGTWDTSQP | |
| | PNPLFGTGTKLTVL | |
| MEDI-3- | QVQLVQSGAEVKKPGASVKVSCKASGYAFTSYYMHWARQAPGQGL | 78 |
| VH | EWMGIINPSGGSTSYAQKFQGRVTMTRDTSTSTVYMELSSLRSEDTA | |
| | VYYCARGKLLKNPWGKGTLVTVSS | |
| MEDI-3- | QSVLTQPPSVSAAPGQKVTISCSGGSSNIGNSYVSWYQQLPGTAPKLLI | 79 |
| VL | YDNNKRPSGIPDRFSGSKSGTSATLAITGLQTGDEADYYCGTWFGTPA | |
| | SNYVFGTGTKLTVL | |
| MEDI-4- | QVQLVQSGAEVKKPGASVKVSCKASGYAFTSYYMHWARQAPGQGL | 76 |
| VH | EWMGIINPSGGSTSYAQKFQGRVTMTRDTSTSTVYMELSSLRSEDTA | |
| | VYYCARGKWWLYNWGKGTLVTVSS | |
| MEDI-4- | QSVLTQPPSVSAAPGQKVTISCSGGSSNIGNSYVSWYQQLPGTAPKLLI | 80 |
| VL | YDNNKRPSGIPDRFSGSKSGTSATLAITGLQTGDEADYYCGTWDTSSP | |
| | PQPIFGTGTKLTVL | |
| MEDI-5- | QVQLVQSGAEVKKPGASVKVSCKASGYAFTSYYMHWARQAPGQGL | 81 |
| VH | EWMGIINPSGGSTSYAQKFQGRVTMTRDTSTSTVYMELSSLRSEDTA | |
| | VYYCARGKWWLYDWGKGTLVTVSS | |
| MEDI-5- | QSVLTQPPSVSAAPGQKVTISCSGGSSNIGNSYVSWYQQLPGTAPKLLI | 80 |
| VL | YDNNKRPSGIPDRFSGSKSGTSATLAITGLQTGDEADYYCGTWDTSSP | |
| | PQPIFGTGTKLTVL | |
| MEDI-6- | QVQLVQSGAEVKKPGASVKVSCKASGYAFTSYYMHWARQAPGQGL | 82 |
| VH | EWMGIINPSGGSTSYAQKFQGRVTMTRDTSTSTVYMELSSLRSEDTA | |
| | VYYCARGKYWMYDWGKGTLVTVSS | |
| MEDI-6- | QSVLTQPPSVSAAPGQKVTISCSGGSSNIGNSYVSWYQQLPGTAPKLLI | 83 |
| VL | YDNNKRPSGIPDRFSGSKSGTSATLAITGLQTGDEADYYCGTWDTSTT | |
| | YHPIFGTGTKLTVL | |
| MEDI-7- | QVQLVQSGAEVKKPGASVKVSCKASGYAFTSYYMHWARQAPGQGL | 84 |
| VH | EWMGIINPSGGSTSYAQKFQGRVTMTRDTSTSTVYMELSSLRSEDTA | |
| | VYYCARGKWWWQYWGKGTLVTVSS | |
| MEDI-7- | QSVLTQPPSVSAAPGQKVTISCSGGSSNIGNSYVSWYQQLPGTAPKLLI | 80 |
| VL | YDNNKRPSGIPDRFSGSKSGTSATLAITGLQTGDEADYYCGTWDTSSP | |
| | PQPIF'GTGTKLTVL | |
| MEDI-8- | QVQLVQSGAEVKKPGASVKVSCKASGYAFTSYYMHWARQAPGQGL | 84 |
| VH | EWMGIINPSGGSTSYAQKFQGRVTMTRDTSTSTVYMELSSLRSEDTA | |
| | VYYCARGKWWWQYWGKGTLVTVSS | |
| MEDI-8- | QSVLTQPPSVSAAPGQKVTISCSGGSSNIGNSYVSWYQQLPGTAPKLLI | 83 |
| VL | YDNNKRPSGIPDRFSGSKSGTSATLAITGLQTGDEADYYCGTWDTSTT | |
| | YHPIFGTGTKLTVL | |
| MEDI-9- | QVQLVQSGAEVKKPGASVKVSCKASGYAFTSYYMHWARQAPGQGL | 76 |
| VH | EWMGIINPSGGSTSYAQKFQGRVTMTRDTSTSTVYMELSSLRSEDTA | |
| | VYYCARGKWWLYNWGKGTLVTVSS | |
| MEDI-9- | QSVLTQPPSVSAAPGQKVTISCSGGSSNIGNSYVSWYQQLPGTAPKLLI | 85 |
| VL | YDNNKRPSGIPDRFSGSKSGTSATLAITGLQTGDEADYYCGTWDTSTT | |
| | MYPLFGTGTKLTVL | |
| MEDI- | QVQLVQSGAEVKKPGASVKVSCKASGYAFTSYYMHWARQAPGQGL | 81 |
| 10-VH | EWMGIINPSGGSTSYAQKFQGRVTMTRDTSTSTVYMELSSLRSEDTA | |
| | VYYCARGKWWLYDWGKGTLVTVSS | |
| MEDI- | QSVLTQPPSVSAAPGQKVTISCSGGSSNIGNSYVSWYQQLPGTAPKLLI | 86 |
| 10-VL | YDNNKRPSGIPDRFSGSKSGTSATLAITGLQTGDEADYYCGTWDTSTV | |
| | LTPIFGTGTKLTVL | |
| MEDI- | QVQLVQSGAEVKKPGASVKVSCKASGYAFTSYYMHWARQAPGQGL | 87 |
| 11-VH | EWMGIINPSGGSTSYAQKFQGRVTMTRDTSTSTVYMELSSLRSEDTA | |
| | VYYCARGKWWFYDWGKGTLVTVSS | |
| MEDI- | QSVLTQPPSVSAAPGQKVTISCSGGSSNIGNSYVSWYQQLPGTAPKLLI | 88 |
| 11-VL | YDNNKRPSGIPDRFSGSKSGTSATLAITGLQTGDEADYYCGTWDTSPS | |
| | MIPLFGTGTKLTVL | |
| MEDI- | QVQLVQSGAEVKKPGASVKVSCKASGYAFTSYYMHWARQAPGQGL | 87 |
| 12-VH | EWMGIINPSGGSTSYAQKFQGRVTMTRDTSTSTVYMELSSLRSEDTA | |
| | VYYCARGKWWFYDWGKGTLVTVSS | |
| MEDI- | QSVLTQPPSVSAAPGQKVTISCSGGSSNIGNSYVSWYQQLPGTAPKLLI | 85 |
| 12-VL | YDNNKRPSGIPDRFSGSKSGTSATLAITGLQTGDEADYYCGTWDTSTT | |
| | MYPLFGTGTKLTVL | |
| MEDI- | QVQLVQSGAEVKKPGASVKVSCKASGYAFTSYYMHWARQAPGQGL | 81 |
| 13-VH | EWMGIINPSGGSTSYAQKFQGRVTMTRDTSTSTVYMELSSLRSEDTA | |
| | VYYCARGKWWLYDWGKGTLVTVSS | |
| MEDI- | QSVLTQPPSVSAAPGQKVTISCSGGSSNIGNSYVSWYQQLPGTAPKLLI | 89 |
| 13-VL | YDNNKRPSGIPDRFSGSKSGTSATLAITGLQTGDEADYYCGTWDTSTT | |
| | LQPLFGTGTKLTVL | |
| MEDI- | QVQLVQSGAEVKKPGASVKVSCKASGYAFTSYYMHWARQAPGQGL | 76 |
| 14-VH | EWMGIINPSGGSTSYAQKFQGRVTMTRDTSTSTVYMELSSLRSEDTA | |
| | VYYCARGKWWLYNWGKGTLVTVSS | |
| MEDI- | QSVLTQPPSVSAAPGQKVTISCSGGSSNIGNSYVSWYQQLPGTAPKLLI | 90 |
| 14-VL | YDNNKRPSGIPDRFSGSKSGTSATLAITGLQTGDEADYYCGTWDTSPP | |
| | TKPLFGTGTKLTVL | |
| MEDI- | QVQLVQSGAEVKKPGASVKVSCKASGYAFTSYYMHWARQAPGQGL | 76 |
| 15-VH | EWMGIINPSGGSTSYAQKFQGRVTMTRDTSTSTVYMELSSLRSEDTA | |
| | VYYCARGKWWLYNWGKGTLVTVSS | |
| MEDI- | QSVLTQPPSVSAAPGQKVTISCSGGSSNIGNSYVSWYQQLPGTAPKLLI | 91 |
| 15-VL | YDNNKRPSGIPDRFSGSKSGTSATLAITGLQTGDEADYYCGTWDTSTH | |
| | RHPLFGTGTKLTVL | |
| MEDI- | QVQLVQSGAEVKKPGASVKVSCKASGYAFTSYYMHWARQAPGQGL | 76 |
| 16-VH | EWMGIINPSGGSTSYAQKFQGRVTMTRDTSTSTVYMELSSLRSEDTA | |
| | VYYCARGKWWLYNWGKGTLVTVSS | |
| MEDI- | QSVLTQPPSVSAAPGQKVTISCSGGSSNIGNSYVSWYQQLPGTAPKLLI | 83 |
| 16-VL | YDNNKRPSGIPDRFSGSKSGTSATLAITGLQTGDEADYYCGTWDTSTT | |
| | YHPIFGTGTKLTVL | |
| MEDI- | QVQLVQSGAEVKKPGASVKVSCKASGYAFTSYYMHWARQAPGQGL | 92 |
| 17-VH | EWMGIINPSGGSTSYAQKFQGRVTMTRDTSTSTVYMELSSLRSEDTA | |
| | VYYCARGKWWWQHWGKGTLVTVSS | |
| MEDI- | QSVLTQPPSVSAAPGQKVTISCSGGSSNIGNSYVSWYQQLPGTAPKLLI | 93 |
| 17-VL | YDNNKRPSGIPDRFSGSKSGTSATLAITGLQTGDEADYYCGTWDTSPV | |
| | DRPIFGTGTKLTVL | |
| MEDI- | QVQLVQSGAEVKKPGASVKVSCKASGYAFTSYYMHWARQAPGQGL | 92 |
| 18-VH | EWMGIINPSGGSTSYAQKFQGRVTMTRDTSTSTVYMELSSLRSEDTA | |
| | VYYCARGKWWWQHWGKGTLVTVSS | |
| MEDI- | QSVLTQPPSVSAAPGQKVTISCSGGSSNIGNSYVSWYQQLPGTAPKLLI | 94 |
| 18-VL | YDNNKRPSGIPDRFSGSKSGTSATLAITGLQTGDEADYYCGTWDTSTT | |
| | PMPVFGTGTKLTVL | |
| MEDI- | QVQLVQSGAEVKKPGASVKVSCKASGYAFTSYYMHWARQAPGQGL | 92 |
| 19-VH | EWMGIINPSGGSTSYAQKFQGRVTMTRDTSTSTVYMELSSLRSEDTA | |
| | VYYCARGKWWWQHWGKGTLVTVSS | |
| MEDI- | QSVLTQPPSVSAAPGQKVTISCSGGSSNIGNSYVSWYQQLPGTAPKLLI | 83 |
| 19-VL | YDNNKRPSGIPDRFSGSKSGTSATLAITGLQTGDEADYYCGTWDTSTT | |
| | YHPIFGTGTKLTVL | |
| MEDI- | QVQLVQSGAEVKKPGASVKVSCKASGYAFTSYYMHWARQAPGQGL | 82 |
| 20-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 95 |
| 20-VL | YDNNKRPSGIPDRFSGSKSGTSATLAITGLQTGDEADYYCGTWDTSTV | |
| | WEWPFGTGTKLTVL | |
| MEDI- | QVQLVQSGAEVKKPGASVKVSCKASGYAFTSYYMHWARQAPGQGL | 96 |
| 21-VH | EWMGIINPSGGSASYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 97 |
| 21-VL | YDNNKRPSGIPDRFSGSKSGTSATLAITGLQTGDEAVYFCGTWDTSTV | |
| | WEWPFGTGTKLTVL | |
| MEDI- | QVQLVQSGAEVKKPGASVKVSCKASGYAFTSYYMHWARQAPGQGL | 82 |
| 22-VH | EWMGIINPSGGSTSYAQKFQGRVTMTRDTSTSTVYMELSSLRSEDTA | |
| | VYYCARGKYWMYDWGKGTLVTVSS | |
| MEDI- | QPVLTQPPSVSAAPGQKVTISCSGGSSNIGNSYVSWYQQLPGTAPKLLI | 98 |
| 22-VL | YDNNKRPSGIPDRFSGSKSGTSATLAITGLQTGDEADYFCGTWDTSTV | |
| | WEWPFGTGTKLTVL | |
| MEDI- | QVQLVQSGAEVRKPGASVKVSCKASGYAFTSYYMHWARQAPGQGL | 99 |
| 23-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNNYVSWYQQLPGTAPKLL | 100 |
| 23-VL | IYDNNKRPPGIPDRFSGSKSGTSATLAITGLQTGDEADYYCGTWDTST | |
| | VWEWPFGTGTKLTVL | |
| MEDI- | QVQLVQSGAEVKKPGASVKVSCKASGYAFTSYYMHWARQAPGQGL | 101 |
| 24-VH | EWMGIINPRGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 102 |
| 24-VL | YDNNKRPSGIPDRFSGSKSGTSATLAITGLQTGDEADYFCGTWDTSTV | |
| | WEWPFGTGTKLTVL | |
| MEDI- | QVQLVQSGAEVKKPGASVKVSCKASGYAFTSYYMHWARQAPGQGL | 103 |
| 25-VH | EWMGIINPRGGSASYAQKFQGRVSMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 104 |
| 25-VL | YDNNKRPSGIPDRFSGSKSGTTATLAITGLQTGDEADYYCGTWVTST | |
| | VWEWPFGTGTKLTVL | |
| MEDI- | QVQLVQSGAEVKKPGASVKVSCKASGYAFTSYYMHWARQAPGQGL | 82 |
| 26-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 102 |
| 26-VL | YDNNKRPSGIPDRFSGSKSGTSATLAITGLQTGDEADYFCGTWDTSTV | |
| | WEWPFGTGTKLTVL | |
| MEDI- | QVQLVQSGAEVRKPGASVKVSCKASGYAFTSYYMHWARQAPGQGL | 105 |
| 27-VH | EWMGIINPSGGSTSYAQKFQGRVTMTRDTSTSTVYMELSSLRPEDTA | |
| | VYYCARGKYWMYDWGKGTQVTVSS | |
| MEDI- | QSVLTQPPLVSAAPGQKVTISCSGGSSNIGNSYVSWYQRLPGTAPKLLI | 106 |
| 27-VL | YDNNKRPSGIPDRFSGSKSGTSATLAITGLQTGDEADYYCGTWDTSTV | |
| | WEWPFGTGTKLTVL | |
| MEDI- | QVQLVQSGAEVKKPGASVKVSCKASGYAFTSYYMHWARQAPGQGL | 107 |
| 28-VH | EWMGIINPSGGSTSYAQKFQGRVTMTRDTSTSTVYMELSSLRSEDTA | |
| | VYYCARGKYWMYDWGNGTLVTVSS | |
| MEDI- | LPVLTQPPSVSAAPGQKVTISCSGGSSSIGNSYVSWYQQLPGAAPKLLI | 108 |
| 28-VL | YDNNKRPSGIPDRFSGFRSGTSATLAITGLQTGDEADYYCGTWDTSPV | |
| | WEWPFGTGTKLTVL | |
| MEDI- | QVQLVQSGAEVKKPGASVKVSCKASGYAFTSYYMHWARQAPGQGL | 109 |
| 29-VH | EWMGIINPSGGSTSYAQKFQGRVTMTRDTSTSTVYMELSSLRSEDTA | |
| | VYYCARGKYWMYDWGKGTRVTVS | S |
| MEDI- | QSVLTQPPSVSAAPGQKVTISCSGGSSNIGNSYVSWYQQLPGTAPKLLI | 110 |
| 29-VL | YDNNKRPSGIPDRFSGSKSGTSATLAITGLQTGDEADYYCGTWDTSPV | |
| | WEWPFGTGTKLTVL | |
| MEDI- | QVQLVQSGAEVKKPGASVKVSCKASGYAFTSYYMHWARQAPGQGL | 82 |
| 30-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQRLPGAAPKLLI | 111 |
| 30-VL | YDNNKRPSGIPDRFSGSKSGTSATLAITGLQTGDEADYYCGTWDTSTV | |
| | WEWPFGTGTKLTVL | |
| MEDI- | QVQLVQSGAEVKKPGASVKVSCKASGYAFTSYYMHWARQAPGQGL | 82 |
| 31-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSSIGNSYVSWYQQLPGTAPKLLI | 112 |
| 31-VL | YDNNKRPSGIPDRFSGSKSGTSATLAITGLQTGDEADYYCGTWATSPV | |
| | WEWPFGTGTKLTVL | |
| MEDI- | QVQLVQSGAEVKKPGASVKVSCKASGYAFTSYYMHWARQAPGQGL | 82 |
| 32-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 113 |
| 32-VL | YDNNKRPSGIPDRFSGSKSGTSATLAITGLQTGDEADYFCGTWDTSTA | |
| | WEWPFGTGTKLTVL | |
| MEDI- | QVQLVQSGAEEKKPGASVKVSCKASGYAFTSYYMHWARQ APGQGL | 114 |
| 33-VH | EWMGIINPSGGSTSYAQKFQGRVTMTRDTSTSTVYMELSSLRSEDTA | |
| | VYYCARGKYWMYDWGKGTLVTVSS | |
| MEDI- | QSALTQPPSVSAAPGQKVTISCSGGSSNIGNSYVSWYQQLPGTAPKLLI | 115 |
| 33-VL | YDNNKRPSGIPDRFSGSKSGTSATLAITGLQTGDEADYFCGTWDTSTV | |
| | WEWPFGTGTKLTVL | |
| MEDI- | QVQLVQSGAEVKKPGASVKVSCKASGYAFTSYYMHWARQAPGQGL | 116 |
| 34-VH | EWMGIINPSGGSTSYAQKFQGRVSMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 102 |
| 34-VL | YDNNKRPSGIPDRFSGSKSGTSATLAITGLQTGDEADYFCGTWDTSTV | |
| | WEWPFGTGTKLTVL | |
| MEDI- | QVQLVQSGAEVKKPGASVKVSCKASGYAFTSYYMHWARQAPGQGL | 82 |
| 35-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 110 |
| 35-VL | YDNNKRPSGIPDRFSGSKSGTSATLAITGLQTGDEADYYCGTWDTSPV | |
| | WEWPFGTGTKLTVL | |
| MEDI- | QVQLVQSGAEVKKPGASVKVSCKASGYAFTSYYMHWARQAPGQGL | 96 |
| 36-VH | EWMGIINPSGGSASYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 117 |
| 36-VL | YDNNKRPSGIPDRFSGSKSGTSATLAITGLQTGDEADYYCGTWDSSTV | |
| | WEWPFGTGTKLTVL | |
| MEDI- | QVQLVQSGAEVKKPGASVKVSCKASGYAFTSYYMHWARQAPGQGL | 118 |
| 37-VH | EWMGIINPRGGSTSYAQKFQGRVAMTRDTSTSTVYMELSSLRPEDTA | |
| | VYYCARGKYWMYDWGKGTLVTVSS | |
| MEDI- | QSVLTQPPSVSAAPGQKVTISCSGGGSSIGNSYVSWYQQLPGTAPKLLI | 119 |
| 37-VL | YDNNKRPSGVPDRFSGSKSGTSATLAITGLQTGDEADYYCGTWDTSP | |
| | VWEWPFGTGTKLTVL | |
| MEDI- | QVQLVQSGAEVKKPGASVKVSCKASGYAFTSYYMHWARQAPGQGL | 96 |
| 38-VH | EWMGIINPSGGSASYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 102 |
| 38-VL | YDNNKRPSGIPDRFSGSKSGTSATLAITGLQTGDEADYFCGTWDTSTV | |
| | WEWPFGTGTKLTVL | |
| MEDI- | QVQLVQSGAEVKKPGASVKVSCKASGYAFTSYYMHWARQAPGQGL | 101 |
| 39-VH | EWMGIINPRGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 120 |
| 39-VL | YDNNKRPSGIPDRFSGSKSGTSATLAITGLQTGDEADYYCGTWDTSTA | |
| | WEWPFGTGTKLTVL | |
| MEDI- | QVQLVQSGAEVKKPGASVKVSCKASGYAFTSYYMHWARQAPGQGL | 82 |
| 40-VH | EWMGIINPSGGSTSYAQKFQGRVTMTRDTSTSTVYMELSSLRSEDTA | |
| | VYYCARGKYWMYDWGKGTLVTVSS | |
| MEDI- | QSVLTQPPSVSAAPGQKVTISCSGGSSNIGNSYVSWYQQLPGTAPKLLI | 117 |
| 40-VL | YDNNKRPSGIPDRFSGSKSGTSATLAITGLQTGDEADYYCGTWDSSTV | |
| | WEWPFGTGTKLTVL | |
| MEDI- | QVQLVQSGAEVRKPGASVKVSCKASGYAFTSYYMHWARQAPGQGL | 121 |
| 41-VH | EWMGIINPSGGSTSYAQKFQGRVTMTRDTSTSTVYMELSSLRPEDTA | |
| | VYYCARGKYWMYDWGKGTLVTVSG | |
| MEDI- | QSVLTQPPSVSAAPGQKVTISCSGGSTNIGNSYVSWYQRLPGTAPKLLI | 122 |
| 41-VL | YDNNKRPPGIPDRFSGSKSGTSATLAITGLQTGDEADYYCGTWDTSTV | |
| | WEWPFGTGTKLTVL | |
| MEDI- | QVQLVQSGAEVKKPGASVKVSCKASGYAFTSYYMHWARQAPGQGL | 123 |
| 42-VH | EWVGIINPSGGSTSYAQKFQGRVTMTRDTSTSTVYMELSSLRSGDTA | |
| | VYYCARGKYWIVIYDWGKGTLVTVSS | |
| MEDI- | QAVLTQPPSVSAAPGQKVTISCSGGSSNIGNSYVSWYQRLPGAAPKLL | 124 |
| 42-VL | IYDNNKRPSGIPDRFSGSKSGTSATLAITGLQTGDEADYYCGTWDTST | |
| | GWEWPFGTGTKLTVL | |
| MEDI- | QVQLVQSGAEVKKPGASVKVSCKASGYAFTSYYMHWVRQAPGQGL | 125 |
| 37GL- | EWMGIINPRGGSTSYAQKFQGRVTMTRDTSTSTVYMELSSLRSEDTA | |
| VH | VYYCARGKYWIVIYDWGKGTLVTVSS | |
| MEDI- | QSVLTQPPSVSAAPGQKVTISCSGGGSSIGNSYVSWYQQLPGTAPKLLI | 126 |
| 37GL- | YDNNKRPSGIPDRFSGSKSGTSATLGITGLQTGDEADYYCGTWDTSPV | |
| VL | WEWPFGTGTKLTVL | |
| MEDI- | SYYMH | 127 |
| 37GL- | | |
| HCDR1 | | |
| MEDI- | IINPRGGSTSYAQKFQG | 128 |
| 37GL- | | |
| HCDR2 | | |
| MEDI- | GKYWMYD | 129 |
| 37GL- | | |
| HCDR3 | | |
| MEDI- | SGGGSSIGNSYVS | 130 |
| 37GL- | | |
| LCDR1 | | |
| MEDI- | DNNKRPS | 131 |
| 37GL- | | |
| LCDR2 | | |
| MEDI- | GTWDTSPVWEWP | 132 |
| 37GL- | | |
| LCDR3 | | |

In certain embodiments, an antibody or antigen-binding fragment thereof of the disclosure comprises a LCVR/HCVR sequence pair of AJOU-90-VL/AJOU-83-VH.

In certain embodiments, an antibody or antigen-binding fragment thereof of the disclosure comprises an HCVR comprising an HCDR1 sequence of AJOU-84-HCDR1, an CHDR2 sequence of AJOU-85-HCDR2, and an HCDR3 sequence of AJOU-32-HCDR3, and an LCVR comprising an LCDR1 of AJOU-96-LCDR1, and LCDR2 of AJOU-60-LCDR2, and an LCDR3 of AJOU-68-LCDR3.

The antibodies recited below in Table 3 are described in more detail in WO2020/096381 and Kim et al. (Scientific Reports. 9: 7772. 2019), incorporated herein by reference in their entireties for all purposes.

TABLE 3

| Sequence | | SEQ ID |
|---|---|---|
| ID | Sequence | NO: |
| AJOU-1- | EVQLLESGGGLVQPGGSLRLSCAVSGFTFSNYAMSWVRQAPGKGLE | 133 |
| VH | WVSAISSGGGNIYYADSVKGRFTISRDNSKNTLYLQMNSLRAEDTAV | |
| | YYCAKLRRYFDYWGQGTLVTVSS | |
| AJOU-2- | EVQLLESGGGLVQPGGSLRLSCAASGFTFSDYAMSWVRQAPGKGLE | 134 |
| VH | WVSAISSGGSSIYYADSVKGRFTISRDNSKNTLHLQMNSLRAEDTAVY | |
| | YCARGPQRSATAVFDYWGQGTLVTVSS | |
| AJOU-3- | EVQLLESGGGLVQPGGSLRLSCAASGFTFSNYAMSWVRQAPGKGLE | 135 |
| VH | WVSWISPNSGNIYYADSVKGRFTISRDNSKNTLYLQMNSLRAEDTAV | |
| | YYCARRPLSAAWSHSSYYNAMDVWGQGTLVTVSS | |
| AJOU-4- | EVQLLESGGGLVQPGGSLRLSCAASGFTFSGYAMSWVRQAPGKGLE | 136 |
| VH | WVSLISHSGSNTYYADSVKGRFTISRDNSKNTLYLQMNSLRAEDTAV | |
| | YYCARPHRAFDYWGQGTLVTVSS | |
| AJOU-5- | EVQLLESGGGLVQPGGSLRLSCAASGFTFSNYAMSWVRQAPGKGLE | 137 |
| VH | WVSGISHGSGSIYYADSVKGRFTISRDNSKNTLYLQMNSLRAEDTAV | |
| | YYCARPHRAFDYWGQGTLVTVSS | |
| AJOU-6- | EVQLLESGGGLVQPGGSLRLSCAASGFTFSNYAMSWVRQAPGKGLE | 138 |
| VH | WVSGISHGNGSIYYADSVKGRFTISRDNSKNTLYLQMNSLRAEDTAV | |
| | YYCAKTGRHFDYWGQGTLVTVSS | |
| AJOU-7- | EVQLLESGGGLVQPGGSLRLSCAASGFTFSNYAMSWVRQAPGKGLE | 139 |
| VH | WVSSISPSGSSIYYADSVKGRFTISRDNSKNTLYLQMNSLRAEDTAVY | |
| | YCARSYRAFDYWGQGTLVTVSS | |
| AJOU-8- | EVQLLESGGGLVQPGGSLRLSCAASGFTFSNYAMSWVRQAPGKGLE | 140 |
| VH | WVSAISPSGGSIYYADSVKGRFTISRDNSKNTLYLQMNSLRAEDTAVY | |
| | YCARAKRAFDYWGQGTLVTVSS | |
| AJOU-9- | EVQLLESGGGLVQPGGSLRLSCAASGFTFSNYAMSWVRQAPGKGLE | 141 |
| VH | WVSAISPGSGSTYYADSVKGRFTISRDNSKNTLYLQMNSLRAEDTAV | |
| | YYCAKFRRHFDYWGQGTLVTVSS | |
| AJOU- | EVQLLESGGGLVQPGGSLRLSCAASGFTFSNYAMSWVRQAPGKGLE | 142 |
| 10-VH | WVSAISSGGGNIYYADSVKGRFTISRDNSKNTLYLQMNSLRAEDTAV | |
| | YYCARVHRAFDYWGQGTLVTVSS | |
| AJOU- | EVQLLESGGGLVQPGGSLRLSCAASGFTFSNYAMSWVRQAPGKGLE | 143 |
| 69-VH | WVSAITSSGRSIYYADSVKGRFTISRDNSKNTLYLQMNSLRAEDTAVY | |
| | YCARVHRAFDYWGQGTLVTVSS | |
| AJOU- | EVQLLESGGGLVQPGGSLRLSCAASGFTFSNYAMSWVRQAPGKGLE | 144 |
| 70-VH | WVSAITSSGANIYYADSVKGRFTISRDNSKNTLYLQMNSLRAEDTAV | |
| | YYCARVHRAFDYWGQGTLVTVSS | |
| AJOU- | EVQLLESGGGLVQPGGSLRLSCAASGFTFSNYAMSWVRQAPGKGLE | 145 |
| 71-VH | WVSAITSSGGNIYYADSVKGRFTISRDNSKNTLYLQMNSLRAEDTAV | |
| | YYCARVHRAFDYWGQGTLVTVSS | |
| AJOU- | EVQLLESGGGLVQPGGSLRLSCAASGFTFSNYAMSWVRQAPGKGLE | 146 |
| 72-VH | WVSAITAGGGSIYYADSVKGRFTISRDNSKNTLYLQMNSLRAEDTAV | |
| | YYCARVHRAFDYWGQGTLVTVSS | |
| AJOU- | EVQLLESGGGLVQPGGSLRLSCAASGFTFSRHAMAWVRQAPGKGLE | 147 |
| 83-VH | WVSAITSSGRSIYYADSVKGRFTISRDNSKNTLYLQMNSLRAEDTAVY | |
| | YCARVHRAFDYWGQGTLVTVSS | |
| AJOU- | QSVLTQPPSASGTPGQRVTISCSGSSSNIGNNYVNWYQQLPGTAPKLLI | 148 |
| 33-VL | YDNSHRPSGVPDRFSGSKSGTSASLAISGLRSEDEADYYCGTWDASLS | |
| | AYVFGGGTKLTVL | |
| AJOU- | QSVLTQPPSASGTPGQRVTISCSGSSSNIGNNNVSWYQQLPGTAPKLLI | 149 |
| 34-VL | YANSKRPSGVPDRFSGSKSGTSASLAISGLRSEDEADYYCGSWDDSLS | |
| | AYVFGGGTKLTVL | |
| AJOU- | QSVLTQPPSAPGTPGQRVTISCTGSSSNIGSNSVNWYQQLPGTAPKLLI | 150 |
| 35-VL | YDDSHRPSGVPDRFSGSKSGTSASLAISGLRSEDEADYYCDAWDSSLS | |
| | AYVFGGGTKLTVL | |
| AJOU- | QSVLTQPPSASGTPGQRVTLSCTGSSSNIGSNYVSWYQQLPGTAPKLLI | 151 |
| 36-VL | YADSQRPSGVPDRFSGSKSGTSASLAISGLRSEDEADYYCGTWDDSLS | |
| | GYVFGGGTKLTVL | |
| AJOU- | QSVLTQPPSASGTPGQRVTISCSSSSSNIGSNYVSWYQQLPGTAPKLLI | 152 |
| 37-VL | YSDSHRPSGVPDRFSGSKSGTSASLAISGLRSEDEADYYCGSWDYSLS | |
| | AYVFGGGTKLTVL | |
| AJOU- | QSVLTQPPSASGTPGQRVTISCTGSSSNIGNNTVSWYQQLPGTAPKLLI | 153 |
| 38-VL | YDNSHRPSGVPDRFSGSKSGTSASLAISGLQSEDEADYYCGSWDYSLS | |
| | AYVFGGGTKLTVL | |
| AJOU- | QSVLTQPPSASGTPGQRVTISCTGSSSNIGNNDVNWYQQLPGTAPKLLI | 154 |
| 39-VL | YYDSQRPSGVPDRFSGSKSGTSASLAISGLRSEDEADYYCATWDASLS | |
| | AYVFGGGTKLTVL | |
| AJOU- | QSVLTQPPSASGTPGQRVTISCSGSSSNIGSNAVNWYQQLPGTAPKLLI | 155 |
| 40-VL | YYDNQRPSGVPDRFSGSKSGTSASLAISGLRSEDEADYYCGTWDDSL | |
| | NGYVFGGGTKLTVL | |
| AJOU- | QSVLTQPPSASGTPGQRVTISCSGSSSNIGNNAVTWYQQLPGTAPKLLI | 156 |
| 41-VL | YDDSHRPSGVPDRFSGSKSGTSASLAISGLRSEDEADYYCGSWDYSLS | |
| | AYVFGGGTKLTVL | |
| AJOU- | QSVLTQPPSASGTPGQRVTISCSGSSSNIGSNTFNWYQQLPGTAPKLLI | 157 |
| 42-VL | YADSHRPSGVPDRFSGSKSGTSASLAISGLRSEDEADYYCGTWDYSLS | |
| | GYVLGGGTKLTVL | |
| AJOU- | QSVLTQPPSASGTPGQRVTISCSGSSSNIGSNTFNWYQQLPGTAPKLLI | 157 |
| 77-VL | YADSHRPSGVPDRFSGSKSGTSASLAIS GLRSEDEADYYCGTWDYSLS | |
| | GYVLGGGTKLTVL | |
| AJOU- | QSVLTQPPSASGTPGQRVTISCSGSSSNIGSNTFNWYQQLPGTAPKLLI | 158 |
| 78-VL | YADSHRPSGVPDRFSGSKSGTSASLAISGLRSEDEADYYCGTWDYSLR | |
| | GYVLGGGTKLTVL | |
| AJOU- | QSVLTQPPSASGTPGQRVTISCSGSSSNIGSNTFNWYQQLPGTAPKLLI | 159 |
| 79-VL | YADSHRPSGVPDRFSGSKSGTSASLAISGLRSEDEADYYCGYWDYSLS | |
| | GYVLGGGTKLTVL | |
| AJOU- | QSVLTQPPSASGTPGQRVTISCSGSSSNIGSNTFNWYQQLPGTAPKLLI | 157 |
| 80-VL | YADSHRPSGVPDRFSGSKSGTSASLAISGLRSEDEADYYCGTWDYSLS | |
| | GYVLGGGTKLTVL | |
| AJOU- | QSVLTQPPSASGTPGQRVTISCSGSSANSRTDGFNWYQQLPGTAPKLLI | 160 |
| 86-VL | YADSHRPSGVPDRFSGSKSGTSASLAISGLRSEDEADYYCGTWDYSLS | |
| | GYVLGGGTKLTVLG | |
| AJOU- | QSVLTQPPSASGTPGQRVTISCSGSAQFGSRDNFNWYQQLPGTAPKLL | 161 |
| 87-VL | IYADSHRPSGVPDRFSGSKSGTSASLAISGLRSEDEADYYCGTWDYSL | |
| | SGYVLGGGTKLTVLG | |
| AJOU- | QSVLTQPPSASGTPGQRVTISCSGSTKQMHNYQFNWYQQLPGTAPKL | 162 |
| 88-VL | LIYADSHRPSGVPDRFSGSKSGTSASLAISGLRSEDEADYYCGTWDYS | |
| | LSGYVLGGGTKLTVLG | |
| AJOU- | QSVLTQPPSASGTPGQRVTISCSGSLLRGENLQFNWYQQLPGTAPKLLI | 163 |
| 89-VL | YADSHRPSGVPDRFSGSKSGTSASLAISGLRSEDEADYYCGTWDYSLS | |
| | GYVLGGGTKLTVLG | |
| AJOU- | QSVLTQPPSASGTPGQRVTISCSGSPLFPDSGSFNWYQQLPGTAPKLLI | 164 |
| 90-VL | YADSHRPSGVPDRFSGSKSGTSASLAISGLRSEDEADYYCGTWDYSLS | |
| | GYVLGGGTKLTVLG | |
| AJOU- | QSVLTQPPSASGTPGQRVTISCSGSAALDLSPSFNWYQQLPGTAPKLLI | 165 |
| 91-VL | YADSHRPSGVPDRFSGSKSGTSASLAISGLRSEDEADYYCGTWDYSLS | |
| | GYVLGGGTKLTVLG | |
| AJOU- | RHAMA | 166 |
| 84- | | |
| HCDR1 | | |
| AJOU- | AITSSGRSIYYADSVKG | 167 |
| 85- | | |
| HCDR2 | | |
| AJOU- | VHRAFDY | 168 |
| 32- | | |
| HCDR3 | | |
| AJOU- | SGSPLFPDSGSFN | 169 |
| 96- | | |
| LCDR1 | | |
| AJOU- | ADSHRPS | 170 |
| 60- | | |
| LCDR2 | | |
| AJOU- | GTWDYSLSGYV | 171 |
| 68- | | |
| LCDR3 | | |

In certain embodiments, an antibody or antigen-binding fragment thereof of the disclosure comprises light chain variable region (LCVR) and heavy chain variable region (HCVR) sequence pairs (LCVR/HCVR) selected from the group consisting of 11/3, 27/19, 43/35, 59/51, 75/67, 91/83, 107/99, 123/115, 155/147, and 171/163.

The antibodies recited below in Table 4 are described in more detail in U.S. Pat. Nos. 7,605,237 and 7,608,693, incorporated herein by reference in their entireties for all purposes.

TABLE 4

| Sequence | | SEQ ID |
|---|---|---|
| ID | Sequence | NO: |
| REGN- | QVQLVQSGAEVKKPGASVKVSCKASGYTFTNYGISWVRQAPGQGLE | 172 |
| VH-3 | WMGWISVYNGKTNYAQKLQGRVTMTTDTSTTTAYMEMRSLRSDDT | |
| | AVYYCARGSGYDLDYWGQGTLVSVSS | |
| REGN- | EVQLVESGGGLVQPGGSLRLSCAASGFTFSSFWMTWVRQAPGKGLE | 173 |
| VH-19 | WVANIKQDGSEKYYVDSVKGRFTISRDNAKNSLYLQMNSLRAEDTA | |
| | VYYCARDPGRTMVRGGIRYYYGMDVWGQGTTVTVSS | |
| REGN- | EVKLAESGGGLVQPGGSLRLSCAASGFTFSSHWMNWVRQAPGKGLE | 174 |
| VH-35 | WVANIKQDGSDKYYVDSVKGRFTISRDNAKNSLYLQLNSLIAEDTAV | |
| | YYCARDRGVRPPRGAFDIWGQGTMVTVSS | |
| REGN- | QVQLVQSGAEVKKPGASVKVSCKASGYTFNSYGISWVRQAPGQGLE | 175 |
| VH-51 | WMGWIRTYNGNTNYAQKLQGRVTMTTDTSTSTAYMELRSLRSDDT | |
| | AVYYCARDEARIVVAGTTPYYYGMDVWGQGTTVTVSS | |
| REGN- | QVQLVESGGGLVQPGGSLRLSCAVSGFTISDHYMSWIRQAPGKGLEW | 176 |
| VH-67 | ISYISSSGSKIYYADSVKGRFTISRDNAKNSLFLQMNSLRAEDTAVYYC | |
| | ARTRQLVGDYWGQGTLVTVSS | |
| REGN- | EVQLVESGGGLVQPGRSLRLSCAASGFTFDNYAMHWVRQAPGKGLE | 177 |
| VH-83 | WVSGIRWNSGSIGYADSVKGRFTISRDNAKNSLYLQMNSLRAEDTAL | |
| | YYCAKEGGYSGYRPGPFFDYWGQGTLVTVSS | |
| REGN- | QVQLVQSGAEVKKPGASVKVSCKASGYTFTNYGISWVRQAPGQGLE | 178 |
| VH-99 | WMGWISVYNGHTNYAQKLQGRVTMTTDTSTSTAYMELRSLRSDDT | |
| | AVYYCARGSGYDFDSWGQGTLVTVSS | |
| REGN- | QVQLVQSGAEVKKPGASVKVSCKASRYTFTSYDINWVRQATGQGLE | 179 |
| VH-115 | WMGWMNPNSGNTGYAQKFQGRVTMTRNTSTSTAYMELSSLRSEDT | |
| | AVYYCARVRRFFDYWGQGTLVTVSS | |
| REGN- | QVQLVQSGPEVKKPGASVKVSCKASGYTFTNYGISWVRQAPGQGLE | 180 |
| VH-147 | WMGWISVYNGNINYAQKLQGRVTMTTDTSTSTAYMDLRSLRSDDTA | |
| | VYYCARGSGYDFDYWGQGTLVTVSS | |
| REGN- | QVQLVQSGAEVKKPGASVKVSCKDSAYTFNRYGISWVRQAPGQGLE | 181 |
| VH-163 | WMGWISAYTGNTVYAQKLQGRVTMTTDNSTSTAYMELRSLRSDDT | |
| | AVYYCARDKSIFGVVRGFDYWGQGTLVTVSS | |
| REGN- | AIQMTQSPSSLSASVGDRVTITCRASQGIRNALGWYQQKPGKAPKLLI | 182 |
| VL-11 | YAASSLQSGVPSRFSGSGSGTDFTLTFSSLQPEDFATYYCLQDFNYPYT | |
| | FGQGTKLEIK | |
| REGN- | DIQMTQSPSSVSASVGDRVTISCRASQGVSSWLAWYQQKPGNAPKLL | 183 |
| VL-27 | ISAASSIQSGVPSRFSGSGSGTDFTLTISSLQPEDFATYYCQQANSFPLT | |
| | FGGGTKVEIK | |
| REGN- | DIQMTQSPSSVSASVGDRVTITCRASQGISSWLAWYQQKPGKAPKLLI | 184 |
| VL-43 | YAASSFQSGVPSRFSGSGSGTDFTLTISSLQPEDFATYFCQQANSFPLTF | |
| | GGGTTVEIK | |
| REGN- | DIQMTQSPSSVSASVGDRVTITCRASQDISIWLAWYQQSPGKAPKLLI | 185 |
| VL-59 | NVASRLQSGVPSRFSGSGSGTDFTLTINSLQPEDFVTYYCQQANSFPIT | |
| | FGQGTRLATK | |
| REGN- | DIQLTQSPSFLSASVGDRVTITCWASQGISSYLAWYQQKPGKAPKLLIF | 186 |
| VL-75 | AASTLQSGVPSRFSGSGSGTEFTLTISSLQPEDFATYYCQQLNSYPLTF | |
| | GGGTKVEIR | |
| REGN- | EIVMTQSPATLSVSPGERATLSCRASQSVNYNLAWYQHKPGQAPRLLI | 187 |
| VL-91 | YGASTRATGIPARFSGSGSGTEFTLTISSLQSEDFAVYYCQQYNNWPL | |
| | TFGGGTKVEIK | |
| REGN- | AIQMTQSSSSLSASVGDRVTITCRASQAIRNALGWYQQKPGKAPKVLI | 188 |
| VL-107 | YAASSLQSGIPSRFSGSGSGTDFTLTISSLQPEDFATYYCLQDYDYPYT | |
| | FGQGTKLEIK | |
| REGN- | DIQLTQSPSFLSASVGDRVTITCWASQGIISYLAWYQQKPGKAPKLLIY | 189 |
| VL-123 | AASTLHSGVPSRFSGSGSGTEFTLTISSLQPEDFATYYCHQLKSYPITFG | |
| | QGTRLEIK | |
| REGN- | AIQMTQSPSSLSASVGDRVTITCRASQDIRNALGWYQQKPGKAPKLLI | 190 |
| VL-155 | YAASSLQSGVPSRFSGSASGTDFTLTISSLQPEDFAAYYCLQDYNYPY | |
| | TFGQGTKLEIK | |
| REGN- | EIVMTQSPVTLSLSPGERATLPCRASQSVSSSLAWYQQKAGQSPRLLI | 191 |
| VL-171 | YGASTRATGIPARFSGSGSGTEFTLTISNLQSEDFAVYYCQQYNNWPL | |
| | TFGGGTKVEIK | |

### Pharmaceutical Compositions

Methods that comprise administering an IL-4R antagonist to a patient, wherein the IL-4R antagonist is contained within a pharmaceutical composition are provided. The pharmaceutical compositions described herein are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The dose of antibody administered to a patient may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical compositions described herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, intra-tracheal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition described herein can be delivered subcutaneously or intravenously with a standard needle and syringe. In addition, with respect to subcutaneous delivery, a pen delivery device (e.g., an autoinjector pen) readily has applications in delivering a pharmaceutical composition described herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition described herein include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few. Examples of large-volume delivery devices (e.g., large-volume injectors) include, but are not limited to, bolus injectors such as, e.g., BD Libertas West SmartDose, Enable Injections, SteadyMed PatchPump, Sensile SenseTrial, YPsomed YpsoDose, Bespak Lapas, and the like.

For direct administration to the sinuses, the pharmaceutical compositions described herein may be administered using, e.g., a microcatheter (e.g., an endoscope and microcatheter), an aerosolizer, a powder dispenser, a nebulizer or an inhaler. The methods include administration of an IL-4R antagonist to a subject in need thereof, in an aerosolized formulation. For example, aerosolized antibodies to IL-4R may be administered to treat asthma in a patient. Aerosolized antibodies can be prepared as described in, for example, U.S. Pat. No. 8,178,098, incorporated herein by reference in its entirety.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant (e.g., polysorbate 80, HCO-50 (polyoxyethylene (50 mol) adduct of hydrogenated castor oil)), etc. As the oily medium, there are employed, e.g., sesame oil, soybean oil, etc., which may be used in combination with a solubilizing agent such as benzyl benzoate, benzyl alcohol, etc. The injection thus prepared is typically filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used as described herein are disclosed, e.g., in U.S. Pat. No. 8,945,559.

### Dosage

The amount of IL-4R antagonist (e.g., anti-IL-4R antibody) administered to a subject according to the methods described herein is, generally, a therapeutically effective amount. As used herein, the phrase “therapeutically effective amount” means an amount of IL-4R antagonist that results in one or more of: (a) a reduction in the incidence of asthma exacerbations; (b) an improvement in one or more asthma-associated parameters (as defined elsewhere herein); and/or (c) a detectable improvement in one or more symptoms or indicia of an upper airway inflammatory condition. A “therapeutically effective amount” also includes an amount of IL-4R antagonist that inhibits, prevents, lessens, or delays the progression of asthma in a subject.

In the case of an anti-IL-4R antibody, a therapeutically effective amount can be from about 0.05 mg to about 700 mg, e.g., about 0.05 mg, about 0.1 mg, about 1.0 mg, about 1.5 mg, about 2.0 mg, about 3.0 mg, about 5.0 mg, about 7.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, or about 700 mg of the anti-IL-4R antibody. In certain embodiments, 300 mg of an anti-IL-4R antibody is administered.

The amount of IL-4R antagonist contained within the individual doses may be expressed in terms of milligrams of antibody per kilogram of subject body weight (i.e., mg/kg). For example, the IL-4R antagonist may be administered to a patient at a dose of about 0.0001 to about 10 mg/kg of subject body weight. For example, the IL-4R antagonist can be administered at a dose of 1 mg/kg, 2 mg/kg, 3 mg/kg, 4 mg/kg, 5 mg/kg or 6 mg/kg.

In certain exemplary embodiments, a subject is a pediatric subject having a body weight of more than 30 kg, and the IL-4R antagonist is administered at a dose of about 150 mg, about 200 mg, about 250 mg, about 300 mg, about 350 mg, about 400 mg, about 450 mg, about 500 mg, about 550 mg, or about 600 mg. In particularly exemplary embodiments, a subject is a pediatric subject having a body weight of more than 30 kg, and the IL-4R antagonist is administered at a dose of about 200 mg.

In certain embodiments, a subject is a pediatric subject having a body weight of more than 30 kg, and the IL-4R antagonist is administered at a dose of about 200 mg every two weeks (q2w). In certain embodiments, a subject is a pediatric subject having a body weight of more than 30 kg, and the IL-4R antagonist is administered at an initial dose of about 300 mg, followed with a dose of about 300 mg every four weeks (q4w), starting 2 weeks after the initial dose.

In certain embodiments, a subject is a pediatric subject having a body weight of 30 kg or more, for example, a body weight of 30 kg to less than 40 kg, 50 kg, or 60 kg, and the IL-4R antagonist is administered at a dose of about 200 mg every two weeks (q2w). In certain embodiments, a subject is a pediatric subject having a body weight of 30 kg or more, and the IL-4R antagonist is administered at an initial dose of about 200 mg every two weeks (q2w), followed with a dose of about 200 mg every two weeks, starting 2 weeks after the initial dose. In certain embodiments, a subject is a pediatric subject having a body weight of 30 kg to less than 60 kg, and the IL-4R antagonist is administered at an initial dose of about 200 mg every two weeks (q2w), followed with a dose of about 200 mg every two weeks, starting 2 weeks after the initial dose. In certain embodiments, a subject is a pediatric subject having a body weight of 30 kg or more, for example, a body weight of 30 kg to less than 40 kg, 50 kg, or 60 kg, and the IL-4R antagonist is administered at an initial dose of about 300 mg, followed with a dose of about 300 mg every four weeks (q4w), starting 1, 2, 3 or 4 weeks after the initial dose. In certain embodiments, a subject is a pediatric subject having a body weight of 30 kg to less than 60 kg, and the IL-4R antagonist is administered at an initial dose of about 300 mg, followed with a dose of about 300 mg every four weeks (q4w), starting 2 or 4 weeks after the initial dose. In certain embodiments, the IL-4R antagonist is administered as subcutaneous injection.

In certain embodiments, a subject is a pediatric subject having a body weight of 60 kg or more and the IL-4R antagonist is administered at a dose of about 200 mg every two weeks (q2w). In certain embodiments, a subject is a pediatric subject having a body weight of 60 kg or more, and the IL-4R antagonist is administered at an initial dose of about 200 mg, followed with a dose of about 200 mg every two weeks (q2w), starting 2 weeks after the initial dose. In certain embodiments, a subject is a pediatric subject having a body weight of 60 kg or more, and the IL-4R antagonist is administered at an initial dose of about 400 mg (e.g., two 200 mg injections), followed by about 200 mg given every other week, starting 2 weeks after the initial dose, or an initial dose of 600 mg (e.g., two 300 mg injections), followed by about 300 mg every two weeks (q2w), starting 2 weeks after the initial dose. In certain embodiments, a subject is a pediatric subject having a body weight of 60 kg or more, and the IL-4R antagonist is administered at an initial dose of about 200 mg, about 300 mg, about 400 mg, about 500 mg, or about 600 mg, followed with a dose of about 200 mg, about 300 mg, or about 400 mg every four weeks (q4w), starting 1, 2, 3, or 4 weeks after the initial dose. In certain embodiments, a subject is a pediatric subject having a body weight of 60 kg or more, and the IL-4R antagonist is administered at an initial dose of about 200 mg, followed with a dose of about 200 mg every two weeks (q2w), starting 2 weeks after the initial dose. In certain embodiments, a subject is a pediatric subject having a body weight of 60 kg or more, with severe asthma and who are on oral corticosteroids, or with severe asthma and co-morbid moderate-to-severe atopic dermatitis or adults with co-morbid severe chronic rhinosinusitis with nasal polyposis, and the IL-4R antagonist is administered at an initial dose of 600 mg, followed by 300 mg every other week (q2w). In certain embodiments, the IL-4R antagonist is administered as subcutaneous injection.

In certain exemplary embodiments, a subject is a pediatric subject having a body weight of 30 kg or less (an optionally a body weight of at least 15 kg), and the IL-4R antagonist is administered at a dose of about 50 mg, about 100 mg, about 150 mg, about 200 mg, about 250 mg, or about 300 mg. In particularly exemplary embodiments, a subject is a pediatric subject having a body weight of 30 kg or less (an optionally a body weight of at least 15 kg), and the IL-4R antagonist is administered at a dose of about 100 mg.

In certain embodiments, a subject is a pediatric subject having a body weight of 30 kg or less (and optionally a body weight of at least 15 kg), and the IL-4R antagonist is administered at a dose of about 100 mg every two weeks (q2w). In certain embodiments, a subject is a pediatric subject having a body weight of 30 kg or less (and optionally a body weight of at least 15 kg), and the IL-4R antagonist is administered at a dose of about 300 mg every four weeks (q4w). In certain embodiments, a subject is a pediatric subject having a body weight of 30 kg or less, and the IL-4R antagonist is administered at an initial dose of about 300 mg, followed with a dose of about 300 mg per four weeks (q4w), starting 2 weeks after the initial dose.

In certain embodiments, a subject is a pediatric subject having a body weight of less than 30 kg (and optionally a body weight of at least 15 kg), and the IL-4R antagonist is administered at a dose of about 100 mg every two weeks (q2w). In certain embodiments, a subject is a pediatric subject having a body weight of less than 30 kg (and optionally a body weight of at least 15 kg), and the IL-4R antagonist is administered at a dose of about 300 mg every four weeks (q4w). In certain embodiments, a subject is a pediatric subject having a body weight of less than 30 kg but at least 15 kg, and the IL-4R antagonist is administered at an initial dose of about 100 mg, followed with a dose of about 100 mg every two weeks (q2w), starting 2 weeks after the initial dose. In certain embodiments, a subject is a pediatric subject having a body weight of less than 30 kg but at least 15 kg, and the IL-4R antagonist is administered at an initial dose of about 300 mg, followed with a dose of about 300 mg every four weeks (q4w), starting 2 or 4 weeks after the initial dose.

In certain exemplary embodiments, an IL-4R antagonist is administered at a concentration of 150 mg/mL using a prefilled device. In certain exemplary embodiments, an IL-4R antagonist is administered at a concentration of 175 mg/mL using a prefilled device.

In certain exemplary embodiments, an IL-4R antagonist is administered at a concentration of 300 mg/2 mL solution in a single-dose pre-filled pen. In certain exemplary embodiments, an IL-4R antagonist is administered at a concentration of 200 mg/1.14 mL solution in a single-dose pre-filled pen. In certain exemplary embodiments, an IL-4R antagonist is administered at a concentration of 300 mg/2 mL solution in a single-dose pre-filled syringe with a needle shield. In certain exemplary embodiments, an IL-4R antagonist is administered at a concentration of 200 mg/1.14 mL solution in a single-dose pre-filled syringe with a needle shield. In certain exemplary embodiments, an IL-4R antagonist is administered at a concentration of 100 mg/0.67 mL solution in a single-dose pre-filled syringe with a needle shield.

In certain embodiments, the methods comprise an initial dose or loading dose of about 100 mg, about 200 mg or about 300 mg of an IL-4R antagonist. In certain embodiments, the methods comprise an initial dose or loading dose of about 100 mg of an IL-4R antagonist. In certain embodiments, the methods comprise one or more secondary doses or maintenance doses of about 100 mg of the IL-4R antagonist.

In certain embodiments, the methods comprise an initial dose or loading dose of about 200 mg, about 400 mg or about 600 mg of an IL-4R antagonist. In certain embodiments, the methods comprise an initial dose or loading dose of about 200 mg of an IL-4R antagonist. In certain embodiments, the methods comprise one or more secondary doses or maintenance doses of about 200 mg of the IL-4R antagonist.

In certain embodiments, the initial dose is about the same as the loading dose. In certain embodiments, the initial dose is about 1.1×, about 1.2×, about 1.3×, about 1.4×, about 1.5×, about 1.6×, about 1.7×, about 1.8×, about 1.9×, about 2.0×, about 2.5×, about 3.0×, or more of the loading dose.

In certain embodiments, an ICS and/or a controller medication is selected from the group consisting of a LABA, an LTRA, a long-acting muscarinic antagonist (LAMA), and a methylxanthine are administered for the duration of administration of the IL-4R antagonist.

In certain embodiments, a subject is a pediatric subject having a body weight of 30 kg or less (an optionally a body weight of at least 15 kg), and the initial dose comprises 100 mg of an anti-IL-4R antibody or antigen-binding fragment thereof, and the one or more secondary doses comprises 100 mg of the antibody or antigen-binding fragment thereof administered every other week (q2w).

In certain embodiments, a subject is a pediatric subject having a body weight of 30 kg or less (an optionally a body weight of at least 15 kg), and the initial dose comprises 200 mg of an anti-IL-4R antibody or antigen-binding fragment thereof, and the one or more secondary doses comprises 100 mg of the antibody or antigen-binding fragment thereof administered every other week (q2w).

In certain embodiments, a subject is a pediatric subject having a body weight of 30 kg or less (an optionally a body weight of at least 15 kg), and the initial dose comprises 300 mg of an anti-IL-4R antibody or antigen-binding fragment thereof, and the one or more secondary doses comprises 100 mg of the antibody or antigen-binding fragment thereof administered every other week (q2w).

In certain embodiments, a subject is a pediatric subject having a body weight of 30 kg or less (an optionally a body weight of at least 15 kg), and the initial dose comprises 200 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 30 kg or less (an optionally a body weight of at least 15 kg), and the initial dose comprises 300 mg of an anti-IL-4R antibody or antigen-binding fragment thereof, and the one or more secondary doses comprises 300 mg of the antibody or antigen-binding fragment thereof administered every other week (q2w), every three weeks (q3w) or every four weeks (q4w).

In certain embodiments, a subject is a pediatric subject having a body weight of less than 30 kg (an optionally a body weight of at least 15 kg), and the initial dose comprises 100 mg of an anti-IL-4R antibody or antigen-binding fragment thereof, and the one or more secondary doses comprises 100 mg of the antibody or antigen-binding fragment thereof administered every two weeks (q2w).

In certain embodiments, a subject is a pediatric subject having a body weight of less than 30 kg (an optionally a body weight of at least 15 kg), and the initial dose comprises 300 mg of an anti-IL-4R antibody or antigen-binding fragment thereof, and the one or more secondary doses comprises 300 mg of the antibody or antigen-binding fragment thereof administered every four weeks (q4w).

In certain embodiments, a subject is a pediatric subject having a body weight of 30 kg or more, such as 30 kg to less than 60 kg, and the initial dose comprises 200 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of or more, such as 30 kg to less than 60 kg, and the initial dose comprises 300 mg of an anti-IL-4R antibody or antigen-binding fragment thereof, and the one or more secondary doses comprises 300 mg of the antibody or antigen-binding fragment thereof administered every four weeks (q4w).

In certain embodiments, a subject is a pediatric subject having a body weight of greater than 30 kg, and the initial dose comprises 200 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of greater than 30 kg, and the initial dose comprises 400 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of greater than 30 kg, and the initial dose comprises 500 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of greater than 30 kg, and the initial dose comprises 600 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of greater than 30 kg, and the initial dose comprises 400 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of greater than 30 kg, and the initial dose comprises 500 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of greater than 30 kg, and the initial dose comprises 600 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 60 kg or more, and the initial dose comprises 200 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of 60 kg or more, and the initial dose comprises 300 mg of an anti-IL-4R antibody or antigen-binding fragment thereof, and the one or more secondary doses comprises 300 mg of the antibody or antigen-binding fragment thereof administered every four weeks (q4w).

In certain embodiments, a subject is a pediatric subject having a body weight of 30 kg to less than 60 kg, and the initial dose comprises 400 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 30 kg to less than 60 kg, and the initial dose comprises 500 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 30 kg to less than 60 kg, and the initial dose comprises 600 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 60 kg or more, and the initial dose comprises 200 mg of an anti-IL-4R antibody or antigen-binding fragment thereof, and the one or more secondary doses comprises 200 mg of the antibody or antigen-binding fragment thereof administered every other week (q2w).

In certain embodiments, a subject is a pediatric subject having a body weight of 60 kg or more, and the initial dose comprises 300 mg of an anti-IL-4R antibody or antigen-binding fragment thereof, and the one or more secondary doses comprises 300 mg of the antibody or antigen-binding fragment thereof administered every four weeks (q4w).

In certain embodiments, a subject is a pediatric subject having a body weight of 60 kg or more, and the initial dose comprises 400 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 60 kg or more, and the initial dose comprises 500 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

In certain embodiments, a subject is a pediatric subject having a body weight of 60 kg or more, and the initial dose comprises 600 mg of an anti-IL-4R antibody or antigen-binding fragment thereof, and the one or more secondary doses comprises 200 mg of the antibody or antigen-binding fragment thereof administered every other week (q2w), every three weeks (q3w) or every four weeks (q4w).

### Combination Therapies

Certain embodiments of the methods described herein comprise administering to the subject one or more additional therapeutic agents (also referred to herein as “one or more additional medicinal products”) in combination with the IL-4R antagonist. As used herein, the expression “in combination with” means that the additional therapeutic agents are administered before, after, or concurrent with the pharmaceutical composition comprising the IL-4R antagonist. In some embodiments, the term “in combination with” includes sequential or concomitant administration of an IL-4R antagonist and a second therapeutic agent. Methods to treat asthma or an associated condition or complication or to reduce at least one exacerbation, comprising administration of an IL-4R antagonist in combination with a second therapeutic agent for additive or synergistic activity, are provided.

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes, or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours, or about 72 hours after the administration of the pharmaceutical composition comprising the IL-4R antagonist. Administration “concurrent” with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

In exemplary embodiments, an additional therapeutic agent administered in combination with the IL-4R antagonist is a background therapy including one or more asthma controller medications. In exemplary embodiments, a background therapy includes one or both of an inhaled corticosteroid (ICS) and a second controller medication. In certain embodiments, the method leads to reduced need of the background therapy. For example, in certain embodiments, the method leads to reduced dose and/or reduced frequency of the background therapy.

In certain embodiments, a controller medication is an ICS that is administered daily in a low dose, a medium dose or a high dose. Suitable ICSs include, but are not limited to: beclometasone dipropionate (chlorofluorocarbon propellant) (100-200 mcg daily (low dose), >200-400 mcg daily (medium dose) or >400 mcg daily (high dose)); beclometasone dipropionate (HFA) (50-100 mcg daily (low dose), >100-200 mcg daily (medium dose) or >200 mcg daily (high dose)); budesonide (DPI) (100-200 mcg daily (low dose), >200-400 mcg daily (medium dose) or >400 mcg daily (high dose)); budesonide (HFA) (100-200 mcg daily (low dose), >200-400 mcg daily (medium dose) or >400 mcg daily (high dose)); budesonide (nebulized solution) (250-500 mcg daily (low dose), >500-1000 mcg daily (medium dose) or >1000 mcg daily (high dose)); ciclesonide (HFA) (80 mcg daily (low dose), >80-160 mcg daily (medium dose) or >160 mcg daily (high dose)); flunisolide (HFA) (160 mcg daily (low dose), >160-<320 mcg daily (medium dose) or 320 mcg daily (high dose)); fluticasone propionate (DPI) (100-200 mcg daily (low dose), >200-400 mcg daily (medium dose) or >400 mcg daily (high dose)); fluticasone propionate (HFA) (100-200 mcg daily (low dose), >200-500 mcg daily (medium dose) or >500 mcg daily (high dose)); fluticasone furoate (100-200 mcg daily (low dose), >200-400 mcg daily (medium dose) or >400 mcg daily (high dose)); mometasone furoate (110 mcg daily (low dose), ≥220-<440 mcg daily (medium dose) or ≥440 mcg daily (high dose)); triamcinolone acetonide (400-800 mcg daily (low dose), >800-1200 mcg daily (medium dose) or >1200 mcg daily (high dose)); and the like.

In certain embodiments, a controller medication is a long-acting β2 agonist (LABA). Suitable LABAs include, but are not limited to, salmeterol, formoterol, bambuterol, clenbuterol, tulobuterol, vilanterol, olodaterol, indacaterol and the like.

In certain embodiments, a controller medication is a leukotriene receptor antagonist (LTRA) or an anti-leukotriene. Suitable LTRAs or anti-leukotrienes include, but are not limited to, montelukast, pranlukast, zafirlukast, zileuton and the like.

In certain embodiments, a controller medication is a long-acting muscarinic antagonist (LAMA). Suitable LAMAs include, but are not limited to, tiotropium, glucopyrronium bromide, aclidinium bromide, umeclidinium and the like.

In certain embodiments, a controller medication is a methylxanthine. Suitable methylxanthines include, but are not limited to, aminophylline, theophylline, dyphylline, oxtryphylline, diprophylline, acebrophylline, bamifylline, doxofylline and the like.

In certain embodiments, two or more controller medications are administered together, e.g., as a metered-dose inhaler (MDI). Exemplary combinations administered by MDI include, but are not limited to, fluticasone propionate/salmeterol, fluticasone propionate/formoterol, fluticasone furoate/vilanterol, budesonide/formoterol, mometasone furoate/formoterol, beclometasone dipropionate/formoterol and the like.

The additional therapeutic agent may be, e.g., another IL-4R antagonist, an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), a TNF antagonist, an IL-8 antagonist, an IL-9 antagonist, an IL-17 antagonist, an IL-5 antagonist, an IgE antagonist, a CD48 antagonist, a leukotriene inhibitor, an anti-fungal agent, an NSAID, a long-acting beta2 agonist (e.g., salmeterol or formoterol), an inhaled corticosteroid (e.g., fluticasone or budesonide), a systemic corticosteroid (e.g., oral or intravenous), methylxanthine, nedocromil sodium, cromolyn sodium, or combinations thereof. For example, in certain embodiments, the pharmaceutical composition comprising an IL-4R antagonist is administered in combination with a combination comprising a long-acting beta2 agonist and an inhaled corticosteroid (e.g., fluticasone+salmeterol (e.g., Advair® (GlaxoSmithKline)); or budesonide+formoterol (e.g., SYMBICORT® (Astra Zeneca))).

In some embodiments, an additional therapeutic agent administered in combination with the IL-4R antagonist is a vaccine. In certain exemplary embodiments, the vaccine is a viral vaccine or a bacterial vaccine. In certain exemplary embodiments, the vaccine is a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHlB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PP SV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RV1 (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html). In some embodiments, the vaccine is for tetanus, diphtheria, pertussis and/or seasonal trivalent/quadrivalent influenza vaccine.

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine is concurrently treated for asthma with an IL-4R antagonist.

In certain embodiments, treatment with an IL-4R antagonist is suspended or terminated prior to treatment with the vaccine. In certain embodiments, treatment with the IL-4R antagonist is suspended about 1 to about 9 (e.g., about 1, about 1½, about 2, about 2½, about 3, about 3½, about 4, about 4½, about 5, about 5½, about 6, about 6½, about 7, about 7½, about 8, about 8½, about 9, or more) weeks prior to administration of the vaccine. In some embodiments, treatment with the IL-4R antagonist is suspended about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, treatment with the IL-4R antagonist is resumed subsequent to treatment with the vaccine. In certain embodiments, treatment with the IL-4R antagonist is resumed about 1 to about 14 (e.g., about 1, about 1½, about 2, about 2½, about 3, about 3½, about 4, about 4½, about 5, about 5½, about 6, about 6½, about 7, about 7½, about 8, about 8½, about 9, about 9½, about 10, about 10½, about 11, about 11½, about 12, about 12½, about 13, about 13½, about 14, about 14½, or more) weeks subsequent to administration of the vaccine. In some embodiments, treatment with the IL-4R antagonist is resumed about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

In certain embodiments, the effectiveness of the IL-4R antagonist is not decreased by administration in combination with the vaccine, or by subsequent administration of the vaccine. In some embodiments, the subject's forced expiratory volume (FEV₁) is stable before and after administration of the vaccine.

In some embodiments, the effectiveness of the vaccine is not decreased by administration in combination with the IL-4R antagonist, or by previous and/or subsequent administration of the IL-4R antagonist. In some embodiments, the subject develops seroprotective neutralization titers to the vaccine when the vaccine is co-administered with the IL-4R antagonist.

In certain exemplary embodiments, a subject is administered a vaccine described herein, wherein before, during, or after administration of the vaccine, the subject is administered at least one dose of IL-4R antagonist.

### Administration Regimens

According to certain embodiments, multiple doses of an IL-4R antagonist may be administered to a subject over a defined time course. Such methods comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks, or months). Methods that comprise sequentially administering to the patient a single initial dose of an IL-4R antagonist, followed by one or more secondary doses of the IL-4R antagonist, and optionally followed by one or more tertiary doses of the IL-4R antagonist, are provided.

Methods comprising administering to a subject a pharmaceutical composition comprising an IL-4R antagonist at a dosing frequency of about four times a week, twice a week, once a week (q1w), once every two weeks (every two weeks is used interchangeably with every other week, bi-weekly or q2w), once every three weeks (tri-weekly or q3w), once every four weeks (monthly or q4w), once every five weeks (q5w), once every six weeks (q6w), once every seven weeks (q7w), once every eight weeks (q8w), once every nine weeks (q9w), once every ten weeks (g10w), once every eleven weeks (q11w), once every twelve weeks (q12w), or less frequently so long as a therapeutic response is achieved, are provided.

In certain embodiments involving the administration of a pharmaceutical composition comprising an anti-IL-4R antibody, once a week dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every two weeks dosing (every two weeks is used interchangeably with every other week, bi-weekly or q2w) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every three weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every four weeks dosing (monthly dosing) of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every five weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every six weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every eight weeks dosing of an amount of about 100 mg, about 200 mg, about 300 mg, about 400 mg, about 500 mg or about 600 mg can be employed. In other embodiments involving the administration of a pharmaceutical composition comprising an anti-IL-4R antibody, once every twelve weeks dosing of an amount of about 100 mg, about 200 mg, about 300 mg, about 400 mg, about 500 mg or about 600 mg can be employed. In certain exemplary embodiments, the route of administration is subcutaneous.

The term “week” or “weeks” refers to a period of (n×7 days)±3 days, e.g., (n×7 days)±2 days, (n×7 days)±1 day, or (n×7 days), wherein “n” designates the number of weeks, e.g. 1, 2, 3, 4, 5, 6, 8, 12 or more.

The terms “initial dose,” “secondary doses,” and “tertiary doses,” refer to the temporal sequence of administration of the IL-4R antagonist. Thus, the “initial dose” is the dose that is administered at the beginning of the treatment regimen (also referred to as the “baseline dose” or “loading dose”); the “secondary doses” are the doses that are administered after the initial dose; and the “tertiary doses” are the doses that are administered after the secondary doses. The initial, secondary, and tertiary doses may all contain the same amount of IL-4R antagonist, or may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”). In one embodiment, the maintenance dose may be lower than the loading dose. For example, one or more initial doses or loading doses of 100 mg or 200 mg of IL-4R antagonist may be administered followed by secondary doses or maintenance doses of about 75 mg to about 400 mg. In one embodiment, the secondary dose/maintenance dose may be equal to the initial dose/loading dose. For example, one or more initial doses/loading doses of 100 mg or 200 mg of IL-4R antagonist may be administered followed by secondary doses/maintenance doses of about 100 mg or about 200 mg, respectively.

In certain embodiments, the initial dose is about 50 mg to about 400 mg of the IL-4R antagonist. In one embodiment, the initial dose is 100 mg of the IL-4R antagonist. In another embodiment, the initial dose is 200 mg of the IL-4R antagonist.

In certain embodiments, the secondary dose(s) are about 50 mg to about 600 mg of the IL-4R antagonist. In one embodiment, the maintenance dose is 100 mg of the IL-4R antagonist. In one embodiment, the maintenance dose is 200 mg of the IL-4R antagonist.

In certain embodiments, an initial dose is three times a maintenance dose. In certain embodiments, an initial dose is two times a maintenance dose. In certain embodiments, an initial dose is equal to a maintenance dose.

In some embodiments, the subject has a body weight of 30 kg or less, such as 15 kg to less than 30 kg, the initial dose comprises 100 mg of the antibody or antigen-binding fragment thereof, and the one or more secondary doses comprises 100 mg of the antibody or antigen-binding fragment thereof administered every other week (every other week is used interchangeably with every two weeks, bi-weekly or q2w).

In some embodiments, the subject has a body weight of 15 kg to less than 30 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject has a body weight of at least 30 kg, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject has a body weight of at least 30 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject has a body weight of 30 kg to less than 60 kg, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject has a body weight of 30 kg to less than 60 kg, the initial dose comprises 300 mg of the antibody or antigen-binding fragment thereof, and the one or more secondary doses comprises 300 mg of the antibody or antigen-binding fragment thereof administered every four weeks (q4w).

In some embodiments, the subject has a body weight of 60 kg or more, the initial dose comprises 200 mg of the antibody or antigen-binding fragment thereof, and the one or more secondary doses comprises 200 mg of the antibody or antigen-binding fragment thereof administered every other week (every other week is used interchangeably with every two weeks, bi-weekly or q2w).

In some embodiments, the subject has a body weight of 60 kg or more, the initial dose comprises 300 mg of the antibody or antigen-binding fragment thereof, and the one or more secondary doses comprises 300 mg of the antibody or antigen-binding fragment thereof administered every four weeks (q4w).

In some embodiments, a subject has uncontrolled moderate-to-severe asthma, and the loading dose comprises 100 mg of the antibody or antigen-binding fragment thereof, and the one or more maintenance doses comprises 100 mg of the antibody or antigen-binding fragment thereof administered every other week.

In some embodiments, a subject has uncontrolled moderate-to-severe asthma, and the loading dose comprises 200 mg of the antibody or antigen-binding fragment thereof, and the one or more maintenance doses comprises 200 mg of the antibody or antigen-binding fragment thereof administered every other week.

In some embodiments, a subject has uncontrolled moderate-to-severe asthma, and the loading dose comprises 300 mg of the antibody or antigen-binding fragment thereof, and the one or more maintenance doses comprises 300 mg of the antibody or antigen-binding fragment thereof administered every four weeks.

In some embodiments, a subject has moderate-to-severe asthma with type 2 inflammation characterized by an eosinophilic phenotype and/or elevated FeNO, and the loading dose comprises 100 mg of the antibody or antigen-binding fragment thereof, and the one or more maintenance doses comprises 100 mg of the antibody or antigen-binding fragment thereof administered every other week.

In some embodiments, a subject has moderate-to-severe asthma with type 2 inflammation characterized by an eosinophilic phenotype and/or elevated FeNO, and the loading dose comprises 200 mg of the antibody or antigen-binding fragment thereof, and the one or more maintenance doses comprises 200 mg of the antibody or antigen-binding fragment thereof administered every other week.

In some embodiments, a subject has moderate-to-severe asthma with type 2 inflammation characterized by an eosinophilic phenotype and/or elevated FeNO, and the loading dose comprises 300 mg of the antibody or antigen-binding fragment thereof, and the one or more maintenance doses comprises 300 mg of the antibody or antigen-binding fragment thereof administered every four weeks.

In some embodiments, a subject has oral corticosteroid-dependent asthma, and the loading dose comprises 100 mg of the antibody or antigen-binding fragment thereof, and the one or more maintenance doses comprises 100 mg of the antibody or antigen-binding fragment thereof administered every other week.

In some embodiments, a subject has oral corticosteroid-dependent asthma, and the loading dose comprises 200 mg of the antibody or antigen-binding fragment thereof, and the one or more maintenance doses comprises 200 mg of the antibody or antigen-binding fragment thereof administered every other week.

In some embodiments, a subject has oral corticosteroid-dependent asthma, and the loading dose comprises 300 mg of the antibody or antigen-binding fragment thereof, and the one or more maintenance doses comprises 300 mg of the antibody or antigen-binding fragment thereof administered every four weeks.

In some embodiments, a subject has asthma with an eosinophilic phenotype, and the loading dose comprises 100 mg of the antibody or antigen-binding fragment thereof, and the one or more maintenance doses comprises 100 mg of the antibody or antigen-binding fragment thereof administered every other week.

In some embodiments, a subject has asthma with an eosinophilic phenotype, and the loading dose comprises 200 mg of the antibody or antigen-binding fragment thereof, and the one or more maintenance doses comprises 200 mg of the antibody or antigen-binding fragment thereof administered every other week.

In some embodiments, a subject has asthma with an eosinophilic phenotype, and the loading dose comprises 300 mg of the antibody or antigen-binding fragment thereof, and the one or more maintenance doses comprises 300 mg of the antibody or antigen-binding fragment thereof administered every four weeks.

In some embodiments, a subject has asthma with a Type 2 inflammatory phenotype, and the loading dose comprises 100 mg of the antibody or antigen-binding fragment thereof, and the one or more maintenance doses comprises 100 mg of the antibody or antigen-binding fragment thereof administered every other week.

In some embodiments, a subject has asthma with a Type 2 inflammatory phenotype, and the loading dose comprises 200 mg of the antibody or antigen-binding fragment thereof, and the one or more maintenance doses comprises 200 mg of the antibody or antigen-binding fragment thereof administered every other week.

In some embodiments, a subject has asthma with a Type 2 inflammatory phenotype, and the loading dose comprises 300 mg of the antibody or antigen-binding fragment thereof, and the one or more maintenance doses comprises 300 mg of the antibody or antigen-binding fragment thereof administered every four weeks.

In some embodiments, a subject has a co-morbid Type 2 inflammatory condition, and the loading dose comprises 100 mg of the antibody or antigen-binding fragment thereof, and the one or more maintenance doses comprises 100 mg of the antibody or antigen-binding fragment thereof administered every other week. In some embodiments, the subject has co-morbid moderate-to-severe atopic dermatitis or severe atopic dermatitis.

In some embodiments, a subject has a co-morbid Type 2 inflammatory condition, and the loading dose comprises 200 mg of the antibody or antigen-binding fragment thereof, and the one or more maintenance doses comprises 200 mg of the antibody or antigen-binding fragment thereof administered every other week. In some embodiments, the subject has co-morbid moderate-to-severe atopic dermatitis or severe atopic dermatitis.

In some embodiments, a subject has a co-morbid Type 2 inflammatory condition, and the loading dose comprises 300 mg of the antibody or antigen-binding fragment thereof, and the one or more maintenance doses comprises 300 mg of the antibody or antigen-binding fragment thereof administered every four weeks. In some embodiments, the subject has co-morbid moderate-to-severe atopic dermatitis or severe atopic dermatitis.

In some embodiments, a subject has severe asthma with type 2 inflammation characterized by raised blood eosinophils and/or raised FeNO, and the loading dose comprises 100 mg of the antibody or antigen-binding fragment thereof, and the one or more maintenance doses comprises 100 mg of the antibody or antigen-binding fragment thereof administered every other week.

In some embodiments, a subject has severe asthma with type 2 inflammation characterized by raised blood eosinophils and/or raised FeNO, and the loading dose comprises 200 mg of the antibody or antigen-binding fragment thereof, and the one or more maintenance doses comprises 200 mg of the antibody or antigen-binding fragment thereof administered every other week.

In some embodiments, a subject has severe asthma with type 2 inflammation characterized by raised blood eosinophils and/or raised FeNO, and the loading dose comprises 300 mg of the antibody or antigen-binding fragment thereof, and the one or more maintenance doses comprises 300 mg of the antibody or antigen-binding fragment thereof administered four three weeks.

In some embodiments, a subject has severe asthma inadequately controlled with medium to high dose inhaled corticosteroids (ICS) plus another medicinal product for maintenance treatment, and the loading dose comprises 100 mg of the antibody or antigen-binding fragment thereof, and the one or more maintenance doses comprises 100 mg of the antibody or antigen-binding fragment thereof administered every other week.

In some embodiments, a subject has severe asthma inadequately controlled with medium to high dose inhaled corticosteroids (ICS) plus another medicinal product for maintenance treatment, and the loading dose comprises 200 mg of the antibody or antigen-binding fragment thereof, and the one or more maintenance doses comprises 200 mg of the antibody or antigen-binding fragment thereof administered every other week.

In some embodiments, a subject has severe asthma inadequately controlled with medium to high dose inhaled corticosteroids (ICS) plus another medicinal product for maintenance treatment, and the loading dose comprises 300 mg of the antibody or antigen-binding fragment thereof, and the one or more maintenance doses comprises 300 mg of the antibody or antigen-binding fragment thereof administered every four weeks.

In one exemplary embodiment, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose” means, in a sequence of multiple administrations, the dose of IL-4R antagonist that is administered to a patient prior to the administration of the very next dose in the sequence with no intervening doses.

The methods may include administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

Methods comprising sequential administration of an IL-4R antagonist and a second therapeutic agent, to a patient to treat asthma or an associated condition are provided. In some embodiments, the methods comprise administering one or more doses of an IL-4R antagonist followed by one or more doses (e.g., 2, 3, 4, 5, 6, 7, 8, or more) of a second therapeutic agent. For example, one or more doses of about 75 mg to about 300 mg of the IL-4R antagonist may be administered after which one or more doses (e.g., 2, 3, 4, 5, 6, 7, 8, or more) of a second therapeutic agent (e.g., an inhaled corticosteroid or a beta2-agonist or any other therapeutic agent, as described elsewhere herein) may be administered to treat, alleviate, reduce or ameliorate one or more symptoms of asthma. In some embodiments, the IL-4R antagonist is administered at one or more doses (e.g., 2, 3, 4, 5, 6, 7, 8, or more) resulting in an improvement in one or more asthma-associated parameters followed by the administration of a second therapeutic agent to prevent recurrence of at least one symptom of asthma. Alternative embodiments pertain to concomitant administration of an IL-4R antagonist and a second therapeutic agent. For example, one or more doses (e.g., 2, 3, 4, 5, 6, 7, 8, or more) of an IL-4R antagonist are administered and a second therapeutic agent is administered at a separate dosage at a similar or different frequency relative to the IL-4R antagonist. In some embodiments, the second therapeutic agent is administered before, after or concurrently with the IL-4R antagonist.

In certain embodiments, the IL-4R antagonist is administered every other week for 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks or more. In other embodiments, the IL-4R antagonist is administered every four weeks for 12 weeks, 16 weeks, 20 weeks, 24 weeks, 28 weeks, 32 weeks, 36 weeks, 40 weeks, 44 weeks, 48 weeks or more. In specific embodiments, the IL-4R antagonist is administered for at least 24 weeks.

Methods for treating a subject having moderate-to-severe uncontrolled asthma comprising administering to the subject a loading dose of an antibody or an antigen-binding fragment thereof that specifically binds to IL-4R are provided. In certain embodiments, the methods comprise administering to the subject a plurality of maintenance doses of the antibody or the antigen-binding fragment thereof, wherein the plurality of maintenance doses are administered during a treatment phase. The treatment phase comprises an induction phase, an OCS reduction phase, and an OCS maintenance phase.

In certain exemplary embodiments, the induction phase comprises a period during which subjects continuously receive their OCS dose(s). In certain exemplary embodiments, the reduction phase comprises a period during which subjects receive a lower OCS dose relative to the dose received during the induction phase. In certain exemplary embodiments, the maintenance phase comprises a period during which a subject receives a certain stable amount or dose(s) of OCS. Alternatively, the maintenance phase comprises a period in which OCS therapy/administration is reduced or eliminated. In certain embodiments, OCS use by the patient is completely eliminated and the patient is steroid free within less than 1 year of treatment with the IL4R antibody or fragment thereof (e.g., within 1 year, 6 months, 3 months or 1 month of initial treatment).

### Treatment Populations

The methods provided herein include administering to a subject in need thereof a therapeutic composition comprising an IL-4R antagonist. The expression “a subject in need thereof” means a human or non-human animal that exhibits one or more symptoms or indicia of asthma, or who has been diagnosed with asthma. For example, “a subject in need thereof” may include, e.g., subjects who, prior to treatment, exhibit (or have exhibited) one or more asthma-associated parameter, such as, e.g., impaired FEV₁ (e.g., less than 2.0 L), impaired FEF_{25-75%}; impaired AM PEF (e.g., less than 400 L/min), impaired PM PEF (e.g., less than 400 L/min), an ACQ5 score of at least 2.5, at least 1 nighttime awakenings per night, and/or a SNOT-22 score of at least 20. In various embodiments, the methods may be used to treat mild, moderate-to-severe (e.g., uncontrolled moderate-to-severe), and severe asthma in patients in need thereof. In certain embodiments, the methods may be used to treat mild, moderate-to-severe, and severe asthma, wherein the patients further exhibit one or more comorbid Type 2 inflammatory conditions. In some embodiments, the patient has asthma and co-morbid atopic dermatitis (e.g., moderate-to-severe atopic dermatitis or severe atopic dermatitis).

In a related embodiment, a “subject in need thereof” may be a subject who, prior to receiving an IL-4R antagonist, has been prescribed or is currently taking a combination of ICS and a second controller medication selected from the group consisting of a long-acting β2 agonist (LABA), a leukotriene receptor antagonist (LTRA), a long-acting muscarinic antagonist (LAMA), and a methylxanthine. For example, methods that comprise administering an IL-4R antagonist to a patient who has been taking a regular course of ICS/second controller medication for two or more weeks immediately preceding the administration of the IL-4R antagonist (such prior treatments are referred to herein as “background treatments” or as “another medicinal product”) are provided. Therapeutic methods in which background treatments are continued in combination with administration of the IL-4R antagonist are provided. In yet other embodiments, the amount of the ICS component, the second controller medication component, or both, is gradually decreased prior to or after the start of IL-4R antagonist administration. In some embodiments, methods to treat patients with uncontrolled asthma for at least ≥12 months are provided. In one embodiment, a patient with uncontrolled asthma may be resistant to treatment by a therapeutic agent, such as a corticosteroid, and may be administered an IL-4R antagonist according to the present methods.

In some embodiments, a “subject in need thereof” is selected from the group consisting of: a subject age 18 years old or older, a subject 12 years or older, a subject age 12 to 17 years old (12 to <18 years old), a subject age 6 to 11 years old (6 to <12 years old), and a subject age 2 to 5 years old (2 to <6 years old). In some embodiments, a “subject in need thereof” is selected from the group consisting of: an adult, an adolescent, and a child. In some embodiments, a “subject in need thereof” is selected from the group consisting of: an adult age 18 years of age or older, an adolescent age 12 to 17 years old (12 to <18 years old), a child age 6 to 11 years old (6 to <12 years old), and a child age 2 to 5 years old (2 to <6 years old). The subject can be less than 2 years of age, e.g., 12 to 23 months, or 6 to 11 months. In particularly exemplary embodiments, a subject is a child 6 to <12 years old (also referred to herein as a “pediatric” subject). In certain embodiments, a subject in need thereof is a child 6 to <12 years old having a body weight of more than 30 kg. In certain embodiments, a subject in need thereof is a child 6 to <12 years old having a body weight of 30 kg or less (and optionally a body weight of at least 15 kg).

In some embodiments, a “subject in need thereof” is a subject who is a current smoker. In some embodiments, the subject is a current smoker who smokes, e.g., cigarettes, cigars, pipes, water pipes, and/or vaporizers (i.e., “vapes”). In some embodiments, the subject is a current smoker who has a smoking history of smoking greater than or equal to 10 packs of cigarettes per year. In some embodiments, the subject is a current smoker and has a smoking history of smoking fewer than 10 packs of cigarettes per year. In some embodiments, the subject is a current smoker and has a smoking history of smoking more than 1, 5, 10, 15, 20, 25, 30, 35, 40, 45, 50 or more packs of cigarettes per year. In some embodiments, the subject is a current smoker who has a smoking history of smoking for 6 months, 1 year, 2 years, 3 years or longer.

In some embodiments, a “subject in need thereof” is a subject who is a former smoker. In some embodiments, the subject is a former smoker who has a history of smoking cigarettes, cigars, pipes, water pipes and/or vapes. In some embodiments, the subject is a former smoker who has a smoking history of smoking greater than or equal to 10 packs of cigarettes per year. In some embodiments, the subject is a former smoker who has a smoking history of smoking fewer than 10 packs per year. In some embodiments, the subject is a former smoker who has a smoking history of smoking more than 1, 5, 10, 15, 20, 25, 30, 35, 40, 45, 50 or more packs of cigarettes per year. In some embodiments, the subject is a former smoker who has a smoking history of smoking for 6 months, 1 year, 2 years, 3 years or longer. In some embodiments, the subject is a former smoker who has ceased smoking for at least 1, 2, 3, 4, 5, 6, 7, 8, 9, 10, 11, or 12 months. In some embodiments, the subject is a former smoker who has ceased smoking for at least 6 months. In some embodiments, the subject is a former smoker that intends to quit permanently.

In some embodiments, a “subject in need thereof” is a subject who is a non-smoker. In some embodiments, a subject is a non-smoker that does not have a history of smoking cigarettes, cigars, pipes, water pipes and/or vapes. In some embodiments, a subject is a non-smoker that does not have a history of smoking tobacco.

In some embodiments, a “subject in need thereof” is a subject who is treated with a vaccine, e.g., a viral vaccine or a bacterial vaccine. In some embodiments, the vaccine is a live vaccine, e.g., a live (e.g., live-attenuated) viral vaccine or a live (e.g., live-attenuated) bacterial vaccine.

Suitable vaccines include, but are not limited to adenovirus, anthrax (e.g., AVA vaccine (BioThrax)), cholera (e.g., Vaxchora), diphtheria (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), hepatitis A (e.g., HepA (Havrix, Vaqta), HepA-HepB (Twinrix)), hepatitis B (e.g., HepB (Engerix-B, Recombivax HB, Heplisav-B), DTaP-HepB-IPV (Pediarix), HepA-HepB (Twinrix)), Haemophilus influenzae type b (Hib) (e.g., Hib (ActHIB, PedvaxHlB, Hiberix), DTaP-IPV/Hib (Pentacel)), human papillomavirus (HPV) (e.g., HPV9 (Gardasil 9)), influenza (flu) (e.g., IIV (also called IIV3, IIV4, RIV3, RIV4 and ccIIV4) (Afluria, Fluad, Flublok, Flucelvax, FluLaval, Fluarix, Fluvirin, Fluzone, Fluzone High-Dose, Fluzone Intradermal), LAIV (FluMist)), Japanese encephalitis (e.g., JE (Ixiaro)), measles (e.g., MMR (M-M-R II), MMRV (ProQuad)), meningococcus (e.g., MenACWY (Menactra, Menveo), MenB (Bexsero, Trumenba)), mumps (e.g., MMR (M-M-R II), MMRV (ProQuad)), pertussis (e.g., DTaP (Daptacel, Infanrix), Tdap (Adacel, Boostrix), DTaP-IPV (Kinrix, Quadracel), DTaP-HepB-IPV (Pediarix), DTaP-IPV/Hib (Pentacel)), pneumococcus (e.g., PCV13 (Prevnar13), PP SV23 (Pneumovax 23)), polio (e.g., Polio (Ipol), DTaP-IPV (Kinrix, Quadracel), DTaP-HepB-IPV (Pediarix), DTaP-IPV/Hib (Pentacel)), rabies (e.g., Rabies (Imovax Rabies, RabAvert)), rotavirus (e.g., RV1 (Rotarix), RV5 (RotaTeq)), rubella (e.g., MMR (M-M-R II), MMRV (ProQuad)), shingles (e.g., ZVL (Zostavax), RZV (Shingrix)), smallpox (e.g., Vaccinia (ACAM2000)), tetanus (e.g., DTaP (Daptacel, Infanrix), Td (Tenivac, generic), DT (generic), Tdap (Adacel, Boostrix), DTaP-IPV (Kinrix, Quadracel), DTaP-HepB-IPV (Pediarix), DTaP-IPV/Hib (Pentacel)), tuberculosis, typhoid fever (e.g., Typhoid Oral (Vivotif), Typhoid Polysaccharide (Typhim Vi)), varicella (e.g., VAR (Varivax), MMRV (ProQuad)), yellow fever (e.g., YF (YF-Vax)) and the like. Suitable vaccines are also listed at the US Centers for Disease Control vaccine list, incorporated herein in its entirety for all purposes (cdc.gov/vaccines/vpd/vaccines-list.html).

In some embodiments, the vaccine is an inactivated vaccine, a recombinant vaccine, a conjugate vaccine, a subunit vaccine, a polysaccharide vaccine, or a toxoid vaccine. In some embodiments, the vaccine is a yellow fever vaccine. In some embodiments, the subject treated with the vaccine concurrently is treated for a Type 2 inflammatory disease with an IL-4R antagonist. In some embodiments, the subject treated with the vaccine concurrently is treated for asthma with an IL-4R antagonist. In some embodiments, the subject suspends treatment with an IL-4R antagonist prior to administration of the vaccine.

In certain embodiments the subject suspends treatment with the IL-4R antagonist about 1 to about 9 (e.g., about 1, about 1½, about 2, about 2½, about 3, about 3½, about 4, about 4½, about 5, about 5½, about 6, about 6½, about 7, about 7½, about 8, about 8½, about 9, or more) weeks prior to administration of the vaccine. In certain embodiments, the subject suspends treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, or about 60 days prior to administration of the vaccine.

In certain embodiments, the subject resumes treatment with the IL-4R antagonist subsequent to treatment with the vaccine. In certain embodiments, the subject resumes treatment with the IL-4R antagonist 1 to 14 (e.g., about 1, about 1½, about 2, about 2½, about 3, about 3½, about 4, about 4½, about 5, about 5½, about 6, about 6½, about 7, about 7½, about 8, about 8½, about 9, about 9½, about 10, about 10½, about 11, about 11½, about 12, about 12½, about 13, about 13½, about 14, about 14½, or more) weeks subsequent to administration of the vaccine. In certain embodiments, the subject resumes treatment with the IL-4R antagonist about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, or about 90 days subsequent to administration of the vaccine.

A normal IgE level in healthy subjects is typically less than about 100 IU/mL (e.g., as measured using the IMMUNOCAP® assay (Phadia, Inc. Portage, Mich.)). Thus, methods comprising selecting a subject who exhibits an elevated serum IgE level, which is a serum IgE level greater than about 100 IU/mL, greater than about 150 IU/mL, greater than about 500 IU/mL, greater than about 700 IU/mL, greater than about 1000 IU/mL, greater than about 1500 IU/mL, greater than about 2000 IU/mL, greater than about 2500 IU/mL, greater than about 3000 IU/mL, greater than about 3500 IU/mL, greater than about 4000 IU/mL, greater than about 4500 IU/mL, or greater than about 5000 IU/mL, and administering to the subject a pharmaceutical composition comprising a therapeutically effective amount of an IL-4R antagonist, are provided.

A normal Aspergillus fumigatus (Af)-specific IgE level in healthy subjects is typically less than about 0.10 kU/L (e.g., as measured using the IMMUNOCAP® assay (Phadia, Inc. Portage, Mich.)). Thus, methods comprising selecting a subject who exhibits an elevated serum IgE level, which is a serum IgE level greater than or equal to about 0.1 kU/L, greater than about 0.35 kU/L, greater than about 0.70 kU/L, greater than about 3.50 kU/L, greater than about 17.50 kU/L, greater than about 50.00 kU/L, or greater than about 100.00 kU/L, and administering to the subject a pharmaceutical composition comprising a therapeutically effective amount of an IL-4R antagonist, are provided.

In certain embodiments, IgE levels (e.g., total IgE levels and/or A. fumigatus-specific IgE levels) are improved relative to baseline, e.g., an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more from baseline.

In certain embodiments, allergen-specific IgG4 levels are improved relative to baseline, e.g., an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more from baseline.

TARC levels in healthy subjects are in the range of 106 ng/L to 431 ng/L, with a mean of about 239 ng/L. (An exemplary assay system for measuring TARC level is the TARC quantitative ELISA kit offered as Cat. No. DDN00 by R&D Systems, Minneapolis, Minn.) Thus, methods comprising selecting a subject who exhibits an elevated TARC level, which is a serum TARC level greater than about 431 ng/L, greater than about 500 ng/L, greater than about 1000 ng/L, greater than about 1500 ng/L, greater than about 2000 ng/L, greater than about 2500 ng/L, greater than about 3000 ng/L, greater than about 3500 ng/L, greater than about 4000 ng/L, greater than about 4500 ng/L, or greater than about 5000 ng/L, and administering to the subject a pharmaceutical composition comprising a therapeutically effective amount of an IL-4R antagonist, are provided. In certain embodiments, TARC levels are improved relative to baseline, e.g., an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more from baseline.

Eotaxin-3 belongs to a group of chemokines released by airway epithelial cells, which is up-regulated by the Th2 cytokines IL-4 and IL-13 (Lilly et al 1999, J. Allergy Clin. Immunol. 104: 786-790). Methods comprising administering an IL-4R antagonist to treat patients with elevated levels of eotaxin-3, such as more than about 100 pg/ml, more than about 150 pg/ml, more than about 200 pg/ml, more than about 300 pg/ml, or more than about 350 pg/ml, are provided. Serum eotaxin-3 levels may be measured, for example, by ELISA. In certain embodiments, serum eotaxin-3 levels are improved relative to baseline, e.g., an improvement of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more from baseline.

Periostin is an extracellular matrix protein involved in the Th2-mediated inflammatory processes. Periostin levels are found to be up-regulated in patients with asthma (Jia et al 2012 J Allergy Clin Immunol. 130:647-654.e10. doi: 10.1016/j.jaci.2012.06.025. Epub 2012 Aug. 1). Methods comprising administering an IL-4R antagonist to treat patients with elevated levels of periostin are provided.

Fractional exhaled NO (FeNO) is a biomarker of bronchial or airway inflammation. FeNO is produced by airway epithelial cells in response to inflammatory cytokines including IL-4 and IL-13 (Alwing et al 1993, Eur. Respir. J. 6: 1368-1370). FeNO levels in healthy adults range from 2 to 30 parts per billion (ppb). An exemplary assay for measuring FeNO is by using a NIOX instrument by Aerocrine AB, Solna, Sweden. The assessment may be conducted prior to spirometry and following a fast of at least an hour. Methods comprising administering an IL-4R antagonist to a subject having asthma, wherein the subject has elevated levels of exhaled NO (FeNO) relative to FeNO levels of a subject without asthma, are provided. Methods comprising administering an IL-4R antagonist to a subject with elevated levels of FeNO, such as more than about 20 ppb, more than about 25 ppb, more than about 30 ppb, more than about 31 ppb, more than about 32 ppb, more than about 33 ppb, more than about 34 ppb, or more than about 35 ppb, are provided.

Carcinoembryogenic antigen (CEA) (also known as CEA cell adhesion molecule 5 [CEACAMS]) is a tumor marker that is found correlated to non-neoplastic diseases of the lung (Marechal et al. 1988, Anticancer Res. 8: 677-680). CEA levels in serum may be measured by ELISA. Methods comprising administering an IL-4R antagonist to patients with elevated levels of CEA, such as more than about 1.0 ng/ml, more than about 1.5 ng/ml, more than about 2.0 ng/ml, more than about 2.5 ng/ml, more than about 3.0 ng/ml, more than about 4.0 ng/ml, or more than about 5.0 ng/ml, are provided.

YKL-40 (named for its N-terminal amino acids tyrosine (Y), lysine (K) and leucine (L) and its molecular mass of 40 kD) is a chitinase-like protein found to be up regulated and correlated to asthma exacerbation, IgE, and eosinophils (Tang et al 2010 Eur. Respir. J. 35: 757-760). Serum YKL-40 levels are measured by, for example, ELISA. Methods comprising administering an IL-4R antagonist to patients with elevated levels of YKL-40, such as more than about 40 ng/ml, more than about 50 ng/ml, more than about 100 ng/ml, more than about 150 ng/ml, more than about 200 ng/ml, or more than about 250 ng/ml, are provided.

Periostin is a secreted matricellular protein associated with fibrosis, and its expression is upregulated by recombinant IL-4 and IL-13 in cultured bronchial epithelial cells and bronchial fibroblasts (Jia et al. (2012) J. Allergy Clin. Immunol. 130:647). In human asthmatic patients periostin expression levels correlate with reticular basement membrane thickness, an indicator of subepithelial fibrosis. Id. Methods comprising administering an IL-4R antagonist to patients with elevated levels of periostin are provided.

IL-5 is an interleukin produced by Type 2 T helper cells and mast cells. It can also be used as a Type 2 inflammation biomarker. In certain exemplary embodiments, a treatment according to the present disclosure reduces the level of IL-5 in a subject.

Urinary leukotriene E4 (LTE4) is a cysteinyl leukotriene involved in inflammation. It is known to be produced by several types of white blood cells, including eosinophils, mast cells, tissue macrophages, and basophils, and recently was also found to be produced by platelets adhering to neutrophils. In certain exemplary embodiments, a treatment according to the present disclosure reduces the level of LTE4 in a subject.

Induced sputum eosinophils and neutrophils are well-established direct markers of airway inflammation (Djukanovic et al. 2002, Eur. Respire. J. 37: 1S-2S). Sputum is induced with inhalation of hypertonic saline solution and processed for cell counts according to methods known in the art, for example, the guidelines of European Respiratory Society.

In some embodiments, the subjects are stratified into the following groups: a blood eosinophil count (high blood eosinophils) ≥300 cells/μL (HEos) or 300-499 cells/μL or ≥500 cells/μL, a blood eosinophil count of 200 to 299 cells/μL (moderate blood eosinophils), or a blood eosinophil count <200 cells/μL (low blood eosinophils), and are administered an anti-IL-4R antibody or antigen binding fragment thereof at a dose or dosing regimen based upon the eosinophil level.

In some embodiments, the subjects are stratified into the following groups: a blood eosinophil count of ≥300 cells/μL, of 300-499 cells/μL, or of ≥500 cells/μL (high blood eosinophils); a blood eosinophil count of ≥150 cells/μL (moderate blood eosinophils); or a blood eosinophil count of <150 cells/μL (low blood eosinophils), and are administered an anti-IL-4R antibody or antigen binding fragment thereof at a dose or dosing regimen based upon the eosinophil level.

In some embodiments, a subject with asthma has “raised eosinophils” relative to a subject that does not have asthma, and is administered an anti-IL-4R antibody or antigen binding fragment thereof. In some embodiments, a subject with asthma has “raised eosinophils” defined by a blood eosinophil count of ≥150 cells/μL (i.e., ≥0.15 Giga/L), a blood eosinophil count of ≥300 cells/μL (i.e., ≥0.3 Giga/L), a blood eosinophil count of 300-499 cells/μL (i.e., 0.300-0.499 Giga/L), or a blood eosinophil count of ≥500 cells/μL (i.e., ≥0.5 Giga/L), and is administered an anti-IL-4R antibody or antigen binding fragment thereof.

In some embodiments, a subject has “eosinophilic phenotype” asthma defined by a blood eosinophil count of ≥150 cells/μL (i.e., ≥0.15 Giga/L), a blood eosinophil count of ≥300 cells/μL (i.e., ≥0.3 Giga/L), a blood eosinophil count of 300-499 cells/μL (i.e., 0.300-0.499 Giga/L), or a blood eosinophil count of ≥500 cells/μL (i.e., ≥0.5 Giga/L), and is administered an anti-IL-4R antibody or antigen binding fragment thereof.

In some embodiments, the subjects are stratified into the following groups: a total baseline serum IgE concentration of ≥30 IU/mL; a total baseline serum IgE concentration of ≥100 IU/mL; a total baseline serum IgE concentration of ≥200 IU/mL; a total baseline serum IgE concentration of ≥300 IU/mL; a total baseline serum IgE concentration of ≥400 IU/mL; a total baseline serum IgE concentration of ≥500 IU/mL; a total baseline serum IgE concentration of ≥600 IU/mL; a total baseline serum IgE concentration of ≥700 IU/mL (e.g., high serum IgE); a total baseline serum IgE concentration of ≥800 IU/mL; a total baseline serum IgE concentration of ≥900 IU/mL; or a total baseline serum IgE concentration of ≥1000 IU/mL (e.g., very high IgE), and are administered an anti-IL-4R antibody or antigen binding fragment thereof at a dose or dosing regimen based upon the IgG concentration.

In some embodiments, the subjects are stratified into the following groups: an allergen-specific IgE (e.g., an A. fumigatus-specific) concentration of ≥0.05 kU/L; an allergen-specific (e.g., an A. fumigatus-specific) IgE concentration of ≥0.10 kU/L; an allergen-specific (e.g., an A. fumigatus-specific) IgE concentration of ≥0.15 kU/L; an allergen-specific (e.g., an A. fumigatus-specific) IgE concentration of ≥0.20 kU/L; an allergen-specific (e.g., an A. fumigatus-specific) IgE concentration of ≥0.25 kU/L; an allergen-specific (e.g., an A. fumigatus-specific) IgE concentration of ≥0.30 kU/L; an allergen-specific (e.g., an A. fumigatus-specific) IgE concentration of ≥0.35 kU/L; an allergen-specific (e.g., an A. fumigatus-specific) IgE concentration of ≥0.40 kU/L; an allergen-specific (e.g., an A. fumigatus-specific) IgE concentration of ≥0.45 kU/L; or an allergen-specific (e.g., an A. fumigatus-specific) IgE concentration of ≥0.50 kU/L, and are administered an anti-IL-4R antibody or antigen binding fragment thereof at a dose or dosing regimen based upon the allergen-specific (e.g., an A. fumigatus-specific) IgE concentration.

In some embodiments, the subjects are stratified into the following groups: a baseline FeNO value of ≥20 ppb; a baseline FeNO value of ≥25 ppb; a baseline FeNO value of ≥50 ppb (e.g., high FeNO); a baseline FeNO value of <25 ppb (e.g., low FeNO); a baseline FeNO value of <50 ppb; or a baseline FeNO value of between about 25 ppb and about 50 ppb, and are administered an anti-IL-4R antibody or antigen binding fragment thereof at a dose or dosing regimen based upon the FeNO value.

In some embodiments, a subject is stratified into a Type 2 inflammatory phenotype group based on one or both of a baseline blood eosinophil count of greater than or equal to 150 cells/μL and a baseline FeNO of greater than or equal to 20 ppb.

In some embodiments, a subject is stratified into a Type 2 inflammatory phenotype group based on one or both of a baseline blood eosinophil count of greater than or equal to 150 cells/μL and a baseline FeNO of greater than or equal to 25 ppb.

In some embodiments, a subject is stratified into an eosinophilic phenotype group based on a baseline blood eosinophil count of greater than or equal to 350 cells/μL.

### Methods for Assessing Pharmacodynamic Asthma-Associated Parameters

Methods for assessing one or more pharmacodynamic asthma-associated parameters in a subject in need thereof, caused by administration of a pharmaceutical composition comprising an IL-4R antagonist, are provided. A reduction in the incidence of an asthma exacerbation (as described above) or an improvement in one or more asthma-associated parameters (as described above) may correlate with an improvement in one or more pharmacodynamic asthma-associated parameters; however, such a correlation is not necessarily observed in all cases.

Examples of “pharmacodynamic asthma-associated parameters” include, for example, the following: (a) biomarker expression levels; (b) serum protein and RNA analysis; (c) induced sputum eosinophils and neutrophil levels; (d) exhaled nitric oxide (FeNO); and (e) blood eosinophil count. An “improvement in a pharmacodynamic asthma-associated parameter” means, for example, a decrease from baseline of one or more biomarkers, such as TARC, eotaxin-3, IgE or allergen-specific IgG4, a decrease in sputum eosinophils or neutrophils, FeNO, periostin or blood eosinophil count. As used herein, the term “baseline,” with regard to a pharmacodynamic asthma-associated parameter, means the numerical value of the pharmacodynamic asthma-associated parameter for a patient prior to or at the time of administration of a pharmaceutical composition described herein.

To assess a pharmacodynamic asthma-associated parameter, the parameter is quantified at baseline and at a time point after administration of the pharmaceutical composition. For example, a pharmacodynamic asthma-associated parameter may be measured at about day 1, about day 2, about day 3, day 4, about day 5, about day 6, about day 7, about day 8, about day 9, about day 10, about day 11, about day 12, about day 14, or at about week 3, about week 4, about week 5, about week 6, about week 7, about week 8, about week 9, about week 10, about week 11, about week 12, about week 13, about week 14, about week 15, about week 16, about week 17, about week 18, about week 19, about week 20, about week 21, about week 22, about week 23, about week 24, or longer, after the initial treatment with the pharmaceutical composition. The difference between the value of the parameter at a particular time point following initiation of treatment and the value of the parameter at baseline is used to establish whether there has been change, such as an “improvement,” in the pharmacodynamic asthma-associated parameter (e.g., an increase or decrease, as the case may be, depending on the specific parameter being measured).

In certain embodiments, administration of an IL-4R antagonist to a patient causes a change, such as a decrease or increase, in expression of a particular biomarker. Asthma-associated biomarkers include, but are not limited to, the following: (a) total IgE; (b) Af-specific IgE; (c) allergen-specific IgG4; (d) thymus and activation-regulated chemokine (TARC); (e) YKL-40; (f) carcinoembryonic antigen in serum; (g) eotaxin-3 in plasma; (h) periostin in serum; and (i) eosinophil levels in serum. For example, administration of an IL-4R antagonist to an asthma patient can cause one or more of a decrease in TARC or eotaxin-3 levels, or a decrease in total serum IgE levels. The decrease can be detected at about week 1, about week 2, about week 3, about week 4, about week 5, or longer following administration of the IL-4R antagonist. Biomarker expression can be assayed by methods known in the art. For example, protein levels can be measured by ELISA (Enzyme Linked Immunosorbent Assay). RNA levels can be measured, for example, by reverse transcription coupled to polymerase chain reaction (RT-PCR).

Biomarker expression, as discussed above, can be assayed by detection of protein or RNA in serum. The serum samples can also be used to monitor additional protein or RNA biomarkers related to response to treatment with an IL-4R antagonist, IL-4/IL-13 signaling, asthma, atopy or eosinophilic diseases (e.g., by measuring soluble IL-4Rα, IL-4, IL-13, periostin). In some embodiments, RNA samples are used to determine RNA levels (non-genetic analysis), e.g., RNA levels of biomarkers; and in other embodiments, RNA samples are used for transcriptome sequencing (e.g., genetic analysis).

### Formulations

In some embodiments, the antibody or antigen binding fragment thereof is formulated in a composition comprising: i) about 150 mg/mL of antibody or an antigen-binding fragment thereof that specifically binds to IL-4R, ii) about 20 mM histidine, iii) about 12.5 mM acetate, iv) about 5% (w/v) sucrose, v) about 25 mM arginine hydrochloride, vi) about 0.2% (w/v) polysorbate 80, wherein the pH of the formulation is about 5.9, and wherein the viscosity of the formulation is about 8.5 cPoise.

In alternative embodiments, the antibody or antigen binding fragment thereof is formulated in a composition comprising: i) about 175 mg/mL of antibody or an antigen-binding fragment thereof that specifically binds to IL-4R, ii) about 20 mM histidine, iii) about 12.5 mM acetate, iv) about 5% (w/v) sucrose, v) about 50 mM arginine hydrochloride, and vi) about 0.2% (w/v) polysorbate 80, wherein the pH of the formulation is about 5.9, and wherein the viscosity of the formulation is about 8.5 cPoise.

In specific embodiments, the antibody or antigen-binding fragment thereof comprises an HCVR comprising the amino acid sequence of SEQ ID NO: 1 and an LCVR comprising the amino acid sequence of SEQ ID NO: 2.

In specific embodiments, the antibody comprises dupilumab. Unless otherwise specified, the term “dupilumab” also includes any biosimilars thereof.

Suitable stabilized formulations are also set forth in U.S. Pat. No. 8,945,559, which is incorporated herein by reference in its entirety for all purposes.

The present invention is further illustrated by the following examples which should not be construed as further limiting. The contents of the figures and all references, patents and published patent applications cited throughout this application are expressly incorporated herein by reference for all purposes.

Furthermore, in accordance with the present invention there may be employed conventional molecular biology, microbiology, and recombinant DNA techniques within the skill of the art. Such techniques are explained fully in the literature. See, e.g., Green & Sambrook, Molecular Cloning: A Laboratory Manual, Fourth Edition (2012) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

### EXAMPLES

The following examples are put forth so as to provide those of ordinary skill in the art with a complete disclosure and description of how to make and use the methods and compositions featured in the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The exemplary IL-4R antagonist used in the following Examples is the human anti-IL-4R antibody named dupilumab (also referred to herein as “mAb1” or DUPIXENT®).

### Example I

### A Randomized, Double-Blind, Placebo-Controlled, Parallel Group Study to Evaluate the Efficacy and Safety of DUPIXENT® in Children 6 to <12 Years of Age with Uncontrolled Persistent Asthma (Phase 3)

### Primary Objective

The primary objective is to evaluate the efficacy of DUPIXENT® in children 6 to <12 years of age with uncontrolled persistent asthma.

### Secondary Objectives

The secondary objectives are: to assess the safety and tolerability of DUPIXENT®; to evaluate the effect of DUPIXENT® in improving patient reported outcomes (PROs), including but not limited to: health related quality of life (HRQoL); to assess the DUPIXENT® systemic exposure and incidence of anti-drug antibodies (ADA); and to evaluate the association between DUPIXENT® treatment and pediatric immune responses to vaccines, e.g., any vaccination for tetanus, diphtheria, pertussis and/or seasonal trivalent/quadrivalent influenza vaccine.

### Exploratory Objectives

Exploratory objectives are: to explore baseline and on-treatment levels of biomarkers for their potential to predict and to associate with a treatment response; to explore the association of genetic profiles (optional) with treatment response or airway disease; to evaluate the proportion of patients requiring increased dose of inhaled corticosteroids (ICS) or step up in the second controller medication regimen; and to evaluate the effect of DUPIXENT® on additional patient reported outcomes (PROs).

### Study Design

### General Design

This is a multinational, multicenter, randomized, double-blind, placebo-controlled, parallel-group study assessing the effect of DUPIXENT® administered subcutaneously (SC) for a maximum of 52 weeks in children 6 to <12 years of age with uncontrolled asthma. The study is assessing the primary efficacy analysis population from an overall uncontrolled persistent asthma population to the subpopulation with evidence of either asthma with an eosinophilic phenotype or, more broadly, asthma with Type 2 inflammatory phenotype.

The clinical trial consists of three periods: 1) screening period (4 [±1] weeks) to determine a patient's eligibility status and establish level of asthma control before randomization; 2) treatment period (52 weeks) to treat with DUPIXENT® or placebo subcutaneous (SC) injection; and 3) post-treatment period (12 weeks) to monitor a patient's status when off study drug treatment for patients who choose not to participate in the one-year long-term extension study.

### Screening Period

Prior to and during the screening period, patients must be on one of the following: stable-dose background therapy of medium-dose ICS with second controller medication (i.e., long-acting β2 agonist (LABA), leukotriene receptor antagonist (LTRA), long-acting muscarinic antagonist (LAMA), or methylxanthines) or high-dose ICS alone or high-dose ICS with second controller, for at least 3 months with a stable dose ≥1 month prior to screening visit 1.

The Screening Period will be of 4 (±1) weeks in duration.

### Randomized Treatment Period

Patients are randomized to either DUPIXENT® or matching placebo administered SC for a maximum treatment duration of 52 weeks.

During the Randomized Treatment Period, patients continue the stable dose(s) of controller medication used during the Screening Period. For patients experiencing a deterioration of asthma during the study, the ICS dose may temporarily be increased up to 4-fold (recorded as a loss of asthma control (LOAC) event) for a maximum of 10 days, as indicated and upon recommendation of the physician and/or Investigator. Treatment may then be changed to systemic corticosteroids (severe exacerbation event) or revert back to the original ICS dose depending on asthma symptom progression.

Patients may be placed on systemic corticosteroids at any time as clinically indicated based on the presence of symptoms consistent with a severe asthma exacerbation event, as per the Investigator's judgment.

If a patient experiences two or more severe asthma exacerbation events anytime during the study, a permanent change (i.e., step up in medium- to high-dose ICS or addition of second controller for patients on high-dose ICS monotherapy) on their stable-dose background controller medication may occur, as indicated and upon recommendation of the physician and/or Investigator.

Patients who permanently discontinue the study medication is asked and encouraged to return to the clinic for study visits and participate in assessments according to the visit schedule until the end of the study (EOS) with a ±5 day window or up to recovery or stabilization of any adverse event (AE). At the time of permanent treatment discontinuation, patients perform the early treatment discontinuation (ETD) visit with all the assessments defined for the end-of-treatment (EOT) visit 28. Patients who permanently discontinue early from treatment is not eligible for the one-year long-term extension study.

For patients who permanently discontinue the study, under exceptional circumstances where there is no possibility for a patient and parent(s)/caregiver(s)/legal guardian(s) to come to the site for the scheduled follow-up visit, a phone contact may be made after Sponsor's approval is given. During that phone contact, at least information about AEs, concomitant medication and asthma exacerbation events must be collected, and the schedule for these calls should still reflect the visit schedule.

Patients who discontinue early from treatment may be asked to return to the clinic to have additional ADA samples collected for analysis based on the overall assessment of antibody titers and clinical presentation at the time of discontinuation.

### Post-Treatment Period

After completing the treatment period, patients are evaluated for 12 weeks (±5 days) in the post-treatment period. During this follow-up period, patients continue treatment with their stable dose of controller medication or it can be modified based on their level of asthma control, as determined by the investigator. Eligible patients who complete the randomized treatment period is offered the opportunity to participate in the one-year long-term extension study with DUPIXENT®. Patients subsequently enrolled in the one-year long-term extension study will not participate in the post-treatment period of this trial.

### Asthma Exacerbations

Two types of asthma exacerbation are defined in this study, as outlined below:

1) A severe exacerbation event during the study is defined as a deterioration of asthma requiring: use of systemic corticosteroids for ≥3 days; or hospitalization or emergency room visit because of asthma, requiring systemic corticosteroids.

2) A LOAC event is defined as any of the following: ≥6 additional reliever puffs of salbutamol/albuterol or levosalbutamol/levalbuterol in a 24-hour period (compared to baseline) on 2 consecutive days; increase in ICS dose ≥4 times than the dose at visit 2; a decrease in AM or PM peak flow of 30% or more on 2 consecutive days of treatment, based on the defined stability limit. The treatment period stability limit is defined as the respective mean AM or PM peak expiratory flow obtained over the last 7 days prior to randomization (day 1); or severe exacerbation event. Two events are considered as different if the interval between their start dates is equal or greater than 28 days.

The reasons (e.g., infections including viral and bacterial, allergen exposure, exercise and others) for any exacerbation event is recorded in the e-CRF.

### Study Population—Main Selection Criteria

### Inclusion Criteria

The following inclusion criteria are used: children 6 to <12 years of age, with an investigator diagnosis of persistent asthma for ≥12 months prior to screening, based on clinical history and examination, pulmonary function parameters according to Global Initiative for Asthma (GINA) 2015 Guidelines and the following criteria: existing background therapy of medium-dose ICS with second controller medication (i.e., LABA, LTRA, LAMA, or methylxanthines) or high-dose ICS alone or high-dose ICS with second controller, for at least 3 months with a stable dose ≥1 month prior to screening visit 1; pre-bronchodilator forced expiratory volume in 1 second (FEV1)≤95% of predicted normal or pre-bronchodilator FEV1/forced vital capacity (FVC) ratio <0.85 at screening and baseline visits; reversibility of at least 10% in FEV1 after the administration of 200 to 400 mcg (2 to 4 puff inhalations with metered-dose inhaler (MDI)) of albuterol/salbutamol or 45 to 90 mcg (2 to 4 puffs with MDI) of levalbuterol/levosalbutamol reliever medication before randomization (Up to 3 opportunities during the same visit are allowed with a maximum of 12 puffs of reliever medication if tolerated by the patient. Note: A maximum of 3 visits to meet the qualifying criterion of reversibility may be made during the screening period and prior to the patient's randomization. Documented reversibility or positive airway hyperresponsiveness to methacholine within 12 months prior to screening V1 is considered acceptable); must have experienced, within one year prior to use of reliever medication (i.e., albuterol/salbutamol or levalbuterol/levosalbutamol), other than as a preventive for exercise induced bronchospasm, on 3 or more days per week, on at least one week during the screening period; sleep awakening due to asthma symptoms requiring use of reliever medication at least once during the screening period; and asthma symptoms 3 or more days per week on at least one week during the screening period.

### Exclusion Criteria

The following exclusion criteria are used: patients <6 or ≥12 years of age; patients <16 kg bw; any other chronic lung disease (cystic fibrosis, bronchopulmonary dysplasia, etc.) which may impair lung function; a subject with any history of life-threatening asthma (e.g., requiring intubation); co-morbid disease that might interfere with the evaluation of investigational medicinal product (IMP); history of malignancy of any kind; inability to follow the procedures of the study (e.g., due to language problems or psychological disorders); anti-immunoglobulin E (IgE) therapy (omalizumab) within 130 days prior to visit 1 or any other biologic therapy/immunosuppressant to treat inflammatory disease or autoimmune disease (e.g., rheumatoid arthritis, inflammatory bowel disease, systemic lupus erythematosus as well as other diseases) within 2 months or 5 half-lives prior to visit 1, whichever is longer; initiation of allergen immunotherapy within 3 months prior to visit 1 or dose change from one month prior to visit 1 or a plan to begin allergen immunotherapy or to change its dose during the screening period or the randomized treatment period; exposure to another investigative antibody within a time period prior to visit 1 that is less than five half-lives of the antibody. In case the half-life is not known, then the minimum interval since exposure to the prior investigative antibody is 6 months. The minimum interval since exposure to any other (non-antibody) investigative study medication is 30 days prior to visit 1; patients receiving medications or therapy that are prohibited as concomitant medications; patients who have previously been treated in any clinical trial of DUPIXENT®; or patients or his/her parent(s)/caregiver(s)/legal guardian(s) is related to the investigator or any sub-investigator, research assistant, pharmacist, study coordinator, other staff thereof directly involved in the conduct of the study.

### Prohibited Concomitant Medication

The following concomitant treatments are not permitted during the screening period or during the randomized treatment period: systemic corticosteroids (SCSs) for diagnoses other than severe exacerbation of asthma and/or high-potency topical steroids within 30 days before screening visit 1, during the screening period, and/or during the randomized treatment phase of this study (intra-articular steroids are not allowed to be used in the above mentioned period); IgE therapy (e.g., omalizumab) within 130 days prior to screening visit 1, or any other biologic therapy/immunosuppressant to treat inflammatory disease or autoimmune disease within 2 months prior to screening visit 1, allergen immunotherapy (except if initiated more than 3 months prior to visit 1 and dose stable 1 month prior to visit 1); intravenous immunoglobulin (IVIG) therapy; live attenuated vaccines (live (attenuated) vaccines are allowed in the screening period, if taken at least 4 weeks prior to the administration of the first dose of investigational medicinal product (i.e., at least 4 weeks prior to baseline visit); in Brazil, for patients in the yellow fever outbreak affected area; asthma relievers other than salbutamol/albuterol or levosalbutamol/levalbuterol: their use is not recommended unless in exceptional circumstances during the study period (e.g., prescribed by a physician not participating in the study); exposure to another antibody within a time period prior to visit 1 that is less than 5 half-lives of the antibody. In case the half-life is not known, then the minimum interval since exposure to the prior investigative antibody is 6 months. The minimum interval since exposure to any other (non-antibody) investigative study medication is 30 days prior to visit 1; any investigational treatment or procedure.

Prohibited live attenuated vaccines include: bacillus Calmette-Guêrin (BCG) antituberculosis vaccine; chickenpox (Varicella); intranasal influenza (FluMist-Influenza); inactive influenza vaccine delivered by injection is permitted; measles (Rubeola); measles-mumps-rubella (MMR) combination; measles-mumps-rubella-varicella (MMRV) combination; mumps; oral polio (Sabin); oral typhoid; rotavirus; rubella; smallpox (Vaccinia); varicella zoster (shingles); and yellow fever.

### Permitted Concomitant Medication

The following concomitant treatments are permitted during the screening period or during the randomized treatment period: antihistamines; dermatological, ocular or intranasal corticosteroids (except for high-potency dermatological corticosteroids); cytochrome P450 (CYP) enzyme substrates. Examples of CYP substrates with a narrow therapeutic range are: theophylline, tizanidine, paclitaxel, warfarin, phenytoin, s-mephenytoin, alfentanil, astemizole, cisapride, cyclosporine, dihydroergotamine, ergotamine, fentanyl, pimozide, quinidine, sirolimus, tacrolimus, terfenadine and thioridazine.

### Expected Number of Patients

Approximately 402 patients are randomized in a 2:1 ratio to receive DUPIXENT® (268) or placebo (134).

### Formulation

DUPIXENT® for children ≤30 kg body weight (bw) at randomization: 150 mg/mL in pre-filled syringe to deliver a once every 2 weeks (q2w) dose of 100 mg in a 0.67 mL subcutaneous injection. DUPIXENT® for children >30 kg bw at randomization: 175 mg/mL in pre-filled syringe to deliver a once q2w dose of 200 mg in a 1.14 mL subcutaneous injection. Placebo: Matching placebo in a prefilled syringe to deliver a once q2w dose of placebo in a 0.67 or 1.14 mL subcutaneous injection for children with ≤30 or >30 kg bw at randomization, respectively. DUPIXENT® or matching placebo in glass pre-filled syringes are dispensed to the patients.

### Route of Administration

The IMP is administered by subcutaneous (SC) injection.

### Dose Regimen

Randomized 2:1 to the following regimens: DUPIXENT®, 200 or 100 mg SC once q2w for children with bw>30 kg or ≤30 kg, respectively; placebo, SC q2w. After randomization, dose regimen is not adjusted for patient's age or weight during the randomized treatment period of this study.

Non-investigational medicinal products (background therapy) are inhaled corticosteroid in combination with a second controller medications.

### Dose Schedule

The IMP is administered every 14±3 days q2w. The doses of investigational product must be separated by ≥11 days to avoid overdose.

The IMP administrations are performed by the investigator or designee at scheduled study site visits following clinic procedures and blood collection. Patients are monitored for a minimum of 30 minutes after each study-site administrated injection of IMP, to assess any injection site reactions (e.g., for any signs or symptoms of a hypersensitivity reaction).

For all visits scheduled only for IMP administration, parent(s)/caregiver(s)/legal guardian(s) may decide to do the injection of IMP at home (i.e., home administration of IMP). These parent(s)/caregiver(s)/legal guardian(s) are trained by the investigator or designee to administer IMP, by demonstration at visit 2, visit 3, and visit 4 (injections performed by Investigator). After parent(s)/caregiver(s)/legal guardian(s) have successfully administered IMP under close supervision of the investigator at visit 5-visit 8 (weeks 6, 8, 10, and 12), the investigator may approve them to perform home administration of IMP at all further visits that do not require a scheduled clinic visit (i.e., at weeks 14, 18, 22, 26, 30, 34, 38, 42, 46, and 50). Patients should be monitored for 30 minutes after home administration of IMP. It is possible to start home administration at any visit following visit 9, provided parent(s)/caregiver(s)/legal guardian(s) have been trained by the investigator or designee to administer IMP by demonstration at not less than 3 visits followed by a successful IMP administration under close supervision of the investigator or designee at not less than 3 visits.

However, if parent(s)/caregiver(s)/legal guardian(s) do not develop the comfort to inject the IMP at home, or the investigator determines that injection by parent(s)/caregiver(s)/legal guardian(s) at home is not appropriate, alternative arrangements may be made: for example for qualified site personnel and/or healthcare professionals (e.g., visiting nurse service) to administer IMP at these timepoints at the patient's home.

For IMP doses not given at the study site, ‘home dosing diary’ (paper format) is provided to record information related to the injections. Such home dosing diaries is kept as source data in the patient's study file.

Parent(s)/caregiver(s)/legal guardian(s) should be instructed to avoid missing any site visits (i.e., IMP doses) or doses of background therapy during the study. For any patient who misses a site-visit (i.e., IMP dose) or doses of background therapy, the parent(s)/caregiver(s)/legal guardian(s) should be reminded to be diligent to avoid missed visits and doses of background therapy thereafter.

The patient(s)/parent(s)/caregiver(s)/legal guardian(s) should continue their scheduled visits for IMP treatment, even if more than two consecutive doses of IMP are missed, or background medication was not taken by the patient(s) for up to two-four days.

The SC injection sites should be alternated among the 4 quadrants of the abdomen (avoiding navel and waist areas), the upper thighs or the upper arms, so that the same site is not injected twice consecutively. For each injection, the anatomic site of administration is recorded in the electronic-case report form (e-CRF) or, as applicable, the home dosing diary.

Detailed instructions for transport, storage, preparation, and administration of IMP are provided to the patient and parent(s)/caregiver(s)/legal guardian(s). Parent(s)/caregiver(s)/legal guardian(s) complete a dosing diary to document compliance with injection of IMP.

### Screening Period

Prior to and during the screening period, patients must be on stable-dose background therapy of medium-dose ICS with a second controller medication (i.e., long-acting β2 agonist (LABA), long acting muscarinic antagonist (LAMA), leukotriene receptor antagonist (LTRA) or methylxanthine) or high-dose ICS alone or high-dose ICS with second controller.

### Randomized Treatment Period

During this period, patients continue taking their controller medication(s). For patients experiencing deterioration of asthma during the study, the ICS dose may temporarily be increased up to 4-fold (recorded as LOAC event) for a maximum of 10 days, as indicated and upon recommendation of the physician and/or investigator. Treatment may then be changed to systemic corticosteroids (severe exacerbation event) or revert back to the original ICS dose depending on asthma symptom progression.

Patients may be placed on SCS at any time as clinically indicated based the presence of symptoms consistent with a severe asthma exacerbation event, as per the Investigator's judgment.

If a patient experiences two or more severe asthma exacerbation events any time during the study, a permanent change (i.e., step up in medium- to high-dose ICS or addition of second controller for patients on high-dose ICS monotherapy) on their stable-dose background controller medication may occur, as indicated and upon recommendation of the physician and/or investigator.

### Post-Treatment Period

Upon completing the randomized treatment period, patients not continuing with the one-year long-term extension study, continue treatment with the controller medication regimen and dose used during the randomized period, which could be adjusted based on medical judgment of the patients' asthma control status.

### Reliever Medication

Patients may use albuterol/salbutamol or levalbuterol/levosalbutamol MDI as reliever medication as needed during the study. Nebulizer solutions may be used as an alternative delivery method.

### Routes of Administration

Oral inhalation by puff inhalation with MDI (e.g., ICS, ICS combination, albuterol/salbutamol or levalbuterol/levosalbutamol; or other background controllers according to label).

### Dose Regimen

ICS: medium to high-dose in combination with a second controller; reliever medication: Albuterol/salbutamol or levalbuterol/levosalbutamol: as needed.

### Endpoints

### Primary Endpoint

Annualized rate of severe exacerbation events during the 52-week placebo-controlled treatment period.

### Key Secondary Efficacy Endpoint

Change from Baseline in pre-bronchodilator % predicted forced expiratory volume in 1 second (FEV₁) at week 12.

### Secondary Endpoints

### Efficacy

Change from baseline in pre-bronchodilator % predicted forced expiratory volume in 1 second (FEV1) at weeks 2, 4, 8, 24, 36 and 52 and other time points assessed.

Time to first severe exacerbation event during 52-week treatment period.

Time to first LOAC during 52-week treatment period.

Change from baseline in other lung function measurements (absolute and relative FEV1, AM/PM peak expiratory flow (PEF), FVC, forced expiratory flow (FEF) 25-75%, post-bronchodilator % predicted FEV1) at weeks 2, 4, 8, 12, 24, 36, 52 and other time points assessed.

The effect of DUPIXENT® on healthcare resource utilization.

Change from baseline at weeks 2, 4, 8, 12, 24, 36, 52, and other time points assessed, in: morning/evening asthma symptom score (electronic diary); PRO: Asthma Control Questionnaire-Interviewer Administered (ACQ-IA), for children 6 to <12 years old, use of reliever medication, number of nocturnal awakenings due to asthma symptoms requiring the use of reliever medication

Change from baseline at weeks 12, 24, 36, 52, 64 in: PRO: Pediatric Asthma Quality of Life Questionnaire with Standardised Activities-Interviewer Administered (PAQLQ(S) IA) score, for children ≥7 to <12 years old at randomization.

### Safety and Tolerability

Adverse events (AEs); vital signs (including height, weight); physical examination; electrocardiogram (ECG); clinical laboratory tests; systemic drug concentration, anti-drug antibodies and IgG responses to vaccination during drug treatment; serum functional DUPIXENT® concentrations; ADA; IgG responses to vaccination with any vaccination for tetanus, diphtheria, pertussis and/or seasonal trivalent/quadrivalent influenza vaccine during DUPIXENT® treatment (may be analyzed as exploratory endpoint if insufficient power).

### Biomarkers

Change from baseline in fractional exhaled nitric oxide (FeNO) at week 12.

### Exploratory Endpoints

Change from baseline and blood biomarkers (thymus and activation regulated chemokine (TARC), serum total immunoglobulin E (IgE)).

Optional genetic analysis of genomic DNA to assess the association of genetic variation with asthma and response to DUPIXENT® treatment.

The proportion of patients requiring a permanent step up in background controller medication after 2 or more severe asthma exacerbation events.

The effect of DUPIXENT® on additional PROs: Pediatric Asthma Caregiver's Quality of Life Questionnaire (PACQLQ) score, for caregivers of children ≥7 years old at randomization; Pediatric Rhinoconjunctivitis Quality of Life Questionnaire-Interviewer Administered (PRQLQ-IA) score, in children 6 to <12 years old, with history of allergic rhinitis); EuroQol 5 dimension youth questionnaire (EQ-5D-Y) for children.

Change from Baseline in antigen-specific IgE, antigen-specific immunoglobulin G subtype 4 (IgG4) and ratio of IgE:IgG4.

Slope of % predicted FEV1

### Criteria for Asthma Exacerbations During the Study

Two types of asthma exacerbation are defined in this study, as outlined below:

1) A severe exacerbation event during the study is defined as a deterioration of asthma requiring: use of systemic corticosteroids for ≥3 days; or hospitalization or emergency room visit because of asthma requiring systemic corticosteroids.

2) A LOAC event is defined as any of the following: ≥6 additional reliever puffs of salbutamol/albuterol or levosalbutamol/levalbuterol in a 24 hour period (compared to baseline) on 2 consecutive days; increase in ICS dose ≥4 times than the dose at visit 2; a decrease in AM or PM peak flow of 30% or more on two consecutive days of treatment, based on the defined stability limit. The Treatment Period stability limit is defined as the respective mean AM or PM peak expiratory flow obtained over the last 7 days prior to randomization (Day1); severe exacerbation event; two events are considered as different if the interval between their start dates is equal or greater than 28 days.

According to certain embodiments, salbutamol/albuterol nebulizer and levosalbutamol/levalbuterol nebulizer use can be converted as shown in Table 5 and Table 6 below. An example of salbutamol/albuterol nebulizer-to-puff conversion: patient received 3 salbutamol/albuterol nebulizer treatments (2.5 mg/treatment) between 7 and 11 AM. Total daily=7.5 mg or 12 puffs. An example of levosalbutamol/levalbuterol nebulizer-to-puff conversion: patient received 3 levosalbutamol/levalbuterol nebulizer treatments (1.25 mg/treatment) between 7 and 11 AM. Total daily=3.75 mg or 12 puffs. After conversion of nebulizer-to-puff, and for every instance that the number of puffs is ≥6 additional puffs of salbutamol/albuterol or levosalbutamol/levalbuterol in a 24-hour period (compared to baseline) on 2 consecutive days in any week, a LOAC event should be documented.

TABLE 5
Salbutamol/albuterol nebulizer use.

| Salbutamol/Albuterol Nebulizer | Number of |
|---|---|
| Solution-Total Daily Dose (mg) | Puffs* |
| 2.5 | 4 |
| 5.0 | 8 |
| 7.5 | 12 |
| 10 | 16 |

*Conversion factor: salbutamol/albuterol nebulizer solution (2.5 mg) corresponds to 4 puffs

TABLE 6
levosalbutamol/levalbuterol nebulizer use.

| Levosalbutamol/Levalbuterol Nebulizer | Number |
|---|---|
| Solution-Total Daily Dose (mg) | of Puffs* |
| 1.25 | 4 |
| 2.5 | 8 |
| 3.75 | 12 |
| 5 | 16 |

*Conversion factor: levosalbutamol/levalbuterol nebulizer solution (1.25 mg) corresponds to 4 puffs

### Assessment Schedule

1. Screening period (4 [±1] weeks).

2. Randomized treatment Period (up to 52 weeks).

3. Post-treatment period (12 weeks).

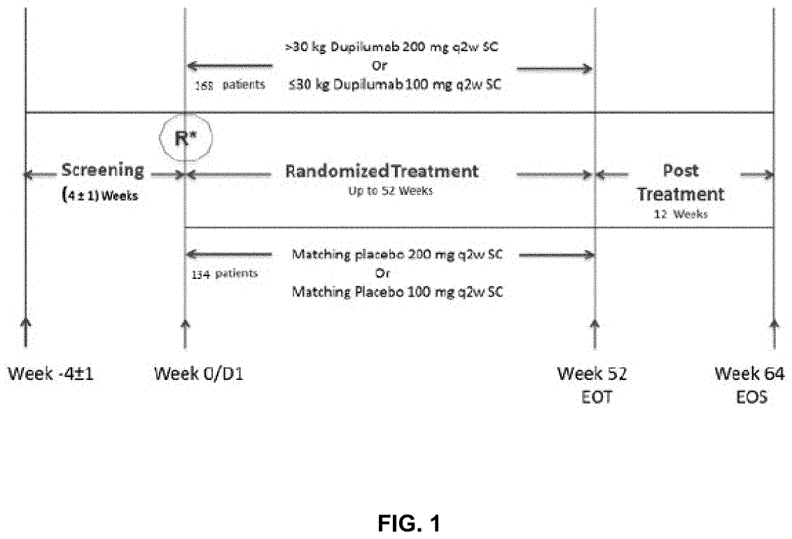

A schematic of the assessment schedule is shown in FIG. 1.

### Study Flow Chart

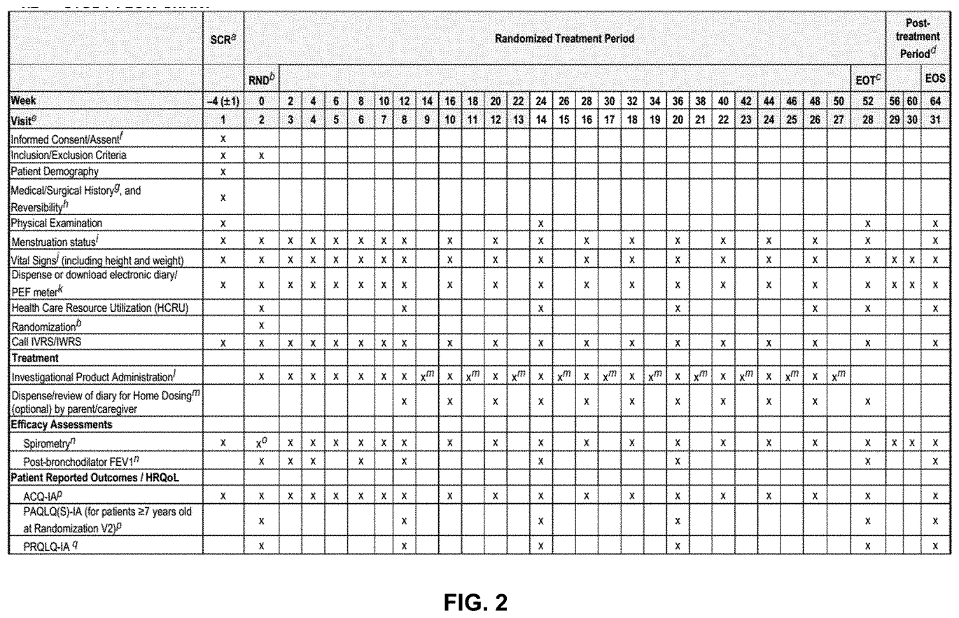

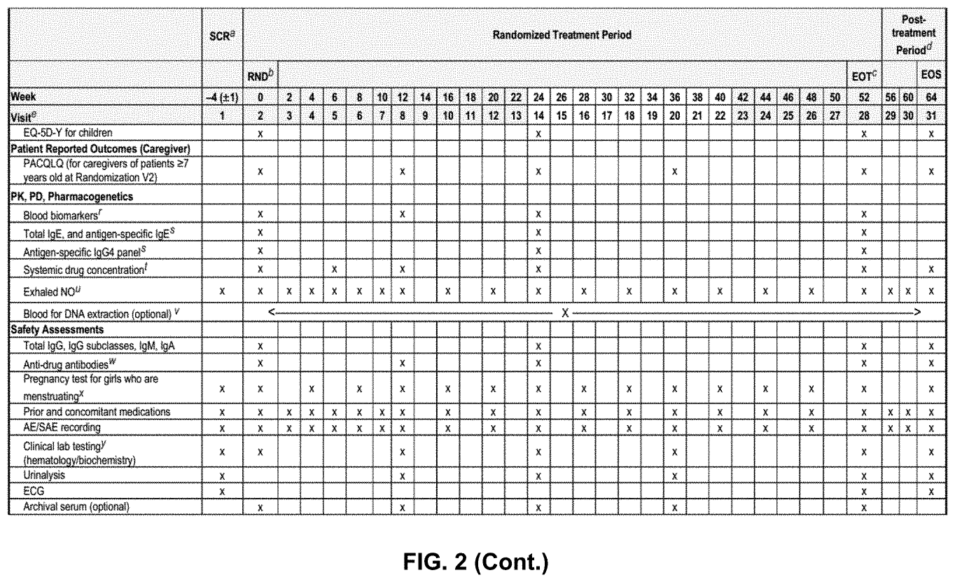

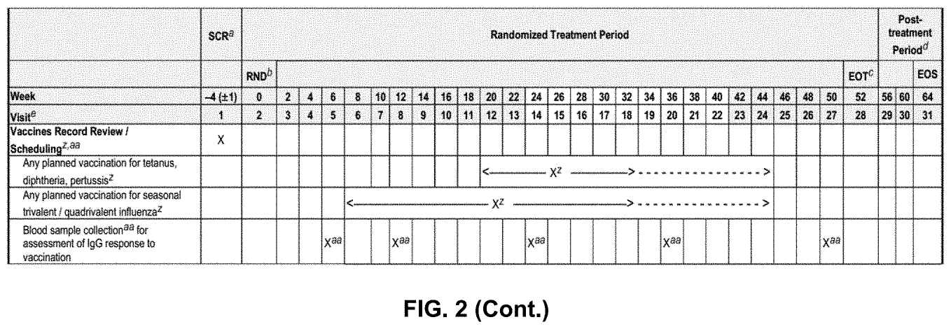

A flow chart of the study is set forth in FIG. 2.

(a) The screening period is 4±1 weeks (21-35 days) in duration to collect baseline data on asthma control and assure eligibility criteria. Prior to and during the screening period, patients must be on one of the following: stable-dose background therapy of medium-dose inhaled corticosteroid (ICS) with second controller medication (i.e., long-acting β2 agonist (LABA), leukotriene receptor antagonist (LTRA), long-acting muscarinic antagonist (LAMA), or methylxanthines) or high-dose ICS alone or high-dose ICS with second controller, for at least 3 months with a stable dose ≥1 month prior to screening visit 1.

(b) Randomization visit (visit 2) is defined as day 1. The randomization is stratified by eosinophil count (<300 cells/μL and ≥300 cells/μL) and stable dose-level of ICS (medium/high) at screening, and by region.

(c) Patients who permanently discontinue the study medication are asked and encouraged to return to the clinic for study visits and participate in assessments according to the visit schedule until the end of the study (EOS) with a ±5 day window or up to recovery or stabilization of any adverse event. At the time of permanent treatment discontinuation, patients perform the early treatment discontinuation (ETD) visit, with all the assessments defined for the end-of-treatment (EOT) visit 28. However, patients who discontinue early from treatment are be eligible for the one-year long-term extension study. For patients who permanently discontinue the study, under exceptional circumstances where there is no possibility for a patient and parent(s)/caregiver(s)/legal guardian(s) to come to the site for the scheduled follow-up visit, a phone contact may be made after Sponsor's approval is given. During that phone contact, at least information about adverse events (AEs), concomitant medication and asthma exacerbation events must be collected, and the schedule for these calls should still reflect the visit schedule. Patients who discontinue early from treatment may be asked to return to the clinic to have additional ADA samples collected for analysis based on the overall assessment of antibody titers and clinical presentation at the time of discontinuation.

(d) Eligible patients who complete the randomized treatment period are offered the opportunity to participate in the 1-year long-term extension study with DUPIXENT®. Patients subsequently enrolled in the one-year long-term extension study do not participate in the post-treatment period of this trial.

(e) The visit windows for all subsequent visits post-randomization on day 1 is ±3 days during the treatment period and ±5 days during the post-treatment period.

(f) Prior to any screening assessments: all patient ≥6 years of age (or above an age determined by the Institutional Review Board (IRB)/Independent Ethics Committee (IEC) and in according with the local regulations and requirements) and their parent(s)/caregiver(s)/legal guardian(s) receive information about the study, on study objective(s) and procedures, to the fullest extent possible, in their language and in terms they are able to understand, and must sign and date the IRB/IEC approved Informed Assent Form (IAF) and Informed Consent Form (ICF), respectively. For girls who have started menstruating, a specific assent form must be obtained. For each of the following two optional assessments, a separate ICF/IAF must be obtained: pharmacogenetic samples at Week 0 prior to investigational medicinal product (IMP) administration, archival serum at various time-points outlined above.

(g) Medical history, asthma-specific medical history (i.e., family history of atopy and IgE-mediated disease (particularly maternal), premature birth and/or, low birthweight, exposure to tobacco smoke, recurring viral infections in early childhood), surgical history.

(h) Reversibility of at least 10% in FEV1 after the administration of 200 to 400 mcg (2 to 4 puff inhalations with metered-dose inhaler [MDI]) of albuterol/salbutamol or 45 to 90 mcg (2 to 4 puffs with MDI) of levalbuterol/levosalbutamol reliever medication before randomization (up to 3 opportunities during the same visit are allowed with a maximum of 12 puffs of reliever medication if tolerated by the patient). Documented reversibility or positive airway hyperresponsiveness to methacholine within 12 months prior to screening V1 is considered acceptable. If the subject does not meet this reversibility criterion at screening V1, up to 2 additional assessment attempts can be performed at any time between screening and baseline visit 2.

(i) A separate assent must be obtained from female patients at the earliest visit when the investigator is notified that the first menses have occurred.

(j) Vital signs, including blood pressure (mmHg), heart rate (beats per minute), respiratory rate (breaths per minute), body temperature (degrees Celsius), height (cm) and body weight (kg) are measured at the screening and randomization visits (visits 1 and 2) and every subsequent visit. Vital signs are measured in the sitting position using the same arm at each visit, and are measured prior to receiving investigational product at the clinic visits.

(k) Electronic diary/PEF meter is used for daily recording of salbutamol/albuterol or levosalbutamol/levalbuterol use, asthma controller drug use, oral steroid requirements, nocturnal awakenings due to asthma symptoms requiring the use of reliever medications, morning and evening asthma symptom NRS scores and AM and PM PEF. This device is dispensed at visit 1 and information is downloaded from this device on the other indicated days.

(l) During the randomized treatment period IMP administrations, every 2 week (q2w), are performed by the investigator at scheduled study site visits (must be separated by at least 11 days) up to week 50. In the first 12 weeks (up to V8), patients are monitored at the study site for a minimum of 30 minutes after injection of IMP, to assess any injection reactions (see Section 8.1.4 for more details). After randomization, dose regimen are not be adjusted for patient's age or weight until the randomized treatment are completed.

(m) Home dosing and training of parent(s)/caregiver(s)/legal guardian(s): for all visits scheduled only for IMP administration (i.e., at weeks 14, 18, 22, 26, 30, 34, 38, 42, 46, and 50), parent(s)/caregiver(s)/legal guardian(s) may decide to do the injection of IMP at home (i.e., home administration of IMP). These parent(s)/caregiver(s)/legal guardian(s) are trained by the investigator or designee to administer IMP, by demonstration at V2, V3, and V4 (injections performed by the investigator). After parent(s)/caregiver(s)/legal guardian(s) have successfully administered IMP under close supervision of the Investigator at V5-V8 (weeks 6, 8, 10, and 12), the investigator may approve them to perform home administration of IMP at all further visits that do not require a scheduled visit. It is possible to start home administration at any visit following V9, provided parent(s)/caregiver(s)/legal guardian(s) have been trained by the investigator or designee to administer IMP by demonstration at not less than 3 visits followed by a successful IMP administration under close supervision of the investigator or designee at not less than 3 visits. However, if parent(s)/caregiver(s)/legal guardian(s) do not develop the comfort to inject the IMP at home, or the Investigator determines that injection by parent(s)/caregiver(s)/legal guardian(s) at home is not appropriate, alternative arrangements may be made: for example for qualified site personnel and/or healthcare professionals (e.g., visiting nurse service) to administer IMP at these timepoints at the patient's home.

(n) Forced expiratory volume (FEV1), PEF, forced vital capacity (FVC), forced expiratory flow between 25% to 75% of the pulmonary volume (FEF_{25-75%}) at all visits; pulmonary function tests should be performed in the morning if possible, but if it could only be done at a different time of the day, the spirometry should be done at approximately the same time of the day at each visit throughout the study. Spirometry is performed after a wash out period of bronchodilators according to their action duration, for example, withholding the last dose of salbutamol/albuterol or levosalbutamol/levalbuterol for at least 6 hours, withholding the last dose of LABA for at least 12 hours, and withholding the last dose of LAMA for at least 24 hours. This is verified before performing the PEF measurements.

(o) Treatment Period stability limits are established for FEV1 and PEF. Period stability limit for PEF is defined as the respective mean AM or PM PEF obtained over the last 7 days prior to visit 2 (day1). There should be at least 4 days' measurement for setting up the stability limit, and the first dosing visit should be rescheduled until data for 4 days are available.

(p) Asthma Control Questionnaire-Interviewer Administered (ACQ-IA, for children 6 to <12 years), ACQ-7 and ACQ-5 scores and Pediatric Asthma Quality of Life Questionnaire With Standardised Activities-Interviewer Administered (PAQLQ(S)-IA) score, for children ≥7 years old at randomization V2, are administered by the interviewer during the study visits at the clinical site. The ACQ-7 score is used to follow up evaluations in all patients. The ACQ-5 (the first 5 items of the ACQ-7) score is used for eligibility evaluation at Screening V1 and Baseline V2 for all patients.

(q) Pediatric rhinoconjunctivitis quality of life questionnaire-Interviewer Administered (PRQLQ-IA): for those patients with comorbid allergic rhinitis, administered by the interviewer during the study visits at the clinical site.

(r) Biomarker set includes serum thymus and activation-regulated chemokine (TARC).

(s) Assessment of total IgE, antigen-specific IgE, antigen-specific IgG4, and ratio of IgE:IgG4.

(t) Systemic drug concentration samples are to be collected prior to dosing and in case of SAE and AESI.

(u) Exhaled nitric oxide assessment is conducted prior to spirometry and following a fast of ≥1 hour.

(v) This is optional, and parent(s) or caregiver(s) or legal guardian(s)/patients must sign a separate ICF/IAF before sampling. For those who consented, the sample can be drawn at week 6, before IMP administration or at any time during the study, taking into consideration limitation in blood collection volume at the time.

(w) ADA samples are collected prior to dosing and in case of SAE and AESI.

(x) For female patients who have commenced menstruating (i.e., are of child-bearing potential) at screening, a urine pregnancy test is mandatory at the screening visit 1 with negative result obtained prior to randomization at visit 2 (week 0) and at every subsequent visits defined in the flowchart. For female patients who happen to commence menstruating after screening, a negative urine dipstick pregnancy test is obtained prior to administration of IMP.

(y) Hematology: hemoglobin, hematocrit, platelet count, total white blood cell (WBC) count with five-part differential count, and total red blood cell count. Serum chemistry: creatinine, blood urea nitrogen, glucose, uric acid, total cholesterol, total protein, albumin, total bilirubin (in case of values above the normal range, differentiation in conjugated and non-conjugated bilirubin), alanine aminotransferase, aspartate aminotransferase, alkaline phosphatase, lactate dehydrogenase, electrolytes (sodium, potassium, chloride), bicarbonate, and creatine phosphokinase. Patients' fasting (considering fasting as no intake of any food or drink except for water for at least 8 hours) or non-fasting status at blood sample collection is recorded on the Central Laboratory Requisition Form. Clinical laboratory testing only at screening visit 1 includes hepatitis screen covering hepatitis B surface antigen (HBs Ag), hepatitis B surface antibody (HBs Ab), hepatitis B core antibody (HBc Ab), hepatitis C virus antibodies (HCV Ab), human immunodeficiency virus (HIV) screen (Anti-HIV-1 and HIV-2 antibodies) and anti-nuclear antibody (ANA).

(z) At screening, parent(s)/caregiver(s)/legal guardian(s) are asked to provide information on their child's vaccination schedule, and assess whether immunizing their children with any vaccination for tetanus, diphtheria, pertussis and/or seasonal trivalent/quadrivalent influenza (as per local medical practice) results in vaccination during the study. The timing of these vaccinations should be adjusted to fit into the IMP treatment period if appropriate. Any planned tetanus, diphtheria and pertussis vaccination should be administered between visit 12 (week 20) and visit 18 (week 32), as administration after visit 18 (week 32) may require an additional blood draw (refer to Section 9.3.1.2) for assessment. Any planned seasonal trivalent/Quadrivalent influenza should be administered between visit 6 (week 8) and visit 18 (week 32) as administration after visit 18 (week 32) may require an additional blood draw (refer to Section 9.3.1.2) for assessment.

(aa) Scheduled blood sample collection for pre- and post-vaccine antibody titers (i.e., for IgG response assessment), for both vaccinations (i.e., any tetanus, diphtheria and pertussis and/or seasonal trivalent/quadrivalent influenza) should be drawn within 8 weeks prior to vaccination and at 3-4 weeks (up to 6 weeks) after the respective vaccination(s). However, all blood titer samples must be drawn between week 6 and week 50 (i.e., visit 5 and visit 27, respectively). Depending on a patient's vaccination schedule during the course of this study, every effort is made to draw pre-vaccination titers at either weeks 6, 12, or 24 (V5, V8, V14) of the randomized treatment period, and to draw post-vaccination titers at either weeks 12, 24, or 36 (V8, V14, V20) of the randomized treatment period.

### Statistical Considerations

### Sample Size Determination

The sample size of this study was based on a comparison between DUPIXENT® versus placebo with regard to the primary endpoint of annualized rate of severe exacerbations over 52 weeks of treatment for the 3 populations of interest: patients with baseline blood eosinophils ≥300 cells/μL, patients with baseline blood eosinophils ≥150 cells/μL, and patients with Type 2 inflammatory phenotype (baseline blood eosinophils ≥150 cells/μL or baseline FeNO≥20 ppb), with assuming the number of severe exacerbations follows a negative binomial distribution and a randomization ratio of 2:1.

For patients with baseline blood eosinophils ≥300 cells/μL, assuming a placebo annualized severe exacerbation rate of 0.8 and a dispersion parameter of 1.5, with approximately 255 patients randomized (170 for DUPIXENT® and 85 for matching placebo group), this study will have approximately 96% power to detect a 60% relative risk reduction (i.e., annualized rate of 0.32 for the DUPIXENT® group) in the annualized rate of severe exacerbations at the 2-tailed significance level of α=0.05 among these patients.

For patients with baseline blood eosinophils ≥150 cells/μL, assuming a placebo annualized severe exacerbation rate of 0.7, and a dispersion parameter of 1.5, with approximately 327 patients randomized (218 for DUPIXENT® and 109 for matching placebo group), the study will have approximately 93% power to detect a 54% relative risk reduction (i.e., annualized rate of 0.322 for the DUPIXENT® group) in the annualized rate of severe exacerbations at the 2-tailed significance level of α=0.05 among these patients.

For patients with Type 2 inflammatory phenotype (baseline blood eosinophils ≥150 cells/μL or baseline FeNO≥20 ppb), assuming a placebo annualized severe exacerbation rate of 0.7, and a dispersion parameter of 1.5, with approximately 345 patients (230 for DUPIXENT® and 115 for matching placebo group), the study will have approximately 94% power to detect a 54% relative risk reduction (i.e., annualized rate of 0.322 for the DUPIXENT® group) in the annualized rate of severe exacerbations at the 2-tailed significance level of α=0.05 among these patients.

The sample size calculation assumes a linear discontinuation rate (20% at one year), thus the average exposure duration for patients is 0.9 year. The assumed relative risk reductions are based on the results in the phase 3 asthma EFC13579 QUEST study.

To achieve target sample size for each of the populations stated above, approximately 402 patients in the overall population (268 for DUPIXENT® and 134 for placebo) need to be randomized assuming approximately 86% of the randomized patients with Type 2 inflammatory phenotype (baseline blood eosinophils ≥150 cells/4, or baseline FeNO≥20 ppb), assuming approximately 81% of the randomized patients have baseline blood eosinophils ≥150 cells/μL, and approximately 64% of the randomized patients have baseline blood eosinophils ≥300 cells/μL.

Patients are randomized (2:1 ratio) to receive DUPIXENT® or matching placebo. After a patient is randomly assigned to DUPIXENT® or matching placebo, the dosage of DUPIXENT® or matching placebo for the patient, 200 or 100 mg SC once q2w, are determined based on body weight >30 kg or ≤30 kg, respectively.

Randomization is stratified by ICS dose (medium-dose versus high-dose) and eosinophil count (<300 cells/μL versus ≥300 cells/μL) at Screening, and by region.

### Analysis Populations

In order to confirm the efficacy of DUPIXENT® with appropriate multiplicity control, there will be two primary analysis populations to evaluate the efficacy endpoints:

1. Population with Type 2 inflammatory phenotype will be defined as randomized patients with baseline blood eosinophils ≥150 cells/4, or baseline FeNO≥20 ppb. This multiplicity control will be applied to the analysis in countries that use the same or similar indication as approved in the EU.

2. Population with baseline blood eosinophil ≥300 cells/μL, which is defined as the randomized patients with baseline blood eosinophil ≥300 cells/μL, will be the primary analysis population that the sponsor uses for US and US reference countries, similar to the approach taken for evaluating these patients in the QUEST study. In addition, patients with baseline blood eosinophils ≥150 cells/μL will be tested in the hierarchy. This multiplicity will be used in countries with the same or similar indication wordings as approved in the US.

The efficacy analyses will be conducted according to the treatment to which they are randomized.

The analysis population for the safety endpoints is the safety population, defined as all patients exposed to study medication, regardless of the amount of treatment administered and regardless of whether they are randomized.

The safety analyses are conducted according to the treatment patients actually received.

### Analysis of the Primary Endpoint

The estimand of the DUPIXENT® treatment effect compares the annualized rate of severe exacerbation for the patients randomized to the DUPIXENT® and placebo arms, regardless of what treatment patients actually received. It assesses the benefits of the treatment policy or strategy relative to placebo. In this primary approach, off-treatment measurements of patients who prematurely discontinue treatment is included for the analysis. Patients who permanently discontinue the study medication are asked and encouraged to return to the clinic for all remaining study visits. If a patient stays in study until the end of 52-week treatment period, all severe exacerbation events that happen up to week 52 are included in the primary analysis, regardless if the patient is on-treatment or not. If a patient withdraws from study prior to the end of 52-week treatment period, all observed severe exacerbation events up to the last contact date are included in the analysis, and the observation duration is defined as from randomization to the last contact date. No imputation is performed for the unobserved events that may happen after study discontinuation and up to week 52.

The annualized rate of severe asthma exacerbation events are analyzed using a negative binomial regression model. The analysis of the primary endpoint is conducted in the Type 2 inflammatory phenotype, baseline blood eosinophils ≥300 cells/μL, baseline blood eosinophils ≥150 cells/μL, baseline FeNO≥20 ppb and full intent-to-treat (ITT) populations using appropriate multiplicity control. When performing the primary endpoint analysis in the Type 2 inflammatory phenotype, baseline blood eosinophils ≥150 cells/μL or the full ITT populations, the model includes the total number of events of each patient occurring during the 52 weeks as the response variable, with the treatment group, age, weight (≤30 kg, >30 kg), region, baseline eosinophil level (<300 cells/μL, ≥300 cells/μL), baseline FeNO level (<20 ppb, ≥20 ppb), baseline ICS dose level (medium/high) and number of severe asthma exacerbation events prior to the study as covariates. When performing the primary endpoint analysis in the baseline blood eosinophils ≥300 cells/μL population, the baseline eosinophil level is removed from the model covariates. When performing the primary endpoint analysis in the baseline FeNO≥20 ppb population, the baseline FeNO level is removed from the model covariates. Severe asthma exacerbation event prior to the study is defined as treatment with a systemic steroid (oral or parenteral) for worsening asthma at least once or hospitalization or emergency medical care visit for worsening asthma (as defined in this protocol). Log transformed observation duration is the offset variable. A supportive analysis to assess the treatment effect of DUPIXENT® if patients adhere to the treatment and background asthma medication as directed is also provided. In this approach, the severe exacerbation events reported after the premature treatment discontinuation are excluded from the analysis. Any measurement obtained after the first permanent step up of background asthma medication is also excluded from the analysis. The supportive analysis is performed in the Type 2 inflammatory phenotype and baseline blood eosinophils ≥300 cells/μL populations and use a negative binomial model with the same set of covariates as specified for the primary analysis in the two populations. This model includes severe exacerbation events occurring during the treatment epoch before any permanent stepping-up of background asthma medication as the response variable and the log transformed duration of the treatment or from randomization to first permanent stepping-up of background asthma medication whichever is shorter is the offset variable.

The analysis of the primary endpoint is conducted in the Type 2 inflammatory phenotype, baseline blood eosinophils ≥300 cells/μL, baseline blood eosinophils ≥150 cells/μL, baseline FeNO≥20 ppb, and full ITT populations using appropriate multiplicity control.

### Multiplicity Considerations

The hypothesis testing on the primary endpoint of annualized severe exacerbation rate is controlled with a two-sided type I error of 0.05 by incorporating a sequential testing procedure as below:

### For US and US Reference Countries

1st: Annualized rate of severe exacerbation events during the 52-week placebo-controlled treatment period based on the patients with baseline blood eosinophils ≥300 cells/μL.

2nd: Annualized rate of severe exacerbation events during the 52-week placebo-controlled treatment period based on the patients with baseline blood eosinophils ≥150 cells/μL.

3rd: Annualized rate of severe exacerbation events during the 52-week placebo-controlled treatment period based on the patients with Type 2 inflammatory phenotype (baseline blood eosinophils ≥150 cells/μL or baseline FeNO≥20 ppb).

### For EU and EU Reference Countries

1st: Annualized rate of severe exacerbation events during the 52-week placebo-controlled treatment period based on the patients with Type 2 inflammatory phenotype (baseline blood eosinophils ≥150 cells/μL or baseline FeNO≥20 ppb).

2nd: Annualized rate of severe exacerbation events during the 52-week placebo-controlled treatment period based on the patients with baseline blood eosinophils ≥150 cells/μL population.

3rd: Annualized rate of severe exacerbation events during the 52-week placebo-controlled treatment period based on the patients with baseline blood eosinophils ≥300 cells/μL population.

Multiplicity control for any secondary endpoints if considered is specified in the SAP. Otherwise, nominal p-values is provided.

### Handling of Missing Data

If patients withdraw from the study before week 52 with severe exacerbation events that may occur after study discontinuation, these patients are considered as patients with missing data on severe exacerbation. Number, reasons and timing of the missing data are summarized by treatment groups. In the primary analysis, all observed data are used regardless of treatment adherence or increase of asthma background medication. No imputation is conducted for the missing severe exacerbation information after a patient prematurely withdraws from the study up to week 52. In addition, sensitivity analyses based on pattern mixture model, placebo based pattern mixture model and tipping point analysis based on the same negative binomial model as being used in the primary analysis may be conducted to assess the robustness of the conclusion of the main model.

### Analysis of Other Secondary Endpoints

The change from baseline for continuous endpoints is analyzed using a mixed-effect model with repeated measures (MMRM) approach. The model includes change from baseline as response variables, and for treatment, age, weight (≤30 kg, >30 kg), region, baseline eosinophil level (<300 cells/μL, ≥300 cells/μL), baseline FeNO level (<20 ppb, ≥20 ppb), baseline ICS dose level (medium/high), visit, treatment-by-visit interaction, baseline value, and baseline-by-visit interaction as covariates; unless otherwise specified (details are documented in SAP). Sex, height, and ethnicity is also included as covariates in the models for spirometry parameters. An unstructured correlation matrix is used to model the within-patient errors. Parameters are estimated using restricted maximum likelihood method using the Newton-Raphson algorithm. Statistical inferences on treatment comparisons for the change from baseline at week 12 are derived from the mixed-effect model with Kenward and Roger degree of freedom adjustment approach. Treatment comparisons at other timepoints, 8, 12, 24, 36 and 52 week and other timepoints in between are also provided from the mixed-effect model for descriptive purpose. Data up to week 52 are included as response variables.

Time to first severe asthma exacerbation event and time to first LOAC is analyzed using a Cox regression model with time-to-event as the dependent variable, and treatment, age, weight (≤30 kg, >30 kg), region, baseline eosinophil level (<300 cells/μL, ≥300 cells/μL), baseline FeNO level (<20 ppb, ≥20 ppb), baseline ICS dose level (medium/high) and number of asthma events prior to the study as covariates. The estimated hazard ratio (DUPIXENT® versus placebo) along with its 95% confidence interval is presented. The Kaplan-Meier method is used to derive the proportion of patients with a severe asthma exacerbation event at weeks 12, 24, 36, and 52, specific to each treatment group.

The safety variables, including AEs, laboratory parameters, vital signs, ECG, and physical examinations are summarized using descriptive statistics. The analysis of safety variables is performed based on the safety population.

### Duration of Study Period

Total duration of study (per patient) is expected to be up to 68±1 weeks:
4 (±1) weeks for screening;
52 weeks of treatment;
12 weeks of post-treatment follow-up.

### Disease-Specific, Daily Efficacy Assessments

### Electronic Diary/PEF Meter

On a daily basis throughout the study, the patient uses an electronic diary/peak expiratory flow (PEF) meter to: measure morning and evening PEF; respond to the morning and evening asthma symptom scale questions; indicate the number of inhalations/day of salbutamol/albuterol or levosalbutamol/levalbuterol for symptom relief; record the number of inhalations/day of background product used; record the number of nocturnal awakenings due to asthma symptoms requiring the use of reliever medication; and record oral steroids use for exacerbation event.

At screening (visit 1), patients and parent(s)/caregiver(s)/legal guardian(s) are issued an electronic diary/PEF meter. Parent(s)/caregiver(s)/legal guardian(s) are instructed on the use of the device, and written instructions on the use of the electronic PEF meter is provided to the parent(s)/caregiver(s)/legal guardian(s).

In addition, the investigator instructs the parent(s)/caregiver(s)/legal guardian(s) on how to record the following variables in the electronic PEF meter: AM PEF performed within 15 minutes after arising (between 5:30 AM and 11:59 AM) prior to taking any albuterol/salbutamol or levalbuterol/levosalbutamol reliever medication); PM PEF performed in the evening (between 5:30 PM and 11:59 PM) prior to taking any albuterol/salbutamol or levalbuterol/levosalbutamol reliever medication); patient/parent(s)/caregiver(s)/legal guardian(s) should try to withhold albuterol/salbutamol or levalbuterol/levosalbutamol reliever medication for at least 6 hours before performing the PEF measurements; three PEF efforts is performed by the patient; all 3 values are recorded by the electronic PEF meter, and the highest value is used for evaluation.

Baseline AM PEF is the mean AM measurement recorded for the 7 days prior to the first dose of investigational product, and baseline PM PEF is the mean PM measurement recorded for the 7 days prior to the first dose of investigational product. Period stability limit is defined as the respective mean AM or PM PEF obtained over the last 7 days prior to day 1. There should be at least 4 days' measurement for setting up the stability limit, and the first dosing visit should be rescheduled until data for 4 days are available.

Baseline reliever use is the mean number of reliever use recorded for the 7 days prior to the first dose of investigational product. Period stability limit is defined as the respective mean AM or PM PEF obtained over the last 7 days prior to day 1. There should be at least 4 days' measurement for setting up the stability limit, and the first dosing visit should be rescheduled until data for 4 days are available for both measurements.

Information derived from the electronic PEF meter is evaluated by the investigator at study visits.

### Asthma Symptom Numerical Rating Scale (NRS) Score

Parent(s)/caregiver(s)/legal guardian(s) record overall symptom scores in an electronic diary/PEF meter twice a day prior to measuring PEF. The patient's overall asthma symptoms experienced during the waking hours are recorded in the evening (PM symptom score). Baseline symptom scores are the mean AM and mean PM scores recorded for the 7 days prior to randomization. The baseline AM/PM symptom score are computed following the same algorithm used for baseline AM/PM PEF. Scores range between 0-4 with 0 indicating more mild symptoms and 4 indicating more severe symptoms. There is no global score, just an AM score and a PM score. A minimal clinically important difference (MCID) of 0.35 is used.

### Morning Diary

0. No asthma symptoms, slept through the night.

1. Slept well, but some complaints in the morning. No nighttime awakenings.

2. Woke up once because of asthma (including early awakening).

3. Woke up several times because of asthma (including early awakening).

4. Bad night, awake most of the night because of asthma.

### Evening Diary

0. Very well, no asthma symptoms.

1. One episode of wheezing, cough, or breathlessness.

2. More than one episode of wheezing, cough, or breathlessness without interference with normal activities.

3. Wheezing, cough, or breathlessness most of the day, which interfered to some extent with normal activities.

4. Asthma very bad. Unable to carry out daily activities as usual.

### Use of Reliever Medicine

The number of salbutamol/albuterol or levosalbutamol/levalbuterol inhalations are recorded daily by the parent(s)/caregiver(s)/legal guardian(s) in an electronic diary/PEF meter. Each patient is reminded that salbutamol/albuterol or levosalbutamol/levalbuterol should be used only as needed for symptoms, not on a regular basis or prophylactically. The baseline number of salbutamol/albuterol or levosalbutamol/levalbuterol inhalations/day is based on the mean of the 7 days prior to randomization.

### Health Care Resource Utilization

The HCRU questionnaire (questions on use of reliever medication, specialist visit, hospitalization, emergency or urgent medical care facility visit, outcome, school day loss, etc.), as integrated part of the e-CRF is administered, and are also be used to assess HCRU in the event of any asthma exacerbation: severe asthma exacerbation event or evidence of LOAC.

### Patient Reported Outcomes, Including Health Related Quality of Life (Secondary Endpoints)

Patients are administered the following PRO questionnaires by their parent(s)/caregiver(s)/legal guardian(s) or with their help. The interviewer-administered versions are only for children: ACQ-IA, pediatric asthma quality of life questionnaire (PAQLQ[S]-IA) and are administered by an interviewer (clinic staff designated by investigator).

### Asthma Control Questionnaire-Interviewer Administered

The ACQ-IA was designed to measure both the adequacy of asthma control and change in asthma control, which occurs either spontaneously or as a result of treatment, and are used for children 6 years to <12 years old at screening.

### ACQ-7-IA (Asthma Control Questionnaire-Interviewer Administered, 7-Question Version)

The Asthma Control Questionnaire-Interviewer Administered, 7-question version (ACQ-7-IA) has seven questions, with the first five items of ACQ-7 (ACQ-5-IA score) addressing the most common asthma symptoms: 1) frequency in past week awoken by asthma during the night, 2) severity of asthma symptoms in the morning, 3) limitation of daily activities due to asthma, 4) shortness of breath due to asthma and 5) wheeze (It includes 2 questions on overall reliever medication use), 6) short-acting bronchodilator use, and—after spirometry assessment—current asthma status, 7) predicted bronchodilator use of FEV1 (pre-bronchodilator use, % and % predicted use).

Patients and/or parent(s)/caregiver(s)/legal guardian(s) are asked to recall how their asthma and/or their child's asthma, respectively, has been during the previous week and to respond to the symptom questions 1) to 6) on a 7-point scale (0=no impairment, 6=maximum impairment).

After spirometry assessment, patients and/or parent(s)/caregiver(s)/legal guardian(s) are asked to recall how their asthma and/or their child's asthma has been during the previous week and to respond to the symptom and bronchodilator use questions on a 7-point scale (0=no impairment, 6=maximum impairment). Clinic staff scores the % predicted FEV1 on a 7-point scale based on the pre-central reading spirometry result displayed immediately after the testing. Then, the questions are equally weighted and the global ACQ-7 score is the mean of the 7 questions and therefore between 0 (totally controlled) and 6 (severely uncontrolled).

Higher score indicates lower asthma control. Patients with a score below 1.0 reflect adequately controlled asthma and patients with scores above 1.0 reflect inadequately controlled asthma. On the 7-point scale of the ACQ-7, a change or difference in score of 0.5 is the smallest change that can be considered clinically important, corresponding to the MCID defined by the developer.

For statistical analysis, ACQ-7 global score is calculated by the sponsor using the BMS post central reading value of the % predicted FEV1 for the question 7 of the questionnaire.

Measurement properties such as reliability and ability to detect change have been documented in the literature.

### ACQ-5-IA (Asthma Control Questionnaire-Interviewer Administered, 5-Question Version)

The ACQ-5-IA are deduced from the responses to the first 5 questions of ACQ-7-IA and are used for children ≥6 years to <12 years old at screening. Higher score indicates lower asthma control. Patients with a score below 1.0 reflect adequately controlled asthma and patients with scores above 1.0 reflect inadequately controlled asthma. On the 7-point scale of the ACQ-5, a change or difference in score of 0.5 is the smallest change that can be considered clinically important, corresponding to the MCID defined by the developer.

Pediatric Asthma Quality of Life Questionnaire with Standardized Activities-Interviewer Administered

The PAQLQ(S)-IA was designed as an interviewer-administered PRO to measure the functional impairments that are most troublesome to children ≥7 years old at randomization visit 2, as a result of their asthma. The instrument is comprised of 23 items, each rated on a 7-point Likert scales from 1 to 7.

The PAQLQ(S)-IA has 3 domains. The domains and the number of items in each domain are as follows: symptoms (10 items); activity limitation (5 items); and emotional function (8 items). A global score is calculated ranging from 1 to 7 and a score by domain. Higher scores indicate better quality of life.

### Other Secondary Endpoints

### Systemic Drug Concentration, Anti-Drug Antibodies, and IgG Responses to Vaccination During Drug Treatment

The following are assessed: serum functional DUPIXENT® concentrations; ADA; and IgG responses to vaccination with any vaccination for tetanus, diphtheria, pertussis and/or seasonal trivalent/quadrivalent influenza vaccine during DUPIXENT® treatment (may be analyzed as exploratory endpoint if insufficient power).

### Systemic Drug Concentration and Anti-Drug Antibodies

### Sampling Time

Pre-dose blood samples are collected for determination of functional DUPIXENT® concentration in serum and anti-DUPIXENT® antibodies (including neutralizing antibodies) on days designated in the study flow chart. The date of collection is recorded in the patient e-CRF. The date and time also are collected on the central laboratory requisition form and entered into the database through data transfers from the central laboratory.

If an SAE or AESI occurs in a patient, blood samples are collected for determination of functional DUPIXENT® concentration, and anti-DUPIXENT® antibody assessment at or near the onset and completion of the occurrence of the event, if possible. The exact date and time of sample collection must be recorded and entered into the database by the central laboratory. An unscheduled systemic drug concentration page in the e-CRF must be completed as well.

Further follow-up of individual patients is considered based on the overall assessment of antibody titers and clinical presentation.

### Humoral Immune Response to Vaccines

Humoral immune responses to standard vaccines (in this study: any vaccination for tetanus, diphtheria, pertussis and/or seasonal trivalent/quadrivalent influenza vaccine) occurring during DUPIXENT® treatment are evaluated for those patients eligible for these vaccinations.

At screening, parent(s)/caregiver(s)/legal guardian(s) are asked to provide information on their child's vaccination record and schedule, and assess whether immunizing their children with any vaccination for tetanus, diphtheria, pertussis and/or seasonal trivalent/quadrivalent influenza (as per local medical practice) result in vaccination during the study.

Any patient who receives planned vaccination for tetanus, diphtheria, pertussis and/or seasonal trivalent/quadrivalent influenza during the study, is scheduled to receive the respective vaccination(s) and to have blood samples for antibody titers drawn before and after the respective vaccination(s).

Scheduled blood sample collection for pre- and post-vaccine antibody titers, for both vaccinations (i.e., any tetanus, diphtheria and pertussis and/or seasonal trivalent/quadrivalent influenza) should be drawn within 8 weeks prior to vaccination and at 3-4 weeks (up to 6 weeks) after the respective vaccination(s). However, all blood titer samples must be drawn between week 6 and week 50 (i.e., visit 5 and visit 27, respectively).

Depending on patient's vaccination schedule during the course of this study, every effort should be made to draw pre-vaccination titers at either weeks 6, 12, or 24 (V5, V8, V14) of the randomized treatment period, and to draw post-vaccination titers at either Weeks 12, 24, 36 or 50 (V8, V14, V20, V27) of the randomized treatment period.

For patient(s) requiring urgent/emergency vaccination with any seasonal trivalent/quadrivalent influenza and/or any tetanus, diphtheria and pertussis vaccine (e.g., flu season approaching, animal bite, emergency room standard procedures, etc.) between week 6 and week 44 (i.e., visit 5 and visit 24, respectively), the actual vaccination(s) may be given by physicians or qualified caregivers outside the study clinic. However, every effort should be made to obtain blood samples for pre- and post-vaccine antibody titers at scheduled draws as described above. Should vaccination be unable to be planned in accordance with other study blood draws (e.g., tetanus vaccination for accidental puncture wounds, etc.) as outlined above, at the discretion of the Investigator and with agreement of patient parents or caregiver, additional blood draws may be performed to obtain pre-vaccination and post-vaccination titers.

### Biomarker Endpoints

Change from baseline in fractional exhaled nitric oxide (FeNO) at week 12 is analyzed. Fractional exhaled nitric oxide (FeNO) is analyzed using a NIOX instrument (Aerocrine AB, Solna, Sweden), or similar analyzer using a flow rate of 50 mL/s, and reported in parts per billion (ppb). This assessment is conducted prior to spirometry and following a fast of at least 1 hour.

### Exploratory Endpoints

Exploratory endpoints are: change from baseline in blood biomarkers (TARC and serum total IgE); genetic analysis of genomic DNA to assess the association of genetic variation with asthma and response to DUPIXENT® treatment; the proportion of patients requiring a permanent step up in background controller medication after 2 or more severe asthma exacerbation events; the effect of DUPIXENT® on additional PROs: (Pediatric Asthma Caregiver's Quality of Life Questionnaire (PACQLQ) score, for caregivers of children ≥7 years old at Randomization Visit 2,

Pediatric Rhinoconjunctivitis Quality of Life Questionnaire-Interviewer Administered (PRQLQ-IA) score, in children 6 to <12 years old with history of allergic rhinitis), EuroQol 5-dimensions questionnaire (EQ-5D-Y) for children); change from Baseline in antigen-specific IgE and antigen-specific IgG4, and ratio of IgE:IgG4; and slope of % predicted FEV1.

### Pharmacodynamics and Phenotyping

Asthma is a heterogeneous disease comprised of multiple phenotypes and endotypes. To assure optimization of treatment in children, a set of biomarkers related to Type 2 inflammation are assessed at baseline and after treatment for their association with therapeutic response. Biomarkers to assess include the levels of serum total IgE (a product of immunoglobulin class switching driven by IL-4), antigen-specific IgEs, serum TARC (CCL17; a ligand of CCR4 receptors that attracts Th2 cells), and FeNO (a marker of airway inflammation) baseline values, including blood eosinophil counts from hematology assays that were used to phenotype patients.

In addition, a possible switching in antigen-specific IgE toward the corresponding antigen-specific IgG4 is assessed in this study to explore the possibility that DUPIXENT® may in part attenuate allergic sensitization.

Patient(s)/parent(s)/caregiver(s)/legal guardian(s), Investigators, and site personnel are blinded and have no access to any assay results for total IgE, antigen-specific IgEs, antigen-specific IgG4, or TARC, while the study is ongoing, as the related efficacy data are not essential for patient care and have the potential for un-blinding the study treatments.

### Serum Biomarkers

Total IgE is measured with a quantitative method (e.g., ImmunoCAP) approved for diagnostic testing.

Antigen-specific IgE and antigen-specific IgG4 is detected using panels of antigens appropriate to the location of the clinical site (quantitative ImmunoCAP test; Phadia).

TARC is assayed with a validated immunoassay.

### Stored DNA Samples for Pharmacogenetics

DNA samples can be used to determine a possible relationship between genetic variation and response to treatment with DUPIXENT® or possible adverse reactions to DUPIXENT® and to study the genetics of asthma. The DNA can be subjected to a genome-wide association study by microarray analysis and/or to whole exome sequencing or whole genome analysis in order to thoroughly explore genetic associations with disease progression or treatment response.

The DNA sample that is extracted, are assigned a second number, a Genetic ID (de-identification code) different from the Subject ID. This “double coding” of these samples is performed to separate a subject's medical information and DNA data. The clinical study data (coded by Subject ID) are stored in the clinical data management system (CDMS), which is a distinct database in a separate environment from the database containing the pharmacogenetic data (coded by Genetic ID). The key linking Subject ID and Genetic ID are maintained by a third party under appropriate access control. The matching of clinical data and pharmacogenetic data, for the purpose of data analysis, is possible only by using this key, which are under strict access control. All data are reported only in coded form in order to maintain confidentiality.

The aliquots of DNA sent to the bioanalytical laboratories for specific genetic testing will be destroyed after completion of that specific analysis and issuance of the related analytical data.

Patients Requiring a Permanent Step Up in Background Controller Medication after 2 or More Severe Asthma Exacerbation Events

For this study, severe asthma exacerbation events should be managed by the investigators based on their medical judgment and applicable national/international asthma management guidelines, and as outlined in this protocol: For patient(s), who experience 2 or more severe asthma exacerbation events anytime during the treatment period, a permanent change (step up in medium- to high-dose ICS or addition of second controller for patients on high-dose ICS monotherapy) on their stable-dose background controller medication may occur, as indicated and according to the respective Investigator's medical judgment and direction. The proportion of all patients with any of these treatment adjustments are compared by treatment arm.

### Other Patient Reported Outcomes Including Health Related Quality of Life (Exploratory Endpoints)

### Pediatric Asthma Caregiver's Quality of Life Questionnaire

The PACQLQ was designed as a 13-item questionnaire for the parent(s)/caregiver(s)/legal guardian(s) of children ≥7 years old and <12 years of age (at randomization visit 2), in order to capture the impact of the child's asthma on their quality of life and which aspects were most troublesome to the parent(s)/caregiver(s)/legal guardian(s) during the time prior to this assessment.

A global score is calculated ranging from 1 to 7 and a score by domain. Higher scores indicate better quality of life.

Pediatric Rhinoconjunctivitis Quality of Life Questionnaire-Interviewer Administered in Patients with Comorbid Allergic Rhinitis

PRQLQ-IA is an interviewer-administered questionnaire developed to measure HRQoL signs and symptoms that are most problematic in children ≥6 years to <12 years old, as a result of perennial or seasonal allergic rhinitis. The 23-item PRQLQ-IA responses are based on 7-point Likert scale with responses ranging from 0 (not troubled) to 6 (extremely troubled). Higher scores indicated more health-related quality of life impairment (lower scores better). The instrument takes approximately 7 minutes to complete. The minimally important difference (MID) of 0.5 has been established as the minimal important difference indicative of a clinically meaningful change.

### Euro QoL (EQ-5D-Y)—for Children

The EQ-5D-Y is completed by children (relates to the quality of life to the child). Those who can read are encouraged to fill the questionnaire by themselves. Those who cannot read, fill it with the help of their adult caregiver (parent/caregiver).

The EQ-5D-Y consists of 2 pages, the EQ-5D-Y descriptive system and the EQ visual analogue scale (VAS; see Appendix J). The descriptive system assesses 5 dimensions but using a child-friendly wording (mobility, looking after myself, doing usual activities, having pain or discomfort, feeling worried, sad or unhappy). Each dimension has 3 levels: no problems, some problems, a lot of problems. The respondent is asked to indicate his/her health state by ticking (or placing a cross) in the box against the most appropriate statement in each of the 5 dimensions. The EQ VAS records the respondent's self-rated health on a vertical, visual analogue scale where the endpoints are labelled ‘The best health you can imagine’ and ‘The worst health you can imagine.’ This information can be used as a quantitative measure of health outcome as judged by the individual respondents. Also, previously published studies by EuroQol Group members showed preliminary evidence of the instrument's feasibility, reliability and validity.

### Efficacy Populations

The full intent-to-treat (ITT) population is defined as all randomized patients.

Type 2 inflammatory phenotype population is defined as the randomized patients with baseline blood eosinophils ≥150 cells/μL or baseline FeNO≥20 ppb.

Baseline blood eosinophils ≥300 cells/4, population is defined as the randomized patients with baseline blood eosinophils ≥300 cells/μL. Baseline blood eosinophils ≥150 cells/4, population is defined as the randomized patients with baseline blood eosinophils ≥150 cells/μL.

All efficacy endpoints are analyzed based on both the Type 2 inflammatory phenotype population and the population with baseline blood eosinophils ≥300 cells/μL.

The sponsor implements two testing hierarchies based on the two different indication labels for the US and US reference countries and the EU and EU reference countries. Accordingly, for the US and US reference countries, the testing hierarchy starts with baseline blood eosinophils ≥300 cells/4, population. For EU and EU reference countries, the testing hierarchy starts with Type 2 inflammatory phenotype population (patients with baseline blood eosinophils ≥150 cells/μL or baseline FeNO≥20 ppb).

The efficacy analyses are conducted according to the treatment to which they are randomized. Selected efficacy endpoints are also be analyzed based on the full ITT population.

### Safety Population

The analysis population for the safety endpoints are safety population defined as all patients exposed to study medication, regardless of the amount of treatment administered and regardless of whether they are randomized. The safety analyses are conducted according to the treatment patients actually received.

Treatment emergent period for safety population is defined as the time between the first administration of study medication to the end of the Post-treatment Period or till the rollover to the 1-year long-term extension study. In addition, randomized patients for whom it is unclear whether they took the study medication are included in the safety population as randomized.

### Systemic Drug Concentration Population

The systemic drug concentration population consists of all patients in the safety population with at least one evaluable functional DUPIXENT® concentration result. Patients are analyzed according to the treatment actually received.

### Anti-Drug Antibody (ADA) Population

The ADA population consists of all patients in the safety population with at least one qualified ADA result in the ADA assay following the first dose of the study medication. Patients are analyzed according to the treatment actually received.

### Analyses of Efficacy Endpoints

Annualized rate of severe asthma exacerbation events during the 52 weeks is the primary efficacy endpoint of this study. Key secondary endpoints include change from baseline in pre-bronchodilator % predicted FEV1 at week 12. Other secondary endpoints include change from baseline in pre-bronchodilator % predicted FEV1 at Weeks 2, 4, 8, 24, 36 and 52 and other time points in between; time to first severe exacerbation event; time to first LOAC event; change from baseline in other lung function measurements (absolute and relative FEV1, AM/PM PEF, FVC, FEF_{25-75%}, post-bronchodilator % predicted FEV1 at weeks 2, 4, 8, 12, 24, 36, 52, and other time-points in between; change from baseline at weeks 2, 4, 8, 12, 24, 36, 52, and other time-points for morning/evening asthma symptom score and nocturnal awakenings (electronic diary), use of reliever medication, and ACQ score. Change from baseline for PAQLQ(S)-IA score, PACQLQ score, PRQLQ-IA score (in those with history of allergic rhinitis) and health care resource utilization, are assessed at weeks 12, 24, 36, 52, 64; and percentage of patients requiring increase in dose or addition of background medication.

In addition to the primary approach to analyze change from baseline in ACQ-IA and PAQLQ(S)-IA total score, supportive responder analyses is also be performed for these endpoints at week 12, 24, 36, 52 and 64.

### Analysis of Primary Efficacy Endpoint(s)

The estimand of the DUPIXENT® treatment effect compares the annualized rate of severe exacerbation for the patients randomized to the DUPIXENT® and placebo arms, regardless of what treatment patients actually received. It assesses the benefits of the treatment policy or strategy relative to placebo. In this primary approach, off-treatment measurements of patients who prematurely discontinue treatment are included for the analysis. Patients who permanently discontinue the study medication are asked and encouraged to return to the clinic for all remaining study visits. If a patient stays in study until the end of 52-week treatment period, all severe exacerbation events that happen up to week 52 are included in the primary analysis, regardless if the patient is on-treatment or not. If a patient withdraws from study prior to the end of 52-week treatment period, all observed severe exacerbation events up to the last contact date are included in the analysis, and the observation duration is defined as from randomization to the last contact date. No imputation is performed for the unobserved events that may happen after study discontinuation and up to week 52.

The annualized rate of severe asthma exacerbation events is analyzed using a negative binomial regression model to confirm the effectiveness of DUPIXENT®. The analysis for the annualized severe exacerbation rate is performed in the Type 2 inflammatory phenotype, baseline blood eosinophils ≥300 cells/μL, baseline blood eosinophils ≥150 cells/μL, baseline FeNO≥20 ppb and full ITT populations using appropriate multiplicity control. When performing the primary endpoint analysis in the Type 2 inflammatory phenotype, baseline blood eosinophils ≥150 cells/μL or the full ITT populations, the model includes the total number of events of each patient occurring during the 52 weeks as the response variable, with the treatment group, age, weight (≤30 kg, >30 kg), region, baseline eosinophil level (<300 cells/≥300 cells/μL), baseline FeNO level (<20 ppb, ≥20 ppb), baseline ICS dose level (medium/high), and number of severe asthma exacerbation events prior to the study as covariates. When performing the primary endpoint analysis in the baseline blood eosinophils ≥300 cells/μL population, the baseline eosinophil level is removed from the model covariates. When performing the primary endpoint analysis in the baseline FeNO≥20 ppb population, the baseline FeNO level is removed from the model covariates. Severe asthma exacerbation event prior to the study is defined as treatment with a systemic steroid (oral or parenteral) for worsening asthma at least once or hospitalization or emergency medical care visit for worsening asthma (as defined in this protocol). Log transformed observation duration is the offset variable.

### Sensitivity Analysis

A supportive analysis to assess the treatment effect of DUPIXENT® if patients adhere to the treatment and background asthma medication as directed is also provided. In this approach, the severe exacerbation events reported after the premature treatment discontinuation are excluded from the analysis. Any measurement obtained after the first permanent stepping-up of background asthma medication is excluded from the analysis. The supportive analysis is performed in the Type 2 inflammatory phenotype and baseline blood eosinophils ≥300 cells/μL populations and use a negative binomial model with the same set of covariates as specified for the primary analysis in the two populations. This model includes severe exacerbation events occurring during the treatment epoch before any permanent stepping-up of background asthma medication as the response variable, and the log transformed duration of the treatment or from randomization to first permanent stepping-up of background asthma medication, whichever is shorter, is the offset variable.

If patients withdraw from the study before week 52 with severe exacerbation events that may occur after study discontinuation are not observed, these patients are considered as patients with missing data on severe exacerbation. Number, reasons and timing of the missing data are summarized by treatment groups. In the primary analysis, all observed data is used regardless of treatment adherence or increase of asthma background medication. No imputation is conducted for the missing severe exacerbation information after a patient prematurely withdraws from the study up to week 52. In addition, sensitivity analyses based on pattern mixture model, placebo based pattern mixture model and tipping point analysis based on the same negative binomial model as being used in the primary analysis can be conducted to assess the robustness of the conclusion of the main model.

### Subgroup Analysis

Subgroup analyses are performed for the primary endpoints, as appropriate, using the same methods by age group, gender, region, race, baseline ICS (medium/high) dose levels, baseline eosinophil level, baseline FeNO level, background controller medication type at randomization, baseline % predicted FEV1, ACQ-7, baseline body weight, atopic medical condition, age of onset of asthma, and number of severe asthma exacerbation events within 1 year prior to the study.

The subgroup analyses (except for the baseline eosinophil levels and baseline FeNO levels) are conducted for both the Type 2 inflammatory phenotype population and baseline blood eosinophils ≥300 cells/μL population, and the subgroup analyses for the baseline blood eosinophil level and baseline FeNO level is performed in the full ITT population.

### Analyses of Secondary Efficacy Endpoints

Analysis of Change from Baseline in Pre-Bronchodilator % Predicted FEV1

The key secondary endpoint, change from baseline in pre-bronchodilator % predicted FEV1 at week 12, is analyzed using a mixed-effect model with repeated measures (MMRM) approach. The analysis for the key secondary endpoint is performed in the Type 2 inflammatory phenotype, baseline blood eosinophils ≥300 cells/μL, baseline blood eosinophils ≥150 cells/μL, baseline FeNO≥20 ppb, and full ITT populations. When performing the key secondary endpoint analysis in the Type 2 inflammatory phenotype, baseline blood eosinophils ≥150 cells/μL, or the full ITT populations, the model include change from baseline as response variables, and for treatment, age, weight (≤30 kg, >30 kg), region, baseline eosinophil level (<300 cells/μL, ≥300 cells/μL), baseline FeNO level (<20 ppb, ≥20 ppb), baseline ICS dose level (medium/high), visit, treatment by-visit interaction, baseline value, and baseline-by-visit interaction as covariates. When performing the analysis in the baseline blood eosinophils ≥300 cells/μL population, the baseline eosinophil level is removed from the model covariates. When performing the analysis in the baseline FeNO≥20 ppb population, the baseline FeNO level is removed from the model covariates. Sex, height and ethnicity are also included as covariates in the models for spirometry parameters. An unstructured correlation matrix is used to model the within-patient errors. Parameters are estimated using restricted maximum likelihood method using the Newton-Raphson algorithm. Statistical inferences on treatment comparisons for the change from baseline at week 12 is derived from the mixed-effect model with Kenward and Roger degree of freedom adjustment approach. Treatment comparisons at other timepoints, 8, 12, 24, 36 and 52 week and other timepoints in between are also provided from the mixed-effect model for descriptive purpose. Data up to week 52 are included as response variables.

### Analysis of Time-to-Event Variables

Time to first severe asthma exacerbation event (and time to first LOAC) are analyzed using a Cox regression model with time-to-event as the dependent variable, and treatment, age, weight (≤30 kg, >30 kg), region, baseline eosinophil level (<300 cells/μL, ≥300 cells/μL), baseline FeNO level (<20 ppb, ≥20 ppb), baseline ICS dose level (medium/high) and number of severe asthma events prior to the study as covariates. The estimated hazard ratio (DUPIXENT® versus placebo) along with its 95% confidence interval is presented. The Kaplan-Meier method is used to derive the proportion of patients with a severe asthma exacerbation event at weeks 12, 24, 36, and 52, specific to each treatment group.

Analysis of Change from Baseline for Other Continuous Variables

The change from baseline for other continuous endpoints is analyzed using MMRM in the same fashion as for the endpoint of pre-bronchodilator % predicted FEV1. The covariates included are treatment, age, weight (≤30 kg, >30 kg), region, baseline eosinophil (<300 cells/μL, ≥300 cells/μL), baseline FeNO level (<20 ppb, ≥20 ppb), baseline ICS dose level (medium/high), visit, treatment-by-visit interaction, corresponding baseline value and baseline-by-visit interaction. Sex and height are included as covariates in the models, if the endpoint belongs to spirometry parameters. Descriptive statistics including number of patients, mean, standard error and LS means are provided for each timepoint. In addition, differences in LS means, the corresponding 95% CI and the p-value are derived from the MMRM model for comparison of DUPIXENT® against placebo at each timepoint.

Analysis of Change from Baseline for Other Categorical Variables

Percentage of patients requiring increase in dose or addition of background medication are analyzed as a categorical variable. Descriptive statistics by treatment group are provided including the number and the percentage of patients in each category. Time to the first time requiring increase in dose or addition of background medication can also be provided by the Kaplan-Meier method if there are a sufficient number of patients requiring increase in dose or addition of background medication.

### Sensitivity Analyses

Sensitivity analyses are only conducted for the key secondary endpoint of change from baseline in pre-bronchodilator % predicted FEV1 at week 12. A supportive analysis is provided by applying the same model for the primary analysis with only on-treatment measurements obtained before any permanent stepping-up of the asthma background medication.

A sensitivity analysis based on LOCF is also provided to assess the robustness of the conclusion from the primary analysis on change from baseline in pre-bronchodilator % predicted FEV1 at week 12 against missing data.

### Subgroup Analysis

To assess the consistency in treatment effects across the subgroup levels, subgroup analyses used in the primary efficacy endpoint are also performed for the key secondary efficacy endpoint of change from baseline in pre-bronchodilator % predicted FEV1 at week 12.

The sensitivity analysis and subgroup analysis (except for the baseline eosinophil levels and baseline FeNO levels) for the key secondary endpoint of change from baseline in pre-bronchodilator % predicted FEV1 at week 12 are conducted in the Type 2 inflammatory phenotype and baseline blood eosinophils ≥300 cells/μL population, and the subgroup analyses for the baseline blood eosinophil level and baseline FeNO level are performed in the full ITT population.

### Multiplicity Considerations

The hypothesis testing on the primary endpoint of annualized severe exacerbation rate is controlled with a two-sided type I error of 0.05 by incorporating a sequential testing procedure as below:

### For US and US Reference Countries:

1st: Annualized rate of severe exacerbation events during the 52-week placebo-controlled treatment period based on the patients with baseline blood eosinophils ≥300 cells/μL.

2nd: Annualized rate of severe exacerbation events during the 52-week placebo-controlled treatment period based on the patients with baseline blood eosinophils ≥150 cells/μL.

3rd: Annualized rate of severe exacerbation events during the 52-week placebo-controlled treatment period based on the patients with Type 2 inflammatory phenotype (baseline blood eosinophils ≥150 cells/μL or baseline FeNO≥20 ppb).

### For EU and EU Reference Countries:

1st: Annualized rate of severe exacerbation events during the 52-week placebo-controlled treatment period based on the patients with Type 2 inflammatory phenotype (baseline blood eosinophils ≥150 cells/μL or baseline FeNO≥20 ppb).

2nd: Annualized rate of severe exacerbation events during the 52-week placebo-controlled treatment period based on the patients with baseline blood eosinophils ≥150 cells/μL.

3rd: Annualized rate of severe exacerbation events during the 52-week placebo-controlled treatment period based on the patients with baseline blood eosinophils ≥300 cells/μL.

Multiplicity control for any secondary endpoints, if considered, is specified in the SAP. Otherwise, nominal p-values are provided.

### Example II

### Liberty Asthma VOYAGE Ph 3 Trial Results

The phase 3, randomized, double-blind, placebo-controlled Liberty Asthma VOYAGE trial evaluated the efficacy and safety of DUPIXENT® in addition to standard-of-care maintenance therapy of medium-dose inhaled corticosteroid (ICS) with a second controller medication or high-dose ICS with or without a second controller medication. The trial enrolled 408 children aged 6 to <12 years old with uncontrolled moderate-to-severe asthma. Primary analyses were based on 259 patients with baseline ≥300 eosinophils/μl or 350 patients with markers of Type 2 inflammation (baseline ≥150 eosinophils/μl or fractional exhaled nitric oxide (FeNO)≥20 ppb). These patient groups align with the patient populations as defined in the U.S. asthma indication and EU asthma indication, respectively, in patients 12 years of age and older. There was no minimum biomarker requirement for enrollment.

During the 52-week treatment period, patients received subcutaneous injections of DUPIXENT® 100 mg or 200 mg every two weeks, based on weight (100 mg for ≤30 kg, 200 mg for >30 kg), or placebo every two weeks.

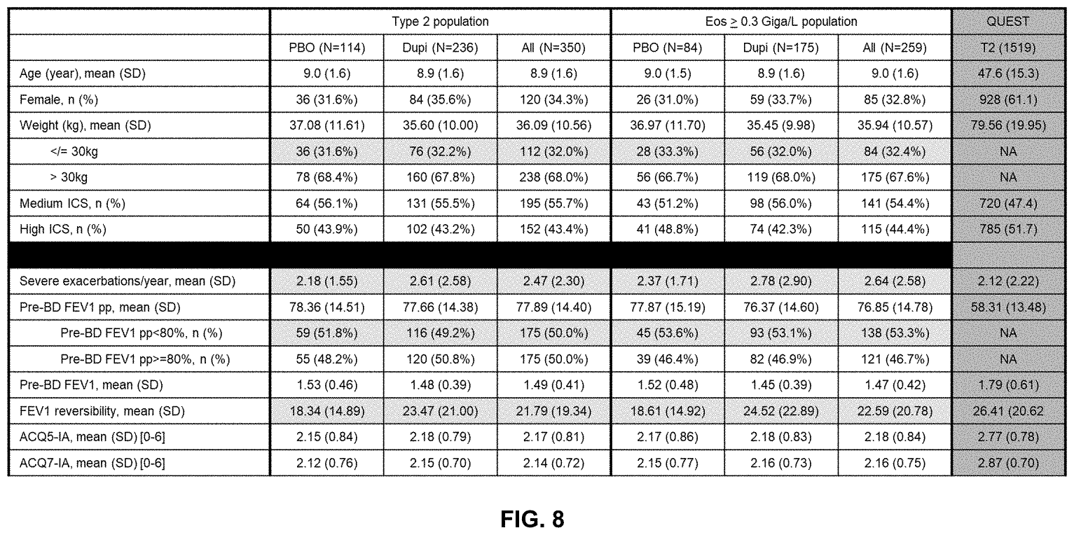

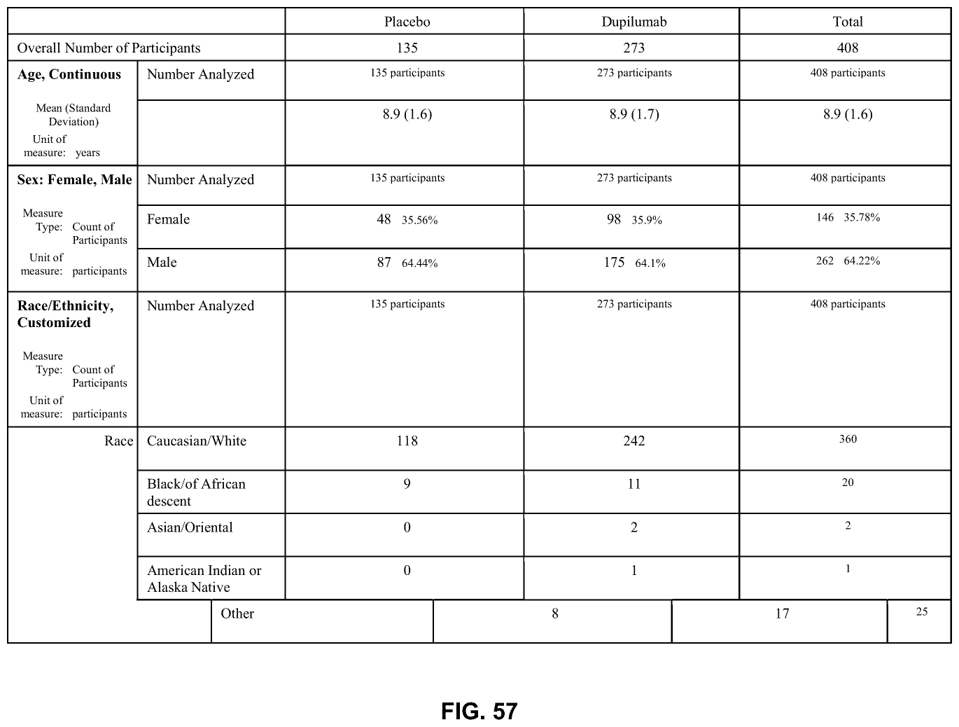

Baseline demographics and disease characteristics of the ITT population are summarized in FIG. 8 and FIG. 57. Approximately ⅔ of the ITT population was male, consistent with pediatric asthma epidemiology. Approximately ⅔ of the ITT population weighed more than 30 kg. The VOYAGE ITT population had higher exacerbations and mean FEV1pp than the ITT population of the QUEST study. The VOYAGE ITT population had a higher bronchodilator responsiveness in DUPIXENT® vs. placebo than the ITT population of the QUEST study.

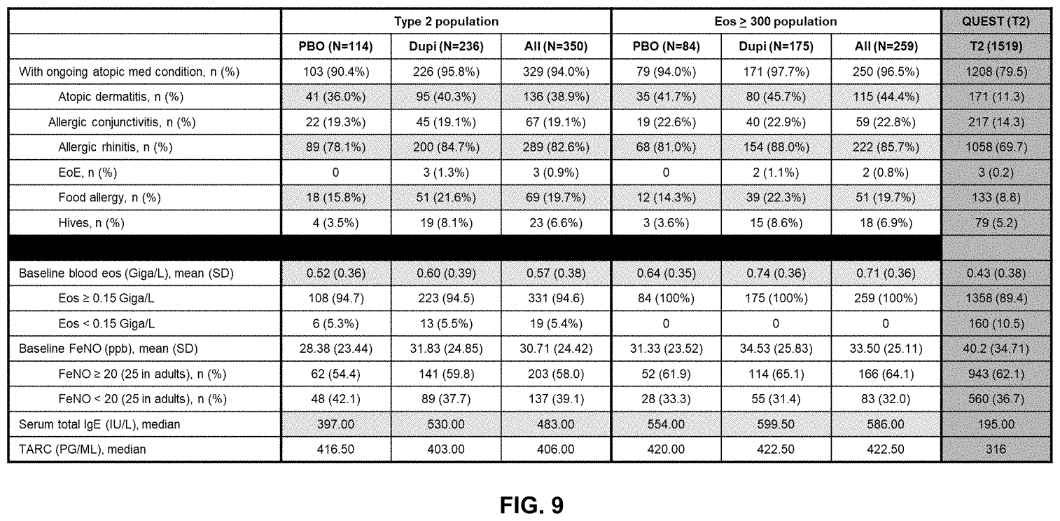

Concurrent atopic conditions and baseline biomarkers of the ITT population are summarized in FIG. 9. Approximately 95% of the ITT population had comorbid Type 2 conditions. The VOYAGE ITT population had higher levels of baseline Type 2 biomarkers than the ITT population of the QUEST study.

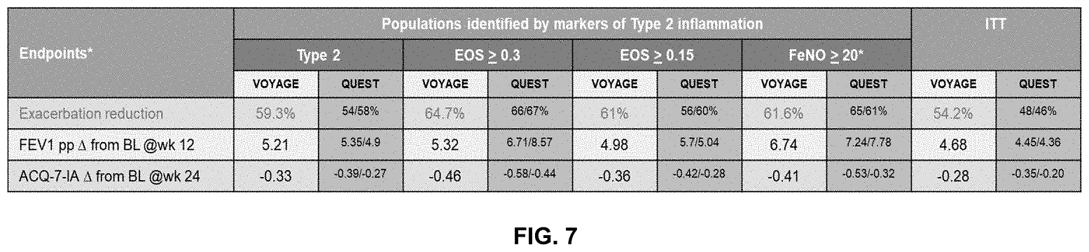

Data further indicated DUPIXENT® has potential to be best-in-class treatment option. DUPIXENT® (dupilumab) significantly reduced severe asthma attacks and showed rapid and sustained improvements in lung function in a pivotal trial in children aged 6-11 with moderate-to-severe asthma. DUPIXENT® significantly reduced severe asthma attacks by 59-65% over one year compared to placebo in children with asthma with Type 2 inflammation who had an eosinophilic phenotype or elevated fractional exhaled nitric oxide (FeNO) (FIG. 7). Unprecedented significant improvement in lung function was observed within two weeks and sustained for up to 52 weeks.

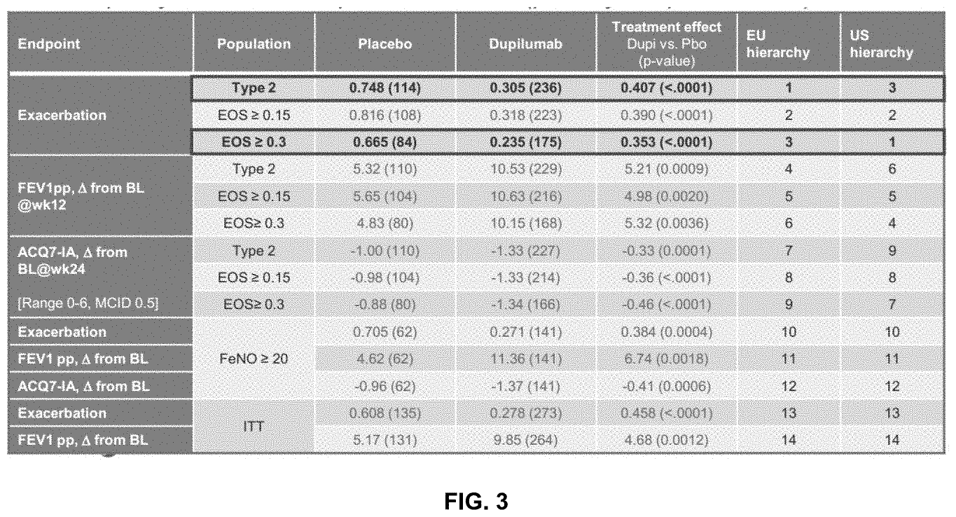

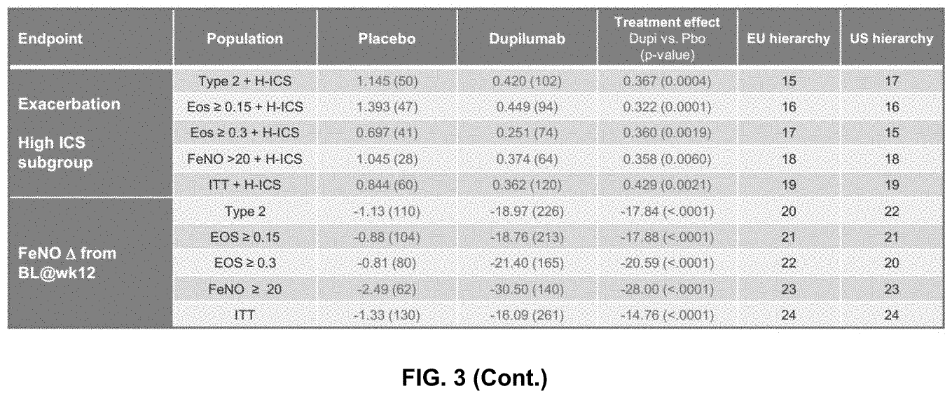

DUPIXENT® (dupilumab) met all endpoints in children aged 6 to 11 years with uncontrolled moderate-to-severe asthma with Type 2 inflammation who had an eosinophilic phenotype or elevated FeNO (FIG. 3). In this broad Type 2 asthma patient population, DUPIXENT®, added to standard-of-care therapy, significantly reduced asthma attacks (exacerbations) and improved lung function, as early as two weeks after the first dose, compared to standard-of-care therapy alone.

### Primary Analyses

The primary analyses were pre-specified to be performed separately in asthma patients with either Type 2 inflammation defined by FeNO≥20 ppb or blood eosinophils (EOS)≥150 cells/μl, or in asthma patients with EOS≥300 cells/μl. More than 90% of children in the trial had at least one concurrent Type 2 inflammatory condition, such as atopic dermatitis or allergic rhinitis.

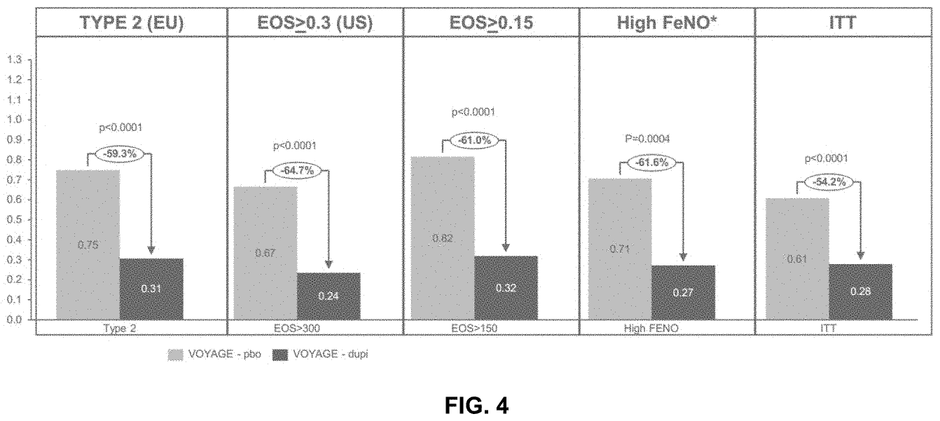

The primary endpoint assessed the annualized rate of severe asthma attacks in two primary populations: patients with markers of Type 2 inflammation (FeNO≥20 ppb or EOS≥150 cells/μl) and patients with EOS≥300 cells/μl. Results showed those treated with DUPIXENT® in addition to standard-of-care therapy experienced: 59% (p<0.0001) and 65% (p<0.0001) average reduction in the rate of severe asthma attacks over one year compared to placebo, respectively (FIG. 4) (0.31 and 0.24 for DUPIXENT® vs. 0.75 and 0.67 for placebo, respectively). For subjects with EOS≥150 cells/μl, high FeNO and the intent-to-treat population, results showed those treated with DUPIXENT® in addition to standard-of-care therapy experienced: 61% (p<0.0001), 62% (p<0.0004) and 54% (p<0.0001), respectively, average reduction in the rate of severe asthma attacks over one year compared to placebo, respectively (FIG. 4).

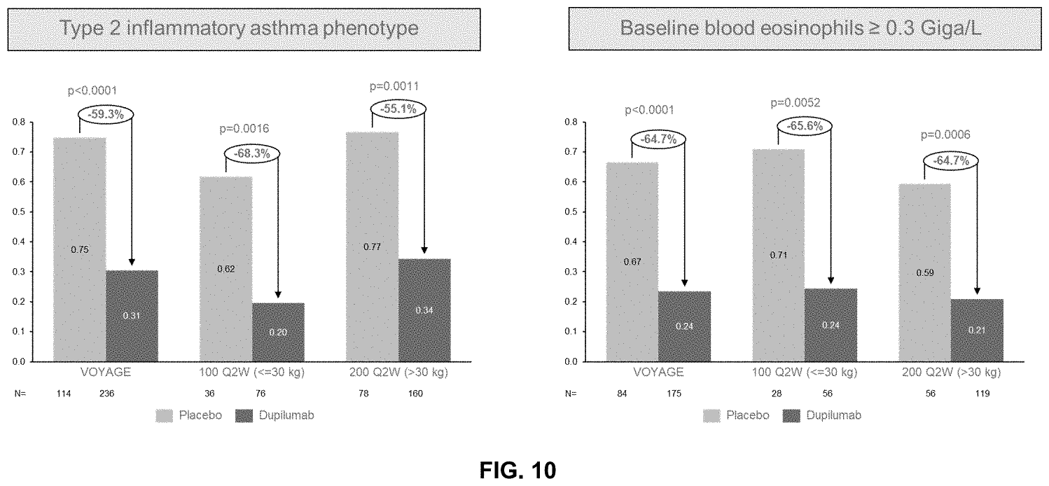

For subjects stratified by weight, there was significant improvement in both weight classes (≤30 kg or >30 kg) for the Type 2 inflammatory phenotype subpopulation and the baseline blood eosinophils ≥0.3 Giga/L subpopulation (FIG. 10).

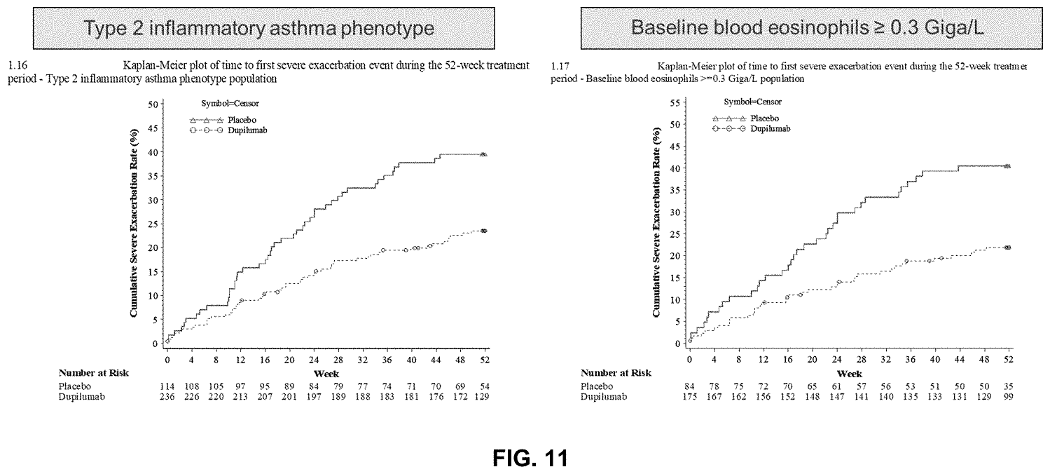

The time to first severe exacerbation for the Type 2 inflammatory phenotype subpopulation and the baseline blood eosinophils ≥0.3 Giga/L subpopulation is shown in FIG. 11. A clear decrease in exacerbation rates was observed within the first four weeks for both subpopulations.

Time to first loss of asthma control (LOAC) event using Kaplan-Meier estimates determined during the 52-week treatment period in a type 2 inflammatory asthma phenotype population was 140.0 (103.00 to 217.00) for DUPIXENT® (236 participants) vs. 63.5 (42.00 to 84.00) for placebo (114 participants). (Median (95% CI), unit of measure=days.) Time to LOAC event using Kaplan-Meier estimates determined during the 52-week treatment period in a baseline blood eosinophil ≥300 cells/microliter population was 135.0 (82.00 to 219.00) for DUPIXENT® (175 participants) vs. 47.5 (38.00 to 84.00) for placebo (84 participants). (Median (95% CI), unit of measure=days.)

A LOAC event was defined as deterioration of asthma during 52-week treatment period resulted in any of the following: >=6 additional reliever puffs of salbutamol/albuterol or levosalbutamol/levalbuterol in 24-hour period (compared to baseline) on 2 consecutive days; increase in ICS dose >=4 times than dose at visit 2 (week 0); a decrease in ante meridiem (AM)/post meridiem (PM) peak flow of 30% or more on 2 consecutive days of treatment, based on defined stability limit (defined as respective mean AM/PM peak expiratory flow obtained over last 7 days prior to randomization (day 1); severe exacerbation event. Time to first LOAC event was date of first severe event—first dose date+1. Kaplan-Meier method was used for analysis.

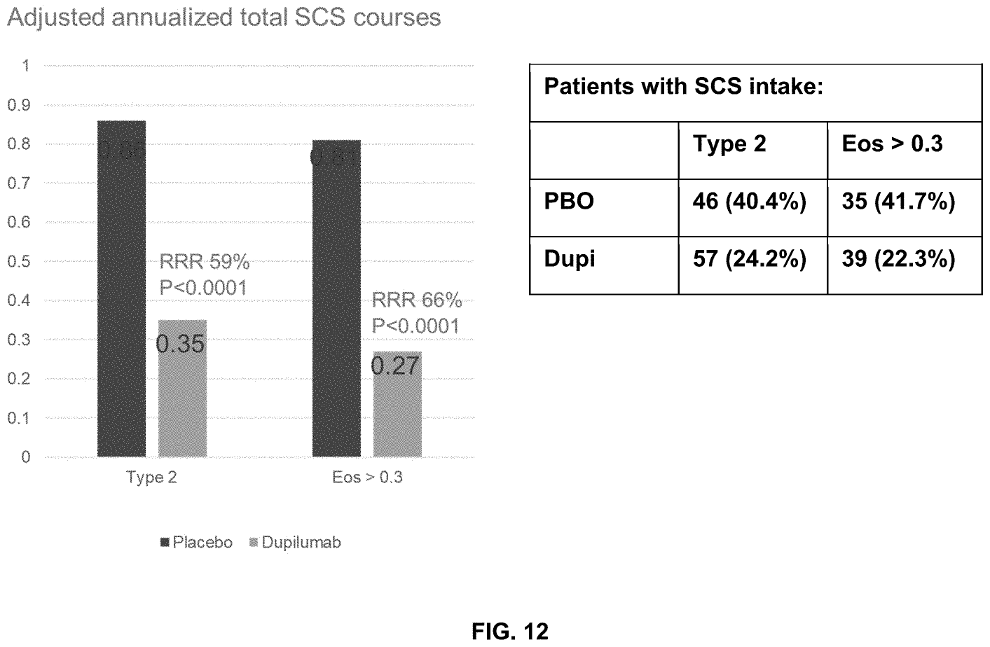

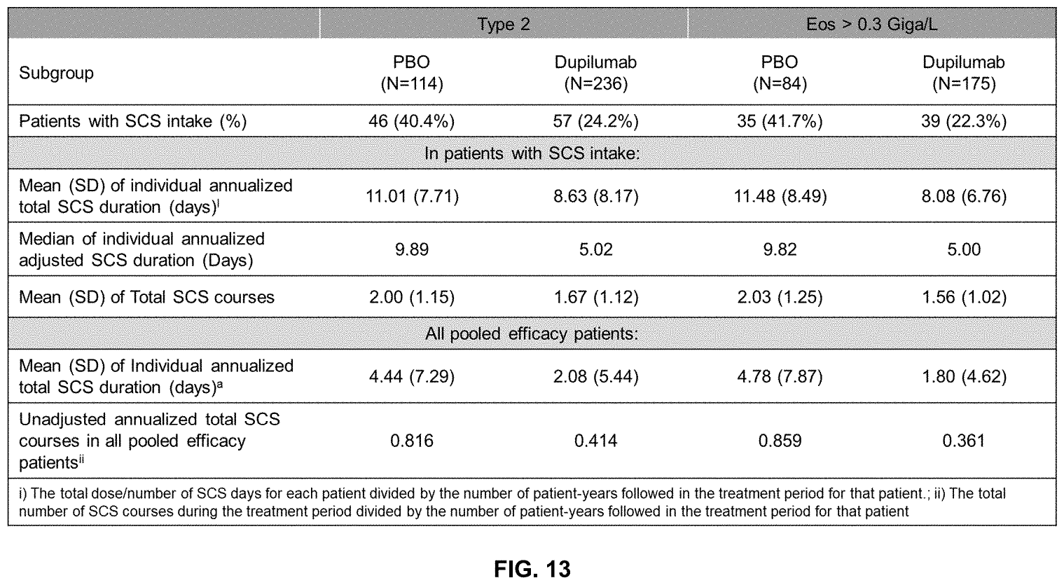

Systemic corticosteroids (SCS) use was analyzed (FIG. 13). A significant reduction in courses of SCS was achieved for both the Type 2 inflammatory phenotype subpopulation and the baseline blood eosinophils ≥0.3 Giga/L subpopulation (FIG. 12).

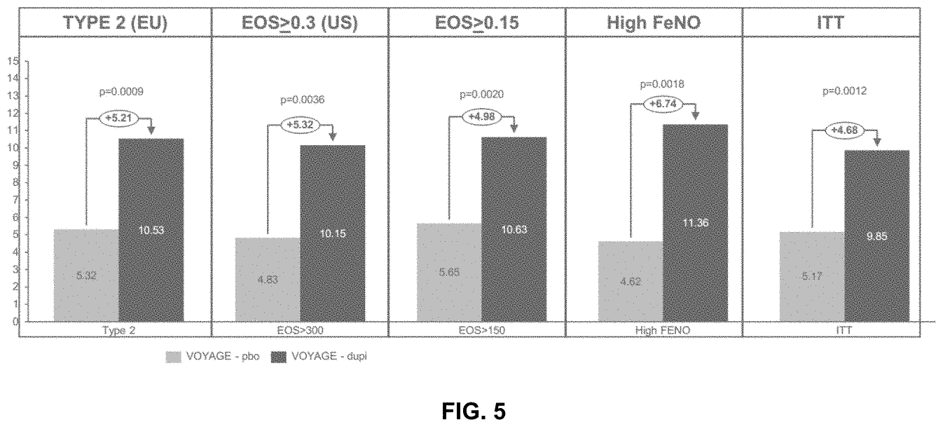

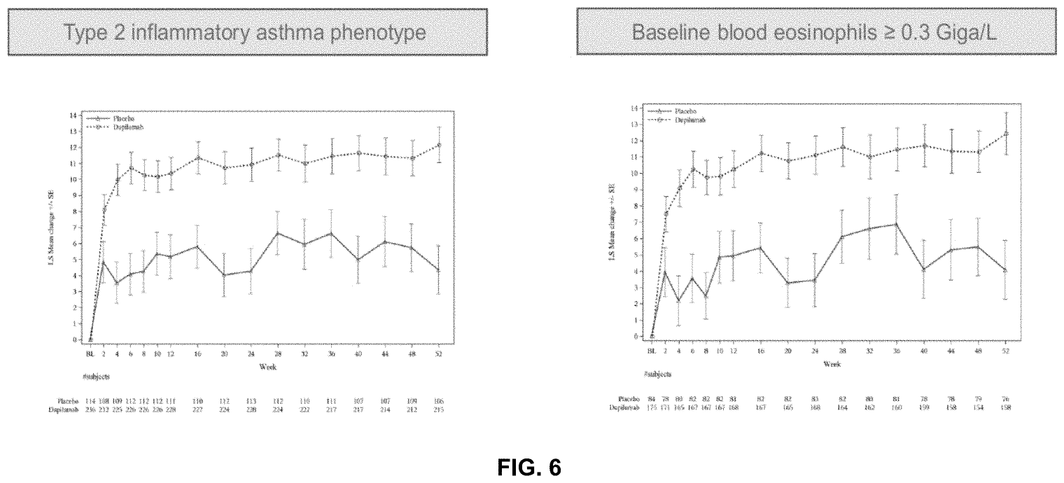

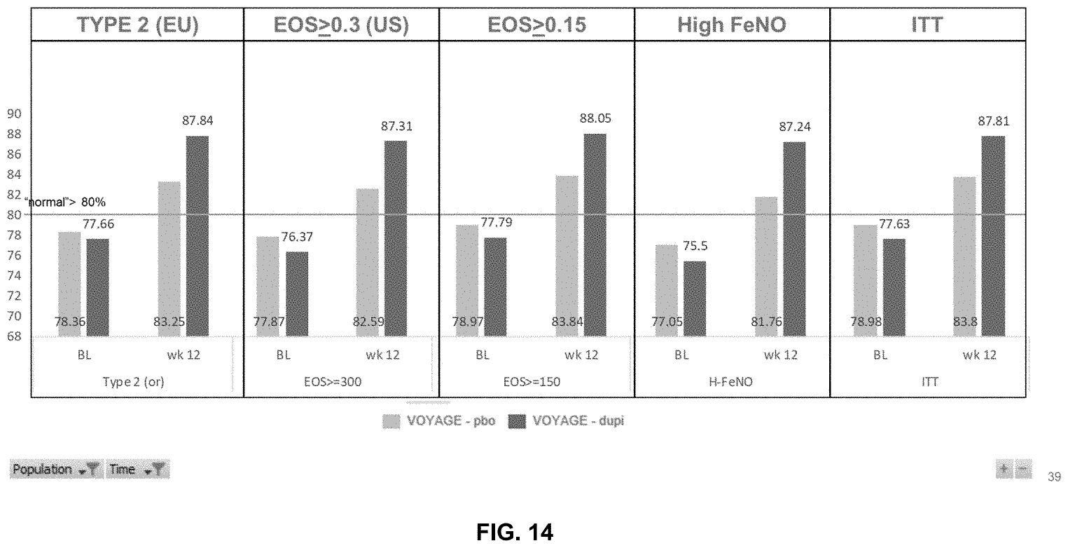

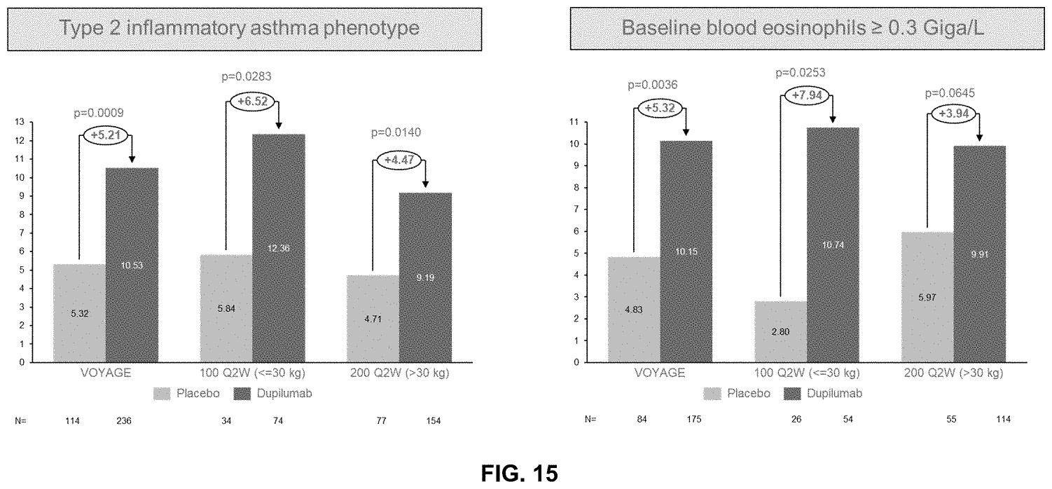

For the key secondary endpoint, change from baseline in pre-bronchodilator percent predicted FEV1 at week 12, the Type 2 inflammation population and EOS≥300 cells/μl population exhibited a improved lung function was observed at 12 weeks compared to baseline by 10.15 and 10.53 percentage points for DUPIXENT® vs. 4.83 and 5.32 percentage points for placebo (least squares mean difference for DUPIXENT® vs. placebo of 5.3 and 5.2, p=0.0036 and p=0.0009), respectively, as measured by percent predicted FEV1 (FEV1pp) (FIG. 5). This clinically meaningful improvement in lung function was observed as early as two weeks and was sustained for up to 52 weeks (FIG. 6, Tables 7-10). Mean FEV1pp improved across all populations (Type 2 (EU); EOS≥0.3 Giga/L (US); EOS≥0.15 Giga/L; high FeNO and ITT) to within normal range (FIG. 14). Significant improvement in FEV1pp was observed across both weight classes (≤30 kg or >30 kg) for the Type 2 inflammatory phenotype subpopulation and the baseline blood eosinophils ≥0.3 Giga/L subpopulation (FIG. 15).

TABLE 7
Absolute change from baseline in pre-bronchodilator FEV1 at weeks 2, 4,
8, 12, 24, 36, 52, in a Type 2 inflammatory asthma phenotype population.

| | Placebo | DUPIXENT ® |
|---|---|---|
| Overall Number of | 110 | 229 |
| Participants Analyzed | | |
| Change at Week 2 | 0.08 (0.03) | 0.14 (0.02) |
| Change at Week 4 | 0.06 (0.03) | 0.18 (0.02) |
| Change at Week 8 | 0.09 (0.03) | 0.21 (0.02) |
| Change at Week 12 | 0.12 (0.03) | 0.22 (0.02) |
| Change at Week 24 | 0.14 (0.03) | 0.27 (0.02) |
| Change at Week 36 | 0.23 (0.03) | 0.33 (0.02) |
| Change at Week 52 | 0.24 (0.03) | 0.41 (0.02) |

Least squares mean (SE), unit of measure: liters.

TABLE 8
Absolute change from baseline in pre-bronchodilator
FEV1 at weeks 2, 4, 8, 12, 24, 36, 52 in a baseline blood
eosinophils >=300 cells per microliter population.

| | Placebo | DUPIXENT ® |
|---|---|---|
| Overall Number of | 80 | 168 |
| Participants Analyzed | | |
| Change at Week 2 | 0.07 (0.03) | 0.13 (0.02) |
| Change at Week 4 | 0.04 (0.03) | 0.17 (0.02) |
| Change at Week 8 | 0.06 (0.03) | 0.20 (0.02) |
| Change at Week 12 | 0.12 (0.03) | 0.22 (0.02) |
| Change at Week 24 | 0.13 (0.03) | 0.28 (0.03) |
| Change at Week 36 | 0.24 (0.04) | 0.33 (0.03) |
| Change at Week 52 | 0.25 (0.04) | 0.42 (0.03) |

Least squares mean (SE), unit of measure: liters.

TABLE 9
Percent change from baseline in pre-bronchodilator FEV1 at weeks 2, 4,
8, 12, 24, 36, 52, in a Type 2 inflammatory asthma phenotype population.

| | Placebo | DUPIXENT ® |
|---|---|---|
| Overall Number of | 110 | 229 |
| Participants Analyzed | | |
| Change at Week 2 | 8.07 (2.00) | 13.38 (1.47) |
| Change at Week 4 | 6.73 (2.10) | 16.01 (1.55) |
| Change at Week 8 | 7.68 (1.87) | 16.33 (1.41) |
| Change at Week 12 | 8.87 (2.10) | 16.94 (1.56) |
| Change at Week 24 | 7.66 (2.12) | 17.61 (1.57) |
| Change at Week 36 | 10.88 (2.85) | 19.19 (2.06) |
| Change at Week 52 | 7.92 (2.81) | 20.06 (2.03) |

Least squares mean (SE), unit of measure: percent change FEV1.

TABLE 10
Absolute change from baseline in pre-bronchodilator
FEV1 at weeks 2, 4, 8, 12, 24, 36, 52 in a baseline blood
eosinophils >=300 cells per microliter population.

| | Placebo | DUPIXENT ® |
|---|---|---|
| Overall Number of | 80 | 168 |
| Participants Analyzed | | |
| Change at Week 2 | 7.55 (2.41) | 12.76 (1.73) |
| Change at Week 4 | 5.81 (2.57) | 15.20 (1.87) |
| Change at Week 8 | 6.09 (2.10) | 16.01 (1.56) |
| Change at Week 12 | 9.56 (2.45) | 17.14 (1.79) |
| Change at Week 24 | 7.44 (2.54) | 18.09 (1.85) |
| Change at Week 36 | 12.15 (3.67) | 19.65 (2.61) |
| Change at Week 52 | 8.50 (3.58) | 20.97 (2.54) |

Least squares mean (SE), unit of measure: percent change FEV1.

For the key secondary endpoint, change from baseline in pre-bronchodilator percent predicted FEV1 at week 12, the EOS≥150 cells/μl population, high FeNO population and the intent-to-treat population exhibited a 5.0 (p=0.0020), 6.7 (p=0.0018) and 4.7 (p=0.0012) percentage point improvement in change from baseline in percent predicted FEV1 (“FEV1pp”) at 12 weeks compared to placebo, respectively (FIG. 5).

In patients with a Type 2 phenotype, DUPIXENT® reduced FeNO levels (LS mean difference vs placebo −17.84; P<0.0001) at week 12 compared with placebo. A similar finding were observed in patients with eosinophils ≥300 cells/μL. (See Table 11.)

Median blood eosinophil values decreased to below the baseline value by week 52 in the DUPIXENT® group.

TABLE 11
Summary of efficacy outcomes in the phase 3 VOYAGE study.

| | | Baseline blood | |
|---|---|---|---|
| Type 2 population^{a} | | eosinophils ≥300 cells/μL | |
| Combined | Combined | Combined | Combined |
| placebo | DUPIXENT ® | placebo | DUPIXENT ® |
| n = 114 | n = 236 | n = 84 | n = 175 |

Primary endpoint

| | | | | |
|---|---|---|---|---|
| Annualized rate of severe asthma | | | | |
| exacerbations during the | | | | |
| treatment period | | | | |
| Mean number of severe | 2.18 (1.55) | 2.61 (2.58) | 2.37 (1.71) | 2.78 (2.90) |
| exacerbations in the year prior | | | | |
| to study (SD) | | | | |
| Estimate (95% CI) | 0.748 | 0.305 | 0.665 | 0.235 |
| | (0.542 to 1.034) | (0.223 to 0.416) | (0.467 to 0.949) | (0.160 to 0.345) |
| Relative risk vs placebo (95% CI) | | 0.407 | | 0.353 |
| | | (0.274 to 0.605) | | (0.222 to 0.562) |
| P-value vs matching placebo | | <0.0001 | | <0.0001 |

Secondary endpoints

| | | | | |
|---|---|---|---|---|
| Pre-bronchodilator FEV₁ pp | | | | |
| Mean value at baseline (SD) | 78.36 (14.51) | 77.66 (14.38) | 77.87 (15.19) | 76.37 (14.60) |
| LS mean change from baseline | 5.32 (1.36) | 10.53 (1.01) | 4.83 (1.54) | 10.15 (1.12) |
| at Week 12 (SE) | | | | |
| LS mean difference vs matching | | 5.21 | | 5.32 |
| placebo at Week 12 (95% CI) | | (2.14 to 8.27) | | (1.76 to 8.88) |
| P-value vs matching placebo | | 0.0009 | | 0.0036 |
| ACQ-7-IA score^{b} | | | | |
| Mean value at baseline (SD) | 2.12 (0.76) | 2.15 (0.70) | 2.15 (0.77) | 2.16 (0.73) |
| LS mean change from baseline | −1.00 (0.07) | −1.33 (0.05) | −0.88 (0.09) | −1.34 (0.06) |
| at Week 24 (SE) | | | | |
| LS mean difference vs matching | | −0.33 | | −0.46 |
| placebo at Week 24 (95% CI) | | (−0.50 to −0.16) | | (−0.67 to −0.26) |
| P-value vs matching placebo | | 0.0001 | | <0.0001 |
| FeNO, ppb | | | | |
| Mean value at baseline (SD) | 28.38 (23.44) | 31.83 (24.85) | 31.33 (23.52) | 34.53 (25.83) |
| LS mean change from baseline | −1.13 (1.43) | −18.97 (1.04) | −0.81 (1.69) | −21.40 (1.21) |
| at Week 12 (SE) | | | | |
| LS mean difference vs matching | | −17.84 | | −20.59 |
| placebo at Week 12 (95% CI) | | (−21.05 to −14.63) | | (−24.60 to −16.59) |
| P-value vs matching placebo | | <0.0001 | | <0.0001 |

^{a}EU primary population, defined as baseline blood eosinophils ≥150/μL or FeNO ≥20 ppb.
^{b}Within-person change in ACQ-7-IA is considered clinically meaningful.
CI, confidence interval;
SD, standard deviation;
SE standard error.

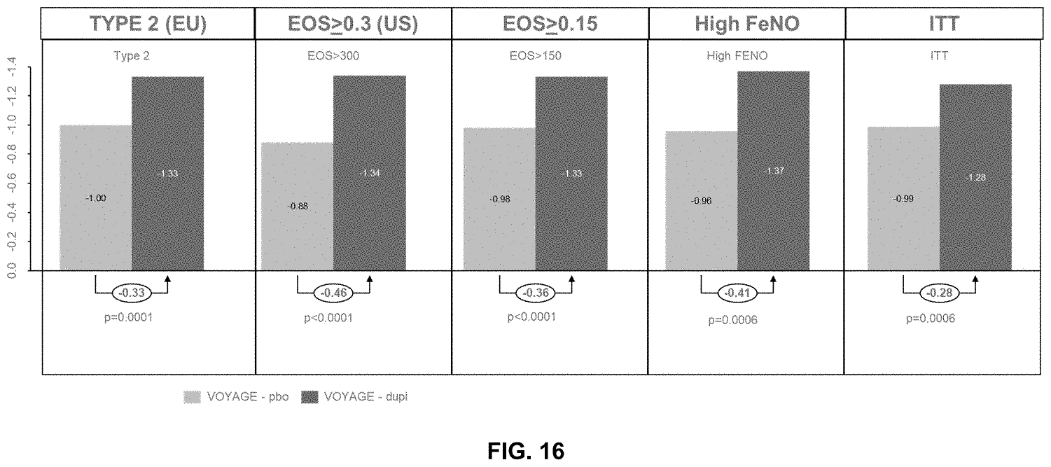

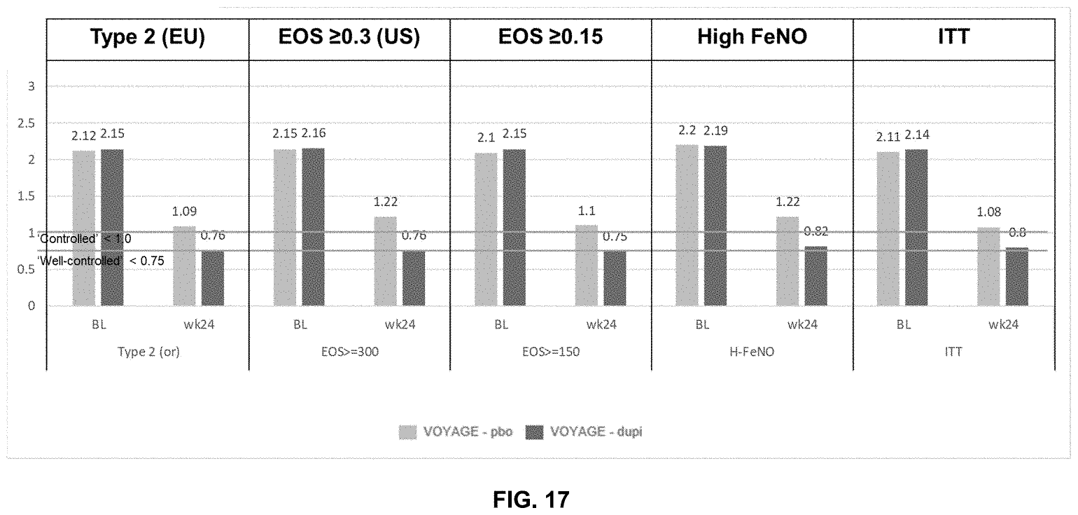

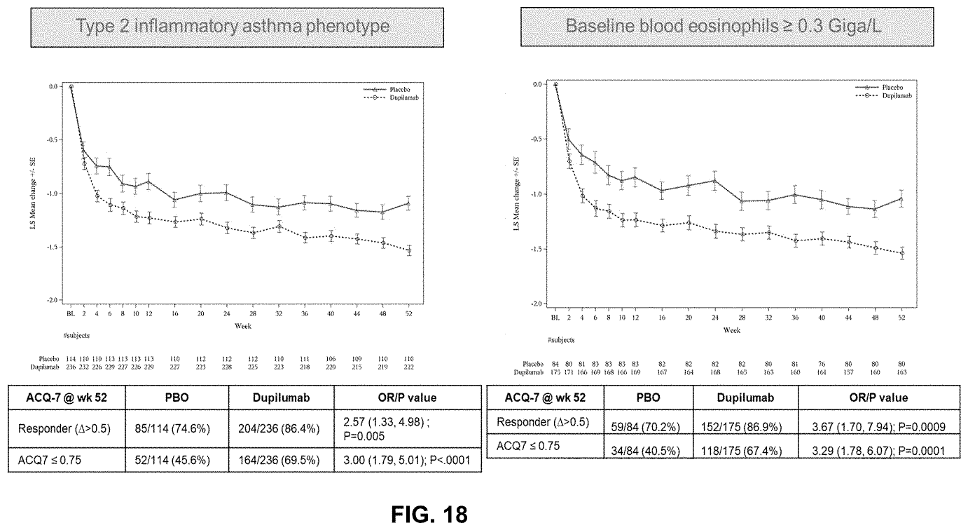

Asthma Control Questionnaire 7 Question (ACQ-7) scores improved compared to placebo across all populations at week 24 (Type 2 (EU); EOS≥0.3 Giga/L (US); EOS≥0.15 Giga/L; high FeNO and ITT) (FIG. 16), and ACQ-7 scores achieved at week 24 were within the range of asthma control (FIG. 17). ACQ-7-IA over 52 weeks is shown in FIG. 18 for both the Type 2 inflammatory phenotype subpopulation and the baseline blood eosinophils ≥0.3 Giga/L subpopulation.

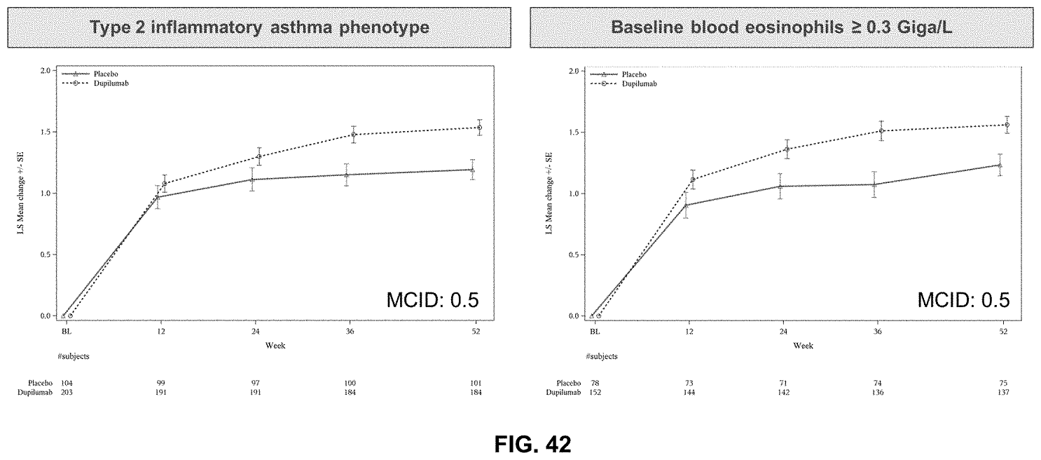

Pediatric Asthma Quality of Life Questionnaire (PAQLQ) scores showed improvement compared to placebo for quality of life for both a Type 2 inflammatory asthma phenotype subpopulation (defined as EOS≥0.150 Giga/L or FeNO≥20 ppb) and a baseline blood eosinophils ≥0.3 Giga/L subpopulation (FIG. 42) that was maintained for 52 weeks.

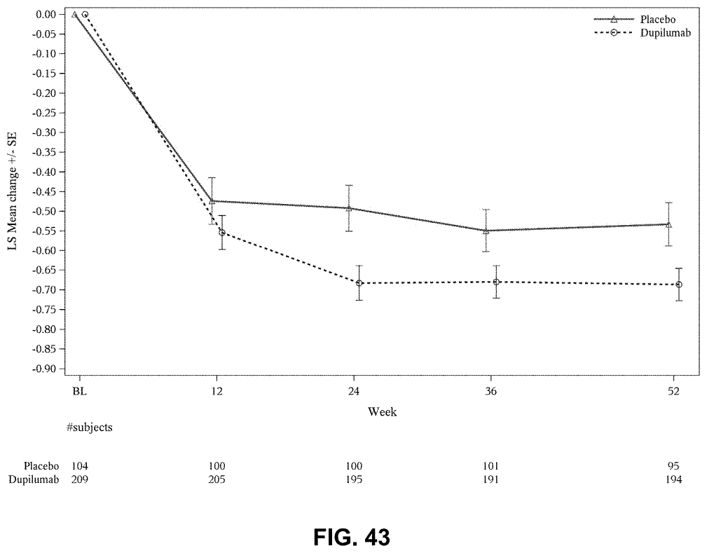

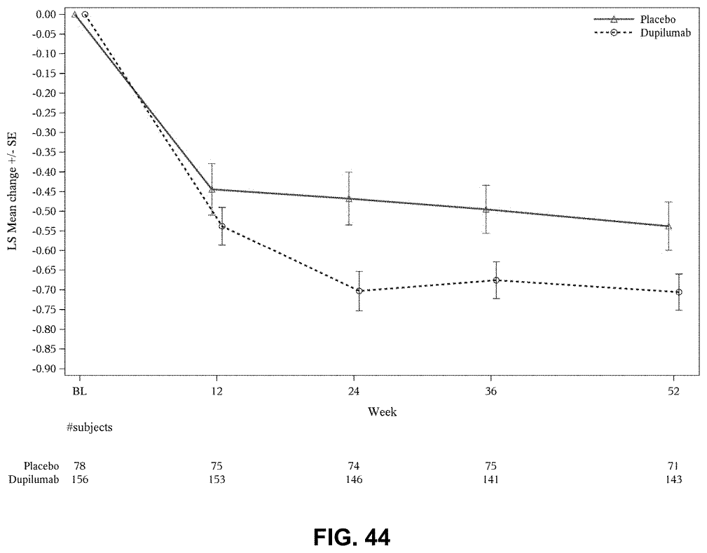

Pediatric Asthma Caregiver's Quality of Life Questionnaire (PACQLQ) scores showed improvement compared to placebo in a Type 2 inflammatory asthma phenotype subpopulation (defined as EOS≥0.150 Giga/L or FeNO≥20 ppb) (FIG. 43) as well as in a baseline blood eosinophils ≥0.3 Giga/L subpopulation (FIG. 44) that was maintained for 52 weeks.

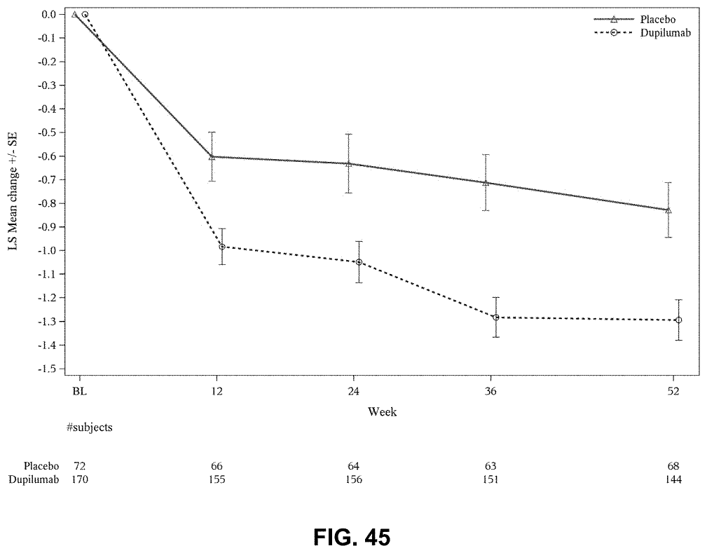

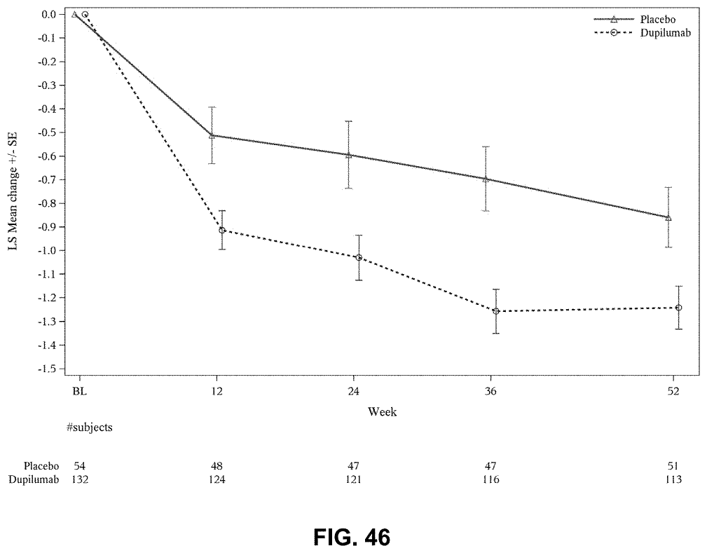

Pediatric Rhinoconjunctivitis Quality of Life Questionnaire (PRQLQ) global scores showed improvement compared to placebo in a Type 2 inflammatory asthma phenotype subpopulation (FIG. 45) as well as in a baseline blood eosinophils ≥0.3 Giga/L subpopulation (FIG. 46) that was maintained for 52 weeks.

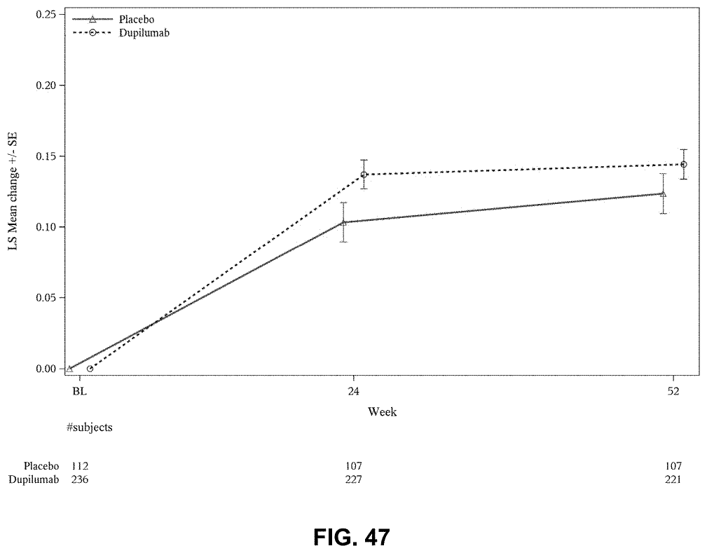

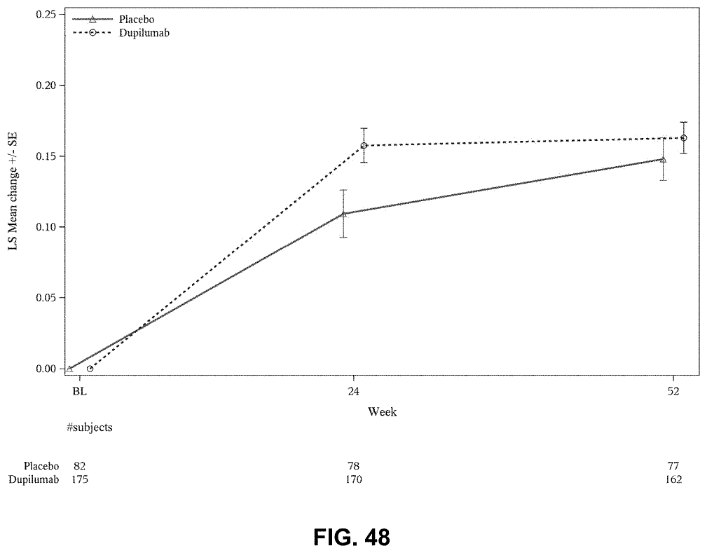

EuroQol EQ-5D-5L scores showed improvement compared to placebo in a Type 2 inflammatory asthma phenotype subpopulation (FIG. 47) as well as in a baseline blood eosinophils ≥0.3 Giga/L subpopulation (FIG. 48) that was maintained for 52 weeks.

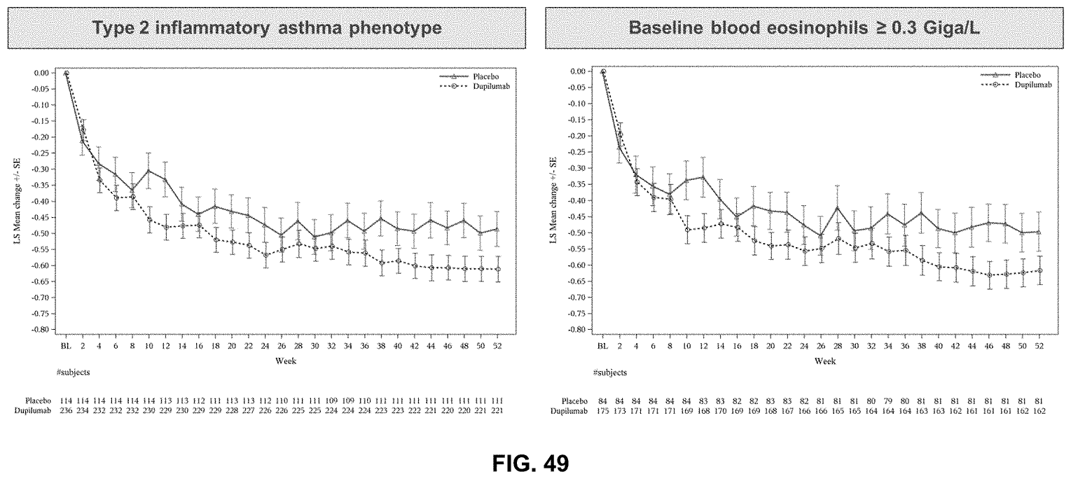

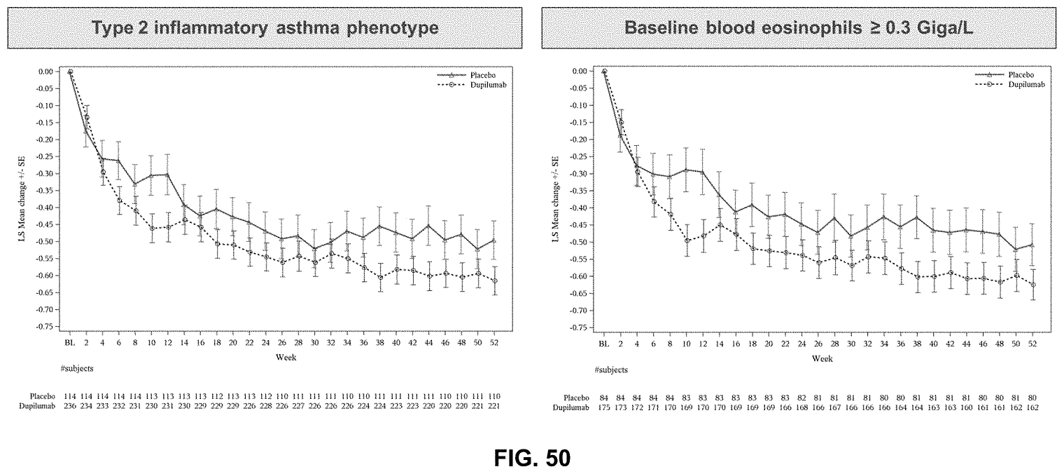

AM symptom scores (FIG. 49) and PM symptoms scores (FIG. 50) were improved compared to placebo, and the improvement was maintained for 52 weeks.

Change from baseline in morning (AM)/evening (PM) peak expiratory flow (PEF) was assessed. (See Tables 12 and 13.)

TABLE 12
Change from baseline in
morning (AM)/evening (PM)
peak expiratory flow (PEF)
at weeks 2, 4, 8, 12, 24, 36, and 52 in a Type 2
inflammatory asthma phenotype population.

| | Placebo | DUPIXENT ® |
|---|---|---|
| Overall Number of | 110 | 228 |
| Participants Analyzed | | |
| AM: Change at Week 2 | 5.57 (2.66) | 6.50 (2.00) |
| AM: Change at Week 4 | 7.10 (3.31) | 12.81 (2.43) |
| AM: Change at Week 8 | 7.37 (3.56) | 18.05 (2.59) |
| AM: Change at Week 12 | 5.37 (3.93) | 19.38 (2.85) |
| AM: Change at Week 24 | 8.76 (4.53) | 23.32 (3.25) |
| AM: Change at Week 36 | 17.70 (4.99) | 26.77 (3.56) |
| AM: Change at Week 52 | 19.88 (5.16) | 31.45 (3.69) |
| PM: Change at Week 2 | 7.62 (2.94) | 10.60 (2.20) |
| PM: Change at Week 4 | 6.92 (3.42) | 15.23 (2.52) |
| PM: Change at Week 8 | 5.52 (3.60) | 20.17 (2.64) |
| PM: Change at Week 12 | 1.71 (3.92) | 19.68 (2.85) |
| PM: Change at Week 24 | 3.78 (4.61) | 22.75 (3.32) |
| PM: Change at Week 36 | 12.55 (5.06) | 24.69 (3.62) |
| PM: Change at Week 52 | 16.46 (5.20) | 28.20 (3.73) |

Least squares mean (SE),
unit of measure: liters/minute.

TABLE 13
Change from baseline in
morning (AM)/evening (PM)
peak expiratory flow (PEF)
at weeks 2, 4, 8, 12, 24, 36, and 52 in a baseline
blood eosinophils >=300 cells per
microliter population.

| | Placebo | DUPIXENT ® |
|---|---|---|
| Overall Number of | 80 | 167 |
| Participants Analyzed | | |
| AM: Change at Week 2 | 5.85 (2.97) | 7.94 (2.21) |
| AM: Change at Week 4 | 5.62 (3.80) | 11.75 (2.76) |
| AM: Change at Week 8 | 5.54 (4.09) | 17.32 (2.96) |
| AM: Change at Week 12 | 4.77 (4.65) | 19.25 (3.34) |
| AM: Change at Week 24 | 4.78 (5.05) | 23.62 (3.61) |
| AM: Change at Week 36 | 14.80 (5.77) | 26.78 (4.08) |
| AM: Change at Week 52 | 19.75 (6.33) | 32.80 (4.49) |
| PM: Change at Week 2 | 5.64 (3.42) | 10.02 (2.53) |
| PM: Change at Week 4 | 4.36 (3.96) | 13.09 (2.89) |
| PM: Change at Week 8 | 0.92 (4.05) | 17.51 (2.96) |
| PM: Change at Week 12 | −1.37 (4.57) | 17.73 (3.30) |
| PM: Change at Week 24 | −1.37 (5.21) | 20.30 (3.74) |
| PM: Change at Week 36 | 7.52 (6.01) | 23.23 (4.26) |
| PM: Change at Week 52 | 14.67 (6.39) | 26.60 (4.54) |

Least squares mean (SE),
unit of measure: liters/minute.

Nocturnal awakenings (FIG. 51) were decreased compared to placebo, and the decrease was maintained for 52 weeks.

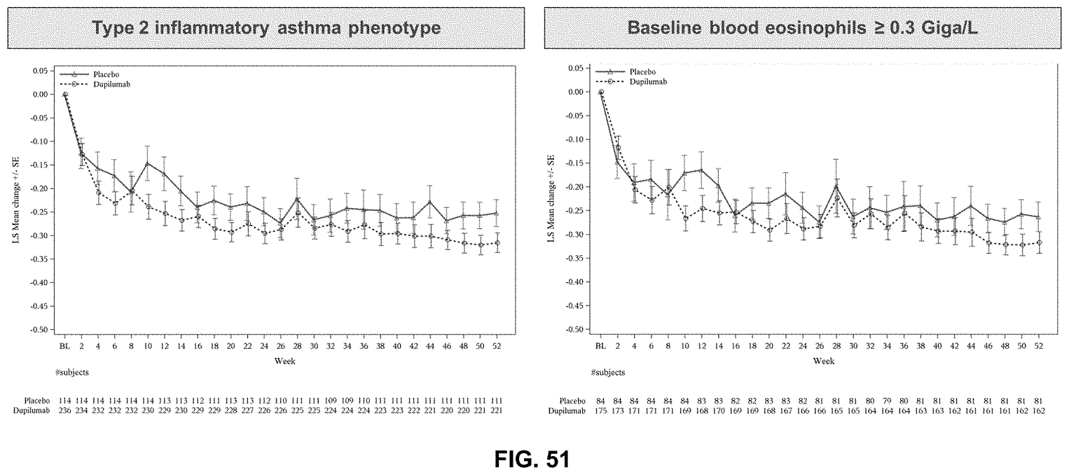

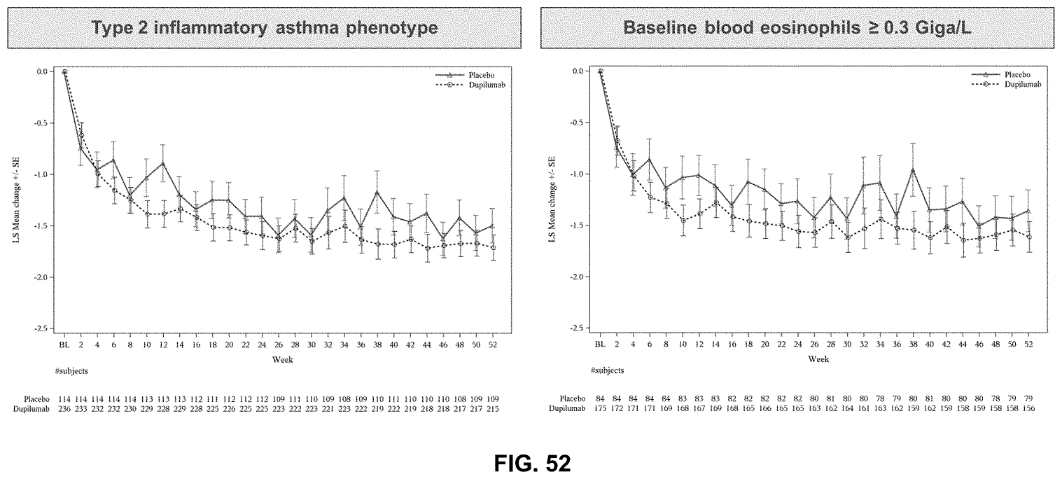

Reliever medication use (FIG. 52) was decreased compared to placebo, and the decrease was maintained for 52 weeks.

Over one year, overall rates of adverse events were 83% for DUPIXENT® and 80% for placebo. Adverse events that were more commonly observed with DUPIXENT® included injection site reactions (18% for DUPIXENT® and 13% for placebo), viral upper respiratory tract infections (12% for DUPIXENT® and 10% for placebo), and eosinophilia (6% for DUPIXENT® and 1% for placebo).

### Biomarker Analyses

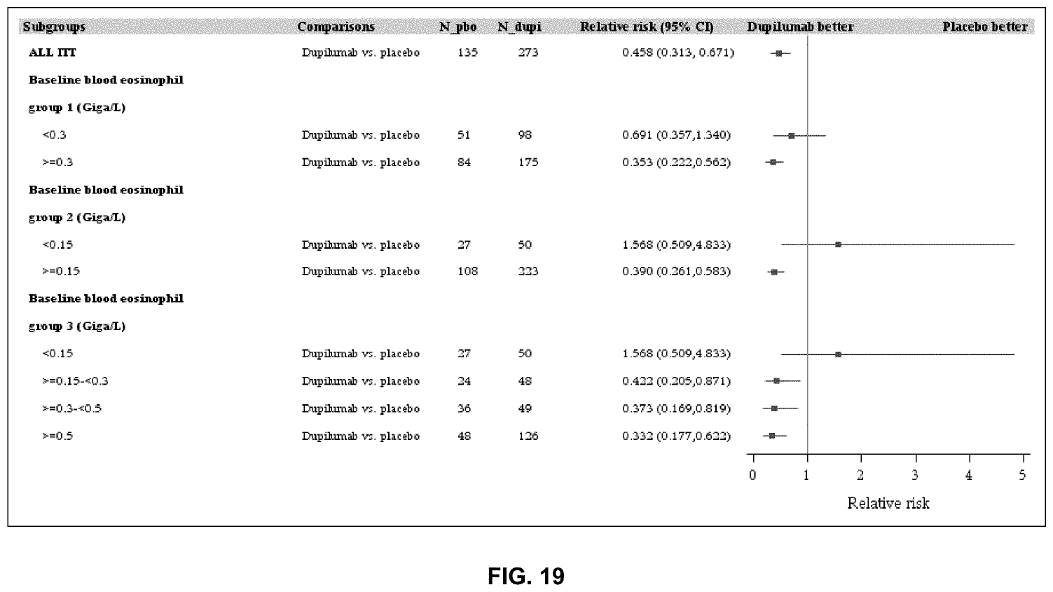

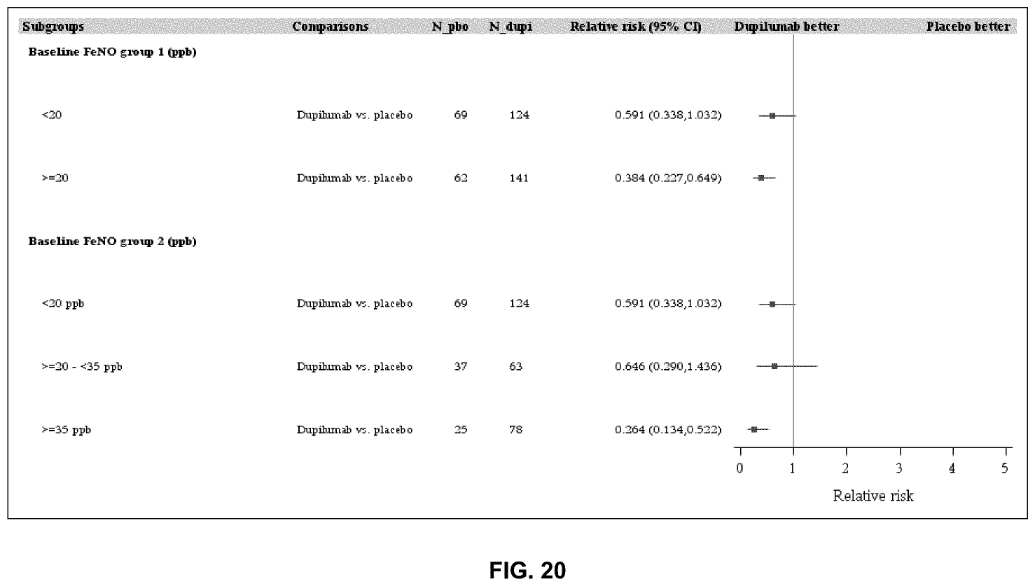

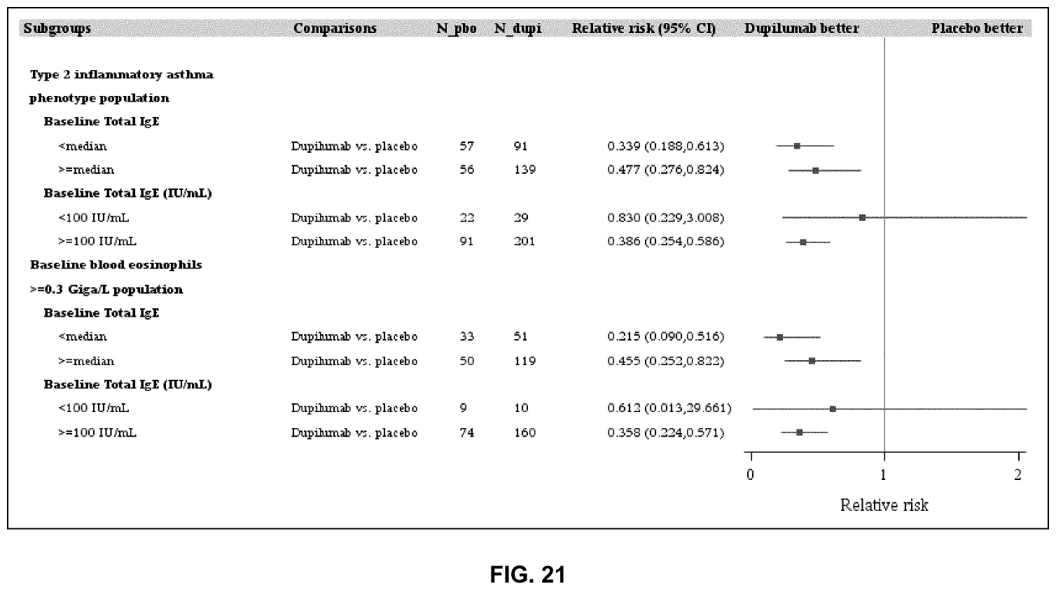

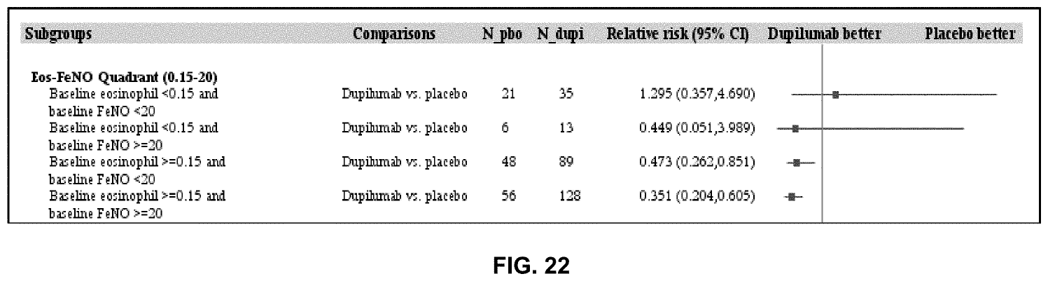

Relative risk of exacerbation in subpopulations stratified by baseline biomarker was assessed and is shown in FIGS. 19-21. DUPIXENT® was determined to be better than placebo for all groups. Quadrant analysis indicated efficacy in Type 2 inflammatory phenotype subpopulations and no efficacy in non-Type 2 inflammatory phenotype subpopulations (FIG. 22).

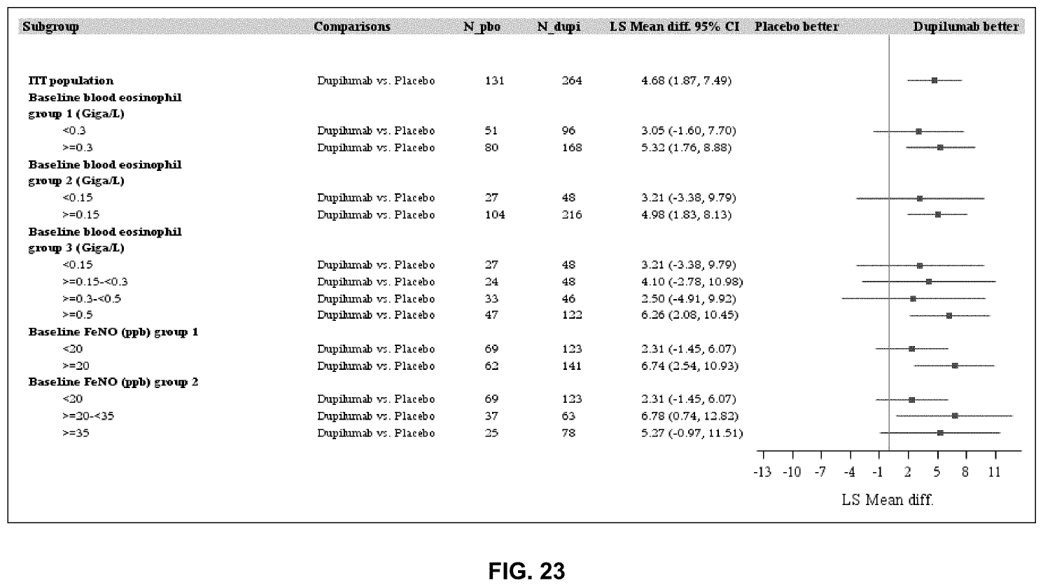

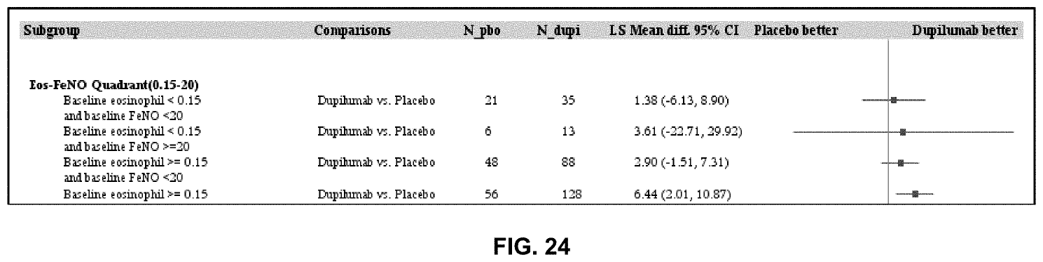

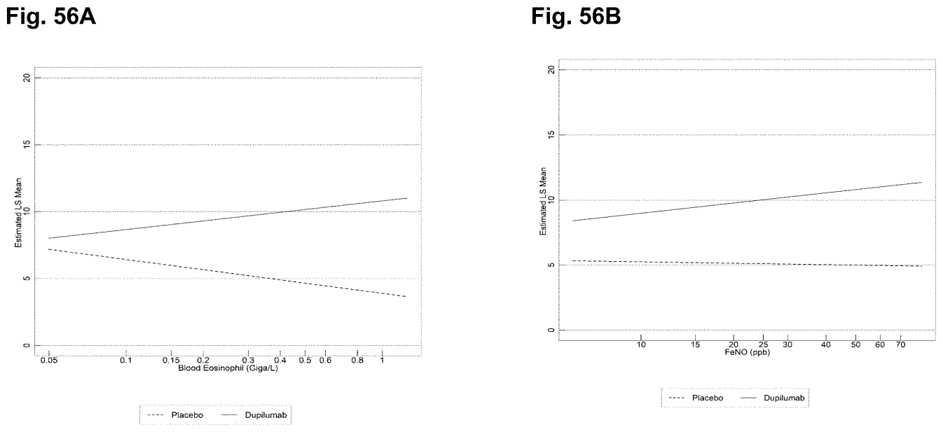

Percent predicted FEV1 (FEV1pp) was assessed and is shown in FIG. 23. FEV1pp quadrant analysis indicated efficacy in all quadrants (FIG. 24). Change from baseline in pre-bronchodilator % predicted FEV1 (%) at week 12 was determined as a function of baseline blood eosinophil (Giga/L) levels and as a function of baseline FeNO (ppb) levels (FIG. 56A-FIG. 56B).

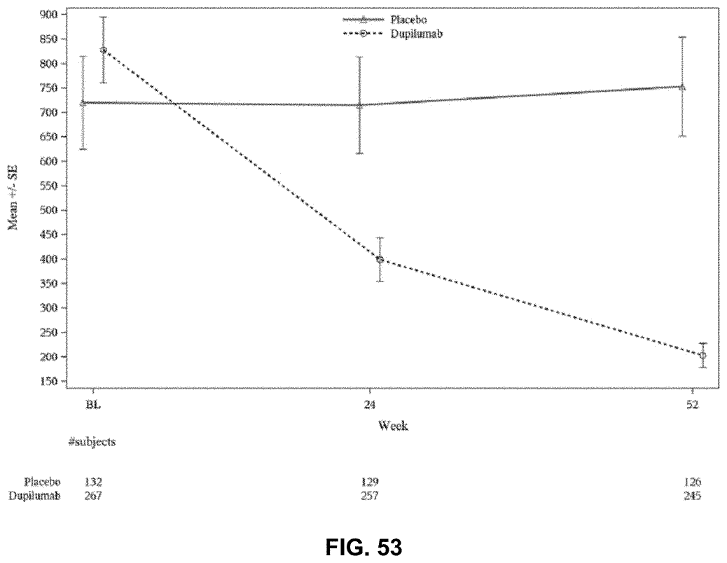

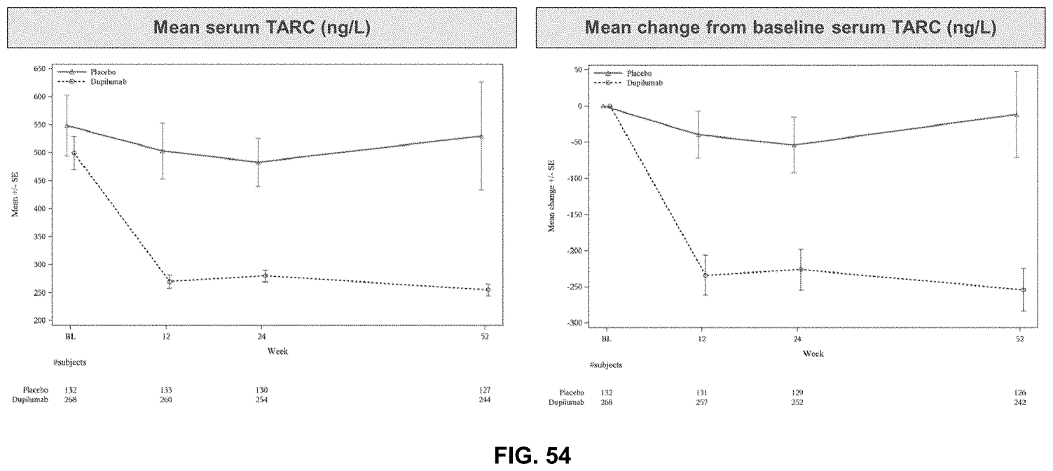

There was a marked decrease in IgE over 52 weeks compared to placebo (FIG. 53). There was a sustained decrease in TARC over 52 weeks compared to placebo (FIG. 54).

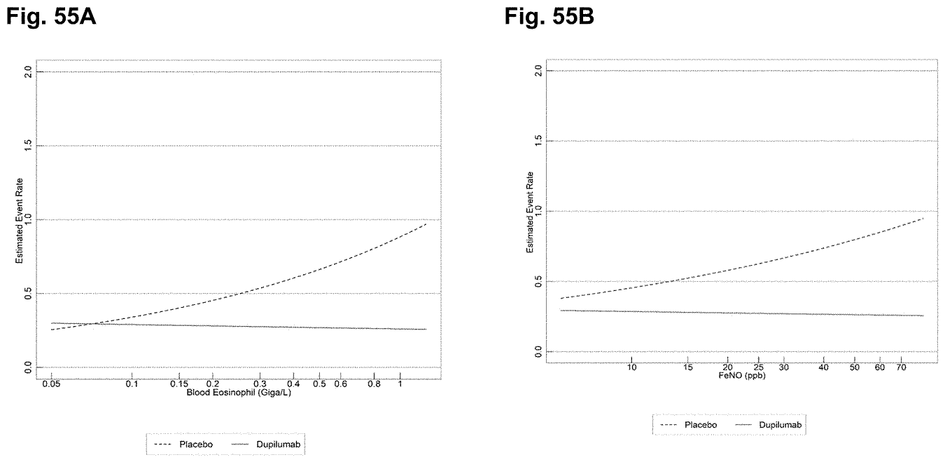

A reduction in estimated annualized event rates of severe exacerbation during 52-week treatment period was shown as a function of baseline blood eosinophil (Giga/L) levels and as a function of baseline FeNO (ppb) levels (FIG. 55A-FIG. 55B).

### Lung Function Analyses

Lung function data was captured for all VOYAGE subjects, including forced expiratory volume in 1 second (FEV₁), forced expiratory volume in 1 second percent predicted (FEV1pp), forced vital capacity (FVC), forced expiratory flow at 25% to 75% of forced vital capacity (FEF_{25-75%}), pre-BD FEV1 and post-BD FEV1.

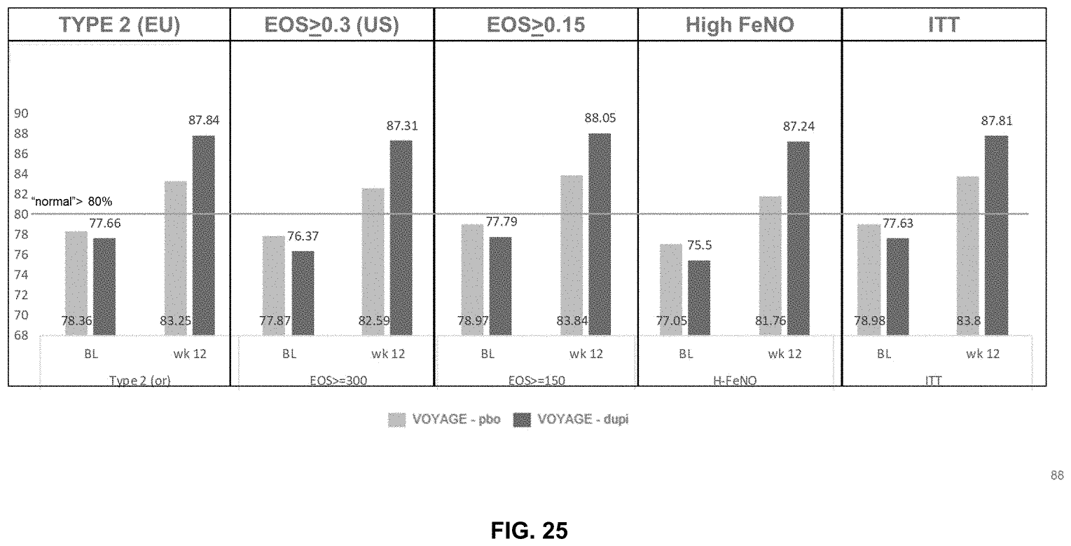

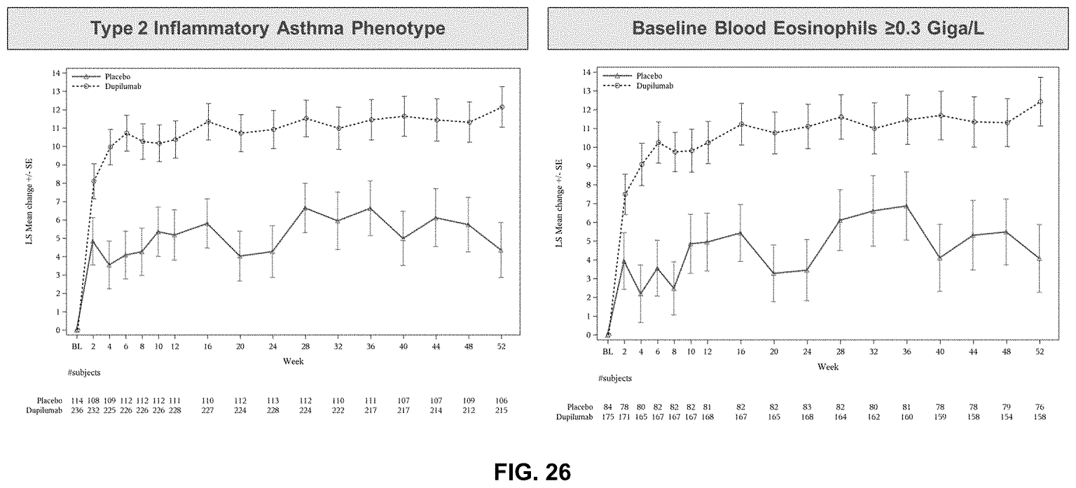

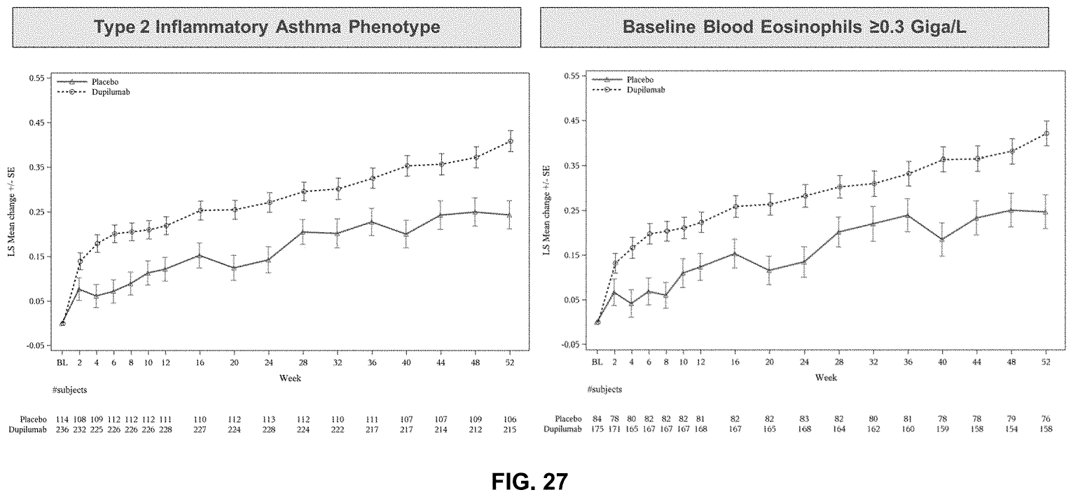

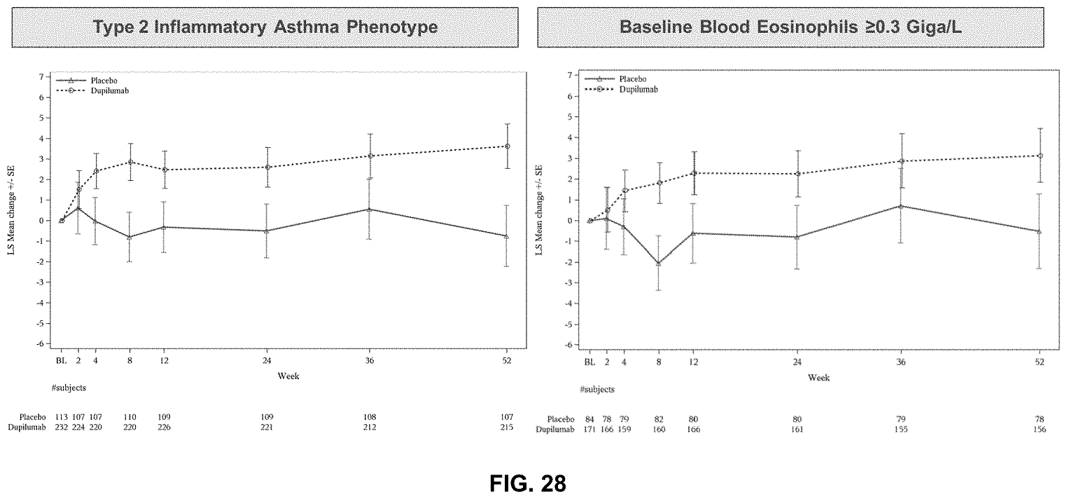

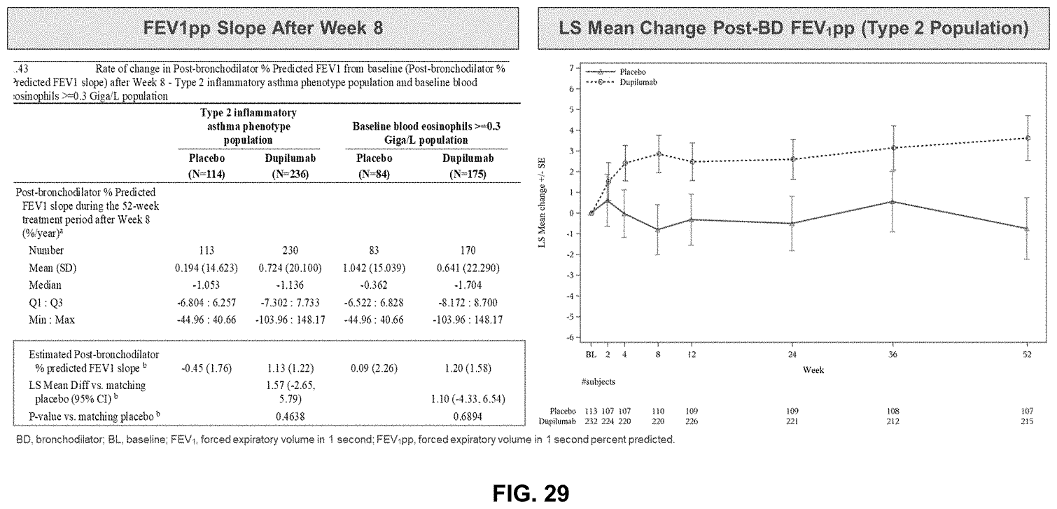

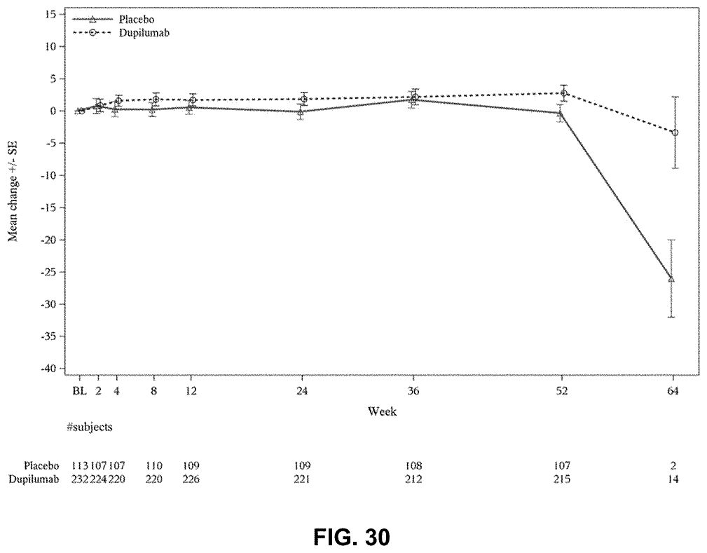

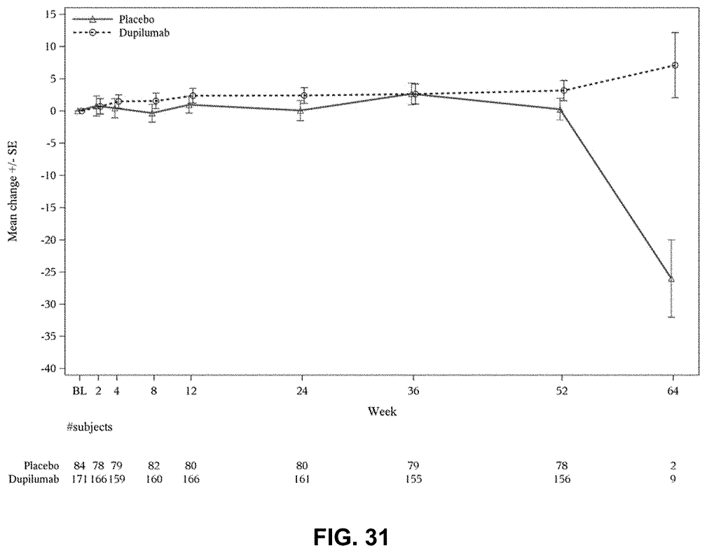

Mean FEV1pp improved across all populations (Type 2 (EU); EOS≥0.3 Giga/L (US); EOS≥0.15 Giga/L; high FeNO and ITT) to within normal range (FIG. 25). A rapid (within 2 weeks) and sustained (over 52 weeks) improvement in lung function was observed (FIG. 26). Improvements were observed for both pre-bronchodilator (pre-BD) FEV1 (FIG. 27) and post-bronchodilator (post-BD) FEV1 (FIG. 28). The mean absolute improvement in post-BD FEV1pp was maintained to 52 weeks (FIG. 29, Tables 14 and 15). A plot of mean change from baseline in post-BD percent predicted FEV1 over time for the Type 2 inflammatory asthma phenotype subpopulation is shown in FIG. 30. A plot of mean change from baseline in post-BD percent predicted FEV1 over time for the baseline blood eosinophils ≥0.3 Giga/L subpopulation is shown in FIG. 31.

TABLE 14
Change from baseline in post-bronchodilator
percent predicted FEV1 at weeks 2,
4, 8, 12, 24, 36, and 52 in a Type 2
inflammatory asthma phenotype population.

| | Placebo | DUPIXENT ® |
|---|---|---|
| Overall Number of | 110 | 230 |
| Participants | | |
| Analyzed | | |
| Change at Week 2 | 0.61 (1.26) | 1.51 (0.92) |
| Change at Week 4 | −0.02 (1.15) | 2.42 (0.86) |
| Change at Week 8 | −0.79 (1.21) | 2.85 (0.90) |
| Change at Week 12 | −0.32 (1.23) | 2.48 (0.91) |
| Change at Week 24 | −0.50 (1.31) | 2.60 (0.96) |
| Change at Week 36 | 0.55 (1.46) | 3.15 (1.07) |
| Change at Week 52 | −0.75 (1.48) | 3.62 (1.08) |

Least squares mean (SE),
unit of measure: liters.

TABLE 15
Change from baseline in post-bronchodilator
percent predicted FEV1 at weeks 2,
4, 8, 12, 24, 36, and 52 in a baseline blood
eosinophils >=300 cells per
microliter population.

| | Placebo | DUPIXENT ® |
|---|---|---|
| Overall Number of | 80 | 169 |
| Participants Analyzed | | |
| Change at Week 2 | 0.10 (1.49) | 0.52 (1.07) |
| Change at Week 4 | −0.29 (1.35) | 1.44 (1.00) |
| Change at Week 8 | −2.05 (1.31) | 1.81 (0.98) |
| Change at Week 12 | −0.61 (1.44) | 2.28 (1.04) |
| Change at Week 24 | −0.80 (1.54) | 2.25 (1.12) |
| Change at Week 36 | 0.72 (1.80) | 2.87 (1.31) |
| Change at Week 52 | −0.52 (1.79) | 3.13 (1.30) |

Least squares mean (SE),
unit of measure: liters.

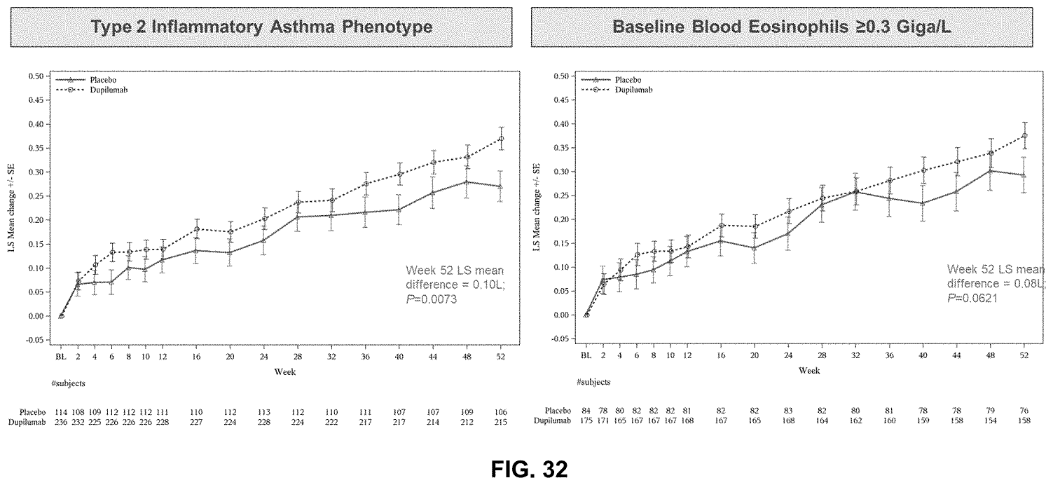

FVC was assessed and results are shown in FIG. 32 for the Type 2 inflammatory asthma phenotype subpopulation and the baseline blood eosinophils ≥0.3 Giga/L subpopulation.

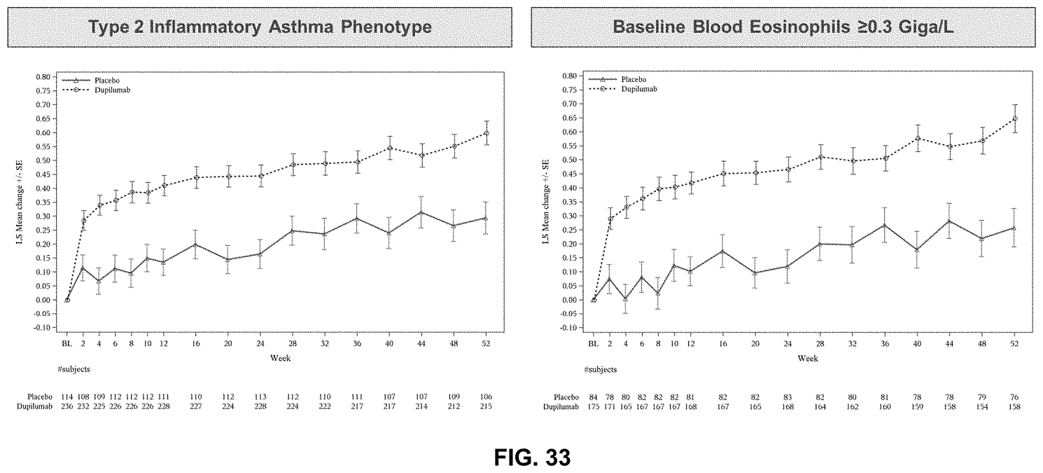

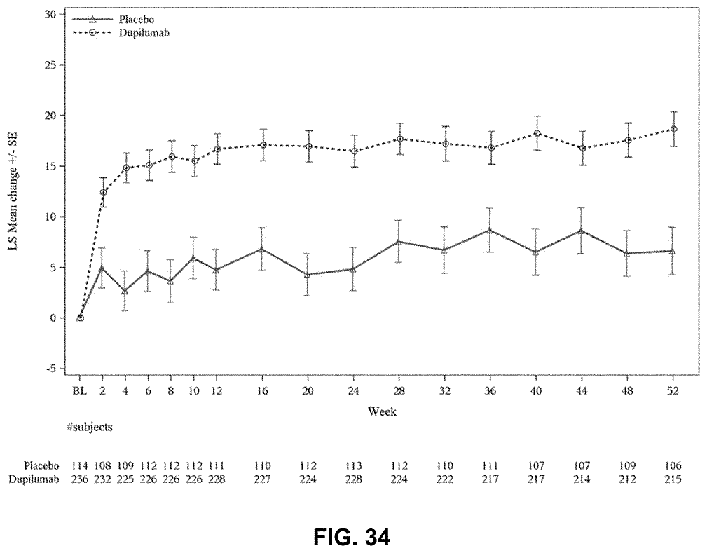

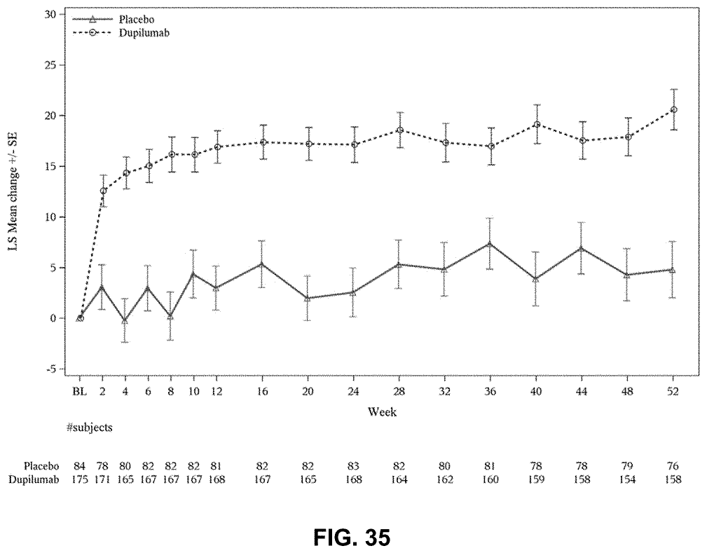

FEF_{25-75%} was assessed and results are shown in FIG. 33 for the Type 2 inflammatory asthma phenotype subpopulation and the baseline blood eosinophils ≥0.3 Giga/L subpopulation. Least squares mean change from baseline in percent predicted FEF_{25-75%} over time for the Type 2 inflammatory asthma phenotype subpopulation is shown in FIG. 34. Least squares mean change from baseline in percent predicted FEF_{25-75%} over time for the baseline blood eosinophils ≥0.3 Giga/L subpopulation is shown in FIG. 35.

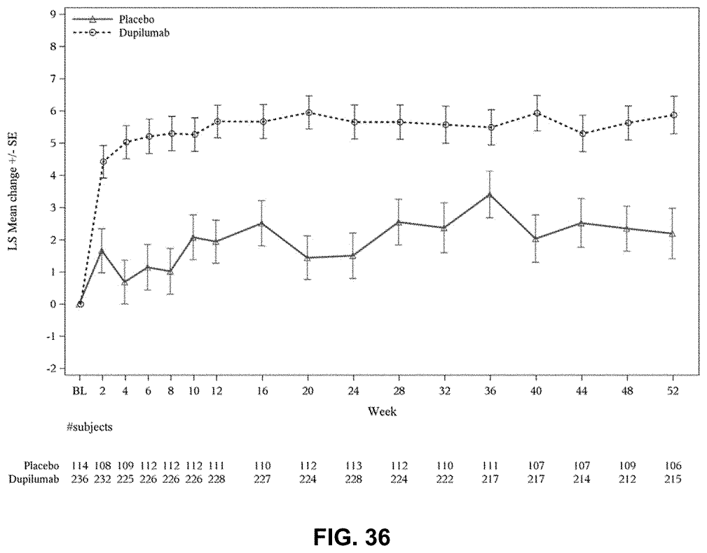

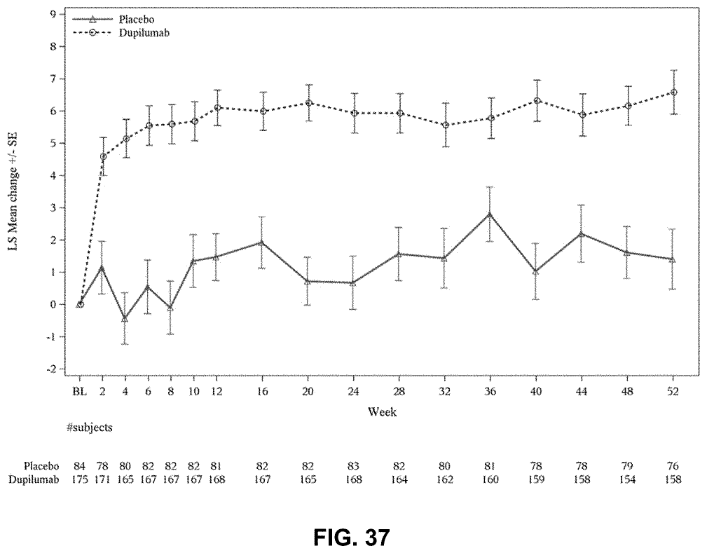

Least squares mean change from baseline in FEV1/FVC percent over time for the Type 2 inflammatory asthma phenotype subpopulation is shown in FIG. 36. Least squares mean change from baseline in FEV1/FVC percent over time for the baseline blood eosinophils ≥0.3 Giga/L subpopulation is shown in FIG. 37.

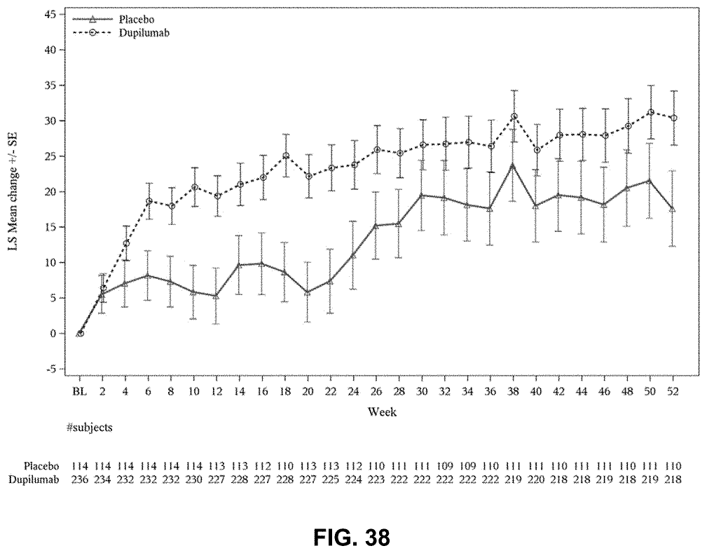

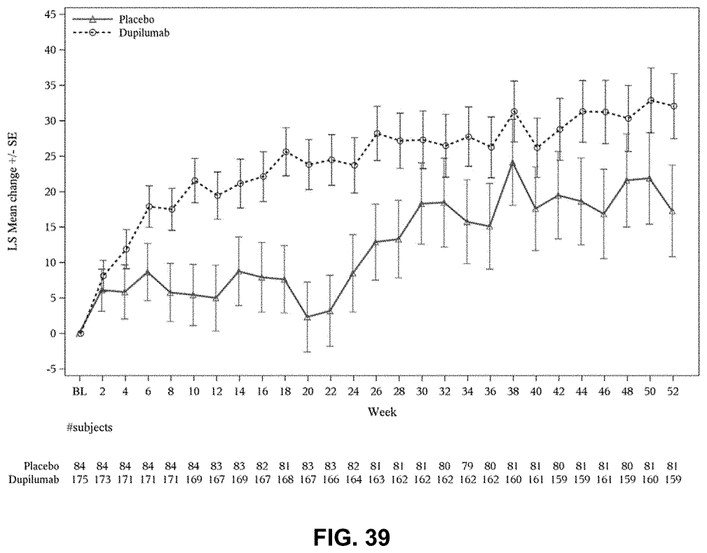

Least squares mean change from baseline in morning peak expiratory flow (AM PEF) over time for the Type 2 inflammatory asthma phenotype subpopulation is shown in FIG. 38. Least squares mean change from baseline in AM PEF over time for the baseline blood eosinophils ≥0.3 Giga/L subpopulation is shown in FIG. 39.

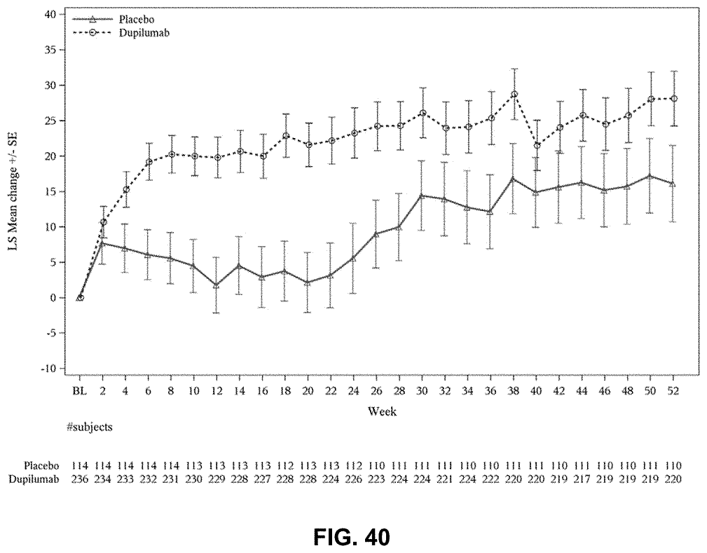

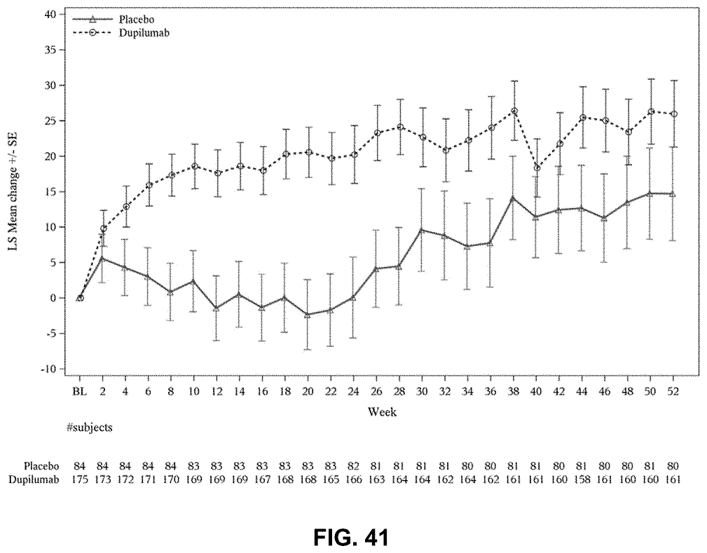

Least squares mean change from baseline in evening peak expiratory flow (PM PEF) over time for the Type 2 inflammatory asthma phenotype subpopulation is shown in FIG. 40. Least squares mean change from baseline in PM PEF over time for the baseline blood eosinophils ≥0.3 Giga/L subpopulation is shown in FIG. 41.

Change from baseline in morning asthma symptom score was determined at weeks 2, 4, 8, 12, 24, 36, and 52 (Tables 16 and 17). The morning asthma symptom score evaluated a participant's overall asthma symptoms experienced during the previous night. It ranged from 0 (no asthma symptoms, slept through the night) to 4 (bad night, awake most of the night because of asthma), where higher scores indicated more severe symptoms. LS means and SE were derived from MMRM model with change from baseline in AM asthma symptom score values up to week 52 as the response variable, and treatment, age, baseline weight group, region, baseline eosinophil level, baseline FeNO level, baseline ICS dose level, visit, treatment by-visit interaction, baseline AM asthma symptom score value and baseline-by-visit interaction as covariates.

TABLE 16
Change from baseline in morning
asthma symptom score at weeks
2, 4, 8, 12, 24, 36, and
52 in a Type 2 inflammatory
asthma phenotype population.

| | Placebo | DUPIXENT ® |
|---|---|---|
| Overall Number of | 110 | 228 |
| Participants Analyzed | | |
| Change at Week 2 | −0.22 (0.04) | −0.18 (0.03) |
| Change at Week 4 | −0.29 (0.05) | −0.34 (0.04) |
| Change at Week 8 | −0.37 (0.05) | −0.39 (0.04) |
| Change at Week 12 | −0.34 (0.05) | −0.48 (0.04) |
| Change at Week 24 | −0.46 (0.05) | −0.56 (0.04) |
| Change at Week 36 | −0.48 (0.05) | −0.56 (0.04) |
| Change at Week 52 | −0.50 (0.05) | −0.61 (0.04) |

Least squares mean (SE),
unit of measure: score on a scale.

TABLE 17
Change from baseline in morning
asthma symptom score at weeks 2, 4, 8, 12, 24,
36, and 52 in a baseline blood
eosinophils >=300 cells
per microliter population.

| | Placebo | DUPIXENT ® |
|---|---|---|
| Overall Number of | 80 | 167 |
| Participants | | |
| Analyzed | | |
| Change at Week 2 | −0.24 (0.05) | −0.20 (0.04) |
| Change at Week 4 | −0.32 (0.06) | −0.34 (0.04) |
| Change at Week 8 | −0.38 (0.06) | −0.40 (0.05) |
| Change at Week 12 | −0.33 (0.06) | −0.48 (0.04) |
| Change at Week 24 | −0.45 (0.06) | −0.55 (0.04) |
| Change at Week 36 | −0.46 (0.06) | −0.56 (0.04) |
| Change at Week 52 | −0.50 (0.06) | −0.62 (0.04) |

Least squares mean (SE),
unit of measure: score on a scale.

Change from baseline in evening asthma symptom score was determined at weeks 2, 4, 8, 12, 24, 36 and 52 (Tables 18 and 19). The evening asthma symptom score evaluated a participant's overall asthma symptoms experienced during the day. It ranged from 0 (very well, no asthma symptoms) to 4 (asthma very bad, unable to carry out daily activities as usual), where lower scores (0) indicated more mild symptoms and higher scores (4) indicated more severe symptoms. LS means and SE were derived from MMRM model with change from baseline in PM asthma symptom score values up to week 52 as response variable, and treatment, age, baseline weight group, region, baseline eosinophil level, baseline FeNO level, baseline ICS dose level, visit, treatment by-visit interaction, baseline PM asthma symptom score value and baseline-by-visit interaction as covariates.

TABLE 18
Change from baseline in evening asthma
symptom score at weeks 2, 4, 8, 12, 24,
36, and 52 in a Type 2 inflammatory
asthma phenotype population.

| | Placebo | DUPIXENT ® |
|---|---|---|
| Overall Number of | 110 | 228 |
| Participants Analyzed | | |
| Change at Week 2 | −0.17 (0.05) | −0.13 (0.03) |
| Change at Week 4 | −0.25 (0.05) | −0.29 (0.04) |
| Change at Week 8 | −0.33 (0.06) | −0.40 (0.04) |
| Change at Week 12 | −0.30 (0.06) | −0.45 (0.04) |
| Change at Week 24 | −0.44 (0.06) | −0.53 (0.04) |
| Change at Week 36 | −0.47 (0.06) | −0.55 (0.04) |
| Change at Week 52 | −0.50 (0.06) | −0.59 (0.04) |

Least squares mean (SE),
unit of measure: score on a scale.

TABLE 19
Change from baseline in evening asthma
symptom score at weeks 2, 4, 8, 12, 24,
36, and 52 in a baseline blood
eosinophils >=300 cells per microliter population.

| | Placebo | DUPIXENT ® |
|---|---|---|
| Overall Number of | 80 | 167 |
| Participants Analyzed | | |
| Change at Week 2 | −0.18 (0.05) | −0.15 (0.04) |
| Change at Week 4 | −0.27 (0.06) | −0.29 (0.04) |
| Change at Week 8 | −0.30 (0.06) | −0.41 (0.05) |
| Change at Week 12 | −0.29 (0.07) | −0.48 (0.05) |
| Change at Week 24 | −0.42 (0.06) | −0.53 (0.05) |
| Change at Week 36 | −0.43 (0.06) | −0.56 (0.05) |
| Change at Week 52 | −0.51 (0.06) | −0.60 (0.04) |

Least squares mean (SE),
unit of measure: score on a scale.

Change from baseline in Asthma Control Questionnaire-Interviewer Administered, 5-question Version (ACQ-5-IA) was determined at weeks 2, 4, 8, 12, 24, 36 and 52 (Tables 20 and 21). ACQ-5-IA had 5 questions, reflecting the top-scoring five asthma symptoms: frequency of nocturnal awakenings, severity of asthma symptoms in the mornings, limitation of daily activities, shortness of breath due to asthma and wheeze. Participants were asked to recall how their asthma had been during the previous week and to respond to each of the five symptom questions on a 7-point scale ranged ranging from 0 (no impairment) to 6 (maximum impairment). ACQ-5-IA total score was mean of the scores of all 5 questions and, therefore, ranged from 0 (totally controlled) to 6 (severely uncontrolled), higher scores indicated lower asthma control. LS means and SE were derived from MMRM model with change from baseline in ACQ-5-IA values up to week 52 as the response variable, and treatment, age, baseline weight group, region, baseline eosinophil level, baseline FeNO level, baseline ICS dose level, visit, treatment by-visit interaction, baseline ACQ-5-IA value and baseline-by-visit interaction as covariates.

TABLE 20
Change from baseline in ACQ-5-IA at
weeks 2, 4, 8, 12, 24, 36 and 52 in a Type 2
inflammatory asthma phenotype population.

| | Placebo | DUPIXENT ® |
|---|---|---|
| Overall Number of | 110 | 227 |
| Participants Analyzed | | |
| Change at Week 2 | −0.72 (0.09) | −0.77 (0.06) |
| Change at Week 4 | −0.86 (0.08) | −1.09 (0.06) |
| Change at Week 8 | −1.04 (0.09) | −1.24 (0.07) |
| Change at Week 12 | −1.04 (0.08) | −1.35 (0.06) |
| Change at Week 24 | −1.18 (0.08) | −1.46 (0.06) |
| Change at Week 36 | −1.25 (0.08) | −1.57 (0.06) |
| Change at Week 52 | −1.30 (0.07) | −1.70 (0.05) |

Least squares mean (SE),
unit of measure: score on a scale.

TABLE 21
Change from baseline in ACQ-5-IA at weeks
2, 4, 8, 12, 24, 36 and 52 in a Type 2
inflammatory asthma phenotype population.

| | Placebo | DUPIXENT ® |
|---|---|---|
| Overall Number of | 80 | 166 |
| Participants Analyzed | | |
| Change at Week 2 | −0.63 (0.11) | −0.76 (0.08) |
| Change at Week 4 | −0.75 (0.10) | −1.10 (0.07) |
| Change at Week 8 | −0.98 (0.10) | −1.27 (0.07) |
| Change at Week 12 | −1.00 (0.10) | −1.37 (0.07) |
| Change at Week 24 | −1.06 (0.09) | −1.48 (0.07) |
| Change at Week 36 | −1.17 (0.09) | −1.59 (0.07) |
| Change at Week 52 | −1.25 (0.09) | −1.71 (0.06) |

Least squares mean (SE),
unit of measure: score on a scale.

Change from baseline in Asthma Control Questionnaire-Interviewer Administered, 7-question Version (ACQ-7-IA) was determined at weeks 2, 4, 8, 12, 24, 36 and 52 (Tables 22 and 23). ACQ-7-IA had seven questions, assessed: frequency of nocturnal awakenings, severity of asthma symptoms in the mornings, limitation of daily activities due to asthma, shortness of breath due to asthma and wheeze, reliever medication use, and FEV1 (% predicted). Participants recalled their previous week asthma and answered 5 symptom questions on 7-point scale ranged ranging from 0 (no impairment) to 6 (maximum impairment). Total score: mean of scores of all 7 questions; ranged from 0 (totally controlled) to 6 (severely uncontrolled), higher score indicated lower asthma control. LS means and SE were derived from MMRM model with change from baseline in ACQ-7-IA values up to week 52 as response variable, and treatment, age, baseline weight group, region, baseline eosinophil level, baseline FeNO level, baseline ICS dose level, visit, treatment by-visit interaction, baseline ACQ-7-IA value and baseline-by-visit interaction as covariates.

TABLE 22
Change from baseline in ACQ-7-IA at weeks
2, 4, 8, 12, 24, 36 and 52 in a Type 2
inflammatory asthma phenotype population.

| | Placebo | DUPIXENT ® |
|---|---|---|
| Overall Number of | 110 | 227 |
| Participants Analyzed | | |
| Change at Week 2 | −0.60 (0.08) | −0.72 (0.06) |
| Change at Week 4 | −0.74 (0.07) | −1.03 (0.05) |
| Change at Week 8 | −0.91 (0.08) | −1.14 (0.06) |
| Change at Week 12 | −0.89 (0.07) | −1.23 (0.05) |
| Change at Week 24 | −0.99 (0.07) | −1.32 (0.05) |
| Change at Week 36 | −1.09 (0.07) | −1.41 (0.05) |
| Change at Week 52 | −1.09 (0.06) | −1.53 (0.05) |

Least squares mean (SE),
unit of measure: score on a scale.

TABLE 23
Change from baseline in ACQ-7-IA at weeks
2, 4, 8, 12, 24, 36 and 52 in a Type 2
inflammatory asthma phenotype population.

| | Placebo | DUPIXENT ® |
|---|---|---|
| Overall Number of | 80 | 166 |
| Participants Analyzed | | |
| Change at Week 2 | −0.50 (0.09) | −0.70 (0.07) |
| Change at Week 4 | −0.64 (0.09) | −1.02 (0.06) |
| Change at Week 8 | −0.83 (0.09) | −1.16 (0.06) |
| Change at Week 12 | −0.85 (0.09) | −1.24 (0.06) |
| Change at Week 24 | −0.88 (0.09) | −1.34 (0.06) |
| Change at Week 36 | −1.01 (0.08) | −1.43 (0.06) |
| Change at Week 52 | −1.04 (0.08) | −1.54 (0.06) |

Least squares mean (SE),
unit of measure: score on a scale.

Change from baseline in number of puffs of reliever medication used per 24 hours at weeks 2, 4, 8, 12, 24, 36 and 52 was assessed (Tables 24 and 25). Participants could be administered salbutamol/albuterol or levosalbutamol/levalbuterol as reliever medication as needed during study. The number of salbutamol/albuterol or levosalbutamol/levalbuterol inhalations were recorded daily by the participants in an electronic diary/PEF meter. If nebulizer solutions were used as an alternative delivery method, nebulizer dose was converted to number of puffs as per following conversion factor: salbutamol/albuterol nebulizer solution (2.5 mg) and levosalbutamol/levalbuterol (1.25 mg) corresponds to 4 puffs. LS means and SE were derived from MMRM model with change from baseline in number of puffs of reliever medication/24 hours values up to week 52 as response variable and treatment, age, baseline weight group, region, baseline eosinophil level, baseline FeNO level, baseline ICS dose level, visit, treatment by-visit interaction, baseline number of puffs of reliever medication/24 hours value and baseline-by-visit interaction as covariates.

TABLE 24
Change from baseline in number of puffs of
reliever medication used per 24 hours
was assessed at weeks 2, 4, 8, 12, 24, 36, and
52 in a Type 2 inflammatory asthma phenotype
population.

| | Placebo | DUPIXENT ® |
|---|---|---|
| Overall Number of | 110 | 228 |
| Participants Analyzed | | |
| Change at Week 2 | −0.78 (0.17) | −0.65 (0.12) |
| Change at Week 4 | −0.99 (0.17) | −1.02 (0.13) |
| Change at Week 8 | −1.24 (0.17) | −1.28 (0.13) |
| Change at Week 12 | −0.93 (0.18) | −1.41 (0.13) |
| Change at Week 24 | −1.43 (0.17) | −1.61 (0.13) |
| Change at Week 36 | −1.37 (0.17) | −1.60 (0.13) |
| Change at Week 52 | −1.59 (0.16) | −1.72 (0.12) |

Least squares mean (SE),
unit of measure: puffs of reliever medication.

TABLE 25
Change from baseline in number of puffs
of reliever medication used per 24 hours
was assessed at weeks 2, 4, 8, 12, 24, 36,
and 52 in a baseline blood eosinophils >=300 cells
per microliter population.

| | Placebo | DUPIXENT ® |
|---|---|---|
| Overall Number of | 80 | 167 |
| Participants Analyzed | | |
| Change at Week 2 | −0.75 (0.19) | −0.67 (0.14) |
| Change at Week 4 | −1.02 (0.20) | −1.01 (0.15) |
| Change at Week 8 | −1.15 (0.20) | −1.29 (0.14) |
| Change at Week 12 | −1.02 (0.20) | −1.39 (0.14) |
| Change at Week 24 | −1.25 (0.20) | −1.53 (0.15) |
| Change at Week 36 | −1.20 (0.21) | −1.49 (0.15) |
| Change at Week 52 | −1.45 (0.19) | −1.58 (0.14) |

Least squares mean (SE),
unit of measure: puffs of reliever medication.

Change from baseline in number of nocturnal awakenings per night was assessed at weeks 2, 4, 8, 12, 24, 36 and 52 (Tables 26 and 27). Participants recorded every morning the number of asthma-related nocturnal awakenings requiring use of rescue medication that occurred during the previous night. LS means and SE were derived from MMRM model with change from baseline in number of nocturnal awakenings values up to week 52 as the response variable, and treatment, age, baseline weight group, region, baseline eosinophil level, baseline FeNO level, baseline ICS dose level, visit, treatment by-visit interaction, baseline number of nocturnal awakenings value and baseline-by-visit interaction as covariates.

TABLE 26
Change from baseline in number of nocturnal
awakenings per night at weeks 2, 4,
8, 12, 24, 36 and 52 in a Type 2
inflammatory asthma phenotype population.

| | Placebo | DUPIXENT ® |
|---|---|---|
| Overall Number of | 110 | 228 |
| Participants | | |
| Analyzed | | |
| Change at Week 2 | −0.13 (0.03) | −0.13 (0.02) |
| Change at Week 4 | −0.16 (0.03) | −0.21 (0.03) |
| Change at Week 8 | −0.21 (0.04) | −0.21 (0.03) |
| Change at Week 12 | −0.17 (0.04) | −0.26 (0.03) |
| Change at Week 24 | −0.23 (0.03) | −0.29 (0.02) |
| Change at Week 36 | −0.25 (0.03) | −0.29 (0.02) |
| Change at Week 52 | −0.26 (0.03) | −0.32 (0.02) |

Least squares mean (SE),
unit of measure: nocturnal awakenings per night.

TABLE 27
Change from baseline in number of nocturnal awakenings per night at
weeks 2, 4, 8, 12, 24, 36 and 52 in a baseline blood eosinophils >= 300
cells per microliter population. Least squares mean (SE), unit of
measure: nocturnal awakenings per night.

| | Placebo | DUPIXENT ® |
|---|---|---|
| Overall Number of | 80 | 167 |
| Participants Analyzed | | |
| Change at Week 2 | −0.15 (0.03) | −0.12 (0.02) |
| Change at Week 4 | −0.19 (0.04) | −0.21 (0.03) |
| Change at Week 8 | −0.22 (0.05) | −0.20 (0.04) |
| Change at Week 12 | −0.16 (0.04) | −0.25 (0.03) |
| Change at Week 24 | −0.21 (0.04) | −0.28 (0.03) |
| Change at Week 36 | 0.25 (0.04) | −0.27 (0.03) |
| Change at Week 52 | −0.26 (0.03) | −0.32 (0.02) |

Change from baseline in Pediatric Asthma Quality of Life (QoL) Questionnaire with Standardized Activities-Interviewer Administered (PAQLQ(S)-IA) scores were assessed at weeks 12, 24, 36 and 52 (Tables 28 and 29). PAQLQ(S)-IA, a disease-specific, interviewer-administered QoL questionnaire designed to measure functional impairments that are most important to children >=7 years with asthma. The PAQLQ(S)-IA comprised 23 items in 3 domains: symptoms (10 items), activity limitation (5 items) and emotional function (8 items). Each item was scored on a 7-point Likert scale (1=maximal impairment to 7=no impairment). Twenty-three items of questionnaire were averaged to produce one overall quality of life score ranging from 1 (severely impaired) to 7 (not impaired at all), higher scores indicated better quality of life. LS means and SE were derived from MMRM model with change from baseline in PAQLQ(S)-IA global score values up to week 52 as the response variable, and treatment, age, baseline weight group, region, baseline eosinophil level, baseline FeNO level, baseline ICS dose level, visit, treatment by-visit interaction, baseline PAQLQ(S)-IA global score value and baseline-by-visit interaction as covariates.

TABLE 28
Change from baseline in PAQLQ(S)-IA scores at weeks 12, 24, 36 and
52 in a Type 2 inflammatory asthma phenotype population. Least
squares mean (SE), unit of measure: score on a scale.

| | Placebo | DUPIXENT ® |
|---|---|---|
| Overall Number of | 102 | 201 |
| Participants Analyzed | | |
| Change at Week 12 | 0.97 (0.09) | 1.08 (0.07) |
| Change at Week 24 | 1.11 (0.09) | 1.30 (0.07) |
| Change at Week 36 | 1.15 (0.09) | 1.48 (0.07) |
| Change at Week 52 | 1.19 (0.08) | 1.53 (0.06) |

TABLE 29
Change from baseline in PAQLQ(S)-IA scores at weeks 12, 24, 36 and
52 in a baseline blood eosinophils >= 300 cells per microliter population.
Least squares mean (SE), unit of measure: score on a scale.

| | Placebo | DUPIXENT ® |
|---|---|---|
| Overall Number of | 76 | 149 |
| Participants Analyzed | | |
| Change at Week 12 | 0.90 (0.10) | 1.11 (0.08) |
| Change at Week 24 | 1.06 (0.10) | 1.36 (0.08) |
| Change at Week 36 | 1.07 (0.10) | 1.51 (0.08) |
| Change at Week 52 | 1.23 (0.09) | 1.56 (0.07) |

Healthcare resource utilization (HCRU), calculated as the number of school days (by children) and work days (by caregiver) missed due to LOAC, was assessed (Tables 30-33). The number of days missed from school by the participant and the number of days missed from work by the caregiver due to a LOAC were collected in the electronic-case report form (eCRF). Cumulative number of missed days (school days for children and work days for caregiver) up to week 52 were computed and summarized using mean and standard deviation (SD). Timeline=from baseline to week 52.

TABLE 30
HCRU-number of missed days due to LOAC in a Type 2 inflammatory
asthma phenotype population. Mean (SD), unit of measure: days.

| | Placebo | DUPIXENT ® |
|---|---|---|
| Overall Number of | 114 | 236 |
| Participants Analyzed | | |
| Children: number of | 2.1 (4.2) | 1.0 (2.3) |
| missed school days | | |
| Caregiver: number of | 0.7 (1.9) | 0.2 (0.8) |
| missed work days | | |

TABLE 31
HCRU-number of missed days due to LOAC in a baseline blood
eosinophils >= 300 cells per microliter population. Mean (SD),
unit of measure: days.

| | Placebo | DUPIXENT ® |
|---|---|---|
| Overall Number of | 84 | 175 |
| Participants Analyzed | | |
| Children: number of | 2.0 (3.5) | 0.9 (2.0) |
| missed school days | | |
| Caregiver: number of | 0.6 (1.8) | 0.2 (0.7) |
| missed work days | | |

TABLE 32
HCRU-percentage of participants/caregivers who had missed school/work
days in a Type 2 inflammatory asthma phenotype population.
Measure type: number; unit of measure: percentage of participants.

| | Placebo | DUPIXENT ® |
|---|---|---|
| Overall Number of | 114 | 236 |
| Participants Analyzed | | |
| Percentage of children | 17.6 | 9.8 |
| Percentage of caregiver | 7 | 0.8 |

TABLE 33
HCRU-percentage of participants/caregivers who had missed school/work
days in a baseline blood eosinophils >= 300 cells per microliter
population. Measure type: number; unit of measure:
percentage of participants.

| | Placebo | DUPIXENT ® |
|---|---|---|
| Overall Number of Participants | 84 | 175 |
| Analyzed | | |
| Percentage of children | 17.9 | 8.6 |
| Percentage of caregiver | 6 | 0.6 |

The number of participants with treatment-emergent adverse events (TEAEs) and the number of participants with treatment-emergent serious adverse events (TESAEs) were assessed (Table 34). An adverse event (AE) was defined as any untoward medical occurrence in a participant who received investigational medicinal product (IMP) that did not necessarily have a causal relationship with treatment. TEAEs were defined as AEs that developed or worsened in grade or became serious during the TEAE period, which was defined as the period from the time of first dose of study drug to the end of the post-treatment period. SAEs were AEs resulting in any of the following outcomes or deemed significant for any other reason: death; life-threatening experience (immediate risk of dying); initial or prolonged inpatient hospitalization; persistent or significant disability/incapacity; congenital anomaly or a medically important event. TEAEs included both SAEs and non-SAEs. Timeline=from baseline to week 64.

TABLE 34
Number of participants with TEAEs and TESAEs.
Measure type: count of participants; unit of measure:
participants.

| | Placebo | DUPIXENT ® |
|---|---|---|
| Overall Number of | 134 | 271 |
| Participants Analyzed | | |
| Any TEAE | 107 (79.85%) | 225 (83.03%) |
| TESAE | 6 (4.48%) | 13 (4.8%) |

Pharmacokinetics were assessed to determine functional DUPIXENT concentration in serum (Table 35). Data for this outcome measure was planned to be collected and analyzed separately for DUPIXENT® 100 mg and 200 mg dose and not planned to be collected and analyzed for placebo arm.

TABLE 35
PK assessment: functional DUPIXENT ® concentration in serum.
Geometric mean (geometric coefficient of variation);
unit of measure: nanograms per mL.

| | | DUPIXENT ® | | DUPIXENT ® | |
|---|---|---|---|---|---|
| | | 100 mg q2w | | 200 mg q2w | |
| Overall Number of | | 91 | | 179 | |
| Participants Analyzed | | | | | |
| Baseline | Number | 90 | participants | 178 | participants |
| | Analyzed | | | | |
| | | 0.00 | (948.683%) | 0.00 | (0.000%) |
| Week 6 | Number | 87 | participants | 172 | participants |
| | Analyzed | | | | |
| | | 28566.81 | (47.114%) | 50269.81 | (46.667%) |
| Week 12 | Number | 90 | participants | 174 | participants |
| | Analyzed | | | | |
| | | 37741.97 | (46.516%) | 63476.17 | (44.444%) |
| Week 24 | Number | 90 | participants | 173 | participants |
| | Analyzed | | | | |
| | | 42283.09 | (47.910%) | 49525.35 | (52.806%) |
| Week 52 | Number | 86 | participants | 171 | participants |
| | Analyzed | | | | |
| | | 41467.97 | (49.368%) | 45295.35 | (56.990%) |
| Week 64 | Number | 4 | participants | 15 | participants |
| | Analyzed | | | | |
| | | 39.00 | (0.000%) | 39.00 | (0.000%) |

The percentage of participants with treatment-emergent antidrug antibodies (ADA) was assessed (Table 36). ADA response was categorized as: treatment-emergent and treatment-boosted response. 1) Treatment-emergent was defined as an ADA positive response in the assay post first dose, when baseline results were negative or missing. 2) Treatment-boosted was defined as an ADA positive response in the assay post-first dose that was greater than or equal to 4-fold over baseline titer levels, when baseline results were positive. The criteria for positive was defined as “30 to >10,000,” where low titer was <1,000; moderate titer was 1,000≤titer≤10,000, and high titer was >10,000. Timeline=from baseline to week 64.

TABLE 36
Percentage of participants with ADA response. Measure type:
number; unit of measure: percentage of participants.

| | | DUPIXENT ® | DUPIXENT ® |
|---|---|---|---|
| | Placebo | 100 mg q2w | 200 mg q2w |
| Overall Number of | 133 | 91 | 178 |
| Participants Analyzed | | | |
| Treatment-emergent ADA | 3.0 | 4.4 | 7.3 |
| Treatment-boosted ADA | 0 | 0 | 0 |

The percentage of participants with seroconversion was assessed (Table 37). Seroconversion was defined as a post-vaccination titer>=40 (1/dilution) for those with a pre-vaccination titer<10 (1/dilution), or a >=4-fold increase in post-vaccination titer for those with a pre-vaccination titer>=10 (1/dilution). Timeline=from baseline to week 64.

TABLE 37
Percentage of participants with seroconversion. Measure type:
number; unit of measure: percentage of participants.

| | Placebo | DUPIXENT ® |
|---|---|---|
| Overall Number of | 134 | 271 |
| Participants Analyzed | | |
| Percentage of | 62.5 | 79.6 |
| Participants With | | |
| Seroconversion | | |

Change from baseline in fractional exhaled nitric oxide was assessed (Tables 38 and 39).

TABLE 38
Change from baseline in fractional exhaled nitric oxide at week 12 in
a Type 2 inflammatory asthma phenotype population. Least squares
mean (SE), unit of measure: parts per billion.

| | Placebo | DUPIXENT ® |
|---|---|---|
| Overall Number of | 103 | 216 |
| Participants Analyzed | | |
| Change From | −1.13 (1.43) | −18.97 (1.04) |
| Baseline-FeNO | | |

TABLE 39
Change from baseline in fractional exhaled nitric oxide at week 12 in a
baseline blood eosinophils >= 300 cells per microliter population. Least
squares mean (SE), unit of measure: parts per billion.

| | Placebo | DUPIXENT ® |
|---|---|---|
| Overall Number of | 75 | 156 |
| Participants Analyzed | | |
| Change From | −0.81 (1.69) | −21.40 (1.21) |
| Baseline FeNP | | |

All adverse events (AEs) were collected from signature of the informed consent form up to end of post-treatment period (i.e., up to week 64) regardless of seriousness or relationship to DUPIXENT®. Reported AEs were treatment emergent AEs that developed/worsened in grade or became serious during the “TEAE period” (from the time of first dose of study drug to the end of post-treatment period, i.e., up to week 64). Analysis was performed on the safety population. (See Tables 40 and 41.)

TABLE 40
Serious adverse effects.

| | Placebo | | DUPIXENT ® | |
|---|---|---|---|---|
| | Affected/At | # | Affected/At | # |
| | Risk (%) | Events | Risk (%) | Events |
| Total | 6/134 (4.48%) | | 13/271 (4.8%) | |

Blood and lymphatic system disorders

| | | | | |
|---|---|---|---|---|
| Eosinophilia ^{A} † | 0/134 (0%) | 0 | 2/271 (0.74%) | 2 |
| Immune | 1/134 (0.75%) | 1 | 0/271 (0%) | 0 |
| Thrombo- | | | | |
| cytopenia ^{A} † | | | | |

Eye disorders

| | | | | |
|---|---|---|---|---|
| Vision Blurred ^{A} † | 0/134 (0%) | 0 | 1/271 (0.37%) | 1 |

Immune system disorders

| | | | | |
|---|---|---|---|---|
| Allergy To | 0/134 (0%) | 0 | 1/271 (0.37%) | 1 |
| Chemicals ^{A} † | | | | |
| Anaphylactic | 1/134 (0.75%) | 1 | 0/271 (0%) | 0 |
| Reaction ^{A} † | | | | |
| Drug | 0/134 (0%) | 0 | 1/271 (0.37%) | 1 |
| Hypersensitivity ^{A} † | | | | |
| Milk Allergy ^{A} † | 0/134 (0%) | 0 | 1/271 (0.37%) | 1 |

Infections and infestations

| | | | | |
|---|---|---|---|---|
| Furuncle ^{A} † | 0/134 (0%) | 0 | 1/271 (0.37%) | 1 |
| Gastroenteritis ^{A} † | 1/134 (0.75%) | 1 | 0/271 (0%) | 0 |
| Gastrointestinal | 1/134 (0.75%) | 1 | 0/271 (0%) | 0 |
| Viral Infection ^{A} † | | | | |
| Lymphadenitis | 1/134 (0.75%) | 1 | 0/271 (0%) | 0 |
| Viral ^{A} † | | | | |
| Parainfluenza Virus | 1/134 (0.75%) | 1 | 0/271 (0%) | 0 |
| Infection ^{A} † | | | | |
| Pharyngitis ^{A} † | 0/134 (0%) | 0 | 1/271 (0.37%) | 1 |
| Pneumonia ^{A} † | 0/134 (0%) | 0 | 1/271 (0.37%) | 1 |

Injury, poisoning and procedural complications

| | | | | |
|---|---|---|---|---|
| Hand Fracture ^{A} † | 0/134 (0%) | 0 | 1/271 (0.37%) | 1 |

Nervous system disorders

| | | | | |
|---|---|---|---|---|
| Headache ^{A} † | 0/134 (0%) | 0 | 1/271 (0.37%) | 1 |
| Partial Seizures ^{A} † | 1/134 (0.75%) | 1 | 0/271 (0%) | 0 |

Respiratory, thoracic and mediastinal disorders

| | | | | |
|---|---|---|---|---|
| Asthma ^{A} † | 0/134 (0%) | 0 | 4/271 (1.48%) | 4 |
| Atelectasis ^{A} † | 1/134 (0.75%) | 1 | 0/271 (0%) | 0 |
| Bronchospasm ^{A} † | 1/134 (0.75%) | 1 | 0/271 (0%) | 0 |

† Indicates events were collected by systematic assessment.
^{A} Term from vocabulary, MedDRA 23.0.

TABLE 41
Other adverse effects. Frequency threshold above which other adverse
events are reported: 5%.

| | Placebo | | DUPIXENT ® | |
|---|---|---|---|---|
| | Affected/At | # | Affected/At | # |
| | Risk (%) | Events | Risk (%) | Events |
| Total | 89/134 | | 161/271 | |
| | (66.42%) | | (59.41%) | |

Blood and lymphatic system disorders

| | | | | |
|---|---|---|---|---|
| Eosinophilia ^{A} † | 1/134 (0.75%) | 1 | 15/271 (5.54%) | 15 |

General disorders

| | | | | |
|---|---|---|---|---|
| Injection Site | 13/134 (9.7%) | 93 | 35/271 (12.92%) | 153 |
| Erythema ^{A} † | | | | |
| Injection Site | 7/134 (5.22%) | 15 | 28/271 (10.33%) | 72 |
| Edema ^{A} † | | | | |

Infections and infestations

| | | | | |
|---|---|---|---|---|
| Bronchitis ^{A} † | 14/134 (10.45%) | 22 | 17/271 (6.27%) | 21 |
| Influenza ^{A} † | 12/134 (8.96%) | 17 | 20/271 (7.38%) | 22 |
| Nasophar- | 29/134 (21.64%) | 45 | 50/271 (18.45%) | 76 |
| yngitis ^{A} † | | | | |
| Pharyngitis ^{A} † | 14/134 (10.45%) | 19 | 23/271 (8.49%) | 27 |
| Sinusitis ^{A} † | 7/134 (5.22%) | 10 | 9/271 (3.32%) | 11 |
| Upper | 18/134 (13.43%) | 30 | 35/271 (12.92%) | 59 |
| Respiratory | | | | |
| Tract | | | | |
| Infection ^{A} † | | | | |
| Viral Upper | 13/134 (9.7%) | 17 | 33/271 (12.18%) | 41 |
| Respiratory | | | | |
| Tract | | | | |
| Infection ^{A} † | | | | |

Nervous system disorders

| | | | | |
|---|---|---|---|---|
| Headache ^{A} † | 10/134 (7.46%) | 17 | 18/271 (6.64%) | 29 |

Respiratory, thoracic and mediastinal disorders

| | | | | |
|---|---|---|---|---|
| Cough ^{A} † | 9/134 (6.72%) | 10 | 15/271 (5.54%) | 17 |
| Rhinitis | 16/134 (11.94%) | 19 | 16/271 (5.9%) | 27 |
| Allergic ^{A} † | | | | |

† Indicates events were collected by systematic assessment.
^{A} Term from vocabulary, MedDRA 23.0.

### Conclusions

Overall, in children 6-11 years old with moderate-to-severe asthma with Type 2 inflammation or with an eosinophilic phenotype, DUPIXENT® added to background standard of care showed overall unprecedented efficacy with significant reduction in exacerbation rate as well as an improvement in lung function and in asthma control. These findings were shown across all populations identified by Type 2 biomarkers. The improvement of all endpoints was rapid with an observable difference from placebo as early as week 2 for lung function, and a separation in time-to-first exacerbation by week 4. There was a rapid and sustained suppression of Type 2 biomarkers such as FeNO, IgE and TARC. Median eosinophil levels remained stable. DUPIXENT® demonstrated a unique set of efficacy data on exacerbations reduction as well as improvement of lung functions among patients with Type 2 inflammation or an eosinophilic phenotype. The higher the Type 2 signature, the better the outcome.

A sustained response was observed as continued improvement was observed for all efficacy endpoints through 52 weeks in the VOYAGE study, which was sustained for up to 2 years in the EXCURSION study.

DUPIXENT® demonstrated a favorable safety profile. DUPIXENT® was well-tolerated, with safety consistent with the known DUPIXENT® safety profile.

### Example III

### DUPIXENT® Efficacy in Children with Uncontrolled, Moderate-to-Severe Type 2 Asthma, with and without Evidence of Allergy in Phase 3 VOYAGE

### Background

Most pediatric asthma patients have Type 2 asthma, which includes the allergic phenotype. In phase 3 VOYAGE, add-on DUPIXENT® 100 mg/200 mg (body weight ≤30 kg/>30 kg) every 2 weeks vs. placebo reduced severe asthma exacerbations by 59.3% (P<0.0001) and improved percent predicted pre-bronchodilator (BD) FEV1 at week 12 (LS mean difference 5.2; P<0.001) in children aged 6 to <12 years with uncontrolled moderate-to-severe Type 2 asthma (baseline blood eosinophils ≥150 cells/μl or FeNO≥20 ppb). This analysis evaluated the efficacy of DUPIXENT® in pediatric patients with Type 2 asthma with or without evidence of allergic asthma (total serum IgE≥30 IU/mL and ≥1 perennial aeroallergen-specific IgE≥0.35 kU/L at baseline).

### Methods

Annualized severe exacerbation rate during the 52-week treatment period was assessed in a post-hoc analysis using a negative binomial model. Change from baseline in pre-BD FEV1 and percent predicted pre-BD FEV1 was assessed at weeks 12 and 52 using mixed-effect models with repeated measures.

### Results

350 pediatric patients with Type 2 asthma were enrolled: 261 had evidence of allergic asthma and 89 did not. Baseline characteristics were similar between subgroups, except levels of Type 2 biomarkers (blood eosinophils, FeNO, serum total IgE) and prevalence of ongoing atopic comorbidities were higher in patients with evidence of allergic asthma. DUPIXENT® vs. placebo significantly reduced annualized severe exacerbation rate in patients with or without evidence of allergic asthma. Change from baseline to weeks 12 and 52 in pre-BD FEV1 and percent predicted pre-BD FEV1 was greater in patients treated with DUPIXENT® vs. placebo in both subgroups. However, significance was not observed in patients without evidence of allergic asthma (Table 42). No significant interaction was observed between the response to DUPIXENT® and evidence of allergic asthma. In the overall safety population, the incidence of TEAEs was similar across treatment groups. The most common TEAE occurring more frequently in the DUPIXENT® group was injection site erythema (12.9% DUPIXENT® vs. 9.7% placebo).

TABLE 42
Efficacy of DUPIXENTO ® vs. placebo in children enrolled in phase 3 VOYAGE with Type 2
asthma with or without evidence of allergic asthma.

| | Evidence of allergic asthma | | | |
|---|---|---|---|---|
| | Combined | Combined | Combined | Combined |
| | placebo | DUPIXENT ® | placebo | DUPIXENT ® |
| | (n = 81) | (n = 180) | (n = 33) | (n = 56) |
| Annualized severe | | | | |
| exacerbation rate | | | | |
| Estimate (95% CI) | 0.62 (0.40-0.94) | 0.24 (0.16-0.36) | 0.80 (0.47-1.37) | 0.39 (0.23-0.68) |
| % reduction | 62% | | 51% | |
| P value vs. placebo | <0.0001 | | 0.0494 | |
| Interaction P value | 0.6011 | | | |
| Change from baseline in % | | | | |
| predicted pre-bronchodilator | | | | |
| FEV₁ | | | | |
| Week 12 | | | | |
| n | 79 | 175 | 32 | 52 |
| LS mean difference vs. | 5.67 (1.98-9.36) | | 4.47 (−1.35-10.30) | |
| matched placebo (95% CI) | | | | |
| P value vs. placebo | 0.0027 | | 0.1302 | |
| Interaction P value | 0.6059 | | | |
| Week 52 | | | | |
| n | 74 | 163 | 32 | 52 |
| LS mean difference vs. | 9.27 (5.16-13.37) | | 3.93 (−2.41-10.26) | |
| matched placebo (95% CI) | | | | |
| P value vs. placebo | <0.0001 | | 0.2209 | |
| Interaction P value | 0.1321 | | | |
| Change from baseline in pre- | | | | |
| bronchodilator FEV₁ | | | | |
| Week 12 | | | | |
| n | 79 | 175 | 32 | 53 |
| LS mean difference vs. | 0.11 (0.04-0.19) | | 0.07 (−0.04-0.18) | |
| matched placebo (95% CI) | | | | |
| P value vs. placebo | 0.0018 | | 0.2317 | |
| Interaction P value | 0.3492 | | | |
| Week 52 | | | | |
| n | 74 | 163 | 32 | 52 |
| LS mean difference vs. | 0.20 (0.12-0.29) | | 0.07 (−0.06-0.21) | |
| matched placebo (95% CI) | | | | |

| | | |
|---|---|---|
| P value vs. placebo | <0.0001 | 0.2660 |

| | |
|---|---|
| Interaction P value | 0.0604 |

### CONCLUSION

A high proportion of pediatric Type 2 patients enrolled in VOYAGE had evidence of allergic asthma. DUPIXENT® demonstrated efficacy in reducing asthma exacerbations in children with Type 2 asthma, with or without evidence of allergic asthma.

1. A method for treating asthma in a subject aged 6 years old and older,
wherein the subject has moderate-to-severe asthma characterized by an eosinophilic phenotype or
wherein the subject has oral corticosteroid-dependent asthma,
comprising administering to the subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively.

2. The method of claim 1, wherein the antibody or antigen-binding fragment thereof is administered to the subject as an initial dose followed by one or more secondary doses.

3. The method of claim 2, wherein the initial dose is about 100 mg and each secondary dose is about 100 mg.

4. The method of claim 3, wherein the subject weighs 15 kg to less than 30 kg.

5. The method of claim 2, wherein the initial dose is about 200 mg and each secondary dose is about 200 mg.

6. The method of claim 5, wherein the subject weighs equal to or greater than 30 kg.

7. The method of claim 4, wherein the antibody or antigen-binding fragment thereof is administered to the subject once every other week (q2w).

8. The method of claim 2, wherein the initial dose is about 300 mg and each secondary dose is about 300 mg.

9. The method of claim 8, wherein the subject weighs 15 kg to less than 30 kg.

10. The method of claim 9, wherein the antibody or antigen-binding fragment thereof is administered to the subject once every four weeks (q4w).

11. The method of claim 1, wherein the subject is less than 12 years old.

12. The method of claim 1, wherein the subject has AN elevated FeNO level.

13. The method of claim 12, wherein the FeNO level is ≥20 ppb or ≥25 ppb.

14. The method of claim 6, wherein the antibody or antigen-binding fragment thereof is administered to the subject once every other week (q2w).

15-27. (canceled)

28. The method of claim 1, wherein the subject's blood eosinophil level is greater than or equal to 150 cells/μL or greater than or equal to 300 cells/μL.

29-31. (canceled)

32. The method of claim 1, wherein the antibody or antigen-binding fragment thereof is administered using an autoinjector, a needle and syringe, or a pen.

33. The method of claim 1, wherein the antibody or antigen-binding fragment thereof is administered subcutaneously.

34. The method of claim 1, wherein the antibody or antigen-binding fragment thereof is an add-on maintenance treatment.

35. The method of claim 1, wherein the treatment results in an improvement in one or more patient-reported outcomes (PROs) selected from the group consisting of Pediatric Asthma Quality of Life Questionnaire (PAQLQ) score, Pediatric Asthma Caregiver's Quality of Life Questionnaire (PACQLQ) score, Pediatric Rhinoconjunctivitis Quality of Life Questionnaire (PRQLQ) score, Pediatric Rhinoconjunctivitis Quality of Life Questionnaire-Interviewer Administered (PRQLQ-IA) score, EuroQol 5-level questionnaire (EQ-5D-5L) score, EuroQol 5 dimension youth questionnaire (EQ-5D-Y) score, Asthma Control Questionnaire-Interviewer Administered, 5-question Version (ACQ-5-IA) score, Asthma Control Questionnaire-Interviewer Administered, 7-question Version (ACQ-7-IA) score, healthcare resource utilization (HCRU) score, morning (AM) symptom score, evening (PM) symptom score, number of nocturnal awakenings, and reliever medication use frequency.

36-153. (canceled)

154. A method for treating asthma in a pediatric subject aged 6 years old to less than 12 years old, wherein the pediatric subject has moderate-to-severe asthma characterized by an eosinophilic phenotype, or wherein the subject has oral corticosteroid-dependent asthma,
comprising administering to the pediatric subject an antibody or an antigen-binding fragment thereof that specifically binds interleukin-4 receptor (IL-4R), wherein the antibody or antigen-binding fragment thereof comprises three heavy chain CDR sequences comprising SEQ ID NOs: 3, 4, and 5, respectively, and three light chain CDR sequences comprising SEQ ID NOs: 6, 7, and 8, respectively,
wherein the antibody or antigen-binding fragment thereof is administered to the pediatric subject as an initial loading dose and one or more maintenance doses, wherein a first maintenance dose is administered to the pediatric subject two to four weeks after the initial loading dose, and
wherein the antibody or an antigen-binding fragment thereof is administered as add-on maintenance treatment.

155. The method of claim 154, wherein the pediatric subject weighs 15 kg to less than 30 kg, and wherein the antibody or antigen-binding fragment thereof is administered to the subject as an initial loading dose of about 100 mg, and one or more maintenance doses of about 100 mg every other week (q2w).

156. The method of claim 154, wherein the pediatric subject weighs 15 kg to less than 30 kg, and wherein the antibody or antigen-binding fragment thereof is administered to the subject as an initial loading dose of about 300 mg, and one or more maintenance doses of about 300 mg every four weeks (q4w).

157. The method of claim 154, wherein the pediatric subject weighs greater than or equal to 30 kg, and wherein the antibody or antigen-binding fragment thereof is administered to the subject as an initial loading dose of about 200 mg, and one or more maintenance doses of about 200 mg every other week (q2w).

158. The method of claim 154, wherein the antibody is dupilumab.


SEQUENCE LISTING (191 sequences)

SEQ ID NO: 1 - protein, 124 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, Artificial Sequence [sequence omitted]

SEQ ID NO: 3 - protein, 8 aa, Artificial Sequence
  source: /note=“Description of Artificial Sequence Synthetic peptide”
GFTFRDYA

SEQ ID NO: 4 - protein, 8 aa, Artificial Sequence
  source: /note=“Description of Artificial Sequence Synthetic peptide”
ISGSGGNT

SEQ ID NO: 5 - protein, 16 aa, Artificial Sequence
  source: /note=“Description of Artificial Sequence Synthetic peptide”
AKDRLSITIRPRYYGL

SEQ ID NO: 6 - protein, 11 aa, Artificial Sequence
  source: /note=“Description of Artificial Sequence Synthetic peptide”
QSLLYSIGYNY

SEQ ID NO: 7 - protein, 3 aa, Artificial Sequence
  source: /note=“Description of Artificial Sequence Synthetic peptide”
LGS

SEQ ID NO: 8 - protein, 9 aa, Artificial Sequence
  source: /note=“Description of Artificial Sequence Synthetic peptide”
MQALQTPYT

SEQ ID NO: 9 - protein, 451 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 219 aa, Artificial Sequence
  source: /note=“Description of Artificial Sequence Synthetic polypeptide”
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 12 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 13 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 14 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 15 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 16 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 17 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 18 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 19 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 20 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 21 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 22 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 23 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 24 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 25 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 26 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 27 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 28 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 29 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 30 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 31 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 32 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 35 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 36 - protein, 115 aa, Artificial Sequence
  source: /note=“Description of Artificial Sequence Synthetic polypeptide”
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 37 - protein, 115 aa, Artificial Sequence
  source: /note=“Description of Artificial Sequence Synthetic polypeptide”
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, Artificial Sequence
  source: /note=“Description of Artificial Sequence Synthetic polypeptide”
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 40 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 41 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 45 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 46 - protein, 115 aa, Artificial Sequence
  source: /note=“Description of Artificial Sequence Synthetic polypeptide”
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 47 - protein, 115 aa, Artificial Sequence
  source: /note=“Description of Artificial Sequence Synthetic polypeptide”
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, Artificial Sequence
  source: /note=“Description of Artificial Sequence Synthetic polypeptide”
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 51 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 52 - protein, 115 aa, Artificial Sequence
  source: /note=“Description of Artificial Sequence Synthetic polypeptide”
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 53 - protein, 115 aa, Artificial Sequence
  source: /note=“Description of Artificial Sequence Synthetic polypeptide”
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 54 - protein, 115 aa, Artificial Sequence
  source: /note=“Description of Artificial Sequence Synthetic polypeptide”
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, Artificial Sequence
  source: /note=“Description of Artificial Sequence Synthetic polypeptide”
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 57 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 58 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 59 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 63 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 64 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 65 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 66 - protein, 5 aa, Artificial Sequence
  source: /note=“Description of Artificial Sequence Synthetic peptide”
RNAMF

SEQ ID NO: 67 - protein, 16 aa, Artificial Sequence
  source: /note=“Description of Artificial Sequence Synthetic peptide”
GIGTGGATSYADSVKG

SEQ ID NO: 68 - protein, 7 aa, Artificial Sequence
  source: /note=“Description of Artificial Sequence Synthetic peptide”
GRYYFDY

SEQ ID NO: 69 - protein, 12 aa, Artificial Sequence
  source: /note=“Description of Artificial Sequence Synthetic peptide”
RASQSVSSSYLA

SEQ ID NO: 70 - protein, 7 aa, Artificial Sequence
  source: /note=“Description of Artificial Sequence Synthetic peptide”
GASSRAT

SEQ ID NO: 71 - protein, 10 aa, Artificial Sequence
  source: /note=“Description of Artificial Sequence Synthetic peptide”
QQYDHSAGWT

SEQ ID NO: 72 - protein, 7 aa, Artificial Sequence
  source: /note=“Description of Artificial Sequence Synthetic peptide”
GASSRAP

SEQ ID NO: 73 - protein, 10 aa, Artificial Sequence
  source: /note=“Description of Artificial Sequence Synthetic peptide”
QQYGSSPPWT

SEQ ID NO: 74 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 75 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 76 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 77 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 78 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 79 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 80 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 81 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 82 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 83 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 84 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 85 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 86 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 90 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 91 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 92 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 93 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 94 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 95 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 96 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 97 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 98 - protein, 111 aa, Artificial Sequence
  source: /note=“Description of Artificial Sequence Synthetic polypeptide”
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 99 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 100 - protein, 111 aa, Artificial Sequence
  source: /note=“Description of Artificial Sequence Synthetic polypeptide”
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 101 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 102 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 103 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 104 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 105 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 106 - protein, 111 aa, Artificial Sequence
  source: /note=“Description of Artificial Sequence Synthetic polypeptide”
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 108 - protein, 111 aa, Artificial Sequence
  source: /note=“Description of Artificial Sequence Synthetic polypeptide”
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 109 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 110 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 111 - protein, 111 aa, Artificial Sequence
  source: /note=“Description of Artificial Sequence Synthetic polypeptide”
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 112 - protein, 111 aa, Artificial Sequence
  source: /note=“Description of Artificial Sequence Synthetic polypeptide”
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 113 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 114 - protein, 116 aa, Artificial Sequence
  source: /note=“Description of Artificial Sequence Synthetic polypeptide”
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 115 - protein, 111 aa, Artificial Sequence
  source: /note=“Description of Artificial Sequence Synthetic polypeptide”
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 116 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 117 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 118 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 119 - protein, 111 aa, Artificial Sequence
  source: /note=“Description of Artificial Sequence Synthetic polypeptide”
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 120 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 121 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 122 - protein, 111 aa, Artificial Sequence
  source: /note=“Description of Artificial Sequence Synthetic polypeptide”
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 123 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 124 - protein, 111 aa, Artificial Sequence
  source: /note=“Description of Artificial Sequence Synthetic polypeptide”
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 125 - protein, 116 aa, Artificial Sequence
  source: /note=“Description of Artificial Sequence Synthetic polypeptide”
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 126 - protein, 111 aa, Artificial Sequence
  source: /note=“Description of Artificial Sequence Synthetic polypeptide”
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 127 - protein, 5 aa, Artificial Sequence
  source: /note=“Description of Artificial Sequence Synthetic peptide”
SYYMH

SEQ ID NO: 128 - protein, 17 aa, Artificial Sequence
  source: /note=“Description of Artificial Sequence Synthetic peptide”
IINPRGGSTSYAQKFQG

SEQ ID NO: 129 - protein, 7 aa, Artificial Sequence
  source: /note=“Description of Artificial Sequence Synthetic peptide”
GKYWMYD

SEQ ID NO: 130 - protein, 13 aa, Artificial Sequence
  source: /note=“Description of Artificial Sequence Synthetic peptide”
SGGGSSIGNSYVS

SEQ ID NO: 131 - protein, 7 aa, Artificial Sequence
  source: /note=“Description of Artificial Sequence Synthetic peptide”
DNNKRPS

SEQ ID NO: 132 - protein, 12 aa, Artificial Sequence
  source: /note=“Description of Artificial Sequence Synthetic peptide”
GTWDTSPVWEWP

SEQ ID NO: 133 - protein, 116 aa, Artificial Sequence
  source: /note=“Description of Artificial Sequence Synthetic polypeptide”
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 134 - protein, 121 aa, Artificial Sequence
  source: /note=“Description of Artificial Sequence Synthetic polypeptide”
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 135 - protein, 127 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 136 - protein, 116 aa, Artificial Sequence
  source: /note=“Description of Artificial Sequence Synthetic polypeptide”
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 137 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 138 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 139 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 140 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 141 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 142 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 143 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 144 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 145 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 146 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 147 - protein, 116 aa, Artificial Sequence
  source: /note=“Description of Artificial Sequence Synthetic polypeptide”
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 148 - protein, 110 aa, Artificial Sequence
  source: /note=“Description of Artificial Sequence Synthetic polypeptide”
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 149 - protein, 110 aa, Artificial Sequence
  source: /note=“Description of Artificial Sequence Synthetic polypeptide”
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 150 - protein, 110 aa, Artificial Sequence
  source: /note=“Description of Artificial Sequence Synthetic polypeptide”
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 151 - protein, 110 aa, Artificial Sequence
  source: /note=“Description of Artificial Sequence Synthetic polypeptide”
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 152 - protein, 110 aa, Artificial Sequence
  source: /note=“Description of Artificial Sequence Synthetic polypeptide”
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 153 - protein, 110 aa, Artificial Sequence
  source: /note=“Description of Artificial Sequence Synthetic polypeptide”
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 154 - protein, 110 aa, Artificial Sequence
  source: /note=“Description of Artificial Sequence Synthetic polypeptide”
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 155 - protein, 110 aa, Artificial Sequence
  source: /note=“Description of Artificial Sequence Synthetic polypeptide”
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 156 - protein, 110 aa, Artificial Sequence
  source: /note=“Description of Artificial Sequence Synthetic polypeptide”
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 157 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 158 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 159 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 160 - protein, 111 aa, Artificial Sequence
  source: /note=“Description of Artificial Sequence Synthetic polypeptide”
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 161 - protein, 111 aa, Artificial Sequence
  source: /note=“Description of Artificial Sequence Synthetic polypeptide”
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 162 - protein, 111 aa, Artificial Sequence
  source: /note=“Description of Artificial Sequence Synthetic polypeptide”
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 163 - protein, 111 aa, Artificial Sequence
  source: /note=“Description of Artificial Sequence Synthetic polypeptide”
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 164 - protein, 111 aa, Artificial Sequence
  source: /note=“Description of Artificial Sequence Synthetic polypeptide”
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 165 - protein, 111 aa, Artificial Sequence
  source: /note=“Description of Artificial Sequence Synthetic polypeptide”
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 166 - protein, 5 aa, Artificial Sequence
  source: /note=“Description of Artificial Sequence Synthetic peptide”
RHAMA

SEQ ID NO: 167 - protein, 17 aa, Artificial Sequence
  source: /note=“Description of Artificial Sequence Synthetic peptide”
AITSSGRSIYYADSVKG

SEQ ID NO: 168 - protein, 7 aa, Artificial Sequence
  source: /note=“Description of Artificial Sequence Synthetic peptide”
VHRAFDY

SEQ ID NO: 169 - protein, 13 aa, Artificial Sequence
  source: /note=“Description of Artificial Sequence Synthetic peptide”
SGSPLFPDSGSFN

SEQ ID NO: 170 - protein, 7 aa, Artificial Sequence
  source: /note=“Description of Artificial Sequence Synthetic peptide”
ADSHRPS

SEQ ID NO: 171 - protein, 11 aa, Artificial Sequence
  source: /note=“Description of Artificial Sequence Synthetic peptide”
GTWDYSLSGYV

SEQ ID NO: 172 - protein, 117 aa, Artificial Sequence
  source: /note=“Description of Artificial Sequence Synthetic polypeptide”
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 173 - protein, 128 aa, Artificial Sequence
  source: /note=“Description of Artificial Sequence Synthetic polypeptide”
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 174 - protein, 122 aa, Artificial Sequence
  source: /note=“Description of Artificial Sequence Synthetic polypeptide”
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 175 - protein, 128 aa, Artificial Sequence
  source: /note=“Description of Artificial Sequence Synthetic polypeptide”
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 176 - protein, 117 aa, Artificial Sequence
  source: /note=“Description of Artificial Sequence Synthetic polypeptide”
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 177 - protein, 124 aa, Artificial Sequence
  source: /note=“Description of Artificial Sequence Synthetic polypeptide”
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 178 - protein, 117 aa, Artificial Sequence
  source: /note=“Description of Artificial Sequence Synthetic polypeptide”
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 179 - protein, 116 aa, Artificial Sequence
  source: /note=“Description of Artificial Sequence Synthetic polypeptide”
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 180 - protein, 117 aa, Artificial Sequence
  source: /note=“Description of Artificial Sequence Synthetic polypeptide”
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 181 - protein, 122 aa, Artificial Sequence
  source: /note=“Description of Artificial Sequence Synthetic polypeptide”
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 182 - protein, 107 aa, Artificial Sequence
  source: /note=“Description of Artificial Sequence Synthetic polypeptide”
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 183 - protein, 107 aa, Artificial Sequence
  source: /note=“Description of Artificial Sequence Synthetic polypeptide”
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 184 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 185 - protein, 107 aa, Artificial Sequence
  source: /note=“Description of Artificial Sequence Synthetic polypeptide”
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 186 - protein, 107 aa, Artificial Sequence
  source: /note=“Description of Artificial Sequence Synthetic polypeptide”
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 187 - protein, 107 aa, Artificial Sequence
  source: /note=“Description of Artificial Sequence Synthetic polypeptide”
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 188 - protein, 107 aa, Artificial Sequence
  source: /note=“Description of Artificial Sequence Synthetic polypeptide”
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 189 - protein, 107 aa, Artificial Sequence
  source: /note=“Description of Artificial Sequence Synthetic polypeptide”
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 190 - protein, 107 aa, Artificial Sequence
  source: /note=“Description of Artificial Sequence Synthetic polypeptide”
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 191 - protein, 107 aa, Artificial Sequence
  source: /note=“Description of Artificial Sequence Synthetic polypeptide”
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK